(19) United States
(12) Patent Application Publication
(10) Pub. No.: US 2024/0168012 A1
(43) Pub. Date: May 23, 2024

(54) METHODS OF DETERMINING POTENCY OF A THERAPEUTIC CELL COMPOSITION

(71) Applicant: Juno Therapeutics, Inc., Seattle, WA (US)

(72) Inventor: Neil HAIG, Seattle, WA (US)

(73) Assignees: Juno Therapeutics, Inc., Seattle, WA (US); Juno Therapeutics, Inc., Seattle, WA (US)

(21) Appl. No.: 18/283,434

(22) PCT Filed: Mar. 21, 2022
(86) PCT No.: PCT/US2022/021225

§ 371 Date: Sep. 21, 2023

Related U.S. Application Data
(60) Provisional application No. 63/164,527, filed on Mar. 22, 2021.

(51) Int. Cl. G01N 33/50

(52) U.S. Cl. CPC G01N 33/505; G01N 33/5011; G01N 2333/70514; G01N 2333/70517

(57) ABSTRACT

The present disclosure relates to methods of determining potency of a therapeutic cell compositions in connection with cell therapy. The cells of the therapeutic cell composition can express recombinant receptors such as chimeric receptors, e.g., chimeric antigen receptors (CARs) or other transgenic receptors such as T cell receptors (TCRs). The methods provide an assay for identifying the potency, including relative potency, of a therapeutic cell composition.

### CROSS-REFERENCE TO RELATED APPLICATIONS

This application claims priority to U.S. provisional application 63/164,527 filed Mar. 22, 2021, the contents of which are incorporated by reference in its entirety for all purposes.

### INCORPORATION BY REFERENCE OF SEQUENCE LISTING

The present application is being filed along with a Sequence Listing in electronic format. The Sequence Listing is provided as a file entitled 73504_2023040_SEQLIST.TXT, created Mar. 21, 2022, which is 84,579 bytes in size. The information in the electronic format of the Sequence Listing is incorporated by reference in its entirety

### FIELD

The present disclosure relates to methods of determining potency of a therapeutic cell composition for use in connection with cell therapy. The cells of the therapeutic cell composition can express recombinant receptors such as chimeric receptors, e.g., chimeric antigen receptors (CARs) or other transgenic receptors such as T cell receptors (TCRs). The methods provide an assay for determining the potency, including the relative potency, of a therapeutic cell composition.

### BACKGROUND

Various immunotherapy and/or cell therapy methods are available for treating diseases and conditions. For example, adoptive cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be beneficial in the treatment of cancer or other diseases or disorders. Improved approaches are needed for characterizing effective therapeutic cell compositions, such as in connection with methods for ex vivo production of the compositions, and for treating a subject with a cell therapy. Provided herein are methods that address such needs.

### SUMMARY

Provided herein is a method of determining potency of a therapeutic cell composition, the method comprising performing a plurality of incubations, each of said plurality of incubations comprising culturing cells of a therapeutic cell composition, said therapeutic composition comprising cells engineered to express a recombinant receptor, with a recombinant receptor stimulating agent, wherein binding of the recombinant receptor stimulating agent to the recombinant receptor stimulates a recombinant receptor-dependent activity in the cell; and each of the plurality of incubations comprises a different titrated ratio of the cells of the therapeutic cell composition to the recombinant receptor stimulating agent; measuring the recombinant receptor-dependent activity from each of the plurality of incubations; and determining, based on the recombinant receptor-dependent activity measured from each of the plurality of incubations, the titrated ratio that results in a specified recombinant receptor-dependent activity, e.g., a half-maximal recombinant receptor-dependent activity. In some of any of the provided embodiments, the method further comprises determining a relative potency of the therapeutic cell composition by comparing the titrated ratio resulting in the specified receptor-dependent activity (e.g., the half-maximal recombinant receptor-dependent activity) of the therapeutic cell composition to a titrated ratio resulting in a specified receptor-dependent activity (e.g., a half-maximal recombinant receptor-dependent activity) of a reference standard.

Also provided herein is a method of determining potency of a therapeutic cell composition, the method comprising performing a plurality of incubations, each of said plurality of incubations comprising culturing cells of a therapeutic cell composition, said therapeutic composition comprising cells engineered to express a recombinant receptor, with a recombinant receptor stimulating agent, wherein: binding of the recombinant receptor stimulating agent to the recombinant receptor stimulates a recombinant receptor-dependent activity in the cell; and each of the plurality of incubations comprises a different titrated ratio of the cells of the therapeutic cell composition to the recombinant receptor stimulating agent; measuring the recombinant receptor-dependent activity from each of the plurality of incubations; and determining a relative potency of the therapeutic cell composition by comparing a specified recombinant receptor-dependent activity of the therapeutic cell composition (e.g., a half-maximal recombinant receptor-dependent activity of the therapeutic cell composition) to a specified recombinant receptor-dependent activity (e.g., a half-maximal recombinant receptor-dependent activity) of a reference standard.

In some of any of the provided embodiments, each of the plurality of incubations comprises culturing a constant number of cells of the therapeutic composition with differing amounts of the recombinant receptor stimulating agent to generate a plurality of different titrated ratios. In some of any of the provided embodiments, each of the plurality of incubations comprises culturing a constant amount of binding molecule, e.g., recombinant receptor stimulating agent, with differing numbers of cells of the therapeutic composition to generate a plurality of different titrated ratios.

In some of any of the provided embodiments, the plurality of incubations are performed for two or more, optionally 3, 4, 5, 6, 7, 8, 9, 10, or more, therapeutic cell compositions. In some of any of the provided embodiments, the two or more therapeutic cell compositions each comprise the same recombinant receptor. In some of any of the provided embodiments, the two or more therapeutic cell compositions each comprise different recombinant receptors. In some of any of the provided embodiments, at least one of the two or more therapeutic cell compositions comprises a different recombinant receptor than the other therapeutic compositions.

In some of any of the provided embodiments, the two or more therapeutic cell compositions are each manufactured using the same manufacturing process. In some of any of the provided embodiments, the two or more therapeutic cell compositions are each manufactured using a different manufacturing process. In some of any of the provided embodiments, at least one of the two or more therapeutic cell compositions is manufactured using a different manufacturing process than those used to manufacture the other therapeutic cell compositions.

In some of any of the provided embodiments, the two or more therapeutic cell compositions are produced from cells from a single subject. In some of any of the provided embodiments, the two or more therapeutic cell compositions are produced from cells from different subjects. In some of any of the provided embodiments, the subject is a healthy subject or a subject having a disease or condition.

In some of any of the provided embodiments, each of the different subjects have the same disease or condition. In some of any of the provided embodiments, each of the different subjects are to be treated with the same therapeutic cell composition for treating a disease or condition in the subject.

In some of any of the provided embodiments, the plurality of incubations is at least three incubations. In some of any of the provided embodiments, the plurality of incubations is at least five incubations. In some of any of the provided embodiments, the plurality of incubations is at least seven incubations. In some of any of the provided embodiments, the plurality of incubations is at least ten incubations.

In some of any of the provided embodiments, the recombinant receptor-dependent activity comprises one or more of a cytokine expression, cytolytic activity, receptor upregulation, receptor downregulation, proliferation, gene upregulation, gene down regulation, or cell health. In some of any of the provided embodiments, the recombinant receptor-dependent activity comprises or is a cytokine expression or production. In some of any of the provided embodiments, the recombinant receptor-dependent activity comprises or is a cytokine expression or production, wherein the cytokine is TNF-alpha, IFNgamma (IFNg), or IL-2. In some of any of the provided embodiments, the recombinant receptor-dependent activity comprises or is a cytolytic activity. In some of any of the provided embodiments, the recombinant receptor-dependent activity comprises or is a receptor downregulation. In some of any of the provided embodiments, the recombinant receptor-dependent activity comprises or is a proliferation. In some of any of the provided embodiments, the recombinant receptor-dependent activity comprises or is a gene upregulation. In some of any of the provided embodiments, the recombinant receptor-dependent activity comprises or is a gene downregulation. In some of any of the provided embodiments, the recombinant receptor-dependent activity comprises or is a cell health. In some of any of the provided embodiments, the recombinant receptor-dependent activity comprises or is a cell health, wherein the cell health comprises one or more of cell death, cell diameter, viable cell concentration, and cell count.

In some of any of the provided embodiments, the recombinant receptor-dependent activity measured at each of the plurality of incubations is normalized to a maximum receptor-dependent activity measured for the therapeutic cell composition.

In some of any of the provided embodiments, the reference standard is a therapeutic cell composition comprising a validated titrated ratio resulting in a specified (e.g., half-maximal) recombinant receptor-dependent activity, a commercially available therapeutic cell composition, a therapeutic cell composition manufactured using a manufacturing process that is identical to a manufacturing process used to manufacture the therapeutic cell composition, a therapeutic cell composition manufactured using a manufacturing process that is different from a manufacturing process used to manufacture the therapeutic cell composition, a therapeutic cell composition comprising an identical recombinant receptor as the therapeutic cell composition, a therapeutic cell composition comprising a different recombinant receptor as the therapeutic cell composition, a therapeutic cell composition manufactured from the same subject, or a therapeutic cell composition manufactured from a different subject.

In some of any of the provided embodiments, the reference standard is one of the two or more therapeutic compositions.

In some of any of the provided embodiments, the recombinant receptor stimulating agent comprises a target antigen or an extracellular domain binding portion thereof, optionally a recombinant antigen, of the recombinant receptor. In some of any of the provided embodiments, the recombinant receptor stimulating agent comprises an extracellular domain binding portion of the antigen and the extracellular domain binding portion comprises an epitope recognized by the recombinant receptor. In some of any of the provided embodiments, the recombinant receptor stimulating agent comprises an antibody specific to an extracellular domain (e.g., an epitope on the extracellular domain) of the recombinant receptor. In some of any of the provided embodiments, the recombinant receptor stimulating agent is an anti-idiotypic antibody specific to an extracellular antigen binding domain of the recombinant receptor.

In some of any of the provided embodiments, the recombinant receptor stimulating agent is immobilized or attached to a solid support. In some of any of the provided embodiments, the solid support is a surface of the vessel, optionally a well of microwell plate, in which the plurality of incubations are performed. In some of any of the provided embodiments, the solid support is a bead.

In some of any of the provided embodiments, the recombinant receptor stimulating agent is an antigen-expressing cell, optionally wherein the cell is a clone, a cell from a cell line, or a primary cell taken from a subject. In some of any of the provided embodiments, the antigen-expressing cell is a cell line. In some of any of the provided embodiments, the cell line is a tumor cell line. In some of any of the provided embodiments, the antigen-expressing cell is a cell that has been introduced, optionally by transduction, to express the antigen of the recombinant receptor.

In some of any of the provided embodiments, the titrated ratio achieves a linear dose-response range of the recombinant receptor-dependent activity of the reference standard. In some of any of the provided embodiments, the titrated ratio comprises a lower asymptote (minimal) recombinant receptor-dependent activity and an upper asymptote (maximal) recombinant receptor-dependent activity of the reference standard.

In some of any of the provided embodiments, the therapeutic cell composition comprises a single cell subtype enriched or purified from a biological sample or a population of mixed cell subtypes, optionally obtained by mixing cell subtypes enriched or purified from a biological sample. In some of any of the provided embodiments, the biological sample comprises a whole blood sample, a buffy coat sample, a peripheral blood mononuclear cell (PBMC) sample, an unfractionated cell sample, a lymphocyte sample, a white blood cell sample, an apheresis product, or a leukapheresis product.

In some of any of the provided embodiments, the therapeutic cell composition comprises primary cells. In some of any of the provided embodiments, the therapeutic cell composition comprises autologous cells from a subject to be treated. In some of any of the provided embodiments, the therapeutic cell composition comprises allogeneic cells. In some of any of the provided embodiments, the therapeutic cell composition comprises CD3+, CD4+, and/or CD8+ T cells. In some of any of the provided embodiments, the therapeutic cell composition comprises or is CD4+ T cells. In some of any of the provided embodiments, the therapeutic cell composition comprises or is CD8+ T cells. In some of any of the provided embodiments, the recombinant receptor is a chimeric antigen receptor (CAR).

In some of any of the provided embodiments, the therapeutic cell composition comprises CD4+ T cells and CD8+ T cells. In some of any of the provided embodiments, the recombinant receptor is a chimeric antigen receptor (CAR).

In some of any of the provided embodiments, the plurality of incubations are performed in a flask, a tube, or a multi-well plate. In some of any of the provided embodiments, the plurality of incubations are each performed individually in a well of a multi-well plate. In some of any of the provided embodiments, the multi-well plate is a 96-well plate, 48-well plate, 12-well plate or 6-well plate.

In some of any of the provided embodiments, the method further comprises determining, based on the titrated ratio that results in a specified (e.g., half-maximal) recombinant receptor-dependent activity, a dose of cells of the therapeutic composition for administering to a subject in need thereof. In some of any of the provided embodiments, the method further comprises determining, based on the relative potency, a dose of cells of the therapeutic composition for administering to a subject in need thereof.

In some of any of the provided embodiments, the subject has a disease or condition. In some of any of the provided embodiments, the disease or condition is cancer.

In some of any of the provided embodiments, the method further comprises determining, based on the relative potency, a manufacturing process that produces an optimal therapeutic cell composition potency, wherein the optimal therapeutic cell composition potency correlates with complete and/or durable response and/or reduced toxicity.

In some of any of the provided embodiments, the method further comprises determining, based on the relative potency, a manufacturing process that produces a therapeutic cell composition with reduced or low variance in potency, wherein the reduced or low variance is determined compared to the variance in a different manufacturing process.

### BRIEF DESCRIPTION OF THE DRAWINGS

FIGS. 1A-1B show cytokine secretion response curves at different target to effector cell ratios (T:E) for three different donors. FIG. 1A shows raw value cytokine secretion curves and FIG. 1B shows the same secretion curves normalized by the upper asymptote (Vmax).

### DETAILED DESCRIPTION

Provided herein are methods for assessing or determining potency of a therapeutic cell composition for cell therapy, including engineered T cell therapy (e.g., therapeutic cell composition), such as for use in connection with monitoring ex vivo processes for producing the cell therapy and for determining doses for the treatment of diseases and conditions, including various cancers. The provided embodiments relate to therapeutic T cell compositions containing engineered T cells such as those engineered to express recombinant proteins such as expressing recombinant receptors designed to recognize and/or specifically bind to molecules associated with the disease or condition and result in a response, such as an immune response against such molecules upon binding to such molecules. The receptors may include chimeric receptors, e.g., chimeric antigen receptors (CARs), and other transgenic antigen receptors including transgenic T cell receptors (TCRs).

Present methods of determining potency of a therapeutic cell composition typically use maximum antigen stimulation of the engineered cells of the therapeutic cell composition. For example, various existing methods measure antigen-specific activity of the cells, e.g., cytokine expression, receptor upregulation or downregulation, of the therapeutic cell composition upon maximal stimulation. The activity measured in response to the stimulation is then compared against all therapeutic cell compositions tested to determine which therapeutic cell composition had the most activity, e.g., recombinant receptor-dependent activity. The therapeutic cell composition having the highest activity may be considered the most potent therapeutic cell composition.

In many cases, use of a saturating level of antigen may not be physiologically relevant. Furthermore, use of a saturating level of antigen fails to capture the sensitivity of a therapeutic cell composition to recombinant receptor stimulation. For example, current assays cannot distinguish the sensitivity of a recombinant receptor to stimulation (e.g., amount or concentration of antigen) required to elicit a detectable activity response. Nor does maximal antigen stimulation allow for elucidation of the activity of a recombinant receptor, e.g., recombinant receptor-dependent activity, to varied stimulation. In short, the current methods of assessing potency in a therapeutic cell composition provide a one dimensional view of therapeutic cell composition potency, and further lack the ability to establish a measure that can capture the sensitivity (e.g., behavior, activity) of the therapeutic cell composition.

The methods provided herein are designed to more comprehensively assess the sensitivity (e.g., behavior, activity) of the therapeutic cell composition. The methods provided herein are designed to provide a more biologically relevant measure of therapeutic cell composition potency. In some embodiments, the potency of a therapeutic cell composition determined according to the methods described herein may be more strongly correlated with safety and efficacy of the therapeutic cell composition. In some embodiments, the potency of a therapeutic cell composition determined according to the methods described herein may provide improved measures of manufacturing control and/or variability, which in turn can allow for improved assessment of stability and activity (e.g., recombinant receptor-dependent activity) of the manufactured therapeutic cell composition.

The provided methods relate to a direct way to compare how a therapeutic cell composition responds to antigen. Unlike existing methods that compare activity to a single target antigen stimulation, which in many cases is a maximum possible stimulation, the provided methods titrate the ratio of target (e.g., antigen or antibody-expressing cells) to effector cells (cells of the therapeutic composition). For example, this ratio can be controlled by maintaining a constant number of effector cells in the assay and by varying the number of target expressing cells. For example, by the provided methods it is possible to determine the number of target-expressing cells and/or the amount of target (e.g., antigen or antibody) necessary to reach a specified receptor-dependent activity. In some embodiments, the target is an antigen of the recombinant receptor. Thus, in some cases, the target-expressing cells are antigen-expressing cells. In some embodiments, the specified receptor-dependent activity is 50% of maximal activity. In some embodiments, the specified receptor-dependent activity is 10%, 20%, 25%, 30%, 40%, 50%, 60%, 70%, 75%, 80%, or 90% of maximal receptor-dependent activity. In some embodiments, the specified receptor-dependent activity is a range as disclosed herein. In some embodiments, the provided methods allow for comparison of therapeutic cell compositions. For example, the therapeutic cell compositions can be assessed by the methods provided herein and a relative potency determined, for example as described herein.

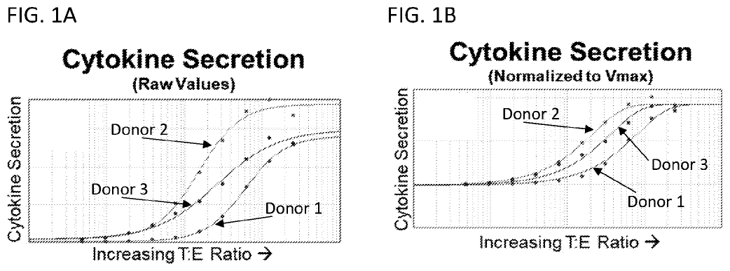

Importantly, it was not known that it would be possible to compare activity between and among different therapeutic cell compositions. In particular, the ability to compare therapeutic cell compositions was not known because it was believed that therapeutic cell compositions have different sensitivity profiles (e.g., responses to recombinant receptor-specific stimulation), for example different minimum and maximum responses. The variables that impact therapeutic cell compositions are much greater than other drug products, such as biologics, such as due to differences in: donors from which the cells are derived or obtained, cells such as differentiation state, antigen-binding ability of a particular recombinant receptor (e.g., CAR), intracellular signaling components of a particular recombinant receptor, percentage or frequency of cells in a composition expressing the recombinant receptor (e.g., CAR), process used to produce the therapeutic composition, and other factors. For example, as shown in FIG. 1A, therapeutic compositions from different donors exhibit different responses to antigen stimulation, such as shown in the response curve. This result thus would indicate that comparing the therapeutic cell compositions may not be possible. It is found herein that by normalizing activity to the upper asymptote of a response curve (e.g., maximum stimulation) that the sensitivity of therapeutic cell compositions could be compared. The provided methods make it possible to measure the sensitivity between and among different cell products, including those that may vary such as a result of being produced by different methods, expressing different antigen receptors, having been produced from different donors, and other variables. Existing methods assess activity at maximum stimulation in part because it was believed to be the only way to account for potential variance.

In some embodiments, the methods provided herein, reduce or eliminate sources of variability. For example, the methods provided herein are robust to variability that may arise due to donor heterogeneity and/or day to day sampling or testing. In some cases, eliminating variability, such as variability due to donor heterogeneity and/or sampling or testing, allows for comparison of therapeutic cell compositions.

The methods provided herein include assay formats including a series of incubations in which different titrated ratios of cells of the therapeutic cell composition and a recombinant receptor stimulating agent are cultured. In provided aspects, the recombinant receptor stimulating agent is an agent that induces or is capable of inducing a signal through an intracellular signaling region of the recombinant receptor. For instance, a recombinant receptor stimulating agent may include an antigen of the recombinant receptor, such as a purified antigen or a recombinant antigen, antigen-expressing cells, or an anti-idiotypic antibody specific to an extracellular antigen binding domain of the recombinant receptor (e.g., scFv), In some embodiments, the methods, including assay formats, provided herein are designed to measure the sensitivity of a therapeutic cell composition by measuring or determining the amount or concentration of the recombinant receptor stimulating agent, for examples as described in Section I-B, needed to stimulate a recombinant receptor-dependent activity in the engineered cells of the therapeutic cell composition. For example, the methods provided herein can determine the level (e.g., amount, concentration) of antigen to stimulate a quantifiable and detectable activity (e.g., recombinant receptor-dependent activity) of the therapeutic cell composition. In some embodiments, the measure of sensitivity includes measurements of the recombinant receptor-dependent activity stimulated by the recombinant receptor stimulating agent binding to the recombinant receptor across a plurality of titrated ratios. The ability of the methods to assess recombinant receptor-dependent activity at different titrated ratios allows determination, estimation, and/or extrapolation of the general activity or behavior of the therapeutic cell composition to recombinant receptor specific stimulation.

The methods provided herein include assays that allow for the assessment of potency of a therapeutic cell composition by measuring the activity of cells of the therapeutic cell composition expressing a recombinant receptor, e.g., a recombinant receptor described herein, in response to stimulation of the recombinant receptor in a series of controlled incubations. For example, the series of incubations may include culturing engineered cells of a therapeutic cell composition expressing a recombinant receptor with a recombinant receptor stimulating agent, for example as described herein (e.g., Section I-B), that when bound to the recombinant receptor stimulates an activity of the recombinant receptor expressed by the cell, e.g., recombinant receptor-dependent activity, at different titrated ratios, where each incubation is a different titrated ratio. In some embodiments, at or at least 3, 4, 5, 6, 7, 8, 9, 10, 11, 12, 13, 14, 15, or more incubations are performed, each incubation containing a different ratio of cells of a therapeutic cell composition to recombinant receptor stimulating agent. In some embodiments, at or at least 3 incubations are performed, each incubation containing a different titrated ratio of cells of a therapeutic cell composition to recombinant receptor stimulating agent. In some embodiments, at or at least 6 incubations are performed, each incubation containing a different titrated ratio of cells of a therapeutic cell composition to recombinant receptor stimulating agent. In some embodiments, at or at least 10 incubations are performed, each incubation containing a different titrated ratio of cells of a therapeutic cell composition to recombinant receptor stimulating agent.

In some embodiments, a constant number of cells of the therapeutic composition is cultured with differing amounts of recombinant receptor stimulating agent to generate a series (e.g., plurality) of different titrated ratios. Alternatively, in some embodiments, a constant amount or concentration of recombinant receptor stimulating agent, e.g., as described in Section I-B, may be incubated with differing numbers of cells of the therapeutic cell composition to generate a series (e.g., plurality) of different titrated ratios. Regardless of how the different titrated ratios are achieved, e.g., by varying the total number of cells of the therapeutic cell composition or the amount of recombinant receptor stimulating agent, using a series (e.g., plurality) of titrated ratios allows for the assessment of recombinant receptor-dependent activity across a range of stimulating conditions. In some embodiments, the range of measurements can used to extract, estimate, and/or determine how engineered cells of a particular therapeutic cell composition respond to different levels of recombinant receptor stimulation.

Any number of measures can be determined, extracted, extrapolated, estimated, and/or inferred from the measured recombinant receptor-dependent activity produced according to methods described herein. Non-limiting examples of measures oinclude titrated ratios at which maximal, minimal, and half-maximal (50%) recombinant receptor-dependent activity occurs, titrated ratios at which a specified percentage (e.g., 10%, 20%, 25%, 30%, 40%, 50%, 60%, 70%, 75%, 80%, or 90%) of maximal recombinant receptor-dependent activity occurs, and titrated ratios that encompass a range of recombinant receptor-dependent activity, for example, 10%-90%, 20%-80%, 30%-70%, 40%-60% of maximal recombinant receptor-dependent activity. In some embodiments, the measured recombinant receptor-dependent activity of a therapeutic cell composition undergoes curve fitting to generate a recombinant receptor-dependent activity curve. In some embodiments, the curve is similar to a dose-response curve. In some embodiments, measures of recombinant receptor-dependent activity and/or ratios at which particular recombinant receptor-dependent activity occurs are extrapolated and/or estimated from the curve. In some embodiments, the recombinant receptor-dependent activity curve may be used to extrapolate values or measures of therapeutic cell composition and/or recombinant receptor stimulating agent at which particular recombinant receptor-dependent activity occurs. In some embodiments, for example when the therapeutic cell composition cell count is held constant and the amount of recombinant receptor stimulating agent is varied, the amount (e.g., mass (e.g., in picograms) in the case of a purified or recombinant target, or number of cells in the case of target-expressing cells) or concentration (e.g., in mass/volume (e.g., in pg/ml) in the case of a purified or recombinant target or number of cells per unit volume (e.g., cells/mL) in the case of target-expressing cells) of recombinant receptor stimulating agent is used to determine maximal, minimal, half-maximal, and ranges at which recombinant receptor-dependent activity occurs. In some embodiments, for example when the therapeutic cell composition cell count is varied and the amount of recombinant receptor stimulating agent is held constant, the number (e.g., count, total) of cells of the therapeutic cell composition is used to determine maximal, minimal, half-maximal, and ranges at which recombinant receptor-dependent activity occurs. In some embodiments, the titrated ratio(s) is used to determine maximal, minimal, half-maximal, and ranges at which recombinant receptor-dependent activity occurs. In some embodiments, the amount or concentration of the recombinant receptor stimulating agent is used to determine the half-maximal recombinant receptor-dependent activity. In some embodiments, the amount (e.g., count) of cells of a therapeutic cell composition is used to determine the half-maximal recombinant receptor-dependent activity. In some embodiments, the titrated ratio is used to determine the half-maximal recombinant receptor-dependent activity. These exemplified measures, as well as others not listed, provide a quantitative description of the therapeutic cell composition which can be used to determine potency and/or relative potency (e.g., potency relative to reference standard, such as described herein (e.g., Section I-D) of the therapeutic cell composition.

In some embodiments, the potency of a therapeutic cell composition is expressed as a value or measure of the titrated ratio, amount of cells of the therapeutic cell composition, and/or amount or concentration of recombinant receptor stimulating agent determined based on the recombinant receptor-dependent activity. In some embodiments, the potency of a therapeutic cell composition is the value or measure of the titrated ratio, amount of cells of the therapeutic cell composition, and/or amount or concentration of recombinant receptor stimulating agent at which the half-maximal value (e.g., 50% of maximum activity) of the recombinant receptor-dependent activity occurs. In some embodiments, the potency of a therapeutic cell composition is the titrated ratio at which the half-maximal value (e.g., 50% of maximum activity) of the recombinant receptor-dependent activity occurs. In some embodiments, the potency of the therapeutic cell composition is the concentration of recombinant receptor stimulating agent at which the half-maximal value of the recombinant receptor-dependent activity occurs. In some embodiments, the half-maximal value of the recombinant receptor-dependent activity reflects the titrated ratio, concentration of recombinant receptor stimulating agent, and/or cell count, at which 50% effective stimulation (ES50) of the therapeutic cell composition occurs, according to the measured recombinant receptor-dependent activity.

In some embodiments, the potency of the therapeutic cell composition is a relative potency. For example, the titrated ratio at which half-maximal recombinant receptor-dependent activity is measured for a therapeutic cell composition can be compared to the titrated ratio at which half-maximal recombinant receptor-dependent activity is measured for a reference standard. It should be appreciated that concentration or amount of recombinant receptor stimulating agent or cell count may be used in place of the titrated ratio, if applicable. In some embodiments, the reference standard is a therapeutic cell composition having a known and/or validated titrated ratio at which half-maximal recombinant receptor-dependent activity occurs. In some embodiments, the reference standard is a commercially available therapeutic cell composition for which a titrated ratio at which half-maximal recombinant receptor-dependent activity occurs has been determined, for example using a method as described herein. In some embodiments, the reference standard is a different therapeutic cell composition for which a titrated ratio at which half-maximal recombinant receptor-dependent activity occurs has been determined, for example using a method as described herein. In some embodiments, the different therapeutic cell composition contains cells that express a recombinant receptor that binds to the same antigen as the test therapeutic cell composition, but has a different receptor structure. In some embodiments, the different therapeutic cell composition contains cells that express the identical recombinant receptor as the test therapeutic cell composition, but the therapeutic cell composition was manufactured using a process different from the process used to manufacture the test therapeutic cell composition. In some embodiments, the relative potency is a ratio determined by dividing the titrated ratio that results in half-maximal value of the test therapeutic cell composition by the titrated ratio that results in half-maximal value of the reference standard. In some embodiments, the relative potency is a percentage determined by dividing the titrated ratio that results in half-maximal value of the test therapeutic cell composition by the titrated ratio that results in half-maximal value of the reference standard and multiplying by 100.

In some cases, normalizing the recombinant receptor-dependent activity of therapeutic cell compositions is useful for determining whether the recombinant receptor-dependent activity for two or more therapeutic cell compositions can be compared. For example, if recombinant receptor-dependent activity for two or more therapeutic compositions is determined, and the maximum and/or minimum recombinant receptor-dependent activity is different for each of the therapeutic cell compositions tested, normalizing the recombinant receptor-dependent activity of each composition to its own maximum value may allow for an assessment of the appropriateness of comparing the recombinant receptor dependent activities.

In some embodiments, the recombinant receptor-dependent activity is normalized to the maximum recombinant receptor-dependent activity value measured. In some embodiments, the recombinant receptor-dependent activity curve is normalized to the maximum recombinant receptor-dependent activity measured. In some embodiments, when recombinant receptor-dependent activity curve is normalized, the maximum activity value is an average over the upper asymptote of the curve. In some embodiments, normalizing the recombinant receptor-dependent activity of the therapeutic cell composition and the reference standard by their respective maximum values facilitates comparison between the test therapeutic cell composition and the reference standard. In some embodiments, normalizing the recombinant receptor-dependent activity of the therapeutic cell composition and the reference standard by their respective maximum values facilitates calculating the relative potency.

In some embodiments, normalizing the recombinant receptor-dependent activity of the therapeutic cell composition and the reference standard by their respective maximum values allows for a parallel line test to be performed. In some embodiments, the results of the parallel line test indicate the ability to compare the therapeutic cell composition and the reference standard.

The methods, including assays, provided herein for assessing potency of a therapeutic cell composition allows for different therapeutic cell compositions, including reference standards, to be compared. The ability to compare therapeutic cell compositions provides a method not only for identifying therapeutic cell compositions with improved, optimal, and/or consistent potencies, but also to: identify candidate therapeutic cell compositions for further development and/or analysis; identify manufacturing processes and procedures that yield therapeutic cell compositions with improved or optimal potency; identify manufacturing procedures or processes that yield therapeutic cell compositions with consistent potency, and/or estimate a variability inherent to a manufacturing procedure; determine a dose of a therapeutic cell composition to be administered to a subject in need thereof, for example a dose that will yield a clinical response without development of toxicity; and/or compare the potency of allogeneic therapeutic cell compositions to autologous therapeutic cell compositions. The methods provided herein are designed to be compatible with a relative potency format that is agnostic as to whether the test therapeutic composition or reference standard are from different donors (e.g., subject), manufacturing processes, and/or therapeutic products.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. CELL POTENCY ASSAY

Provided herein are methods of assessing potency of a therapeutic cell composition, for example a therapeutic T cell composition containing T cells (e.g., CD3+, CD4+, CD8+ T cells) engineered to express a recombinant receptor (e.g., CAR), e.g., as described in Section I-A, using an assay including a plurality of incubations, where each of the plurality of incubations includes culturing cells of the therapeutic cell composition containing cells engineered to express a recombinant receptor with a recombinant receptor stimulating agent, for example an antigen, antigen-expressing cell, or antibody (e.g., anti-idiotype antibody) as described, e.g., in Section I-B, that is recognized or able to be bound by the recombinant receptor to stimulate a recombinant receptor-dependent activity in the cell. In some embodiments, the recombinant receptor-dependent activity is an activity of the cell that is elicited in response to stimulation of its recombinant receptor. In some embodiments, the recombinant receptor-dependent activity is an activity such as, for example, cytokine expression, cytolytic activity, receptor upregulation or downregulation, gene upregulation or downregulation, cytolytic activity, proliferative activity, and/or measures of cell health, e.g., as described in Section I-C. In some embodiments, each of the plurality of incubations contains a different titrated ratio of the cells of the therapeutic cell composition to the recombinant receptor stimulating agent. In some embodiments, each of the plurality of incubations includes culturing a constant number of cells of the therapeutic composition with differing amounts of recombinant receptor stimulating agent to generate a plurality of different titrated ratios. In some embodiments, each of the plurality of incubations includes culturing a differing number of cells of the therapeutic composition with a constant amount or concentration of recombinant receptor stimulating agent to generate a plurality of different titrated ratios. In some embodiments, at or at least 3, 4, 5, 6, 7, 8, 9, 10, 11, 12, 13, 14, 15, or more incubations are performed, each incubation containing a different ratio of cells of a therapeutic cell composition to recombinant receptor stimulating agent. In some embodiments, at or at least 3 incubations are performed, each incubation containing a different ratio of cells of a therapeutic cell composition to recombinant receptor stimulating agent. In some embodiments, at or at least 6 incubations are performed, each incubation containing a different ratio of cells of a therapeutic cell composition to recombinant receptor stimulating agent. In some embodiments, at or at least 10 incubations are performed, each incubation containing a different ratio of cells of a therapeutic cell composition to recombinant receptor stimulating agent. In some embodiments, the recombinant receptor-dependent activity from each of the plurality of incubations is measured. In some embodiments, the recombinant receptor-dependent activity from each of the plurality of incubations is measured as described in Section I-C, e.g., by fluorescence, flow cytometry, ELISA. In some embodiments, the measurements of the recombinant receptor-dependent activity are fit by curve to produce a recombinant receptor-dependent activity curve, for example as described above. In some embodiments, based on the recombinant receptor-dependent activity measured from each of the plurality of incubations, the titrated ratio that results in a half-maximal recombinant receptor-dependent activity is determined. In some embodiments, the titrated ratio that results in a half-maximal recombinant receptor-dependent activity is inferred, extrapolated, or estimated from the recombinant receptor-dependent activity curve. In some embodiments, the recombinant receptor-dependent activity curve is normalized to the maximum recombinant receptor-dependent activity measured. In some embodiments, the titrated ratio that results in half-maximal recombinant receptor-dependent activity is the potency of the therapeutic cell composition. In some embodiments, a concentration or amount of recombinant receptor stimulating agent or a cell count may be reported in place of the titrated ratio, if applicable.

In some embodiments, the titrated ratio that results in half-maximal recombinant receptor-dependent activity is compared to a titrated ratio that results in half-maximal recombinant receptor-dependent activity in a reference standard. For example, the titrated ratio of the therapeutic cell composition that results in half-maximal recombinant receptor-dependent activity is divided by a titrated ratio that results in half-maximal recombinant receptor-dependent activity in a reference standard, for example determined according to the methods described herein, to yield a relative potency. In some embodiments, the relative potency is expressed as a ratio. In some embodiments, the relative potency is expressed as a percentage.

The methods provided herein for determining potency may be performed with replication. For example, an assay may be performed 2, 3, 4, 5, or more times. In some embodiments, replicates are used to confirm accuracy and/or precision of the assay, including the consistency of measured of recombinant receptor-dependent activity and/or determined potency and/or relative potency. In some embodiments, a single assay is conducted by performing the assay on a particular therapeutic cell composition in duplicate or triplicate. In some embodiments, the assay is performed in duplicate. In some embodiments, the assay is performed in triplicates. In some cases where the assay is performed, for example, in duplicate or triplicate, the measured recombinant receptor-dependent activity from each of the replicates is used to provide a statistical measure of the recombinant receptor-dependent activity. For example, in some cases, an average, median, standard deviation, and/or variance of each measure of the recombinant receptor-dependent activity is determined. In some embodiments, an average of each measure of the recombinant receptor-dependent activity is determined. In some embodiments, a standard deviation of each measure of the recombinant receptor-dependent activity is determined. In some embodiments, the average measure of recombinant receptor-dependent activity are fit using a mathematical model to produce a recombinant receptor-dependent activity curve. In some embodiments, the curve is normalized to the average maximal value. In some embodiments, the average titrated ratio that results in half-maximal recombinant receptor-dependent activity is the potency of the therapeutic cell composition. In some embodiments, an average concentration or amount of recombinant receptor stimulating agent or a cell count may is reported in place of the titrated ratio, if applicable. In some embodiments, the potency of the therapeutic cell composition is a relative potency determined by taking an average titrated ratio that results in half-maximal recombinant receptor-dependent activity and comparing the average titrated ratio to a single or average titrated ratio that results in half-maximal recombinant receptor-dependent activity in a reference standard. In some embodiments, the relative potency is the average potency of the therapeutic cell composition divided by the single or average potency of the standard reference. In some embodiments, the relative potency is expressed as a ratio. In some embodiments, the relative potency is expressed as a percentage.

The assay provided herein may be performed in any vessel(s) suitable for a plurality of incubations. In some embodiments, the assay is performed in a flasks. In some embodiments, the assay is performed in tubes, e.g., microcentrifuge tubes, PCR tubes, tubes. In some embodiments, the assay is performed in multiwell plates. For instance, the multi-well plate is a 6-well plate, 12-well plate, 24-well plate, 48-well plate or 96-well plate. In particular embodiments, the assay is performed or carried out in a 12-well plate.

The conditions under which the incubation with the therapeutic cell composition and recombinant receptor stimulating agent are cultured can include one or more of particular media, temperature, oxygen content, carbon dioxide content, time, and/or agents, e.g., nutrients, amino acids, antibiotics, ions. The duration of the plurality of incubations is contemplated to be commensurate with at least the minimal amount of time for a potential recombinant receptor dependent activity to be detected (e.g., measured). For example, the amount of time needed to accurately measure cytokine expression may be longer than for measurement of gene expression. It is further contemplated that within a type of activity, e.g., cytokine expression, there may be a difference in time for a particular cytokine to be measured compared to another. In some embodiments, the plurality of incubations are performed for at, about, or at least 1, 2, or 3 days. In some embodiments, the plurality of incubations are performed for at, about, or at least 1 or 2 days. In some embodiments, the plurality of incubations are performed for at, about, or at least 24, 36, 48, 60, or 72 hours. In some embodiments, the plurality of incubations are performed for at, about, or at least 24 or 48 hours. In some embodiments, the plurality of incubations are performed for between at or about 24 hours and at or about 72 hours. In some embodiments, the plurality of incubations are performed for between at or about 24 hours and at or about 48 hours. In some embodiments, the plurality of incubations are performed for at, about, or at least 10, 15, 20, 25, 30, 35, 40, 45, 50, 55, or 60 minutes. In some embodiments, the plurality of incubations are performed for at, about, or at least 30 minutes. In some embodiments, the plurality of incubations are performed for at, about, or at least 60 minutes. In some embodiments, the plurality of incubations are performed for at or about between 10 and 60, 20 and 60, 30 and 60, 40 and 60, 50 and 60 minutes.

In some embodiments, the plurality of incubations performed at a temperature from about 25 to about 38° C., such as from about 30 to about 37° C., for example at or about 37° C.±2° C. In some embodiments, the plurality of incubations are performed with a CO₂ level from about 2.5% to about 7.5%, such as from about 4% to about 6%, for example at or about 5%±0.5%. In some embodiments, the plurality of incubations are performed at a temperature of or about 37° C. and/or at a CO₂ level of or about 5%.

A. Therapeutic Cell Composition

The methods provided herein are directed to assessing the potency of a therapeutic cell composition, e.g., therapeutic T cell composition, for example manufactured by any process. In some embodiments, the methods provided herein may be used to assess a therapeutic cell composition manufactured according to a process described herein (e.g., Section II). In some embodiments, the potencies and/or relative potencies of a plurality of therapeutic cell compositions manufactured by any process may be assessed according to the methods provided herein. In some embodiments, the plurality of therapeutic cell compositions assessed are produced by an identical manufacturing process. In some embodiments, the plurality of therapeutic cell compositions are manufactured by the identical manufacturing process but comprise different recombinant receptors. In some embodiments, the target is an antigen of the recombinant receptor. Thus, in some cases, the target-expressing cells are antigen-expressing cells. In some embodiments, the different recombinant receptors all bind the same target, e.g., target antigen. In some embodiments, the different recombinant receptors bind different targets, e.g., target antigen. In some embodiments, the plurality of therapeutic cell compositions assessed are produced by a different manufacturing process. In some embodiments, the plurality of therapeutic cell compositions are manufactured by different manufacturing process but comprise identical recombinant receptors. In some embodiments, the plurality of therapeutic cell compositions are manufactured by different manufacturing process and comprise different recombinant receptors. In some embodiments, the different recombinant receptors all bind the same antigen. In some embodiments, the plurality of therapeutic cell compositions are manufactured from a single subject. In some embodiments, the plurality of therapeutic cell compositions are manufactured from different subjects. In some embodiments, the subject is a healthy donor. In some embodiments, the subject has a disease or condition, e.g., cancer. The methods provided herein are capable of allowing comparison of potency and/or relative potency between therapeutic cell compositions, including references standards that are therapeutic cell compositions, regardless of the method of manufacture.

In some embodiments, the therapeutic cell compositions are produced or manufactured in connection with a process that produces or generates a therapeutic cell composition containing engineered T cells from one or more input populations, such as input populations obtained, selected, or enriched from a single biological sample (see, e.g., Section II-A). In certain embodiments, the therapeutic cell composition contains cells that express a recombinant receptor, e.g., CAR, TCR. In particular embodiments, the cells of the therapeutic cell composition are suitable for administration to a subject as a therapy, e.g., an autologous cell therapy, allogeneic cell therapy. The methods provided herein may be used to assess the potency and/or relative potency of the therapeutic cell composition for cell therapy.

In some embodiments, a process for generating or producing a therapeutic cell composition of engineered T cells includes some or all of the steps of: collecting or obtaining a biological sample; isolating, selecting, or enriching input cells from the biological sample; cryofreezing and storing and then thawing the input cells; selecting and stimulating input cells of interest, e.g., T cells, e.g., CD3+, CD4+, CD8+ T cells; genetically engineering the stimulated cells to express or contain a recombinant polynucleotide, e.g., a polynucleotide encoding a recombinant receptor such as a CAR; formulating the cultivated cells in an output composition; and cryofreezing and storing the formulated output cells until the cells are released for infusion and or administration to a subject. In some embodiments, the methods of manufacturing the therapeutic cell composition do not include a step to expand or increase the number of cells during the process, such as by cultivating the cells in a bioreactor under conditions where the cells expand, such as to a threshold amount that is at least 2, 3, 4, 5, or more times the amount, level, or concentration of the cells as compared to the input population. In some embodiments, the methods of manufacturing the therapeutic cell composition include a step to expand or increase the number of cells during the process, such as by incubation or cultivating the cells in a bioreactor under conditions where the cells expand, such as to a threshold amount that is at least 2, 3, 4, 5, or more times the amount, level, or concentration of the cells as compared to the input population. In some embodiments, genetically engineering the cells is or includes steps for transducing the cells with a viral vector, such as by spinoculating the cells in the presence of viral particles and then incubating the cells under static conditions in the presence of the viral particles. For example, see Section II-C.

In certain embodiments, the total duration of the process for generating engineered cells, from the initiation of the stimulation to collecting, harvesting, or formulating the cells is, is about, or is less than 36 hours, 42 hours, 48 hours, 54 hours, 60 hours, 72 hours, 84 hours, 96 hours, 108 hours, or 120 hours. In some embodiments, the total duration of the provided process for generating engineered cells, from the initiation of the stimulation to collecting, harvesting, or formulating the cells is between or between about 36 hours and 120 hours, 48 hours and 96 hours, or 48 hours and 72 hours, inclusive. In particular embodiments, the amount of time to complete the provided process as measured from the initiation of incubation to harvesting, collecting, or formulating the cells is, is about, or is less than 48 hours, 72 hours, or 96 hours. In particular embodiments, the amount of time to complete the provided process as measured from the initiation of incubation to harvesting, collecting, or formulating the cells is 48 hours±6 hours, 72 hours±6 hours, or 96 hours±6 hours.

In some embodiments, the entire manufacturing process is performed with a single population of enriched T cells, e.g., CD3+, CD4+, and CD8+ T cells. In certain embodiments, the manufacturing process is performed with two or more input populations of enriched T cells that are combined prior to and/or during the process to generate or produce a single therapeutic cell composition of enriched T cells (e.g., therapeutic cell composition containing CD4+ and CD8+ T cells). In some embodiments, the enriched T cells are or include engineered T cells, e.g., T cells transduced to express a recombinant receptor.

In some embodiments, the duration or amount of time required to complete the provided process, as measured from the isolation, enrichment, and/or selection input cells (e.g., CD4+ or CD8+ T cells) from a biological sample to the time at which engineered cells of a therapeutic cell composition are collected, formulated, and/or cryoprotected is, is about, or is less than 48 hours, 72 hours, 96 hours, 120 hours, 4 days, 5 days, 7 days, or 10 days. In some embodiments, the duration or amount of time required to complete the provided process, as measured from the isolation, enrichment, and/or selection input cells (e.g., CD4+ or CD8+ T cells) from a biological sample to the time at which the engineered cells are collected, formulated, and/or cryoprotected is, is about 4 to 5 days. In some embodiments, the duration or amount of time required to complete the provided process, as measured from the isolation, enrichment, and/or selection input cells (e.g., CD4+ or CD8+ T cells) from a biological sample to the time at which the engineered cells are collected, formulated, and/or cryoprotected is or is about 5 days. In some embodiments, the duration or amount of time required to complete the provided process, as measured from the isolation, enrichment, and/or selection input cells (e.g., CD4+ or CD8+ T cells) from a biological sample to the time at which the engineered cells are collected, formulated, and/or cryoprotected is, is less than 5 days. In some embodiments, the duration or amount of time required to complete the provided process, as measured from the isolation, enrichment, and/or selection input cells (e.g., CD4+ or CD8+ T cells) from a biological sample to the time at which the engineered cells are collected, formulated, and/or cryoprotected is or is about 4 days. In some embodiments, isolated, selected, or enriched cells are not cryoprotected prior to the stimulation, and the duration or amount of time required to complete the provided process, as measured from the isolation, enrichment, and/or selection input cells to the time at which the engineered cells are collected, formulated, and/or cryoprotected is, is about, or is less than 48 hours, 72 hours, 96 hours, or 120 hours.

In certain embodiments, the therapeutic cell compositions manufactured from a population of cells, e.g., CD4+ and CD8+ T cells or CD3+ T cells, that were isolated, enriched, or selected from a biological sample. In some aspects, the time to produce or generate from a therapeutic cell composition from when the biological sample is collected from a subject is within a shortened amount of time as compared to other methods or processes.

In some embodiments, at least 30%, at least 40%, at least 45%, at least 50%, at least 55%, at least 60%, at least 65%, at least 70%, at least 75%, at least 80%, at least 85%, or at least 90%, at least 95%, of the cells of the therapeutic cell composition express the recombinant receptor. In certain embodiments, at least 50% of the cells of the therapeutic cell composition express the recombinant receptor. In certain embodiments, at least 30%, at least 40%, at least 45%, at least 50%, at least 55%, at least 60%, at least 65%, at least 70%, at least 75%, at least 80%, at least 85%, at least 90%, or at least 95%, of the CD3+ T cells of the therapeutic cell composition express the recombinant receptor. In some embodiments, at least 50% of the CD3+ T cells of the therapeutic cell composition express the recombinant receptor. In particular embodiments, at least at least 30%, at least 40%, at least 45%, at least 50%, at least 55%, at least 60%, at least 65%, at least 70%, at least 75%, at least 80%, at least 85%, at least 90%, at least 95%, at least 97%, at least 99%, or more than 99% of the CD4+ T cells of the therapeutic cell composition express the recombinant receptor. In particular embodiments, at least 50% of the CD4+ T cells of the therapeutic cell composition express the recombinant receptor. In some embodiments, at least at least 30%, at least 40%, at least 45%, at least 50%, at least 55%, at least 60%, at least 65%, at least 70%, at least 75%, at least 80%, at least 85%, at least 90%, at least 95%, at least 97%, at least 99%, or more than 99% of the CD8+ T cells of the therapeutic cell composition express the recombinant receptor. In certain embodiments, at least 50% of the CD8+ T cells of the therapeutic cell composition express the recombinant receptor.

In particular embodiments, a majority of the cells of the therapeutic cell composition are naïve-like, central memory, and/or effector memory cells. In particular embodiments, a majority of the cells of the therapeutic cell composition are naïve-like or central memory cells. In some embodiments, a majority of the cells of the therapeutic cell composition are positive for one or more of CCR7 or CD27 expression. In certain embodiments, the cells of the therapeutic cell composition have a greater portion of naïve-like or central memory cells that output populations generated from alternative processes, such as processes that involve expansion.

In certain embodiments, the cells of the therapeutic cell composition have a low portion and/or frequency of cells that are exhausted and/or senescent. In particular embodiments, the cells of the output population have a low portion and/or frequency of cells that are exhausted and/or senescent. In some embodiments, less than 40%, less than 35%, less than 30%, less than 25%, less than 20%, less than 15%, less than 10%, less than 5%, or less than 1% of the cells of the therapeutic cell composition are exhausted and/or senescent. In certain embodiments, less than 25% of the cells of the therapeutic cell composition are exhausted and/or senescent. In certain embodiments, less than less than 10% of the cells of the output population are exhausted and/or senescent. In particular embodiments, the cells have a low portion.

In some embodiments, the cells of the therapeutic cell composition have a low portion and/or frequency of cells that are negative for CD27 and CCR7 expression, e.g., surface expression. In particular embodiments, the cells of the therapeutic cell composition have a low portion and/or frequency of CD27− CCR7− cells. In some embodiments, less than 40%, less than 35%, less than 30%, less than 25%, less than 20%, less than 15%, less than 10%, less than 5%, or less than 1% of the cells of the therapeutic cell composition are CD27− CCR7− cells. In certain embodiments, less than 25% of the cells of the therapeutic cell composition are CD27− CCR7− cells. In certain embodiments, less than less than 10% of the cells of the therapeutic cell composition are CD27− CCR7− cells. In embodiments, less than 5% of the cells of the therapeutic cell composition are CD27− CCR7− cells.

In some embodiments, the cells of the therapeutic cell composition have a high portion and/or frequency of cells that are positive for one or both of CD27 and CCR7 expression, e.g., surface expression. In some embodiments, the cells of the therapeutic cell composition have a high portion and/or frequency of cells that are positive for one or both of CD27 and CCR7. In some embodiments, at least 50%, at least 60%, at least 70%, at least 75%, at least 80%, at least 85%, at least 90%, at least 95%, or greater than 95% of the cells of the therapeutic cell composition are positive for one or both of CD27 and CCR7. In various embodiments, at least 50%, at least 60%, at least 70%, at least 75%, at least 80%, at least 85%, at least 90%, at least 95% or greater than 95% of the CD4+CAR+ cells of the therapeutic cell composition are positive for one or both of CD27 and CCR7. In some embodiments, at least 50%, at least 60%, at least 70%, at least 75%, at least 80%, at least 85%, at least 90%, at least 95% or greater than 95% of the CD8+CAR+ cells of the therapeutic cell composition are positive for one or both of CD27 and CCR7.

In certain embodiments, the cells of the therapeutic cell composition have a high portion and/or frequency of cells that are positive for CD27 and CCR7 expression, e.g., surface expression. In some embodiments, the cells of the therapeutic cell composition have a high portion and/or frequency of CD27+CCR7+ cells. In some embodiments, at least 50%, at least 60%, at least 70%, at least 75%, at least 80%, at least 85%, at least 90%, at least 95%, or greater than 95% of the cells of the therapeutic cell composition are CD27+CCR7+ cells. In various embodiments, at least 50%, at least 60%, at least 70%, at least 75%, at least 80%, at least 85%, at least 90%, at least 95% or greater than 95% of the CD4+CAR+ cells of the therapeutic cell composition are CD27+CCR7+ cells. In some embodiments, at least 50%, at least 60%, at least 70%, at least 75%, at least 80%, at least 85%, at least 90%, at least 95% or greater than 95% of the CD8+CAR+ cells of the therapeutic cell composition are CD27+CCR7+ cells.

In certain embodiments, the cells of the therapeutic cell composition have a low portion and/or frequency of cells that are negative for CCR7 and positive for CD45RA expression, e.g., surface expression. In some embodiments, the cells of the therapeutic cell composition have a low portion and/or frequency of CCR7-CD45RA+ cells. In particular embodiments, less than 40%, less than 35%, less than 30%, less than 25%, less than 20%, less than 15%, less than 10%, less than 5%, or less than 1% of the cells of the therapeutic cell composition are CCR7-CD45RA+ cells. In some embodiments, less than 25% of the cells of the output population (e.g., therapeutic cell composition) are CCR7-CD45RA+ cells. In particular embodiments, less than less than 10% of the cells of the output population (e.g., therapeutic cell composition) are CCR7-CD45RA+ cells. In certain embodiments, less than 5% of the cells of the therapeutic cell composition are CCR7-CD45RA+ cells.

In some embodiments, the therapeutic cell manufacturing processes differ such that alternative manufacturing processes can be compared, for example by comparing the potencies of the differently manufactured therapeutic cell compositions. For example, in some embodiments, the alternative process may contain a step for expanding the cells. In some embodiments, the alternative process may not contain a step for expanding the cells. In some embodiments, the alternative process includes separate steps for cell selection and stimulation. In some embodiments, the alternative process includes a single step for cell selection and stimulation. In some embodiments, the alternative process may differ in one or more specific aspects, but otherwise contains similar or the same features, aspects, steps, stages, reagents, and/or conditions of the process associated with the provided methods. In some embodiments, the alternative process differs in a manner that includes, but is not limited to, one or more of; including different reagents and/or media formulations; presence of serum during the incubation, transduction, transfection, and/or cultivation; different cellular makeup of the input population, e.g., ratio of CD4+ to CD8+ T cells; different stimulating conditions and/or a different stimulatory reagent; different ratio of stimulatory reagent to cells; different vector and/or method of transduction; different timing or order for incubating, transducing, and/or transfecting the cells; absence or difference of one or more recombinant cytokines present during the incubation or transduction (e.g., different cytokines or different concentrations), or different timing for harvesting or collecting the cells.

In some embodiments, the cell of the therapeutic cell composition are engineered to express recombinant receptors, such as CARs or TCRs (see, e.g., Section III), that specifically bind to a ligand, such as one associated with a disease or condition, e.g., associated with or expressed on a cell of a tumor or cancer. In some embodiments, the recombinant receptor contains an extracellular ligand-binding domain that specifically binds to an antigen. In some embodiments, the recombinant receptor is a CAR that contains an extracellular antigen-recognition domain that specifically binds to an antigen. In some embodiments, the ligand, such as an antigen, is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary recombinant receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the genetically engineered antigen receptors include a CAR as described in U.S. Pat. No. 7,446,190, and those described in International Patent Application Publication No.: WO/2014055668 A1.

In some embodiments, the engineered cells of the therapeutic cell composition contain a recombinant receptor (e.g., CAR), that binds to a tumor antigen. In some embodiments, the recombinant receptor specifically recognizes and/or targets an antigen associated with the cancer and/or present on a universal tag. In some embodiments, the antigen recognized or targeted by the recombinant receptor is B cell maturation antigen (BCMA), ROR1, carbonic anhydrase 9 (CAIX), Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, optionally a human antigen of any of the foregoing; a pathogen-specific antigen. In some embodiments, the antigen recognized and/or targeted by the recombinant receptor is selected from the group consisting of Notch 1, Notch 2, Notch 3, Notch 4, cell surface associated Mucin 1 (MUC1), Ephrin B2, Betaglycan (TGFBR3), CD43, CD44, CSF1R, CX3CR1, CXCL16, Delta1, E-cadherin, N-cadherin, HLA-A2, IFNaR2, IL1R1, IL1R2, IL6R, and amyloid precursor protein (APP).

In some embodiments the antigen recognized and/targeted by the recombinant receptor is B Cell Maturation Antigen (BCMA). Exemplary antigen-binding domains, and CARs containing such antigen-binding domains, that target or specifically bind BCMA are known, see e.g., WO 2016/090320, WO2016090327, WO2010104949A2 and WO2017173256. In some embodiments, the antigen binding domain is an scFv that contains a VH and a VL derived from an antibody or an antibody fragment specific to BCMA. In some embodiments, the antibody or antibody fragment that binds BCMA is or contains a VH and a VL from an antibody or antibody fragment set forth in International Patent Applications, Publication Number WO 2016/090327 and WO 2016/090320.

As described above, the assay may include a plurality of incubations, where each incubation is a culture containing a different titrated ratio of engineered cells of a therapeutic cell composition to a recombinant receptor stimulating agent, or vice versa, able to stimulate a recombinant receptor of the engineered cell to stimulate a recombinant receptor-dependent activity. It is within the level of a skilled artisan to empirically determine the precise range or amount of cells of a therapeutic cell composition and receptor stimulating agent to achieve a titrated response in the assay. For instance, the number or amount will depend on the particular format of the assay, such as the size of the vessel in which the assay is carried out. It is understood that the amounts will be less when the assay is performed in a vessel with a smaller surface area than in a vessel with a larger surface area. Typically the amount of cells is one in which the cells are sub-confluent, such as no more than 25% confluent or 50% confluent. Further, the particular range of ratios can be empirically determined depending on the particular antigen and the target cells being employed. For instance, the ratio chosen is one that includes a linear dose-response increase in recombinant receptor-dependent activity across the plurality of titrated amounts of a reference standard. In some embodiments, the ratio is chosen to also include a lower asymptote of receptor-dependent activity and an upper asymptote of receptor-dependent activity that represent a minimum and a maximum responses, respectively, of a reference standard.

In some embodiments, the number of engineered cells of the therapeutic cell composition is varied while the amount or concentration of binding molecule is held constant to generate different ratios. In some embodiments, the number of cells of the therapeutic cell composition is titrated from at or about 1×10⁴ to about 1×10⁶ cells, about 1×10⁴ to about 9×10⁵ cells, about 1×10⁴ to about 8×10⁵ cells, about 1×10⁴ to about 7×10⁵ cells, about 1×10⁴ to about 6×10⁵ cells, about 1×10⁴ to about 5×10⁵ cells, about 1×10⁴ to about 4×10⁵ cells, about 1×10⁴ to about 3×10⁵ cells, about 1×10⁴ to about 2×10⁵ cells, about 1×10⁴ to about 1×10⁵ cells, about 1×10⁴ to about 9×10⁴ cells, about 1×10⁴ to about 8×10⁴ cells, about 1×10⁴ to about 7×10⁴ cells, about 1×10⁴ to about 6×10⁴ cells, about 1×10⁴ to about 5×10⁴ cells, about 1×10⁴ to about 4×10⁴ cells, about 1×10⁴ to about 3×10⁴ cells, about 1×10⁴ to about 2×10⁴ cells across incubations. In some embodiments, the number of cells of the therapeutic cell composition is titrated from at or about 1×10⁴ to about 1×10⁵ cells, 1×10⁴ to about 8×10⁴ cells, 1×10⁴ to about 6×10⁴ cells, 1×10⁴ to about 4×10⁴ cells, 1×10⁴ to about 2×10⁴ cells across the plurality of incubations. In some embodiments, the number of cells of the therapeutic cells composition is titrated from at or about 10,000 to at or about 1,000,000 cells across the plurality of incubations. In some embodiments, the number of cells of the therapeutic cells composition is titrated from at or about 10,000 to at or about 500,000 cells across the plurality of incubations. In some embodiments, the number of cells of the therapeutic cells composition is titrated from at or about 10,000 to at or about 250,000 cells across the plurality of incubations. In some embodiments, the number of cells of the therapeutic cells composition is titrated from at or about 10,000 to at or about 200,000 cells across the plurality of incubations. In some embodiments, the number of cells of the therapeutic cells composition is titrated from at or about 10,000 to at or about 150,000 cells across the plurality of incubations. In some embodiments, the number of cells of the therapeutic cell composition is titrated from at or about 10,000 to at or about 100,000 cells across the plurality of incubations. In some embodiments, the number of cells of the therapeutic cell composition is titrated from at or about 10,000 to at or about 50,000 cells across the plurality of incubations. In some embodiments, the number of cells of the therapeutic cell composition in any of the foregoing is a total number of cells, a total number of viable cells, a total number of CAR+ cells, a total number of CD8+ cells, a total number of CD4+ cells, a total number of CD3+ cells, a total number of CD8+/CAR+ cells, a total number of CD4+/CAR+ cells, or a total number of CD3+/CAR+ cells.

In some embodiments, the number of cells of the therapeutic cell composition is held constant and the amount of recombinant receptor stimulating agent is titrated across the plurality of incubations. In some embodiments, the constant number of cells of the therapeutic cell composition is an amount between about 1×10⁴ to about 1×10⁶ cells, about 1×10⁴ to about 9×10⁵ cells, about 1×10⁴ to about 8×10⁵ cells, about 1×10⁴ to about 7×10⁵ cells, about 1×10⁴ to about 6×10⁵ cells, about 1×10⁴ to about 5×10⁵ cells, about 1×10⁴ to about 4×10⁵ cells, about 1×10⁴ to about 3×10⁵ cells, about 1×10⁴ to about 2×10⁵ cells, about 1×10⁴ to about 1×10⁵ cells, about 1×10⁴ to about 9×10⁴ cells, about 1×10⁴ to about 8×10⁴ cells, about 1×10⁴ to about 7×10⁴ cells, about 1×10⁴ to about 6×10⁴ cells, about 1×10⁴ to about 5×10⁴ cells, about 1×10⁴ to about 4×10⁴ cells, about 1×10⁴ to about 3×10⁴ cells, about 1×10⁴ to about 2×10⁴ cells across the plurality of incubations. In some embodiments, the constant number of cells of the therapeutic cell composition is an amount from at or about 1×10⁴ to about 1×10⁵ cells, 1×10⁴ to about 8×10⁴ cells, 1×10⁴ to about 6×10⁴ cells, 1×10⁴ to about 4×10⁴ cells, 1×10⁴ to about 2×10⁴ cells across the plurality of incubations. In some embodiments, the constant number of cells of the therapeutic cells composition is an amount from at or about 10,000 to at or about 1,000,000 cells across the plurality of incubations. In some embodiments, the constant number of cells of the therapeutic cells composition is an amount from at or about 10,000 to at or about 500,000 cells across the plurality of incubations. In some embodiments, the constant number of cells of the therapeutic cells composition is an amount from at or about 10,000 to at or about 250,000 cells across the plurality of incubations. In some embodiments, the constant number of cells of the therapeutic cells composition is an amount from at or about 10,000 to at or about 150,000 cells across the plurality of incubations. In some embodiments, the constant number of cells of the therapeutic cell composition is an amount from at or about 10,000 to at or about 100,000 cells across the plurality of incubations. In some embodiments, the constant number of cells of the therapeutic cell composition is an amount from at or about 10,000 to at or about 50,000 cells across incubations across the plurality of incubations. In some embodiments, the constant number of cells of the therapeutic cell composition is at or about 5,000; 10,000; 20,000; 30,000; 40,000; 50,000; 60,000; 70,000; 80,000; 90,000; or 100,000 cells across the plurality of incubations. In some embodiments, the constant number of cells of the therapeutic cell composition is at or about 5,000; 10,000; 20,000; 30,000; 40,000; or 50,000 cells across the plurality of incubations. In some embodiments, the constant number of cells of the therapeutic cell composition is at or about 20,000; 30,000; 40,000; or 50,000 cells across the plurality of incubations. In some embodiments, the constant number of cells of the therapeutic cell composition is at or about 50,000 cells across the plurality of incubations. In some embodiments, the constant number of cells of the therapeutic cell composition in any of the foregoing is a total number of cells, a total number of viable cells, a total number of CAR+ cells, a total number of CD8+ cells, a total number of CD4+ cells, a total number of CD3+ cells, a total number of CD8+/CAR+ cells, a total number of CD4+/CAR+ cells, or a total number of CD3+/CAR+ cells.

B. Recombinant Receptor Stimulating Agent

The methods of assessing potency provided herein include means of stimulating the recombinant receptors (e.g., CARs, TCRs) of the engineered cells of the therapeutic cell composition. It is contemplated that any means suitable for stimulating the recombinant receptor that is also capable of being quantified and delivered, such as to produce different ratios of cells of the therapeutic cell composition to the stimulating means may be used. In some embodiments, the means of stimulation of the recombinant receptor is achieved by a recombinant receptor stimulating agent able to bind to and stimulate an intracellular signal by the recombinant receptor to produce a recombinant receptor-dependent activity, such as described in Section I-C. Exemplary recombinant receptor stimulating agents include antigens (e.g., purified or recombinant antigens) of the recombinant receptor, antibodies such as anti-idiotype antibodies, and antigen-expressing cells.

As described above, in some embodiments, the plurality of incubations of different titrated ratios of cells of the therapeutic cell composition to the recombinant receptor stimulating agent may be accomplished by culturing a constant number of cells (e.g., viable, CAR+, CD4+, CD8+, CD3+, CD4+/CAR+, CD8+/CAR+, CD3+/CAR+ cells) of the therapeutic cell composition with a varying or titrated amount (e.g., concentration, mass) of recombinant receptor stimulating agent. In some embodiments, the amount or concentration of the recombinant receptor stimulating agent is varied or titrated at or about 10,000 fold, 5,000 fold, 1,000 fold, 1,500 fold, 500 fold, 250 fold, 200 fold, 150 fold, 100 fold, 75 fold, 50 fold, 25 fold, or 10 fold across the plurality of incubations. In some embodiments, the amount or concentration of the recombinant receptor stimulating agent is varied or titrated between at or about 10,000 to 100 fold, 5,000 to 100 fold, or 1,000 to 100 fold across the plurality of incubations. In some embodiments, the amount or concentration of the recombinant receptor stimulating agent is varied at or about 5,000 fold, 1,000 fold, 1,500 fold, or 500 fold across the plurality of incubations. In some embodiments, the amount or concentration of the recombinant receptor stimulating agent is varied or titrated at or about 5,000 fold across the plurality of incubations. In some embodiments, the amount or concentration of the recombinant receptor stimulating agent is varied or titrated at or about 1,000 fold across the plurality of incubations. In some embodiments, the amount or concentration of the recombinant receptor stimulating agent is varied or titrated at or about 500 fold across the plurality of incubations.

1. Binding Molecules and Surface Immobilization

In particular embodiments, the recombinant receptor stimulating agent is composed of a binding molecule (or target) that is able to be bound by the recombinant receptor. In some embodiments, the binding molecule is immobilized on a surface support. In provided embodiments, the binding molecule may be an antigen or a portion of an antigen of the recombinant receptor (e.g., extracellular portion of an antigen) or an antibody (e.g., an anti-idiotypic antibody) specific to the recombinant receptor. For instance, the binding molecule (e.g., antigen or binding portion thereof, or anti-idiotypic antibody) may be immobilized or bound to a surface support, such as a non-cell particle, wherein recombinant receptor-expressing cells (e.g., CAR-T cells) of the therapeutic composition, e.g., titrated amount of cells, are contacted with the surface support. In some embodiments, a particle described herein (e.g., bead particle) provides a solid support or matrix to which the binding molecule (e.g., an antigen or binding portion thereof, or an anti-idiotypic antibody), can be bound or attached in a manner that permits an interaction between the binding molecule and a cell, in particular binding between the binding molecule and a recombinant receptor, e.g., a CAR, expressed on the surface of the cell. In particular embodiments, the interaction between the conjugated or attached binding molecule and the cell mediates stimulation of the recombinant receptor, including one or more recombinant receptor-dependent activities such as activation, expansion, cytokine production, cytotoxicity activity or other activity as described, see e.g., Section I.C.

In certain embodiments, the surface support is a particle (e.g., a bead particle) to which the binding molecule (e.g., an antigen or binding portion thereof, or an anti-idiotypic antibody) is immobilized or attached. In some embodiments, the surface support is a solid support. In some examples, the solid support is a bead, and the antigen or portion is immobilized on the bead. In some embodiments, the solid support is the surface of a well or plate, e.g., a cell culture plate. In some embodiments, the surface support is a soluble oligomeric particle, and the antigen is immobilized on the surface of the soluble oligomeric particle. Examples of surface supports for immobilization or attachment of an agent (e.g., binding molecule) for recognition or binding to a recombinant receptor may be found in published International application WO 2019/027850, which is incorporated by reference for all purposes.

In particular embodiments, the surface support is a particle that may include a colloidal particle, a microsphere, nanoparticle, a bead, such as a magnetic bead, or the like. In some embodiments, the particles or beads are biocompatible, i.e. non-toxic. In certain embodiments the particles or beads are non-toxic to cultured cells, e.g., cultured T cells. In particular embodiments, the particles are monodisperse. In certain embodiments, “monodisperse” encompasses particles (e.g., bead particles) with size dispersions having a standard deviation of less than 5%, e.g., having less than a 5% standard deviation in diameter.

In some embodiments, the particle or bead is biocompatible, i.e., composed of a material that is suitable for biological use. In some embodiments, the particles, e.g., beads, are non-toxic to cultured cells, e.g., cultured T cells. In some embodiments, the particles, e.g., beads, may be any particles which are capable of attaching binding molecules in a manner that permits an interaction between the binding molecule and a cell. In certain embodiments, the particles, e.g., beads, may be any particles that can be modified, e.g., surface functionalized, to allow for the attachment of a binding molecule at the surface of the particle. In some embodiments, the particles, e.g., beads, are composed of glass, silica, polyesters of hydroxy carboxylic acids, polyanhydrides of dicarboxylic acids, or copolymers of hydroxy carboxylic acids and dicarboxylic acids. In some embodiments, the particles, e.g., beads, may be composed of or at least partially composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, particles, e.g., beads, can be quantum dots, or composed of quantum dots, such as quantum dot polystyrene particles, e.g., beads. Particles, e.g., beads, including mixtures of ester and anhydride bonds (e.g., copolymers of glycolic and sebacic acid) may also be employed. For example, particles, e.g., beads, may comprise materials including polyglycolic acid polymers (PGA), polylactic acid polymers (PLA), polysebacic acid polymers (PSA), poly(lactic-co-glycolic) acid copolymers (PLGA), [rho]poly(lactic-co-sebacic) acid copolymers (PLSA), poly(glycolic-co-sebacic) acid copolymers (PGSA), etc. Other polymers that particles, e.g., beads, may be composed of include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to binding molecules such as polypeptide antigen or antibodies.

In some embodiments, the particle is a bead that has a diameter of greater than 0.001 μm, greater than 0.01 μm, greater than 0.05 μm, greater than 0.1 μm, greater than 0.2 μm, greater than 0.3 μm, greater than 0.4 μm, greater than 0.5 μm, greater than 0.6 μm, greater than 0.7 μm, greater than 0.8 μm, greater than 0.9 μm, greater than 1 μm, greater than 2 μm, greater than 3 μm, greater than 4 μm, greater than 5 μm, greater than 6 μm, greater than 7 μm, greater than 8 μm, greater than 9 μm, greater than 10 μm, greater than 20 μm, greater than 30 μm, greater than 40 μm, greater than 50 μm, greater than 100 μm, greater than 500 μm, and/or greater than 1,000 μm. In some embodiments, the particles or beads have a diameter of between or between about 0.001 μm and 1,000 μm, 0.01 μm and 100 μm, 0.1 μm and 10, μm, 0.1 and 100 μm, 0.1 μm and 10 μm, 0.001 μm and 0.01 μm, 0.01 μm and 0.1 μm, 0.1 μm and 1 μm, 1 μm and 10 μm, 1 μm and 2 μm, 2 μm and 3 μm, 3 μm and 4 μm, 4 μm and 5 μm, 1 μm and 5 μm, and/or 5 μm and 10 μm, each inclusive. In certain embodiments, the particles or beads have a mean diameter of 1 μm and 10 μm, each inclusive. In certain embodiments, the particles, e.g., beads, have a diameter of or of about 1 μm. In particular embodiments, the particles, e.g., beads, have a mean diameter of or of about 2.8 μm. In some embodiments, the particles, e.g., beads, have a diameter of or of about 4.8 μm.

The particles (e.g., bead particles) used in the methods described herein can be produced or obtained commercially. Particles, e.g., beads, including methods of producing particles, e.g., beads, are well known in the art. See, for example, U.S. Pat. Nos. 6,074,884; 5,834,121; 5,395,688; 5,356,713; 5,318,797; 5,283,079; 5,232,782; 5,091,206; 4,774,265; 4,654,267; 4,554,088; 4,490,436; 4,452,773; U.S. Patent Application Publication No. 20100207051; and Sharpe, Pau T., Methods of Cell Separation, Elsevier, 1988. Commercially available particles, e.g., beads, (e.g., bead particles) include, but are not limited to, ProMag™ (PolySciences, Inc.); COMPEL™ (PolySciences, Inc.); BioMag® (PolySciences, Inc.), including BioMag® Plus (PolySciences, Inc.) and BioMag® Maxi (Bang Laboratories, Inc.); M-PVA (Cehmagen Biopolymer Technologie AG); SiMAG (Chemicell GmbH); beadMAG (Chemicell GmbH); MagaPhase® (Cortex Biochem); Dynabeads® (Invitrogen), including Dynabeads® M-280 Sheep Anti-rabbit IgG (Invitrogen), Dynabeads® FlowComp™ (e.g., Dynabeads® FlowCompTMHuman CD3, Invitrogen), Dynabeads® M-450 (e.g., Dynabeads® M-450 Tosylactivated, Invitrogen), Dynabeads® Untouched™ (e.g., Dynabeads® Untouched™ Human CD8 T Cells, Invitrogen), and Dynabeads® that bind, expand and/or activate T cells (e.g., Dynabeads® Human T-Activator CD3/CD28 for T Cell Expansion and Activation, Invitrogen); Estapor® M (Merk Chimie SAS); Estapor® EM (Merk Chimie SAS); MACSiBeads™ Particles (e.g., anti-biotin MACSiBead Particles, Miltenyi Biotec, catalog #130-091-147); Streptamer® Magnetic Beads (IBA BioTAGnology); Strep-Tactin® Magnetic Beads (IBA BioTAGnology); Sicastar®-M (Micormod Partikeltechnologie GmbH) Micromer®-M (Micromod Partikeltechnologie); MagneSil™ (Promega GmbH); MGP (Roche Applied Science Inc.); Pierce™ Protein G Magnetic Beads (Thermo Fisher Scientific Inc.); Pierce™ Protein A Magnetic Beads (Thermo Fisher Scientific Inc.); Pierce™ Protein A/G Magnetic Beads (Thermo Fisher Scientific Inc.); Pierce™ NHS-Activated Magnetic Beads (Thermo Fisher Scientific Inc.); Pierce™ Protein L Magnetic Beads (Thermo Fisher Scientific Inc.); Pierce™ Anti-HA Magnetic Beads (Thermo Fisher Scientific Inc.); Pierce™ Anti-c-Myc Magnetic Beads (Thermo Fisher Scientific Inc.); Pierce™ Glutathione Magnetic Beads (Thermo Fisher Scientific Inc.); Pierce™ Streptavidin Magnetic Beads (Thermo Fisher Scientific Inc.); MagnaBind™ Magnetic Beads (Thermo Fisher Scientific Inc.); Sera-Mag™ Magnetic Beads (Thermo Fisher Scientific Inc.); Anti-FLAG® M2 Magnetic Beads (Sigma-Aldrich); SPHERO™ Magnetic Particles (Spherotech Inc.); and HisPur™ Ni-NTA Magnetic Beads (Thermo Fisher Scientific Inc.).

In certain embodiments, the antigen or an extracellular domain portion thereof is bound to the particle (e.g., bead) via a covalent chemical bond. In particular embodiments, a reactive group or moiety of an amino acid of the antigen or extracellular domain portion thereof is conjugated directly to a reactive group or moiety on the surface of the particle by a direct chemical reaction. In certain embodiments, an amino acid carboxyl group (e.g., a C-terminal carboxyl group), hydroxyl, thiol, or amine group (such as an amino acid side chain group) of the antigen or extracellular binding portion thereof is conjugated directly to a hydroxyl or carboxyl group of a PLA or PGA polymer, a terminal amine or carboxyl group of a dendrimer, or a hydroxyl, carboxyl or phosphate group of a phospholipid on the surface of the particle by direct chemical reaction. In some embodiments, a conjugating moiety conjugates, e.g., covalently binds, to both the binding molecule and the particle, thereby linking them together.

In certain embodiments, the surface of the particle comprises chemical moieties and/or functional groups that allow attachment (e.g., covalent, non-covalent) of the binding molecule (e.g., polypeptide antigen or antibody). In particular embodiments, the particle surfaces contain exposed functional groups. Suitable surface exposed functional groups include, but are not limited to, carboxyl, amino, hydroxyl, sulfate groups, tosyl, epoxy, and chloromethyl groups. In some embodiments, the binding molecule is a polypeptide and is conjugated to the surface-exposed functional groups. In some embodiments, the surface exposed functional group must be activated, i.e., it must undergo a chemical reaction to yield an intermediate product capable of directly binding a polypeptide. For example, a carboxyl group of the polypeptide molecule may be activated with the agents described above to generate intermediate esters capable of directly binding to surface exposed amino groups of the particle. In other examples, free amine groups on the surface of a support surface (e.g., bead) may be covalently bound to antigen peptides and proteins, or antigen peptide or protein fusion proteins, using sulfosuccinimidyl (4-iodoacetyl)aminobenzoate (sulfo-SLAB) chemistry. In still other particular embodiments, a polypeptide binding molecule is covalently attached to the particle, e.g., a bead particle, at a surface exposed functional group that does not require activation by an agent prior to forming a covalent attachment. Examples of such functional groups include, but are not limited to, tosyl, epoxy, and chloromethyl groups.

In some embodiments, a non-covalent bond between a ligand bound to the antigen peptide or protein and an anti-ligand attached to the surface support (e.g., bead) may conjugate the antigen to the support (e.g., bead). In some embodiments, a biotin ligase recognition sequence tag may be joined to the C-terminus of an antigen peptide or protein, and this tag may be biotinylated by biotin ligase. The biotin may then serve as a ligand to non-covalently conjugate the antigen peptide or protein to avidin or streptavidin which is adsorbed or otherwise bound to the surface of the carrier as an anti-ligand. Alternatively, if the binding molecule (e.g., antigen) are fused to an immunoglobulin domain bearing an Fc region, as described herein, the Fc domain may act as a ligand, and protein A, either covalently or non-covalently bound to the surface of the surface support (e.g., bead), may serve as the anti-ligand to non-covalently conjugate the antigen peptide or protein to the carrier. Other means are well known in the art which may be employed to non-covalently conjugate binding molecules (e.g., antigen or anti-idiotypic antibody) to a surface support (e.g., beads), including metal ion chelation techniques (e.g., using a poly-His tag at the C-terminus of the binding molecule, e.g., antigen, and a Ni-coated surface support), and these methods may be substituted for those described here.

In some embodiments, the binding molecule (e.g., antigen or anti-idiotyptic antibody) is conjugated to the particle by a linker. In certain embodiments, the linkers can include, but are not limited to, a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particular coupling agents include N-succinimidyl-3-(2-pyridyldithio)propionate (SPDP) and N-succinimidyl-4-(2-pyridylthio)pentanoate (SPP) to provide for a disulfide linkage.

a. Antigen

In some embodiments, the recombinant receptor stimulating agent is or includes an antigen, e.g., a recombinant antigen or fragment thereof. For instance, the recombinant receptor stimulating agent may be an antigen that is immobilized or bound to a surface support, such as a microwell plate, a solid particle (e.g., bead) or an oligomeric particle, e.g., as described above. In some embodiments, the antigen is a polypeptide, or a variant or fragment of a polypeptide that is expressed on the surface of a cell that is associated with a disease, for example, a cancer cell and/or a tumor cell. It is understood that the antigen is an antigen that is recognized or bound by an extracellular domain of the recombinant receptor. A skilled artisan can determine the antigen and format of the antigen (e.g., cell expressed or immobilized on a solid surface) sufficient to stimulate the recombinant receptor.

In some embodiments, the antigen is or includes ανβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the antigen is or comprises a portion of a polypeptide antigen that is recognized by or bound by a recombinant receptor, e.g., a CAR. In particular embodiments, the portion of an antigen is a region that contains an epitope that is recognized by or bound by a recombinant receptor, e.g., a CAR. In certain embodiments, the portion of the polypeptide antigen contains, about, or contains at least 10, 15, 20, 25, 30, 35, 40, 50, 55, 60, 65, 70, 75, 80, 85, 90, 95, 100, 110, 120, 130, 140, 150, 160, 170, 180, 190, 200, 250, 300, 400, or 500 amino acids, in some cases contiguous amino acids, of the polypeptide that is recognized by or bound by a recombinant receptor and or a CAR. In certain embodiments, the polypeptide portion comprises an amino acid sequence of the epitope that is recognized by the recombinant receptor and/or CAR.

In certain embodiments, the antigen or portions is a polypeptide variant that contains, contains about, or contains at least 50%, 55%, 60%, 65%, 70%, 75%, 80%, 85%, 90%, 95%, 97%, 98%, 99%, or 99.5% amino acid sequence identity to a polypeptide that is bound by and/or recognized by recombinant receptor and/or CAR.

In certain embodiments, the extracellular domain of the recombinant receptor (e.g., CAR) is specific for or binds to BCMA and the antigen is BCMA or is an extracellular domain portion of BCMA. In some embodiments, the BCMA polypeptide is a mammalian BCMA polypeptide. In particular embodiments, the BCMA polypeptide is a human BCMA polypeptide. In some embodiments, the BCMA antigen is or comprises an extracellular domain of BCMA or a portion thereof comprising an epitope recognized by an antigen receptor, e.g., CAR. In certain embodiments, the BCMA antigen is or comprises a polypeptide with an amino acid sequence with at least 70%, 75%, 80%, 85%, 86%, 87%, 88%, 89%, 90%, 91%, 92%, 93%, 94%, 95%, 96%, 97%, 98%, 99% or more sequence identity to SEQ ID NO: 13 or a fragment thereof containing at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, or at least 180 contiguous amino acids of SEQ ID NO: 13. In some embodiments, the BCMA antigen is or includes the sequence set forth in SEQ ID NO: 13 or a portion thereof that is or contains an epitope recognized by an antigen receptor, e.g., CAR.

In certain embodiments, the extracellular domain of the recombinant receptor (e.g., CAR) is specific for or binds to ROR1 and the antigen is ROR1 or is an extracellular domain portion of ROR1. In certain embodiments, the ROR1 polypeptide is mammalian. In particular embodiments, the ROR1 polypeptide is human. In some embodiments, the antigen is an extracellular domain of ROR1 or a portion thereof comprising an epitope recognized by an antigen receptor, e.g., CAR. In some embodiments, the antigen is a polypeptide with an amino acid sequence with at least 70%, 75%, 80%, 85%, 86%, 87%, 88%, 89%, 90%, 91%, 92%, 93%, 94%, 95%, 96%, 97%, 98%, 99% or more sequence identity to SEQ ID NO: 19 or a fragment thereof containing at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, or at least 180 contiguous amino acids of SEQ ID NO: 19. In some embodiments, the ROR1 antigen comprises the sequence set forth in SEQ ID NO: 19 or a portion thereof comprising an epitope recognized by an antigen receptor, e.g., CAR.

In certain embodiments, the extracellular domain of the recombinant receptor (e.g., CAR) is specific for or binds to CD22 and the antigen is CD22 or is an extracellular domain portion of CD22. In certain embodiments, the CD22 polypeptide is mammalian. In particular embodiments, the CD22 polypeptide is human. In some embodiments, the antigen is an extracellular domain of CD22 or a portion thereof comprising an epitope recognized by an antigen receptor, e.g., CAR. In some embodiments, the antigen is a polypeptide with an amino acid sequence with at least 70%, 75%, 80%, 85%, 86%, 87%, 88%, 89%, 90%, 91%, 92%, 93%, 94%, 95%, 96%, 97%, 98%, 99% or more sequence identity to SEQ ID NO: 14 or a fragment thereof containing at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, or at least 180 contiguous amino acids of SEQ ID NO: 14. In some embodiments, the CD22 antigen comprises the sequence set forth in SEQ ID NO: 14 or a portion thereof comprising an epitope recognized by an antigen receptor, e.g., CAR.

In certain embodiments, the extracellular domain of the recombinant receptor (e.g., CAR) is specific for or binds to CD19 and the antigen is CD19 or is an extracellular domain portion of CD19. In certain embodiments, the CD19 polypeptide is mammalian. In particular embodiments, the CD19 polypeptide is human. In some embodiments, the antigen is an extracellular domain of CD19 or a portion thereof comprising an epitope recognized by an antigen receptor, e.g., CAR. In some embodiments, the antigen is a polypeptide with an amino acid sequence with at least 70%, 75%, 80%, 85%, 86%, 87%, 88%, 89%, 90%, 91%, 92%, 93%, 94%, 95%, 96%, 97%, 98%, 99% or more sequence identity to SEQ ID NO: 15 or a fragment thereof containing at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, or at least 180 contiguous amino acids of SEQ ID NO: 15. In some embodiments, the CD19 antigen comprises the sequence set forth in SEQ ID NO: 15 or a portion thereof comprising an epitope recognized by an antigen receptor, e.g., CAR.

In some embodiments, the antigen or portion thereof may be formatted as a multimer, e.g., a dimer, comprising two or more polypeptide antigens, or portion or variant thereof, that is recognized and/or bound by a recombinant receptor, such as an antigen receptor (e.g., a CAR). In some embodiments, the polypeptide antigen, or portion thereof, are identical. In certain embodiments, the polypeptide antigen is linked, directly or indirectly, to a region or domain, e.g., a multimerization domain, that promotes or stabilizes interaction between two or more polypeptide antigens via complementary interactions between the domains or regions. In some embodiments, providing the polypeptide antigen as a multimer, e.g., dimer, provides for a multivalent interaction between the antigen or extracellular domain portion thereof and the antigen-binding domain of the antigen receptor, e.g., CAR, which, in some aspects, can increase the avidity of the interaction. In some embodiment, an increased avidity may favor stimulatory or agonist activity of antigen receptor, e.g., CAR, by the antigen or extracellular domain portion thereof conjugated to the bead.

In some embodiments, a polypeptide is joined directly or indirectly to a multimerization domain. Exemplary multimerization domains include the immunoglobulin sequences or portions thereof, leucine zippers, hydrophobic regions, hydrophilic regions, and compatible protein-protein interaction domains. The multimerization domain, for example, can be an immunoglobulin constant region or domain, such as, for example, the Fc domain or portions thereof from IgG, including IgG1, IgG2, IgG3 or IgG4 subtypes, IgA, IgE, IgD and IgM and modified forms thereof. In particular embodiments, the polypeptide antigen is linked, directly or indirectly, to an Fc domain. In some embodiments, the polypeptide is a fusion polypeptide comprising the polypeptide antigen or portion thereof and the Fc domain.

In particular embodiments, an antigen or extracellular domain portion thereofis a fusion polypeptide that comprises an Fc domain. In some embodiments, the Fc domain is composed of the second and third constant domains (i.e., CH2 and CH3 domains) of the heavy chain of an IgG, IgA or IgD isotype, e.g., CH2 or CH3 of IgG, IgA and IgD isotypes. In some embodiments, the Fc domain is composed of three heavy chain constant domain (i.e., CH2, CH3, and CH4 domains) of an IgM or IgE isotype. In some embodiments, the Fc domain may further include a hinge sequence or portion thereof. In certain aspects, the Fc domain contains part or all of a hinge domain of an immunoglobulin molecule plus a CH2 and a CH3 domain. In some cases, the Fc domain can form a dimer of two polypeptide chains joined by one or more disulfide bonds. In some embodiments, the Fc domain is derived from an immunoglobulin (e.g., IgG, IgA, IgM, or IgE) of a suitable mammal (e.g., human, mouse, rat, goat, sheep, or monkey). In some embodiments, the Fc domain comprises C_{H}2 and C_{H}3 domains of IgG. In certain embodiments, the Fc domain is fused to the C-terminal of the polypeptide antigen. In particular embodiments, the Fc domain is fused to the N-terminal of the polypeptide antigen.

In some embodiments, the Fc domain is an IgG Fc domain, or a portion or variant thereof. In some embodiments, the Fc domain is a human IgG Fc domain, or a portion or a variant thereof, that comprises an amino acid sequence set forth in SEQ ID NO: 16 or an amino acid sequence that is at least 70%, 75%, 80%, 85%, 86%, 87%, 88%, 89%, 90%, 91%, 92%, 93%, 94%, 95%, 96%, 97%, 98%, 99% sequence identity to the sequence set forth in SEQ ID NO: 16. In particular embodiments, the Fc domain is a wild-type human IgG Fc domain, or a portion or variant thereof. In particular embodiments, the Fc domain is a variant of the wild-type human IgG1 Fc domain.

In some embodiments, the fusion polypeptide comprises a variant Fc domain. In certain embodiments, the variant human IgG Fc domain contains a mutation, e.g., a substitution, deletion, or insertion, that reduces, decreases, and/or diminishes pairing between the Fc domain and a light chain. In some embodiments, the variant human IgG Fc domain contains a mutation that reduces the binding affinity between the Fc domain and an Fc Receptor. In particular embodiments, the variant human IgG Fc domain contains a mutation that reduces, decreases, and/or diminishes the interactions, or the probability or likelihood of an interaction, between the Fc domain and an Fc Receptor. In some embodiments, the variant human IgG Fc domain contains a mutation that reduces the binding affinity between the Fc domain and a protein of the complement system. In particular embodiments, the variant human IgG Fc domain contains a mutation that reduces, decreases, and/or diminishes the interactions, or the probability or likelihood of an interaction, between the Fc domain and a protein of the complement system.

In some embodiments, the antigen or portion thereof is linked to a variant human IgG1 Fc domain. In some embodiments, the variant human IgG Fc domain contains a cystine to serine substitution in the hinge region of the Fc domain. In some embodiments, the variant human IgG Fc domain contains a leucine to alanine substitution in the hinge region of the Fc domain. In particular embodiments, the variant human IgG Fc domain contains a glycine to alanine substitution in the hinge region. In certain embodiments, the variant human IgG Fc domain contains an alanine to a serine substitution in the CH2 region of the Fc domain. In some embodiments, the variant human IgG Fc domain comprises a proline to serine substitution in the CH2 region of the Fc domain. In some embodiments, the variant human IgG Fc domain comprises an amino acid sequence as set forth by SEQ ID NO: 17.

In some embodiments, the antigen or extracellular domain portion thereof is provided as a fusion polypeptide comprising an Fc domain, wherein the Fc domain is present at the C-terminus of the fusion polypeptide.

In some embodiments, the antigen and the multimerization domain, such as Fc domain, are connected by a linker, such as an amino acid linker. In certain embodiments, the antigen is fused to the N-terminus of an amino acid linker, and the multimerization domain, such as Fc domain, is fused to the C-terminus of the linker. Although amino acid linkers can be any length and contain any combination of amino acids, the linker length may be relatively short (e.g., ten or fewer amino acids) to reduce interactions between the linked domains. The amino acid composition of the linker also may be adjusted to reduce the number of amino acids with bulky side chains or amino acids likely to introduce secondary structure. Suitable amino acid linkers include, but are not limited to, those up to 3, 4, 5, 6, 7, 10, 15, 20, or 25 amino acids in length. Representative amino acid linker sequences include GGGGS (SEQ ID NO: 22), and linkers comprising 2, 3, 4, or 5 copies of GGGGS (SEQ ID NO: 22).

In some embodiments, the the antigen is provided as an extracellular domain of BCMA, e.g., human BCMA, fused to an Fc domain (BCMA-Fc). In particular embodiments, the BCMA-Fc antigen contains all or a portion of the amino acid sequence set forth in SEQ ID NO: 18 or a sequence of amino acids that exhibits at least 70%, 75%, 80%, 85%, 86%, 87%, 88%, 89%, 90%, 91%, 92%, 93%, 94%, 95%, 96%, 97%, 98%, 99% to SEQ ID NO: 18, and that comprises an epitope recognize by an antigen receptor, e.g., CAR.

In some embodiments, the antigen is provided as an extracellular domain of ROR1, e.g., human ROR1, fused to an Fc domain (ROR1-Fc). In certain embodiments, the ROR-1-Fc antigen contains all or a portion of the amino acid sequence set forth in SEQ ID NO: 20 or a sequence of amino acids that exhibits at least 70%, 75%, 80%, 85%, 86%, 87%, 88%, 89%, 90%, 91%, 92%, 93%, 94%, 95%, 96%, 97%, 98%, 99% to SEQ ID NO: 20 and that comprises an epitope recognize by an antigen receptor, e.g., CAR.

In particular embodiments, the antigen is provided as an extracellular domain of CD22, e.g., human CD22, fused to an Fc domain (e.g., CD22-Fc). In certain embodiments, the CD22-Fc antigen contains all or a portion of he amino acid sequence set forth in SEQ ID NO: 21 or a sequence of amino acids that exhibits at least 70%, 75%, 80%, 85%, 86%, 87%, 88%, 89%, 90%, 91%, 92%, 93%, 94%, 95%, 96%, 97%, 98%, 99% to SEQ ID NO: 21 and that comprises an epitope recognize by an antigen receptor, e.g., CAR.

In some embodiments, an Fc fusion of an antigen or an extracellular binding domain thereof is linked or attached to the surface support as a dimer formed by two Fc fusion polypeptides containing the polypeptide antigen or portion thereof an Fc domain. In some embodiments, the resulting polypeptide antigen-Fc fusion protein, e.g., BCMA-Fc, ROR1-Fc, CD22-Fc, or CD19-Fc, can be expressed in host cells, e.g., transformed with the expression vectors, whereby assembly between Fc domains can occurs by interchain disulfide bonds formed between the Fc moieties to yield a dimeric, such as divalent, polypeptide antigen fusion protein. In some embodiments, the host cell is a mammalian cell line. Exemplary of mammalian cells for recombinant expression of proteins include HEK293 cells or CHO cells or derivatives thereof. In some aspects, the nucleic acid encoding the Fc fusion protein further includes a signal peptide for secretion from the cell. In an exemplary embodiment, the signal peptide is CD33 (e.g., set forth in SEQ ID NO: 12).

In some embodiments, the cell of the therapeutic cell composition expresses a CAR that binds to or recognizes a universal tag that can be fused to an antibody or a fragment or variant thereof. In particular embodiments, cells expressing such CARs are able to specifically recognize and kill target cells, for example tumor cells, that have been bound by antibodies that have been fused with the universal tag. One example includes, but is not limited to, anti-FITC CAR expressing T cells that can bind to and/or recognize various human cancer cells when those cells are bound by cancer-reactive FITC-labeled antibodies. Thus, in some embodiments, the same CAR that binds to the universal tag is useful for the treatment of different cancers, provided there are available antibodies that recognize antigens associated with the cancers that contain the universal tag. In particular embodiments, a particle (e.g., a bead particle) comprises a surface exposed binding molecule that comprises universal tag binding molecule that is able to be bound by or recognized by reccombinant receptor, e.g., CAR. In certain embodiments, the binding molecule is a universal tag or a portion thereof bound or recognized by the antigen receptor, e.g., CAR. Particular embodiments contemplate that any polypeptide domain that can be fused to an antibody, or an antigen binding fragment or variant thereof, that does not prevent the antibody from binding to its respective target is suitable for use as a universal tag. In some embodiments, a particle is bound to a binding molecule that comprises a universal tag, or a portion thereof, selected from the group consisting of: FITC, streptavidin, biotin, histidine, dinitrophenol, peridinin chlorophyll protein complex, green fluorescent protein, PE, HRP, palmitoylation, nitrosylation, alkalanine phosphatase, glucose oxidase, and maltose binding protein.

b. Antibodies

In some aspects, the binding molecule is an antibody or antigen binding fragment thereof that specifically recognizes a recombinant receptor, e.g., CAR. In some embodiments of these aspects, the antibody or antigen binding fragment specifically recognizes the extracellular portion of the recombinant receptor (e.g., specifically binds an epitope on the extracellular portion of the recombinant receptor), e.g., CAR.

In some aspects, the binding molecule is an anti-idiotype antibody or antigen-binding fragment thereof (“anti-IDs”) that specifically recognizes a recombinant receptor, for example a recombinant receptor, e.g., CAR, as described in Section III. In particular, an anti-idiotype antibody targets the antigen binding site of another antibody, such as the scFv of the extracellular antigen binding domain of a CAR. In some embodiments, the anti-ID is able to bind to the recombinant receptor to stimulate a recombinant receptor-dependent activity. Exemplary anti-idiotype antibodies against antigen-specific CARs are known. These include, but are not limited to, anti-idiotypic antibodies directed against a CD22-directed CAR, see e.g., PCT Publication No. WO2013188864; CD19-directed CAR, see e.g., PCT Publication No. WO 2018/023100; a GPRC5D-directed CAR, see e.g., PCT Application No. PCT/US2020/063497; and a BCMA-directed CAR, see e.g., PCT Application No. PCT/US2020/063492. The anti-idiotypic antibody can be immobilized or attached to a surface support (e.g., bead) as described above for use as a recombinant receptor stimulating agent against cells expressing the recombinant receptor (e.g., CAR) targeted by the anti-idiotypic antibody.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

The term “anti-idiotype antibody” refers to an antibody, including antigen-binding fragments thereof, that specifically recognizes, is specifically targeted to, and/or specifically binds to an idiotope of an antibody, such as an antigen-binding fragment. The idiotopes of an antibody may include, but are not necessarily limited to, residues within one or more of complementarity determining region(s) (CDRs) of the antibody, variable regions of the antibody, and/or partial portions or portions of such variable regions and/or of such CDRs, and/or any combination of the foregoing. The CDR may be one or more selected from the group consisting of CDR-H1, CDR-H2, CDR-H3, CDR-L1, CDR-L2, and CDR-L3. The variable regions of the antibody may be heavy chain variable regions, light chain variable regions, or a combination of the heavy chain variable regions and the light chain variable regions. The partial fragments or portions of the heavy chain variable regions and/or the light chain variable regions of the antibody may be fragments including 2 or more, 5 or more, or 10 or more contiguous amino acids, for example, from about 2 to about 100, from about 5 to about 100, from about 10 to about 100, from about 2 to about 50, from about 5 to about 50, or from about 10 to about 50 contiguous amino acids within the heavy chain variable regions or the light chain variable regions of the antibody; the idiotope may include multiple non-contiguous stretches of amino acids. The partial fragments of the heavy chain variable regions and the light chain variable regions of the antibody may be fragments including 2 or more, 5 or more, or 10 or more contiguous amino acids, for example, from about 2 to about 100, from about 5 to about 100, from about 10 to about 100, from about 2 to about 50, from about 5 to about 50, or from about 10 to about 50 contiguous amino acids within the variable regions, and in some embodiments contain one or more CDRs or CDR fragments. The CDR fragments may be consecutive or non-consecutive 2 or more, or 5 or more amino acids within the CDR. Therefore, the idiotopes of the antibody may be from about 2 to about 100, from about 5 to about 100, from about 10 to about 100, from about 2 to about 50, from about 5 to about 50, or from about 10 to about 50 contiguous amino acids containing one or more CDR or one or more CDR fragments within the heavy chain variable regions or the light chain variable regions of the antibody. In another embodiment, the idiotopes may be a single amino acid which is located at the variable regions of the antibody, for example, CDR sites.

In some embodiments, the idiotope is any single antigenic determinant or epitope within the variable portion of an antibody. In some cases it can overlap the actual antigen-binding site of the antibody, and in some cases it may comprise variable region sequences outside of the antigen-binding site of the antibody. The set of individual idiotopes of an antibody is in some embodiments referred to as the “idiotype” of such antibody.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), A1-Lazikani et al., (1997) JMB 273, 927-948 (“Chothia” numbering scheme), MacCallum et al., J. Mol. Biol. 262: 732-745 (1996), “Antibody-antigen interactions: Contact analysis and binding site topography,” J. Mol. Biol. 262, 732-745.” (“Contact” numbering scheme), Lefranc M P et al., “IMGT unique numbering for immunoglobulin and T cell receptor variable domains and Ig superfamily V-like domains,” Dev Comp Immunol, 2003 January; 27(1): 55-77 (“IMGT” numbering scheme), and Honegger A and Plückthun A, “Yet another numbering scheme for immunoglobulin variable domains: an automatic modeling and analysis tool,” J Mol Biol, 2001 Jun. 8; 309(3): 657-70, (“Aho” numbering scheme).

The boundaries of a given CDR or FR may vary depending on the scheme used for identification. For example, the Kabat scheme is based structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Table 1, below, lists exemplary position boundaries of CDR-L1, CDR-L2, CDR-L3 and CDR-H1, CDR-H2, CDR-H3 as identified by Kabat, Chothia, and Contact schemes, respectively. For CDR-H1, residue numbering is listed using both the Kabat and Chothia numbering schemes. FRs are located between CDRs, for example, with FR-L1 located between CDR-L1 and CDR-L2, and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | Contact |
|---|---|---|---|
| CDR-L1 | L24--L34 | L24--L34 | L30--L36 |
| CDR-L2 | L50--L56 | L50--L56 | L46--L55 |
| CDR-L3 | L89--L97 | L89--L97 | L89--L96 |
| CDR-H1 | H31--H35B | H26--H32 . . . 34 | H30--H35B |
| (Kabat Numbering¹) | | | |
| CDR-H1 | H31--H35 | H26--H32 | H30--H35 |
| (Chothia Numbering²) | | | |
| CDR-H2 | H50--H65 | H52--H56 | H47--H58 |
| CDR-H3 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., “CDR-H1, CDR-H2), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given V_{H} or V_{L} amino acid sequence, it is understood that such a CDR has a sequence of the corresponding CDR (e.g., CDR-H3) within the variable region, as defined by any of the aforementioned schemes. In some embodiments, specified CDR sequences are specified.

Likewise, unless otherwise specified, a FR or individual specified FR(s) (e.g., FR-H1, FR-H2), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, or Contact method. In other cases, the particular amino acid sequence of a CDR or FR is given.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150: 880-887 (1993); Clarkson et al., Nature 352: 624-628 (1991).

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv); and multispecific antibodies formed from antibody fragments. In particular embodiments, the antibodies are single-chain antibody fragments comprising a variable heavy chain region and/or a variable light chain region, such as scFvs.

Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all framework regions (FRs) amino acid residues are derived from human FRs. In some embodiments, the humanized forms of a non-human antibody, e.g., a murine antibody, are chimeric antibodies that contain minimal sequences derived from non-human immunoglobulin. In certain embodiments, the humanized antibodies are antibodies from non-human species having one or more complementarily determining regions (CDRs) from the non-human species and a framework region (FR) from a human immunoglobulin molecule. In some embodiments, a humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity. (See, e.g., Queen, U.S. Pat. No. 5,585,089 and Winter, U.S. Pat. No. 5,225,539.) Such chimeric and humanized monoclonal antibodies can be produced by recombinant DNA techniques known in the art.

In certain embodiments, a humanized antibody is a human immunoglobulin (recipient antibody) in which residues from a heavy chain variable region of the recipient are replaced by residues from a heavy chain variable region of a non-human species (donor antibody) such as mouse, rat, rabbit, or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. In some embodiments, a nucleic acid sequences encoding human variable heavy chains and variable light chains are altered to replace one or more CDR sequences of the human (acceptor) sequence by sequence encoding the respective CDR in the nonhuman antibody sequence(donor sequence). In some embodiments, the human acceptor sequence may comprise FR derived from different genes. In particular embodiments, a humanized antibody will contain substantially all of at least one, and typically two, variable domains, in which all or substantially all of the hypervariable loops correspond to those of a non-human immunoglobulin, and all or substantially all of the FRs are those of a human immunoglobulin sequence. In some embodiments, the humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409, incorporated by reference herein. In some embodiments, provided herein are humanized anti-idiotype antibodies.

In particular embodiments, an antibody, e.g., an anti-idiotype antibody, is humanized. In certain embodiments, the antibody is humanized by any suitable known means. For example, in some embodiments, a humanized antibody can have one or more amino acid residues introduced into it from a source which is non-human. These non-human amino acid residues are often referred to as “import” residues, which are typically taken from an “import” variable domain. In particular embodiments, humanization can be essentially performed by following the method of Winter and co-workers (Jones et al. (1986) Nature 321:522-525; Riechmann et al. (1988) Nature 332:323-327; Verhoeyen et al. (1988) Science 239:1534-1536), such as by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In certain embodiments, the humanized antibody is a human antibody in which some hypervariable region residues and possibly some FR residues are substituted by residues from analogous sites in rodent antibodies.

Sequences encoding full length antibodies can be subsequently obtained by joining the rendered variable heavy and variable light chain sequences to human constant heavy chain and constant light chain regions. Suitable human constant light chain sequences include kappa and lambda constant light chain sequences. Suitable human constant heavy chain sequences include IgG1, IgG2 and sequences encoding IgG1 mutants which have rendered immune-stimulating properties. Such mutants may have a reduced ability to activate complement and/or antibody dependent cellular cytotoxicity and are described in U.S. Pat. No. 5,624,821; WO 99/58572, U.S. Pat. No. 6,737,056. A suitable constant heavy chain also includes an IgG1 comprising the substitutions E233P, L234V, L235A, A327G, A330S, P331S and a deletion of residue 236. In another embodiment, the full length antibody comprises an IgA, IgD, IgE, IgM, IgY or IgW sequence.

Suitable human donor sequences can be determined by sequence comparison of the peptide sequences encoded by the mouse donor sequences to a group of human sequences, preferably to sequences encoded by human germ line immunoglobulin genes or mature antibody genes. A human sequence with a high sequence homology, preferably with the highest homology determined may serve as the acceptor sequence to for the humanization process.

In addition to the exchange of human CDRs for mouse CDRs, further manipulations in the human donor sequence may be carried out to obtain a sequence encoding a humanized antibody with optimized properties (such as affinity of the antigen).

Furthermore the altered human acceptor antibody variable domain sequences may also be rendered to encode one or more amino acids (according to the Kabat numbering system) of position 4, 35, 38, 43, 44, 46, 58, 62, 64, 65, 66, 67, 68, 69, 73, 85, 98 of the light variable region and 2, 4, 36, 39, 43, 45, 69, 70, 74, 75, 76, 78, 92 of the heavy variable region corresponding to the non-human donor sequence (Carter and Presta, U.S. Pat. No. 6,407,213)

In particular embodiments, it is generally desirable that antibodies be humanized with retention of high affinity for the antigen and other favorable biological properties. To achieve this goal, in some embodiments, the humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and imported sequences so that the desired antibody characteristic, such as increased affinity for the target antigen(s), is achieved. In general, the hypervariable region residues are directly and most substantially involved in influencing antigen binding.

In particular embodiments, choice of human variable domains, both light and heavy, to be used in making the humanized antibodies can be important to reduce antigenicity. According to the so-called “best-fit” method, the sequence of the variable domain of a rodent antibody is screened against the entire library of known human variable-domain sequences. The human sequence which is closest to that of the rodent is then accepted as the human framework for the humanized antibody. See, e.g., Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies. See, e.g., Carter et al. (1992) Proc. Natl. Acad. Sci. USA, 89:4285; Presta et al. (1993) J. Immunol., 151:2623.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

2. Target-Expressing Cells

In some embodiments, the recombinant receptor stimulating agent is a cell that expresses the target recognized by the antigen receptor, i.e. the recombinant receptor stimulating agents is a target-expressing cells. In some embodiments, the target is an antigen of the recombinant receptor and thus, in some cases, the target-expressing cells are antigen-expressing cells. In some embodiments, the recombinant receptor stimulating agent is an antigen-expressing cell, such as a cell expressing an antigen as described above.

In certain embodiments, the cells, e.g., target-expressing cells, such as antigen-expressing cells are exogenous, heterologous, and/or autologous to a subject. In some embodiments, the cells are exogenous to the subject.

In certain embodiments, the target-expressing cells, express a target that is bound by and/or recognized by the recombinant receptor. In some embodiments, the target is an antibody and the target-expressing cells express the antibody. In some embodiments, the target-expressing cells are tumor cells. In particular embodiments, the target-expressing cells are primary cells.

In some embodiments, the target is an antigen recognized by the recombinant receptor and the target-expressing cells are antigen-expressing cells. In certain embodiments, the antigen-expressing cells, express an antigen that is bound by and/or recognized by the recombinant receptor. In some embodiments, the antigen-expressing cells are tumor cells. In particular embodiments, the antigen-expressing cells are primary cells. In some embodiments, the cell line is an immortal cell line. In particular embodiments, the antigen expressing cells are cancerous cells and/or tumor cells. In some embodiments, the antigen-expressing cells are derived from a cancer cell and/or a tumor cells, e.g., human cancer cells and/or human tumor cells. In some embodiments, the antigen-expressing cells are cells from a cancer cell line, optionally a human cancer cell line. In some embodiments, the antigen-expressing cells are cell from a tumor cell line, optionally a human tumor cell line.

In particular embodiments, the antigen-expressing cells are tumor cells. In some embodiments, the antigen-expressing cells are circulating tumor cells, e.g., neoplastic immune cells such as neoplastic B cells (or cells derived from neoplastic B cells).

In particular embodiments, the antigen-expressing cells express an integrin (avb6 integrin), B cell maturation antigen (BCMA), B7-H3, B7-H6, carbonic anhydrase 9 (CA9, also known as CAIX or G250), a cancer-testis antigen, cancer/testis antigen IB (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD 133, CD13 8, CD171, chondroitin sulfate proteoglycan 4 (CSPG4), epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, fetal acetylcholine receptor, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-AIA1), Human leukocyte antigen A2 HLA-A2), IL-22 receptor alpha (IL-22Ra), IL-13 receptor alpha 2 (IL-13Ra2), kinase insert domain receptor (kdr), kappa light chain, L I cell adhesion molecule (LI-CAM), CE7 epitope of L I-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (KG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome, tautomerase, dopachrome deltaisomerase or DCT), vascular endothelial growth factor receptor (VEGFR), vascular endothelial growth factor receptor 2 (VEGFR2), Wilms Tumor 1 (WT-1), or a combination thereof. In some embodiments, the antigen-expressing cells express a pathogen-specific or pathogen-expressed antigen, or an antigen associated with a universal tag, and/or biotinylated molecules, and/or molecules expressed by HIV, HCV, HBV or other pathogens. In particular embodiments, the antigen expressing cells express one or more antigens associated with a B cell malignancy, such as any of a number of known B cell markers. In certain embodiments, the antigen-expressing cells express CD20, CD19, CD22, ROR1, CD45, CD21, CD5, CD33, Igkappa, Iglambda, CD79a, CD79b, CD30 or a combination thereof. In some embodiments, the antigen expression-cells express CD19, e.g., human CD19.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens. In certain embodiments, the antigen-expressing cells are, or are derived from, a tumor cell. In some embodiments, the tumor cell is cancerous. In particular embodiments the tumor cells is non-cancerous. In some embodiments, the tumor cell is or is derived a circulating B cell, such as a circulating B cell capable of forming a tumor in vivo. In some embodiments, the tumor cell is or is derived from a circulating B cell that is a neoplastic, tumorigenic, or cancerous B cell.

In certain embodiments, the tumor cell is, or is derived from, a human cancer cell. In some embodiments, the tumor cell is derived from a cell of a(n) AIDS-related cancer, a breast cancer, a cancer of the digestive/gastrointestinal tract, an anal cancer, an appendix cancer, a bile duct cancer, a colon cancer, a colorectal cancer, an esophageal cancer, a gallbladder cancer, islet cell tumors, pancreatic neuroendocrine tumors, a liver cancer, a pancreatic cancer, a rectal cancer, a small intestine cancer, a stomach (gastric) cancer, an endocrine system cancer, an adrenocortical carcinoma, a parathyroid cancer, a pheochromocytoma, a pituitary tumor, a thyroid cancer, an eye cancer, an intraocular melanoma, a retinoblastoma, a bladder cancer, a kidney (renal cell) cancer, a penile cancer, a prostate cancer, a transitional cell renal pelvis and ureter cancer, a testicular cancer, a urethral cancer, a Wilms' tumor or other childhood kidney tumor, a germ cell cancer, a central nervous system cancer, an extracranial germ cell tumor, an extragonadal germ cell tumor, an ovarian germ cell tumor, a gynecologic cancer, a cervical cancer, an endometrial cancer, a gestational trophoblastic tumor, an ovarian epithelial cancer, a uterine sarcoma, a vaginal cancer, a vulvar cancer, a head and neck cancer, a hypopharyngeal cancer, a laryngeal cancer, a lip and oral cavity cancer, a metastatic squamous neck cancer, a nasopharyngeal cancer, an oropharyngeal cancer, a paranasal sinus and nasal cavity cancer, a pharyngeal cancer, a salivary gland cancer, a throat cancer, a musculoskeletal cancer, a bone cancer, a Ewing's sarcoma, a gastrointestinal stromal tumors (GIST), an osteosarcoma, a malignant fibrous histiocytoma of bone, a rhabdomyosarcoma, a soft tissue sarcoma, a uterine sarcoma, a neurologic cancer, a brain tumor, an astrocytoma, a brain stem glioma, a central nervous system atypical teratoid/rhabdoid tumor, a central nervous system embryonal tumors, a central nervous system germ cell tumor, a craniopharyngioma, an ependymoma, a medulloblastoma, a spinal cord tumor, a supratentorial primitive neuroectodermal tumors and pineoblastoma, a neuroblastoma, a respiratory cancer, a thoracic cancer, a non-small cell a lung cancer, a small cell lung cancer, a malignant mesothelioma, a thymoma, a thymic carcinoma, a skin cancer, a Kaposi's sarcoma, a melanoma, or a Merkel cell carcinoma, or any equivalent human cancer thereof.

In particular embodiments, the tumor cell is derived from a non-hematologic cancer, e.g., a solid tumor. In certain embodiments, the tumor cell is derived from a hematologic cancer. In certain embodiments, the tumor cell is derived from a cancer that is a B cell malignancy or a hematological malignancy. In particular embodiments, the tumor cell is derived from a non-Hodgkin lymphoma (NHL), an acute lymphoblastic leukemia (ALL), a chronic lymphocytic leukemia (CLL), a diffuse large B-cell lymphoma (DLBCL), acute myeloid leukemia (AML), or a myeloma, e.g., a multiple myeloma (MM), or any equivalent human cancer thereof. In some embodiments, the antigen-expressing cell is a neoplastic, cancerous, and/or tumorigenic B cell. Multiple tumor cell lines are known and available and can be selected depending on the antigen recognized by the particular recombinant receptor (e.g., CAR).

Any of a number of tumor cell lines are known and available. Tumor cell lines are known that express particular tumor antigens or surface expression of a tumor antigen can be readily determined or measured by as skilled artisan using any of a variety of techniques, such as by flow cytometry. Exemplary tumor cell lines include, but are not limited to, lymphoma cells (Raji; Daudi; Jeko-1; BJAB; Ramos; NCI-H929; BCBL-1; DOHH-2, SC-1, WSU-NHL, JVM-2, Rec-1, SP-53, RL, Granta 519, NCEP-1, CL-01), leukemia cells (BALL-1, RCH-ACV, SUP-B15); cervical carcinoma cells (33A; CaSki; HeLa), lung carcinoma cells (NCI-H358; A549, H1355, H1975, Calu-1, H1650 and H727), breast cells, (Hs-578T; ZR-75-1; MCF-7; MCF-7/HER2; MCF10A; MDA-MB-231; SKBR-3, BT-474, MDA-MB-231); ovarian cells (ES-2; SKOV-3; OVCAR3; HEY1B); multiple myeloma cells (U266, NCI-H929, RPMI-8226, OPM2, LP-1, L363, MM.1S, MM.1R, MC/CAR, JJN3, KMS11, AMO-1, EJM; MOLP-8). For instance, exemplary CD19-expressing cell lines include, but are not limited to, Raji, Daudi and BJAB; exemplary CD20-expressing cell lines include Daudi, Ramos and Raji; exemplary CD22-expressing cell lines include, but are not limited to, Ramos, Raji, A549, H727, and H1650; exemplary Her2-expressing cell lines include SKOV3, BT-474 and SKBR-3; exemplary BCMA-expressing cell lines include, but are not limited to, RPMI-8226, NCI-H929, MM1S, MM1R and KMS11; exemplary GPRC5D-expressing cell lines include, but are not limited to, AMO-1, EJM, NCI-H929, MM.1S, MM1.12, MOLP-8, and OPM-2; exemplary ROR1-expressing cell lines include, but are not limited to, A549, MDA-MB-231, H1975, BALL-1 and RCH-ACV.

In some embodiments, the target-expressing cell line is a cell line that has been transduced to express the target of the recombinant receptor. In some embodiments, the target is a tumor antigen. In particular embodiments, the antigen-expressing cell line is a cell line that has been transduced to express the tumor antigen. This cell line may be a mammalian cell line, including, but not limited to, human cell lines. In some embodiments, the human cell line may be K562, U937, 721.221, T2, and C1R cells. For instance, the K562 chronic myeloid leukemia cell line may be introduced with a nucleic acid encoding the tumor antigen. In some embodiments, the cell line can be engineered with plasmid vectors or messenger RNAs (mRNAs) that encode the tumor antigen of interest. In some embodiments, the introduction can be by lentivial-based transduction. In some embodiments the cell line (e.g., K562 cells) stably expresses the exogenous nucleic acid encoding the tumor antigen. In some embodiments the exogenous nucleic acid may be integrated into the genome of the cell line (e.g., K562 cell). In some embodiments the exogenous nucleic acid may be integrated into the genome of the cell line (e.g., K562 cell) at a particular locus. In some embodiments, the exogenous nucleic acid may be integrated into the genome of the cell line (e.g., K562 cell) at a genomic safe harbour (GSH). A GSH is a site which supports stable integration and expression of exogenous nucleic acid while minimising the risk of unwanted interactions with the host cell genome (see e.g., Sadelain et al., Nat Rev Cancer. (2011) 12(1):51-8). Several safe GSHs for stable integration of exogenous nucleic acid in human cells have been identified, including AAVS1, a naturally occurring site of integration of AAV virus on chromosome 19; CCR5 gene a chemokine receptor gene also known as an HIV-1 coreceptor; and the human ortholog of the mouse Rosa26 locus (see e.g., Papapetrou and Schambach Mol Ther. (2016) 24(4): 678-684).

In some embodiments, the target-expressing cells are varied or titrated across the plurality of incubation at a varied ratios compared to a fixed amount of the cells of the therapeutic composition expressing the recombinant receptor (effector cells). In some embodiments, titrated amount is from 100:1 to 0.001 ratio of target-expressing target cells to effector T cells (T:E), such as a titrated amount from 50:1 to 0.050 T:E ratio, from 25:1 to 0.025 T:E ratio, from 12:1 to 0.012:1 T:E ratio, from 10:1 to 0.010 T:E ratio or from 5:1 to 0.5 T:E ratio. In some embodiments, the ratio is or is about from a 12:1 to 0.012:1 T:E ratio. The particular range of ratios can be empirically determined depending on the particular target and the target cells being employed. For instance, the ratio chosen is one that includes a linear dose-response increase in recombinant receptor-dependent activity across the plurality of titrated amounts. In some embodiments, the ratio is chosen to also include a lower asymptote of receptor-dependent activity and an upper asymptote of receptor-dependent activity that represent a minimum and a maximum responses, respectively.

For instance, the target is an antigen of the recombinant receptor. In some embodiments, the antigen-expressing cell are varied or titrated across the plurality of incubation at a varied ratios compared to a fixed amount of the cells of the therapeutic composition expressing the recombinant receptor (effector cells). In some embodiments, titrated amount is from 100:1 to 0.001 ratio of antigen-expressing target cells to effector T cells (T:E), such as a titrated amount from 50:1 to 0.050 T:E ratio, from 25:1 to 0.025 T:E ratio, from 12:1 to 0.012:1 T:E ratio, from 10:1 to 0.010 T:E ratio or from 5:1 to 0.5 T:E ratio. In some embodiments, the ratio is or is about from a 12:1 to 0.012:1 T:E ratio. The particular range of ratios can be empirically determined depending on the particular antigen and the target cells being employed. For instance, the ratio chosen is one that includes a linear dose-response increase in recombinant receptor-dependent activity across the plurality of titrated amounts. In some embodiments, the ratio is chosen to also include a lower asymptote of receptor-dependent activity and an upper asymptote of receptor-dependent activity that represent a minimum and a maximum responses, respectively.

C. Measuring Recombinant Receptor-Dependent Activity

The methods for assessing potency provided herein include measuring activity of the therapeutic cell compositions in response to stimulation of recombinant receptors of the engineered cells of the therapeutic cell composition. As described above, the provided assays allow for measuring recombinant receptor-dependent activity in response to a recombinant receptor stimulating agent, such as described in Section I-B, from a plurality of incubating conditions, where each incubation comprises a different titrated ratio of cells of the therapeutic cell composition to the recombinant receptor stimulating agent.

In particular embodiments, it is contemplated that a recombinant receptor-dependent activity, e.g., a CAR dependent activity, is an activity that occurs in an engineered cell that expresses a recombinant receptor which does not and/or cannot occur in a cell that does not express the recombinant receptor. In some embodiments, the recombinant receptor dependent activity is an activity that depends on an activity or presence of the recombinant receptor. The recombinant receptor-dependent activity may be any cellular process that is directly or indirectly influenced by the expression and/or presence of the recombinant receptor or by a change in activity, such as receptor stimulation, of the recombinant receptor. In some embodiments, the recombinant receptor dependent activity may include, but is not limited to cellular processes such as cell division, DNA replication, transcription, protein synthesis, membrane transport, protein translocation, and/or secretion, or it may be an immune cell function, e.g., a cytolytic activity. In certain embodiments, recombinant receptor dependent activity may be measured by a change in the confirmation of the CAR receptor, the phosphorylation of an intracellular signaling molecule, degradation of a protein, transcription, translation, translocation of a protein, and/or production and secretion of a factor, such as a protein, or growth factor, cytokine. In certain embodiments, the recombinant receptor is a CAR. In certain embodiments, the recombinant receptor is a TCR.

In some embodiments, the recombinant receptor-dependent activity, e.g., a CAR dependent activity is a measurement of a factor, e.g., an amount or concentration, or a change in the amount or concentration following stimulation of the therapeutic cell composition with a recombinant receptor stimulating agent. In certain embodiments, the factor may be a protein, a phosphorylated protein, a cleaved protein, a translocated protein, a protein in an active confirmation, a polynucleotide, an RNA polynucleotide, an mRNA, and/or an shRNA. In some embodiments, the measurement may include, but is not limited to, an increase or decrease of kinase activity, protease activity, phosphatase activity, cAMP production, ATP metabolism, translocation, e.g., a nuclear localization of a protein, an increase in transcriptional activity, an increase in translational activity, production and/or secretion of a soluble factor, cellular uptake, ubiquitination, and/or protein degradation.

In some embodiments, the factor is a soluble factor that is secreted, such as a hormone, a growth factor, a chemokine, and/or a cytokine.

In some embodiments, the recombinant receptor-dependent activity, e.g., a CAR dependent activity is a response to stimulation with a recombinant receptor stimulating agent. In certain embodiments, the cells are incubated in the presence of a recombinant receptor stimulating agent able to stimulate recombinant receptor-dependent activity, and the activity is or includes at least one aspect of a response to the stimulation. A response may include, but is not limited to, an intracellular signaling event, such as an increased activity of a receptor molecule, an increased kinase activity of one or more kinases, an increase in the transcription of one or more genes, increased protein synthesis of one or more proteins, and/or an intracellular signaling molecule e.g., an increased kinase activity of a protein. In some embodiments, the response (e.g., recombinant receptor-dependent activity) is associated with an immune activity, and may include, but is not limited to, production and/or section of a soluble factor, e.g., a cytokine, an increase in antibody production, and/or an increase in cytolytic activity.

In some embodiments, the recombinant receptor-dependent activity is assessed by measuring, detecting, or quantifying recombinant receptor-dependent activity to a stimulus (e.g., recombinant receptor stimulating agent), i.e. at least one activity that is initiated, triggered, supported, prolonged, and/or caused by the stimulus (e.g., recombinant receptor stimulating agent). In certain embodiments, the cells of the therapeutic cell composition are cultured with a recombinant receptor stimulating agent, where interaction or binding of the recombinant receptor stimulating agent to the recombinant receptor stimulates, such as induces, a recombinant receptor-dependent activity that is specific to cells that express the recombinant receptor. In certain embodiments, the recombinant receptor-dependent activity occurs in cells that express the recombinant receptor, but does not occur, or only minimally occurs, in cells that do not express the receptor. In particular embodiments, the recombinant receptor is a CAR. In some embodiments, the activity is a CAR dependent activity.

The conditions under which stimulation of the recombinant receptor of the engineered cells, e.g., immune cells or T cells, by the recombinant receptor stimulating agent can include one or more of particular media, temperature, oxygen content, carbon dioxide content, time, agents, e.g., nutrients, amino acids, antibiotics, ions. In some embodiments, the recombinant receptor-dependent activity is determined by whether or not a soluble factor, e.g., a cytokine or a chemokine, is produced or secreted.

In some embodiments, the recombinant receptor-dependent activity is specific to cells that express a recombinant receptor. In some embodiments, the recombinant receptor-dependent activity is specific to cells that express a recombinant receptor, and does not occur in cells that lack expression of the recombinant receptor. In certain embodiments, the recombinant receptor is a CAR, and the activity is a CAR dependent activity. In particular embodiments, the recombinant receptor-dependent activity is not present in cells that lack expression of the recombinant receptor under the same conditions where the activity is present in cells that express the recombinant receptor. In certain embodiments, the CAR dependent activity is about 10%, about 20%, about 30%, about 40%, about 50%, about 60% about 70%, about 75%, about 80%, about 85%, about 90%, about 95%, about 97%, about 98%, about 99%, or about 99% less than the CAR dependent activity in CAR cells under the same conditions.

In some embodiments, the recombinant receptor-dependent activity is specific to cells that express a recombinant receptor, e.g., a CAR, and the activity is produced by stimulation with a recombinant receptor stimulating agent that is specific to cells of a therapeutic cell composition that express the recombinant receptor. In some embodiments, the recombinant receptor is a CAR, and a CAR specific stimulation stimulates, triggers, initiates, induces, and/or prolongs an activity in CAR+ cells, but does not stimulate, trigger, initiate, induce, and/or prolong the activity in CAR− cells. In some embodiments, the CAR dependent activity is about 10%, about 20%, about 30%, about 40%, about 50%, about 60% about 70%, about 75%, about 80%, about 85%, about 90%, about 95%, about 97%, about 98%, about 99%, or about 99% less in CAR− cells than in the CAR+ cells following stimulation by the CAR specific stimulus.

In certain embodiments, the activity is a recombinant receptor dependent, e.g., a CAR dependent activity that is stimulated by a recombinant receptor stimulating agent, such as described in Section I-B, that is specific for the recombinant receptor. In some embodiments, the recombinant receptor stimulating agent, e.g., a CAR specific agent, includes an antigen or an epitope thereof that is bound by and/or recognized by the recombinant receptor, e.g., the CAR. In some embodiments, the recombinant receptor stimulating agent includes an antibody, e.g., an anti-idiotypic antibody (anti-ID), or an active fragment, variant, or portion thereof, that binds to the recombinant receptor. In certain embodiments, the recombinant receptor stimulating agent is a cell that expresses the antigen on its surface. In certain embodiments, the recombinant receptor stimulating agent is a cell that expresses the antibody on its surface. In some embodiments, the cell is from a cell line, such as described in Section I-B-2. In some embodiments, the cell line is a tumor cell line. In some embodiments, the cell expresses a tumor antigen.

In some embodiments, the recombinant receptor-dependent activity is measured in the therapeutic cell composition containing cells expressing a recombinant receptor, e.g., a CAR, and the measurement is compared to one or more controls. In certain embodiments, the control is a similar or identical composition of cells that was not stimulated. For example, in some embodiments, the recombinant receptor-dependent activity is measured in a cell composition following or during incubation with a recombinant receptor stimulating agent, and the resulting measurement is compared to a control measurement of the activity from the similar or identical cell composition that is not incubated with the recombinant receptor stimulating agent. In some embodiments, both the therapeutic cell composition and the control cell composition contain cells that express the recombinant receptor. In some embodiments, the control is taken from a similar cell composition that does not contain cells that express the recombinant receptor, e.g., CAR+ cells. Thus, in some embodiments, a therapeutic cell composition that contains recombinant receptor expressing cells and a control cell composition that does not contain recombinant receptor expressing cells are contacted with a recombinant receptor stimulating agent. In certain embodiments, the control is a measurement from the same cell composition that expresses a recombinant receptor that is taken prior to any stimulation. In certain embodiments, a control measurement is obtained to determine a background signal, and control measurement is subtracted from the measurement of the activity. In some embodiments, the measurement of the activity in the cell composition is divided by the control measurement, to obtain a value that is a ratio of the activity over a control level. In some embodiments, all recombinant receptor-dependent activity measurements are adjusted or normalized to a control measurement, for example where a recombinant receptor stimulating agent was not cultured with cells of the therapeutic cell composition. In some embodiments, adjustment or normalization of the measurements to a control condition provides a more accurate measure of the recombinant receptor-dependent activity.

In particular embodiments, the recombinant receptor-dependent activity is or includes the production and/or secretion of a soluble factor. In some embodiments, the recombinant receptor-dependent activity, e.g., a CAR, dependent activity, is or includes the production and/or secretion of a soluble factor. In certain embodiments, the soluble factor is a cytokine or a chemokine.

Suitable techniques for the measurement of the production or secretion of a soluble factor are known in the art. Production and/or secretion of a soluble factor can be measured by determining the concentration or amount of the extracellular amount of the factor, or determining the amount of transcriptional activity of the gene that encodes the factor. Suitable techniques include, but are not limited to assays such as an immunoassay, an aptamer-based assay, a histological or cytological assay, an mRNA expression level assay, an enzyme linked immunosorbent assay (ELISA), immunoblotting, immunoprecipitation, radioimmunoassay (RIA), immunostaining, flow cytometry assay, surface plasmon resonance (SPR), chemiluminescence assay, lateral flow immunoassay, inhibition assay or avidity assay, protein microarrays, high-performance liquid chromatography (HPLC), Meso Scale Discovery (MSD) electrochemiluminescence and bead based multiplex immunoassays (MIA). In some embodiments, the suitable technique may employ a detectable binding reagent that specifically binds the soluble factor.

In particular embodiments, the measurement of the soluble factor, e.g., cytokine, is measured by ELISA (enzyme-linked immunosorbent assay). ELISA is a plate-based assay technique designed for detecting and quantifying substances such as peptides, cytokines, antibodies and hormones. In an ELISA, the soluble factor must be immobilized to a solid surface and then complexed with an antibody that is linked to an enzyme. Detection is accomplished by assessing the conjugated enzyme activity via incubation with a substrate to produce a detectable signal. In some embodiments, the recombinant receptor-dependent activity is measured with an ELISA assay.

In some embodiments, the recombinant receptor-dependent activity is a secretion or production of the soluble factor (e.g., cytokine). In certain embodiments, production or secretion is stimulated in a therapeutic cell composition that contains recombinant receptor expressing cells, e.g., CAR expressing cells, by a recombinant receptor stimulating agent capable of binding to the recombinant receptor to stimulate a recombinant receptor-dependent activity, e.g., a CAR-dependent activity. In some embodiments, the recombinant receptor stimulating agent includes an antigen or an epitope thereof that is specific to the recombinant receptor; is a cell that expresses the antigen; or includes an antibody or a portion or variant thereof that binds to and/or recognizes the recombinant receptor; or a combination thereof (see e.g., Section I-B above). In certain embodiments, the recombinant receptor stimulating agent is a recombinant protein that comprises the antigen or epitope thereof that is bound by or recognized by the recombinant receptor.

In certain embodiments, the recombinant receptor-dependent activity is a soluble factor production and/or secretion, which is measured by incubating the therapeutic cell composition that contains cells expressing the recombinant receptor, e.g., a CAR, with a recombinant receptor stimulating agent, such as described in Section I-B. In certain embodiments, the soluble factor is a cytokine or a chemokine. In some embodiments, cells of the therapeutic cell composition that contain recombinant receptor expressing cells are incubated in the presence of a recombinant receptor stimulating agent for an amount of time, and the production and/or secretion of the soluble factor is measured at one or more time points during the incubation. In some embodiments, the cells are incubated with the recombinant receptor stimulating agent for up to or about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 19 hours, about 20 hours, about 21 hours, about 22 hours, about 23 hours, about 24 hours, about 48 hours, or for a duration of time between 1 hour and 4 hours, between 1 hour and 12 hours, between 12 hours and 24 hours, each inclusive, or for more than 24 hours and the amount of a soluble factor, e.g., a cytokine is detected.

In some embodiments, the recombinant receptor stimulating agent is a particle (e.g., bead) that is attached or immobilized with an antigen or portion thereof recognized by the recombinant receptor or with an antibody, e.g., an anti-idiotypic antibody, specific to an extracellular domain (e.g., an extracellular antigen-binding domain (e.g., scFv)) of the recombinant receptor. In some embodiments, the recombinant receptor (e.g., a CAR) and a constant number of the cells of the therapeutic cell composition are incubated with the particles at a plurality of ratios of cells of the therapeutic cell composition to the particles, such as including at or about 1:100, 1:75, 1:50, 1:40, 1:30, 1:20, 1:15, 1:14, 1:13, 1:12, 1:11, 1:10, 1:9, 1:8, 1:7, 1:6, 1:5, 1:4, 1:3, 1:2, 1:1, 1:0.5, 1:0.4, 1:0.3, 1:0.2, or 1:0.1, ora range between any of the foregoing, such as at a ratio between 1:1 and 1:10 or 1:0.2 to 1:12, each inclusive. In some embodiments, the plurality of ratios includes any or all of the ratios provided herein. In some embodiments, the recombinant receptor stimulating agent is a cell that expresses an antigen recognized by the recombinant receptor. In some embodiments, the recombinant receptor is a CAR, and a titrated number of the cells of the therapeutic cell composition are incubated with a constant number of such particles at a plurality of ratios of cells of the therapeutic cell composition to the particles, such as at or about 1:100, 1:75, 1:50, 1:40, 1:30, 1:20, 1:15, 1:14, 1:13, 1:12, 1:11, 1:10, 1:9, 1:8, 1:7, 1:6, 1:5, 1:4, 1:3, 1:2, 1:1, 1:0.5, 1:0.4, 1:0.3, 1:0.2, or 1:0.1, or a range between any of the foregoing, such as at a ratio between 1:1 and 1:10 or 1:0.2 to 1:12, each inclusive. In some embodiments, the plurality of ratios includes any or all of the ratios provided herein.

In some embodiments, the recombinant receptor stimulating agent is a cell that expresses a target (e.g., an antigen or antibody) recognized by the recombinant receptor. In some embodiments, the recombinant receptor (e.g., a CAR) and a constant number of the cells of the therapeutic cell composition are incubated with the cells at a plurality of ratios of cells of the therapeutic cell composition to the cells expressing the target (e.g., antigen or antibody) including at or about 1:100, 1:75, 1:50, 1:40, 1:30, 1:20, 1:15, 1:14, 1:13, 1:12, 1:11, 1:10, 1:9, 1:8, 1:7, 1:6, 1:5, 1:4, 1:3, 1:2, 1:1, 1:0.5, 1:0.4, 1:0.3, 1:0.2, or 1:0.1, or a range between any of the foregoing, such as at a ratio between 1:1 and 1:10 or 1:0.2 to 1:12, each inclusive. In some embodiments, the plurality of ratios includes any or all of the ratios provided herein. In some embodiments, the recombinant receptor is a CAR, and a titrated number of the cells of the therapeutic cell composition are incubated with a constant number of cells expressing target (e.g., antigen or antibody) at a plurality of ratios of cells of the therapeutic cell composition to the cells expressing the antigen including at or about 1:100, 1:75, 1:50, 1:40, 1:30, 1:20, 1:15, 1:14, 1:13, 1:12, 1:11, 1:10, 1:9, 1:8, 1:7, 1:6, 1:5, 1:4, 1:3, 1:2, 1:1, 1:0.5, 1:0.4, 1:0.3, 1:0.2, or 1:0.1, or a range between any of the foregoing, such as at a ratio between 1:1 and 1:10 or 1:0.2 to 1:12, each inclusive. In some embodiments, the plurality of ratios includes any or all of the ratios provided herein.

In some embodiments, between about 1×10² and about 1×10⁴, between about 1×10³ and about 1×10⁵, between about 1×10⁴ and about 1×10⁶, between about 1×10⁵ and about 1×10⁷, between about 1×10⁶ and about 1×10⁸, between about 1×10⁷ and about 1×10⁹, and between about 1×10⁸ and about 1×10¹⁰ cells of the cell composition, each inclusive, are incubated with a constant amount or concentration of recombinant receptor stimulating agent.

In some embodiments, the cells of the therapeutic cell composition are incubated with the recombinant receptor stimulating agent, in a volume of cell media. It is understood that the precise volume can be empirically determined and is a function of the surface area of the vessel (e.g., multiwall plate) in which the assay is being carried out. In certain embodiments, the cells are incubated with the recombinant receptor stimulating agent in a volume of at least or about 1 μL, at least or about 10 μL, at least or about 25 μL, at least or about 50 μL, at least or about 100 μL, at least or about 500 μL, at least or about 1 mL, at least or about 1.5 mL, at least or about 2 mL, at least or about 2.5 mL, at least or about 5 mL, at least or about 10 mL, at least or about 20 mL, at least or about 25 mL, at least or about 50 mL, at least or about 100 mL, or greater than 100 mL. In certain embodiments, the cells are incubated with the recombinant receptor stimulating agent in a volume that falls between about 1 μL, and about 100 μL, between about 100 μL and about 500 μL, between about 500 μL, and about 1 mL, between about 500 μL, and about 1 mL, between about 1 mL and about 10 mL, between about 10 mL and about 50 mL, or between about 10 mL and about 100 mL, each inclusive. In certain embodiments, the cells are incubated with the recombinant receptor stimulating agent in a volume of between about 100 μL and about 1 mL, inclusive. In particular embodiments, the cells are incubated with the recombinant receptor stimulating agent in a volume of about 500 μL. In some embodiments, the multi-well plate is a 6-well plate and the volume is at or about 1 mL to at or about 3 mL. In some embodiments, the multi-well plate is a 12-well plate and the volume is at or about 1 mL to at or about 2 mL. In some embodiments, the multi-well plate is a 24-well plate and the volume is at or about 0.5 mL to at or about 1 mL. In some embodiments, the multi-well plate is a 48-well plate and the volume is at or about 0.2 mL to at or about 0.4 mL. In some embodiments, the multi-well plate is a 96-well plate and the volume is at or about 0.1 mL to at or about 0.2 mL.

In some embodiments, a constant number of cells of the therapeutic cell composition are incubated with a concentration of recombinant receptor stimulating agent that varies between about 1 fmol and about 1 pmol, between about 1 pmol and about lnmol, between about 1 nmol and about 1 between about 1 pmol and about 1 mmol, or between about 1 mmol and 1 mol, each inclusive. In particular embodiments, a constant number of cells of the therapeutic cell composition are incubated with a concentration of recombinant receptor stimulating agnet that varies between about 1 fM and about 1 pM, between about 1 pM and about 1 nM, between about 1 nM and about 1 μM, between about 1 μM and about 1 mM, or between about 1 mM and 1 mol, each inclusive. Exemplary units include, but are not limited to pg/mL, pg/(mL/hr), pg(mL×cell), pg/(mL×hr×cell), and pg/(mL×hr×10⁶ cells).

In certain embodiments, the measurement of the recombinant receptor-dependent activity, e.g., the CAR-dependent activity is the amount or concentration, or a relative amount or concentration, of the soluble factor in the therapeutic cell composition at a time point during or at the end of the incubation for each of the plurality of ratios tested. In particular embodiments, the measurement is subtracted by or normalized to a control measurement. In some embodiments, the control measurement is a measurement from the same cell composition taken prior to the incubation. In particular embodiments the control measurement is a measurement taken from an identical control cell composition that was not incubated with the binding molecule. In certain embodiments, the control is a measurement taken at an identical time point during incubation with the bind molecule from a cell composition that does not contain recombinant receptor positive cells.

In some embodiments, the measurement is a normalized ratio of the amount or concentration as compared to the control. In particular embodiments, the measurement is the amount or concentration of the soluble factor per an amount of time, e.g., per minute or per hour. In some embodiments, the measurement is an amount or concentration of the soluble factor per cell or per a set or reference number of cells, e.g., per 100 cells, per 10³ cells, per 10⁴ cells, per 10⁵ cells, per 10⁶ cells, etc. In certain the measurement is the amount or concentration of the soluble factor per an amount of time, per cell or per reference number of cells. In some embodiments, the measurement is the amount or concentration of the soluble factor per cell that expresses the recombinant receptor. In certain embodiments, the measurement is the amount or concentration of the soluble factor per amount of time (e.g., per minute or per hour) per cell that expresses the recombinant receptor, CAR+ cell, of the therapeutic cell composition. In some embodiments, the measurement is the amount or concentration of the soluble factor per an amount of time per amount or concentration of the recombinant receptor or recombinant receptor stimulating agent. In some embodiments, the measurement is an amount or concentration of the soluble factor per cell or per a set or reference number of cells per amount or concentration of the recombinant receptor stimulating agetn. In some embodiments, the measurement is the amount or concentration of the soluble factor per an amount of time, per amount or concentration of the recombinant receptor or recombinant receptor stimulating agent, per cell or per reference number of cells. In some embodiments, the measurement is the amount or concentration of the soluble factor per amount or concentration of the recombinant receptor or recombinant receptor stimulating agent, per cell that expresses the recombinant receptor. In certain embodiments, the measurement is the amount or concentration of the soluble factor per amount of time, per amount or concentration of the recombinant receptor or recombinant receptor stimulating agent, per amount of CAR+ cells of the therapeutic cell composition.

In particular embodiments, the recombinant receptor- or CAR-dependent activity is the production or secretion of two or more soluble factors. In certain embodiments, the recombinant receptor- or CAR-dependent activity is the production or secretion of two, three, four, five, six, seven, eight, nine, ten, or more than ten soluble factors. In some embodiments, the measurements of the two, three, four, five, six, seven, eight, nine, ten, or more than ten soluble factors are combined into an arithmetic mean or a geometric mean. In certain measurements, measurement of the recombinant receptor-dependent activity is the secretion of or composites of two, three, four, five, six, seven, eight, nine, ten, or more than ten soluble factors.

In particular embodiments, the measurement of the recombinant receptor-dependent activity is transformed, e.g., by a logarithmic transformation. In certain embodiments, the measurement of the recombinant receptor activity is transformed by a common log (log₁₀(x)), a natural log (ln(x)) or a binary log (log₂(x)). In some embodiments, the measurement of the recombinant receptor-dependent activity is a composite of measurement of the production or secretion of two more soluble factors. In some embodiments, two or more measurements of production or secretion of soluble factors are transformed prior to being combined into a composite measurement. In particular embodiments, the measurement of the recombinant receptor-dependent activity is transformed prior to normalization to a reference measurement. In certain embodiments, the measurement of the recombinant receptor-dependent activity is transformed prior to normalization to a reference measurement. In some embodiments, normalization of the recombinant receptor-dependent activity is to maximum recombinant receptor-dependent activity measured from the plurality of incubations.

In certain embodiments, the soluble factor is a cytokine. Cytokines are a large group of small signaling molecules that function extensively in cellular communication. Cytokines are most often associated with various immune modulating molecules that include interleukins, chemokines, and interferons. Alternatively cytokines may be characterized by their structure, which are categorized in four families, the four alpha helix family that includes the IL-2 subfamily, the IFN subfamily, and the IL-10 subfamily; the IL-1 family, the IL-17 family, and cysteine-knot cytokines that include members of the transforming growth factor beta family. In some embodiments, the recombinant receptor-dependent activity is the production or secretion of one or more soluble factors that include interleukins, interferons, and chemokines. In particular embodiments, the recombinant receptor-dependent activity, e.g., CAR-dependent activity is the production or secretion of one or more of an IL-2 family member, an IFN subfamily member, an IL-10 subfamily member; an IL-1 family member, an IL-17 family member, a cysteine-knot cytokine, and/or a member of the transforming growth factor beta family.

In particular embodiments, the recombinant receptor-dependent or CAR-dependent activity is the production and/or secretion of one or more of IL-1, IL-1(3, IL-2, sIL-2Ra, IL-3, IL-5, IL-6, IL-7, IL-8, IL-10, IL-12, IL-13, IL 27, IL-33, IL-35, TNF, TNF alpha, CXCL2, CCL2, CCL3, CCL5, CCL17, CCL24, PGD2, LTB4, interferon gamma (IFN-γ), granulocyte macrophage colony stimulating factor (GM-CSF), macrophage inflammatory protein (MIP)-1a, MIP-1b, Flt-3L, fracktalkine, and/or IL-5. In certain embodiments, the CAR dependent activity production or secretion of a Th17 cytokine. In some embodiments, the Th17 cytokine is GMCSF. In some embodiments, the CAR dependent activity comprises production or secretion of a Th2 cytokine, wherein the Th2 cytokine is IL-4, IL-5, IL-10, or IL-13.

In certain embodiments, the recombinant receptor- or CAR-dependent activity is the production or secretion of a proinflammatory cytokine. Proinflammatory cytokines play a role in initiating the inflammatory response and to regulate the host defense against pathogens mediating the innate immune response. Proinflammatory cytokines include, but are not limited to, interleukins (IL), interleukin-1-beta (IL-1), interleukin-3 (IL-3), interleukin-5 (IL-5), interleukin-6 (IL-6), interleukin-13 (IL-13), tumor necrosis factor (TNF), CXC-chemokine ligand 2 (CXCL2), CC-chemokine ligand 2 (CCL2), CC-chemokine ligand 3 (CCL3), CC-chemokine ligand 5 (CCL5), CC-chemokine ligand 17 (CCL17), CC-chemokine ligand 24 (CCL24), prostaglandin D2 (PGD2) and leukotriene B4 (LTB4) as well as IL-33.). In some embodiments, the recombinant receptor- or CAR-dependent activity is production and or secretion of an interleukin and/or a TNF family member. In particular embodiments, the recombinant receptor- or CAR-dependent activity is production and or secretion of IL-1, IL-6, IL-8, and IL-18, TNF-alpha or a combination thereof.

In particular embodiments, the recombinant receptoractivity (e.g. CAR-dependent activity) is secretion of IL-2, IFN-gamma, TNF-alpha or a combination thereof. In some embodiments, the recombinant receptor activity (e.g. CAR-dependent activity) is secretion of IL-2. In some embodiments, the recombinant receptor activity (e.g. CAR-dependent activity) is secretion of IFN-gamma. In some embodiments, the recombinant receptor activity (e.g. CAR-dependent activity) is secretion of TNF-alpha.

In particular embodiments, the recombinant receptor-dependent activity is cytolytic (cytotoxic) activity of the therapeutic cell composition. In some embodiments, recombinant receptor-dependent cytolytic activity is assessed by exposing, incubating, and/or contacting cells expressing the recombinant receptor, or a cell composition containing cells that express the recombinant receptor, with a varying amount of target cells that express the antigen and/or an epitope that is bound by and/or recognized by the recombinant receptor. The cytolytic activity can be measured by directly or indirectly measuring the target cell number over time. For example, the target cells may be incubated with a detectable marker prior to being incubated with recombinant receptor expressing cells, such a marker that is detectable then the target cell is lysed, or a detectable marker that is detectable in viable target cells. These readouts provide direct or indirect of target cell number and/or target cell death, and can be measured at different time points during the assay. A reduction of target cell number and/or an increase of target cell death indicate the cytolytic activity of the cells. Suitable methods for performing cytolytic assays are known in the art, and include, but are not limited to chromium-51 release assays, non-radioactive chromium assays, flow cytometric assays that use fluorescent dyes such as carboxyfluorescein succinimidyl ester (CFSE), PKH-2, and PKH-26.

In certain embodiments, the recombinant receptor—, e.g., CAR, dependent cytolytic activity is measured by incubating the cell composition that contains cells expressing the recombinant receptor with target cells that express an antigen or an epitope thereof the is bound by or recognized by the recombinant receptor. In certain embodiments, the recombinant receptor is a CAR. In some embodiments, the cells of the therapeutic cell composition are incubated with cells expressing antigen at ratios including 10:1, about 5:1, about 4:1, about 3:1, about 2:1, about 1:1, about 1:2, about 1:3, about 1:4, about 1:5, about 1:6, about 1:7, about 1:8, about 1:9, or about 1:10, or at a ratio between 10:1 and 1:1, 3:1 and 1:3, or 1:1 and 1:10, each inclusive. In some embodiments, the cells of the cell composition are incubated with the target cells at ratios of CAR+ cells of the therapeutic cell composition to target cells including about 10:1, about 5:1, about 4:1, about 3:1, about 2:1, about 1:1, about 1:2, about 1:3, about 1:4, about 1:5, about 1:6, about 1:7, about 1:8, about 1:9, or about 1:10, or at a ratio between 10:1 and 1:1, 3:1 and 1:3, or 1:1 and 1:10, each inclusive.

In certain embodiments, cells of the therapeutic cell composition are incubated with the target cells for up to or about 1 hour, about 2 hours, about 3 hours, about 4 hours, about 5 hours, about 6 hours, about 8 hours, about 12 hours, about 18 hours, about 24 hours, about 48 hours, or greater than 48 hours. In some embodiments, a constant number of cells of the therapeutic cell composition are incubated with the cells expressing antigen for about 18 hours, 19 hours, 20 hours, 21 hours, 22 hours, 23 hours, or 24 hours. In some embodiments, a constant number of cells between about 1×10² and about 1×10⁴, between about 1×10³ and about 1×10⁵, between about 1×10⁴ and about 1×10⁶, between about 1×10⁵ and about 1×10⁷, between about 1×10⁶ and about 1×10⁸, between about 1×10⁷ and about 1×10⁹, or between about 1×10⁸ and about 1×10¹⁰ cells of the therapeutic cell composition, each inclusive, are incubated with a varying number of the antigen-expressing cells to generate a plurality of ratios. In certain embodiments, a constant amount of cells between about 1×10² and about 1×10⁴, between about 1×10³ and about 1×10⁵, between about 1×10⁴ and about 1×10⁶, between about 1×10⁵ and about 1×10⁷, between about 1×10⁶ and about 1×10⁸, between about 1×10⁷ and about 1×10⁹, or between about 1×10⁸ and about 1×10¹⁰ CAR+ cells of the therapeutic cell composition, each inclusive, are incubated with a varying number of antigen-expressing cells to generate a plurality of ratios.

In some embodiments, the measurement of the activity is compared to a control. In certain embodiments, the control is a culture of antigen-expressing cells that are not incubated with the cell composition. In some embodiments, the control is a measurement from a control cell composition that does not contain CAR+ cells that are incubated with the antigen-expressing cells at the same ratio.

In certain embodiments, the measurement of the cytolytic activity assay is the number of antigen-expressing cells that are viable at a time point during or at the end of the incubation for each ratio tested. In certain embodiments, the measurement is an amount of a marker of target cell death, e.g., chromium-51, that is released during the incubation. In some embodiments, the measurement is an amount of target cell death that is determined by subtracting the amount of target cells in the co-incubation at a given time point from the amount of target cells of the control that was incubated alone. In some embodiments, the measurement is the percentage of target cells that remain at a time point compared to the starting amount of target cells. In particular embodiments, the measurement is the amount of cells killed over an amount of time. In certain embodiments, the measurement is the amount of cells killed per each cell of the cell composition. In some embodiments, the measurement is the amount of cells killed per cell, or the amount of cells killed per a set number or reference of cells, for example but not limited to, the amount of target cells killed per 100 cells, per 10³ cells, per 10⁴ cells, per 10⁵ cells, per 10⁶ cells, per 10⁷ cells, per 10⁸ cells, per 10⁹ cells, or per 10¹⁰ cells of the composition. In particular embodiments, the measurement is the amount of cells killed per each CAR+ cell or a reference or set number thereof, of the cell composition. In certain embodiments, the measurement is the amount of cells killed over an amount of time per cell of the cell composition. In particular embodiments, the measurement is the amount of cells killed over an amount of time per CAR+ cells of the therapeutic cell composition.

In some embodiments, the recombinant receptor-dependent activity is upregulation of a gene in cells of the therapeutic cell composition. In some embodiments, recombinant receptor-dependent gene upregulation activity is assessed by exposing, incubating, and/or contacting cells expressing the recombinant receptor, or a cell composition containing cells that express the recombinant receptor, with a varying amount of recombinant receptor stimulating agent that binds to and stimulates the recombinant receptor. The upregulation of gene activity can be measured by directly or indirectly number over time.

In some embodiments, the recombinant receptor-dependent activity is downregulation of a gene in cells of the therapeutic cell composition. In some embodiments, recombinant receptor-dependent gene downregulation activity is assessed by exposing, incubating, and/or contacting cells expressing the recombinant receptor, or a cell composition containing cells that express the recombinant receptor, with a varying amount of recombinant receptor stimulating agent that binds to and stimulates the recombinant receptor. The downregulation of gene activity can be measured by directly or indirectly number over time.

In some embodiments, the recombinant receptor-dependent activity is upregulation of a receptor in cells of the therapeutic cell composition. In some embodiments, recombinant receptor-dependent receptor upregulation activity is assessed by exposing, incubating, and/or contacting cells expressing the recombinant receptor, or a cell composition containing cells that express the recombinant receptor, with a varying amount of recombinant receptor stimulating agent that binds to and stimulates the recombinant receptor. The upregulation of receptor activity can be measured by directly or indirectly number over time.

In some embodiments, the recombinant receptor-dependent activity is downregulation of a receptor in cells of the therapeutic cell composition. In some embodiments, recombinant receptor-dependent receptor downregulation activity is assessed by exposing, incubating, and/or contacting cells expressing the recombinant receptor, or a cell composition containing cells that express the recombinant receptor, with a varying amount of recombinant receptor stimulating agent that binds to and stimulates the recombinant receptor. The downregulation of receptor activity can be measured by directly or indirectly number over time.

In some embodiments, the measurements of the recombinant receptor-dependent activity are fit using a mathematical model to produce a recombinant receptor-dependent activity curve. Curve fitting may, in some cases, allow for inference or extrapolation of behavior, e.g., recombinant receptor-dependent activity, of the therapeutic cells composition. It is contemplated that any method known in the art to performing curve fitting may be used. In some embodiments, the curve is a sigmoid. In some embodiments, based on the recombinant receptor-dependent activity measured from each of the plurality of incubations, the titrated ratio that results in a half-maximal recombinant receptor-dependent activity is determined. In some embodiments, the titrated ratio that results in a half-maximal recombinant receptor-dependent activity is inferred, extrapolated, or estimated from the recombinant receptor-dependent activity curve. In some embodiments, the recombinant receptor-dependent activity curve is normalized to the maximum recombinant receptor-dependent activity measured. In some embodiments, the recombinant receptor-dependent activity curve is normalized to the upper asymptote of the curve, optionally a range of values of the upper asymptote.

In some embodiments, the methods including assays as described herein may be performed in duplicate or triplicate, or more, to verify the measurements of recombinant receptor-dependent activity. In some cases where the assay is performed, for example, in duplicate, triplicate, or more, the measured recombinant receptor-dependent activity from each of the replicates is used to provide a descriptive statistical measure of the recombinant receptor-dependent activity. For example, in some cases, an average (e.g., arithmetic mean), median, standard deviation, and/or variance of each measure of the recombinant receptor-dependent activity is determined for each of the plurality of ratios test. In some embodiments, an average of each measure of the recombinant receptor-dependent activity is determined. In some embodiments, a standard deviation of each measure of the recombinant receptor-dependent activity is determined. In some embodiments, the average measure of recombinant receptor-dependent activity are fit using a mathematical model to produce or estimate a recombinant receptor-dependent activity curve. In some embodiments, the curve is normalized to the average maximal value. In some embodiments, the curve is normalized to the upper asymptote, optionally an average of a range of values of the upper asymptote.

The measures described herein may be used with reference to a reference standard, such as a reference standard described herein, e.g., Section I-D-1.

D. Determining Potency of a Therapeutic Cell Composition

The methods provided herein allow for determining a potency of a therapeutic cell composition. It is contemplated that the assays described herein may be used to assess the potency of a therapeutic cell composition manufactured by processes such as those described herein (e.g., Section-II), as well as any other manufacturing process that allows for cells of the therapeutic cell composition manufactured to be cultured in an assay comprising a plurality of incubations, where each incubation includes culturing different ratios of the cells of the therapeutic composition with a recombinant receptor stimulating agent able to stimulate a recombinant receptor-dependent activity in the therapeutic cell composition. In some embodiments, 1, 2, 3, 4, 5, 6, 7, 8, 9, 10, or more therapeutic cell compositions may be assessed according to the methods provided herein.

By taking measurements of the recombinant receptor-dependent activity at each of the plurality of ratios tested, the potency of the therapeutic cell composition may be determined. In some embodiments, the measurements are composites determined by taking an arithmetic mean or median across duplicates, triplicates, or more replicates. In some embodiments, the standard deviation and/or variance of the measurements may be determined. In some embodiments, one or more measurements, including composite measurements, of the recombinant receptor-dependent activity of the therapeutic cell composition in response to the recombinant receptor stimulating agent can be used to determine a potency of a therapeutic cell composition. In some embodiments, the recombinant receptor-dependent activity can be any as described in Section I-C. In some embodiments, recombinant receptor stimulating agent can be any as described in Section I-B.

In some embodiments, the plurality of incubations at different ratios produces a plurality of measurements to which a curve fitting method may be applied. In some embodiments, the plurality of measurements includes composite measurements (e.g., means or medians). For example, the recombinant receptor-dependent activity measurements can be fit with a curve, e.g., a sigmoid, to allow the inference, extrapolation, or estimation of the behavior (e.g., sensitivity) of the therapeutic cell composition. In some embodiments, a curve fitted to the measurements may be used to estimate behavior (e.g., sensitivity) of the therapeutic composition which was not directly examined during the assay. For example, the curve may be used to estimate a lower asymptote; a minimal value; a loss of detection of recombinant receptor-dependent activity; a specified percentage (e.g., 10%, 20%, 25%, 30%, 40%, 50%, 60%, 70%, 75%, 80%, or 90%) of maximal value; a half-maximal value (e.g., 50% recombinant receptor-dependent activity); a range of 10%-90%, 20%-80%, 30%-70%, or 40%-60% of maximal recombinant receptor-dependent activity(i.e., maximal activity as described below); an upper asymptote; and a maximal value and the ratios at which each of the values or ranges occur.

It is contemplated that any measure, ratio at half-maximal, range, maximal, minimal, asymptote, and composite measures thereof) may be used to determine the potency of the therapeutic cell composition. In some embodiments, the potency is a relative potency.

1. Potency

In some embodiments, the potency of the therapeutic cell composition is defined as the ratio at which one or more or a range of recombinant receptor-dependent activity measurements occurs. In some embodiments, the one or more or range of measurements are composite measurements, such as a mean or median determined from replicated experiments. In some embodiments, the measurements and ratios are determined from a recombinant receptor-dependent activity curve of the measured recombinant receptor-dependent activity. In some embodiments, the measured recombinant receptor-dependent activity is normalized to a maximum activity measured for the therapeutic composition. In some embodiments, the recombinant receptor-dependent activity curve is normalized to a maximum recombinant receptor-dependent activity measured for the therapeutic cell composition. In some embodiments, the recombinant receptor-dependent activity curve is normalized to an upper asymptote of the recombinant receptor-dependent activity measured for the therapeutic cell composition, optionally an average of measured values across the asymptote.

In some embodiments, the potency of a therapeutic cell composition is the range of ratios over which 10%-90% recombinant receptor-dependent activity occurs, or vice versa. In some embodiments, the range of ratios over which 10%-90% recombinant receptor-dependent activity occurs is estimated from a recombinant receptor-dependent activity curve. In some embodiments, for example when the recombinant receptor-dependent activity measures or recombinant receptor-dependent activity curve are normalized, the range of recombinant receptor-dependent activity value range is from 0.1-0.9 or 10%-90%.

In some embodiments, the potency of a therapeutic cell composition is the range of ratios over which 20%-80% recombinant receptor-dependent activity occurs, or vice versa. In some embodiments, the range of ratios over which 20%-80% recombinant receptor-dependent activity occurs is estimated from a recombinant receptor-dependent activity curve. In some embodiments, for example when the recombinant receptor-dependent activity measures or recombinant receptor-dependent activity curve are normalized, the range of recombinant receptor-dependent activity value range is from 0.2-0.8 or 20%-80%.

In some embodiments, the potency of a therapeutic cell composition is the range of ratios over which 30%-70% recombinant receptor-dependent activity occurs, or vice versa. In some embodiments, the range of ratios over which 30%-70% recombinant receptor-dependent activity occurs is estimated from a recombinant receptor-dependent activity curve. In some embodiments, for example when the recombinant receptor-dependent activity measures or recombinant receptor-dependent activity curve are normalized, the range of recombinant receptor-dependent activity value range is from 0.3-0.7 or 30%-70%.

In some embodiments, the potency of a therapeutic cell composition is the range of ratios over which 40%-60% recombinant receptor-dependent activity occurs, or vice versa. In some embodiments, the range of ratios over which 40%-60% recombinant receptor-dependent activity occurs is estimated from a recombinant receptor-dependent activity curve. In some embodiments, for example when the recombinant receptor-dependent activity measures or recombinant receptor-dependent activity curve are normalized, the range of recombinant receptor-dependent activity value range is from 0.4-0.6 or 40%-60%.

In some embodiments, the potency of a therapeutic cell composition is the ratio at which the half-maximal recombinant receptor-dependent activity occurs. In some embodiments, the half-maximal value and ratio at which the half-maximal value occurs is estimated from a recombinant receptor-dependent activity curve. In some embodiments, for example when the recombinant receptor-dependent activity measures or recombinant receptor-dependent activity curve are normalized, the half-maximal recombinant receptor-dependent activity value is 0.5 or 50%.

In some embodiments, for example when the recombinant receptor-dependent activity curve is fit by a sigmoid, a linear portion of the curve is determined. In some embodiments, the potency is a measurement and corresponding ratio from the linear portion of the curve. In some embodiments, the half-maximal value measurement and ratio are determined from the linear portion of the curve.

2. Relative Potency

The methods provided herein allow for determination of a potency of a therapeutic cell composition relative to a different therapeutic cell composition, e.g., reference standard. This type of potency may be referred to as a relative potency. For example, a therapeutic cell composition assessed according the methods provided herein may be compared to a different therapeutic cell composition (e.g., reference standard, for example as described below), for example assessed according to the methods provided herein to determine how the potencies of the therapeutic cell compositions relate to one another. This offers an advantage in that multiple therapeutic cell compositions can be compared to determine which composition has a highest potency or optimal potency. In some embodiments, an optimal potency is a potency that can elicit a therapeutic effect, e.g., durable response, progression free survival, in a subject. In some embodiments, an optimal potency is a potency that does not result in toxicity in a subject. In some embodiments, an optimal potency is a potency that can elicit a therapeutic effect, e.g., durable response, progression free survival, and not result in toxicity in a subject.

In some embodiments, the relative potency of the therapeutic cell composition is defined as the ratio(s) at which one or more or a range of recombinant receptor-dependent activity measurements occurs for the therapeutic cell composition compared to the ratio(s) at which one or more or a range of recombinant receptor-dependent activity measurements occurs for the reference standard. In some embodiments, the one or more or range of measurements for one or both the therapeutic cell composition and reference standard are composite measurements, such as a mean or median determined from replicated experiments. In some embodiments, the measurements and ratios for the therapeutic cell composition and the reference standard are determined from a recombinant receptor-dependent activity curve of the measured recombinant receptor-dependent activity for compositions, respectively. In some embodiments, the measured recombinant receptor-dependent activity for the therapeutic cell composition and the reference standard is normalized to a maximum activity measured for the therapeutic composition and reference standard, respectively. In some embodiments, the recombinant receptor-dependent activity curve for the therapeutic cell composition and the reference standard is normalized to a maximum recombinant receptor-dependent activity measured for the therapeutic cell composition and reference standard, respectively. In some embodiments, the recombinant receptor-dependent activity curve for the therapeutic cell composition and the reference standard is normalized to an upper asymptote of the recombinant receptor-dependent activity measured for the therapeutic cell composition and reference standard, respectively, optionally an average of measured values across the asymptote.

In some embodiments, the relative potency of a therapeutic cell composition is the range of ratios over which 10%-90% recombinant receptor-dependent activity occurs, or vice versa, compared to the range over which 10%-90% recombinant receptor-dependent activity occurs, or vice versa, for the standard reference. In some embodiments, the range of ratios over which 10%-90% recombinant receptor-dependent activity occurs for the therapeutic cell composition and the reference standard is estimated from a recombinant receptor-dependent activity curve for the therapeutic cell composition and the reference standard, respectively. In some embodiments, for example when the recombinant receptor-dependent activity measures or recombinant receptor-dependent activity curve for the therapeutic cell composition and the reference standard are normalized, the range of recombinant receptor-dependent activity value range is from 0.1-0.9 or 10%-90%.

In some embodiments, the relative potency of a therapeutic cell composition is the range of ratios over which 20%-80% recombinant receptor-dependent activity occurs, or vice versa, compared to the range over which 20%-80% recombinant receptor-dependent activity occurs, or vice versa, for the standard reference. In some embodiments, the range of ratios over which 20%-80% recombinant receptor-dependent activity occurs for the therapeutic cell composition and the reference standard is estimated from a recombinant receptor-dependent activity curve for the therapeutic cell composition and the reference standard, respectively. In some embodiments, for example when the recombinant receptor-dependent activity measures or recombinant receptor-dependent activity curve for the therapeutic cell composition and the reference standard are normalized, the range of recombinant receptor-dependent activity value range is from 0.2-0.8 or 20%-80%.

In some embodiments, the relative potency of a therapeutic cell composition is the range of ratios over which 30%-70% recombinant receptor-dependent activity occurs, or vice versa, compared to the range over which 30%-70% recombinant receptor-dependent activity occurs, or vice versa, for the standard reference. In some embodiments, the range of ratios over which 30%-70% recombinant receptor-dependent activity occurs for the therapeutic cell composition and the reference standard is estimated from a recombinant receptor-dependent activity curve for the therapeutic cell composition and the reference standard, respectively. In some embodiments, for example when the recombinant receptor-dependent activity measures or recombinant receptor-dependent activity curve for the therapeutic cell composition and the reference standard are normalized, the range of recombinant receptor-dependent activity value range is from 0.3-0.7 or 30%-70%.

In some embodiments, the relative potency of a therapeutic cell composition is the range of ratios over which 40%-60% recombinant receptor-dependent activity occurs, or vice versa, compared to the range over which 40%-60% recombinant receptor-dependent activity occurs, or vice versa, for the standard reference. In some embodiments, the range of ratios over which 40%-60% recombinant receptor-dependent activity occurs for the therapeutic cell composition and the reference standard is estimated from a recombinant receptor-dependent activity curve for the therapeutic cell composition and the reference standard, respectively. In some embodiments, for example when the recombinant receptor-dependent activity measures or recombinant receptor-dependent activity curve for the therapeutic cell composition and the reference standard are normalized, the range of recombinant receptor-dependent activity value range is from 0.4-0.6 or 40%-60%.

In some embodiments, the relative potency of a therapeutic cell composition is the ratio at which the half-maximal recombinant receptor-dependent activity occurs compared to the ratio at which the half-maximal recombinant receptor-dependent activity occurs for the reference standard. In some embodiments, the half-maximal value and ratio at which the half-maximal value occurs for the therapeutic cell composition and the reference standard is estimated from a recombinant receptor-dependent activity curve for the therapeutic cell composition and the reference standard, respectively. In some embodiments, for example when the recombinant receptor-dependent activity measures or recombinant receptor-dependent activity curve for the therapeutic cell composition and reference standard are normalized, the half-maximal recombinant receptor-dependent activity value is 0.5 or 50%.

In some embodiments, for example when the recombinant receptor-dependent activity curve for therapeutic cell composition and the reference standard are fit by a sigmoid, a linear portion of the curves is determined. In some embodiments, the relative potency is a comparison of the measurement and corresponding ratio from the linear portion of the curve of the therapeutic cell composition and the measurement and corresponding ratio from the linear portion of the curve of the reference standard. In some embodiments, the half-maximal value measurement and ratio for the therapeutic cell composition and reference standard are determined from the linear portion of the curve.

In some embodiments, the comparison between the measurements, such as described above, for the therapeutic cell composition and the reference composition is a division. For example, the ratio at which half-maximal recombinant receptor-dependent activity occurs for the therapeutic cell composition is divided by the ratio at which half-maximal recombinant receptor-dependent activity occurs for the reference standard. In some embodiments, the relative potency is expressed as a ratio. In some embodiments, the relative potency is expressed as a percentage.

In some embodiments, for example when the recombinant receptor-dependent activity curve for therapeutic cell composition and the reference standard are fit by a sigmoid and normalized as described above, the relative potency is the difference between the curves. In some embodiments, the difference between the curves is measured for the linear portion of the normalized curves. In some embodiments, normalization of the recombinant receptor-dependent activity curves, e.g., sigmoid curves, for therapeutic cell composition and the reference standard, may be used to directly compare the recombinant receptor-dependent activity curve for therapeutic cell composition and the reference standard.

a. Reference Standards

Particular embodiments contemplate that a measurement of a recombinant receptor-dependent activity (e.g., CAR+ dependent activity) for a therapeutic cell composition can be compared to a reference measurement, (i.e. a reference measure) of a reference standard to, for example, determine a relative potency. In particular embodiments, the reference measurement is a predetermined measurement, or value thereof, of the recombinant receptor-dependent activity of the reference standard. In some embodiments, the recombinant receptor-dependent activity of the reference standard is assessed according to the methods disclosed herein. In some embodiments, the reference standard is a therapeutic cell composition for which titrated ratios resulting in a recombinant receptor-dependent activity have been validated. In some embodiments, the reference standard is a therapeutic cell composition for which titrated ratios resulting in a recombinant receptor-dependent activity have been validated and a curve, e.g., sigmoid, has been fit to the measured activity to generate recombinant receptor-dependent activity curve. In some embodiments, the recombinant receptor-dependent activity curve for the reference standard is normalized. In some embodiments, the recombinant receptor-dependent activity curve is normalized to a maximal measured recombinant receptor-dependent activity. In some embodiments, the recombinant receptor-dependent activity curve is normalized to an upper asymptote of the recombinant receptor-dependent activity curve. In some embodiments, the recombinant receptor-dependent activity curve is normalized to an average value calculated over the upper asymptote of the recombinant receptor-dependent activity curve. In some embodiments, the reference standard is a therapeutic cell composition comprising a validated titrated ratio resulting in a half-maximal recombinant receptor-dependent activity. In some embodiments, the validated titrated ratio resulting in a half-maximal recombinant receptor-dependent activity is determined from the recombinant receptor-dependent activity curve.

In some embodiments, the reference standard is a commercially available therapeutic cell composition. In some embodiments, the reference standard is a therapeutic cell composition manufactured using a manufacturing process that is identical to a manufacturing process used to manufacture the therapeutic cell composition to which it is compared. In some embodiments, the reference standard is a therapeutic cell composition manufactured using a manufacturing process that is different from a manufacturing process used to manufacture the therapeutic cell composition to which it is compared. In some embodiments, the reference standard is a therapeutic cell composition comprising an identical recombinant receptor as the therapeutic cell composition to which it is compared. In some embodiments, the reference standard is a therapeutic cell composition comprising a different recombinant receptor as the therapeutic cell composition to which it is compared. In some embodiments, the reference standard is a therapeutic cell composition manufactured from the same subject to which it is compared. In some embodiments, the reference standard is a therapeutic cell composition manufactured from a different subject from which the therapeutic cell composition it which it is compared is manufactured. In some embodiments, the reference standard is a therapeutic cell composition derived from a healthy subject. In some embodiments, the reference standard is derived from a subject having a disease or condition. In some embodiments, the reference standard is derived from a subject having cancer. In some embodiments, the reference standard may be a combination of one or more of those described above.

In some embodiments, the reference standard has been administered to a subject. In particular embodiments, administration of the reference standard to the subject was observed and was determined to result in an acceptable safety profile following administration to a subject. In particular embodiments, administration of the reference standard did not result in any severe toxicity. In certain embodiments, administration of the reference standard did not result in any severe neurotoxicity. In particular embodiments, the reference standard is a therapeutic cell composition that was associated with grade 4 or lower, grade 3 or lower, grade 2 or lower, grade 1 or lower, or grade 0 score for neurotoxicity. In some embodiments, the reference standard is associated with acceptable safety profiles. In particular embodiments, the acceptable safety profile is an absence of observed grade 1 or higher, observed grade 2 or higher, observed grade 3 or higher, or grade 4 or higher, neurotoxicity. In certain embodiments, the reference standard is associated with an acceptable safety profile of an absence of an observed grade 3 or higher neurotoxicity. In particular embodiments, the reference standard is associated with an acceptable safety profile of an absence an observed grade 3 or higher neurotoxicity.

In certain embodiments, the reference standard has been observed or determined to result in a desired efficacy following administration to a subject. In certain embodiments, the subject has a disease or condition expressing or associated with the antigen as the subjects that were administered the reference standard. In particular embodiments, reference standard has been observed or determined to result in a complete response (CR). In particular embodiments, reference standard has been observed or determined to result in a durable response.

### II. METHODS FOR GENERATING ENGINEERED T CELLS

In some embodiments, the methods of potency of a therapeutic cell composition provided herein can be used in connection with generating a therapeutic composition of engineered cells (e.g., output composition), such as engineered CD4+ T cells and/or engineered CD8+ T cells, that express a recombinant protein, e.g., a recombinant receptor such as a T cell receptor (TCR) or a chimeric antigen receptor (CAR). In some embodiments, the methods provided herein are used in connection with manufacturing, generating, or producing a cell therapy, and may be used in connection with additional processing steps, such as steps for the isolation, separation, selection, activation or stimulation, transduction, washing, suspension, dilution, concentration, and/or formulation of the cells. In some embodiments, the methods of generating or producing engineered cells, e.g., engineered CD4+ T cells and/or engineered CD8+ T cells, include one or more of isolating cells from a subject, preparing, processing, incubating under stimulating conditions, and/or engineering (e.g., transducing) the cells. In some embodiments, the method includes processing steps carried out in an order in which: input cells, e.g., primary cells, are first isolated, such as selected or separated, from a biological sample; input cells are incubated under stimulating conditions, engineered with vector particles, e.g., viral vector particles, to introduce a recombinant polynucleotide into the cells, e.g., by transduction or transfection; cultivating the engineered cells, e.g., transduced cells, such as to expand the cells; and collecting, harvesting, and/or filling a container with all or a portion of the cells for formulating the cells in an output composition. In some embodiments, CD4+ and CD8+ T cells are manufactured independently from one another, e.g., in separate input compositions, but the process for manufacturing includes the same processing steps. In some embodiments, CD4+ and CD8+ T cells are manufactured together, e.g., in the same input composition. In some embodiments, the cells of the generated output composition (e.g., therapeutic cell composition) are re-introduced into the same subject, before or after cryopreservation. In some embodiments, the output compositions of engineered cells (e.g., therapeutic cell composition) are suitable for use in a therapy, e.g., an autologous cell therapy, allogeneic cell therapy. Exemplary manufacturing methods are described in published international patent application, publication no. WO 2019/089855, the contents of which are incorporated herein by reference in their entirety.

A. Samples and Cell Preparations

In particular embodiments, the provided methods are used in connection with isolating, selecting, and/or enriching cells from a biological sample to generate one or more input compositions of enriched cells, e.g., T cells. In some embodiments, the provided methods include isolation of cells or compositions thereof from biological samples, such as those obtained from or derived from a subject, such as one having a particular disease or condition or in need of a cell therapy or to which cell therapy will be administered. In some aspects, the subject is a human, such as a subject who is a patient in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca“/Mg” free PBS. In certain embodiments, components of a blood cell sample are removed and the cells directly resuspended in culture media.

In some embodiments, the preparation methods include steps for freezing, e.g., cryopreserving, the cells, either before or after isolation, selection and/or enrichment and/or incubation for transduction and engineering, and/or after cultivation and/or harvesting of the engineered cells. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. In some embodiments, the cells are frozen, e.g., cryofrozen or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryofrozen or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to or to about −80° C. at a rate of or of about 1° per minute and stored in the vapor phase of a liquid nitrogen storage tank.

In some embodiments, isolation of the cells or populations includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components. In some embodiments, the methods include density-based cell separation methods, such as the preparation of white blood cells from peripheral blood by lysing the red blood cells and centrifugation through a Percoll or Ficoll gradient.

In some embodiments, at least a portion of the selection step includes incubation of cells with a selection reagent. The incubation with a selection reagent or reagents, e.g., as part of selection methods which may be performed using one or more selection reagents for selection of one or more different cell types based on the expression or presence in or on the cell of one or more specific molecules, such as surface markers, e.g., surface proteins, intracellular markers, or nucleic acid. In some embodiments, any known method using a selection reagent or reagents for separation based on such markers may be used. In some embodiments, the selection reagent or reagents result in a separation that is affinity- or immunoaffinity-based separation. For example, the selection in some aspects includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

In some aspects of such processes, a volume of cells is mixed with an amount of a desired affinity-based selection reagent. The immunoaffinity-based selection can be carried out using any system or method that results in a favorable energetic interaction between the cells being separated and the molecule specifically binding to the marker on the cell, e.g., the antibody or other binding partner on the solid surface, e.g., particle. In some embodiments, methods are carried out using particles such as beads, e.g., magnetic beads, that are coated with a selection agent (e.g., antibody) specific to the marker of the cells. The particles (e.g., beads) can be incubated or mixed with cells in a container, such as a tube or bag, while shaking or mixing, with a constant cell density-to-particle (e.g., bead) ratio to aid in promoting energetically favored interactions. In other cases, the methods include selection of cells in which all or a portion of the selection is carried out in the internal cavity of a centrifugal chamber, for example, under centrifugal rotation. In some embodiments, incubation of cells with selection reagents, such as immunoaffinity-based selection reagents, is performed in a centrifugal chamber. In certain embodiments, the isolation or separation is carried out using a system, device, or apparatus described in International Patent Application, Publication Number WO2009/072003, or US 20110003380 A1. In one example, the system is a system as described in International Publication Number WO2016/073602.

In some embodiments, by conducting such selection steps or portions thereof (e.g., incubation with antibody-coated particles, e.g., magnetic beads) in the cavity of a centrifugal chamber, the user is able to control certain parameters, such as volume of various solutions, addition of solution during processing and timing thereof, which can provide advantages compared to other available methods. For example, the ability to decrease the liquid volume in the cavity during the incubation can increase the concentration of the particles (e.g., bead reagent) used in the selection, and thus the chemical potential of the solution, without affecting the total number of cells in the cavity. This in turn can enhance the pairwise interactions between the cells being processed and the particles used for selection. In some embodiments, carrying out the incubation step in the chamber, e.g., when associated with the systems, circuitry, and control as described herein, permits the user to effect agitation of the solution at desired time(s) during the incubation, which also can improve the interaction.

In some embodiments, at least a portion of the selection step is performed in a centrifugal chamber, which includes incubation of cells with a selection reagent. In some aspects of such processes, a volume of cells is mixed with an amount of a desired affinity-based selection reagent that is far less than is normally employed when performing similar selections in a tube or container for selection of the same number of cells and/or volume of cells according to manufacturer's instructions. In some embodiments, an amount of selection reagent or reagents that is/are no more than 5%, no more than 10%, no more than 15%, no more than 20%, no more than 25%, no more than 50%, no more than 60%, no more than 70% or no more than 80% of the amount of the same selection reagent(s) employed for selection of cells in a tube or container-based incubation for the same number of cells and/or the same volume of cells according to manufacturer's instructions is employed.

In some embodiments, for selection, e.g., immunoaffinity-based selection of the cells, the cells are incubated in the cavity of the chamber in a composition that also contains the selection buffer with a selection reagent, such as a molecule that specifically binds to a surface marker on a cell that it desired to enrich and/or deplete, but not on other cells in the composition, such as an antibody, which optionally is coupled to a scaffold such as a polymer or surface, e.g., bead, e.g., magnetic bead, such as magnetic beads coupled to monoclonal antibodies specific for CD4 and CD8. In some embodiments, as described, the selection reagent is added to cells in the cavity of the chamber in an amount that is substantially less than (e.g., is no more than 5%, 10%, 20%, 30%, 40%, 50%, 60%, 70% or 80% of the amount) as compared to the amount of the selection reagent that is typically used or would be necessary to achieve about the same or similar efficiency of selection of the same number of cells or the same volume of cells when selection is performed in a tube with shaking or rotation. In some embodiments, the incubation is performed with the addition of a selection buffer to the cells and selection reagent to achieve a target volume with incubation of the reagent of, for example, 10 mL to 200 mL, such as at least or about at least or about 10 mL, 20 mL, 30 mL, 40 mL, 50 mL, 60 mL, 70 mL, 80 mL, 90 mL, 100 mL, 150 mL or 200 mL. In some embodiments, the selection buffer and selection reagent are pre-mixed before addition to the cells. In some embodiments, the selection buffer and selection reagent are separately added to the cells. In some embodiments, the selection incubation is carried out with periodic gentle mixing condition, which can aid in promoting energetically favored interactions and thereby permit the use of less overall selection reagent while achieving a high selection efficiency.

In some embodiments, the total duration of the incubation with the selection reagent is from 5 minutes to 6 hours or from about 5 minutes to about 6 hours, such as 30 minutes to 3 hours, for example, at least or about at least 30 minutes, 60 minutes, 120 minutes or 180 minutes.

In some embodiments, the incubation generally is carried out under mixing conditions, such as in the presence of spinning, generally at relatively low force or speed, such as speed lower than that used to pellet the cells, such as from 600 rpm to 1700 rpm or from about 600 rpm to about 1700 rpm (e.g., at or about or at least 600 rpm, 1000 rpm, or 1500 rpm or 1700 rpm), such as at an RCF at the sample or wall of the chamber or other container of from 80 g to 100 g or from about 80 g to about 100 g (e.g., at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, such process is carried out within the entirely closed system to which the chamber is integral. In some embodiments, this process (and in some aspects also one or more additional step, such as a previous wash step washing a sample containing the cells, such as an apheresis sample) is carried out in an automated fashion, such that the cells, reagent, and other components are drawn into and pushed out of the chamber at appropriate times and centrifugation effected, so as to complete the wash and binding step in a single closed system using an automated program.

In some embodiments, after the incubation and/or mixing of the cells and selection reagent and/or reagents, the incubated cells are subjected to a separation to select for cells based on the presence or absence of the particular reagent or reagents. In some embodiments, the separation is performed in the same closed system in which the incubation of cells with the selection reagent was performed. In some embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

Such separation steps can be based on positive selection, in which the cells having bound the reagents, e.g., antibody or binding partner, are retained for further use, and/or negative selection, in which the cells having not bound to the reagent, e.g., antibody or binding partner, are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

In some embodiments, the process steps further include negative and/or positive selection of the incubated and cells, such as using a system or apparatus that can perform an affinity-based selection. In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively. Multiple rounds of the same selection step, e.g., positive or negative selection step, can be performed. In certain embodiments, the positively or negatively selected fraction subjected to the process for selection, such as by repeating a positive or negative selection step. In some embodiments, selection is repeated twice, three times, four times, five times, six times, seven times, eight times, nine times or more than nine times. In certain embodiments, the same selection is performed up to five times. In certain embodiments, the same selection step is performed three times.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types. In certain embodiments, one or more separation steps are repeated and/or performed more than once. In some embodiments, the positively or negatively selected fraction resulting from a separation step is subjected to the same separation step, such as by repeating the positive or negative selection step. In some embodiments, a single separation step is repeated and/or performed more than once, for example, to increase the yield of positively selected cells, to increase the purity of negatively selected cells, and/or to further remove the positively selected cells from the negatively selected fraction. In certain embodiments, one or more separation steps are performed and/or repeated two times, three times, four times, five times, six times, seven times, eight times, nine times, ten times, or more than ten times. In certain embodiments, the one or more selection steps are performed and/or repeated between one and ten times, between one and five times, or between three and five times. In certain embodiments, one or more selection steps are repeated three times.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some embodiments, such cells are selected by incubation with one or more antibody or binding partner that specifically binds to such markers. In some embodiments, the antibody or binding partner can be conjugated, such as directly or indirectly, to a solid support or matrix to effect selection, such as a magnetic bead or paramagnetic bead. For example, CD3+, CD28+ T cells can be positively selected using CD3/CD28 conjugated magnetic beads (e.g., DYNABEADS® M-450 CD3/CD28 T Cell Expander, and/or ExpACT® beads).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ T cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al., (2012) Blood. 1:72-82; Wang et al. (2012) J Immunother. 35(9):689-701. In some embodiments, combining TCM-enriched CD8+ T cells and CD4+ T cells further enhances efficacy.

In embodiments, memory T cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L.

Such selections in some aspects are carried out simultaneously and in other aspects are carried out sequentially, in either order. In some aspects, the same CD4 expression-based selection step used in preparing the CD8+ T cell population or subpopulation, also is used to generate the CD4+ T cell population or sub-population, such that both the positive and negative fractions from the CD4-based separation are retained and used in subsequent steps of the methods, optionally following one or more further positive or negative selection steps. In some embodiments, the selection for the CD4+ T cell population and the selection for the CD8+ T cell population are carried out simultaneously. In some embodiments, the CD4+ T cell population and the selection for the CD8+ T cell population are carried out sequentially, in either order. In some embodiments, methods for selecting cells can include those as described in published U.S. App. No. US20170037369. In some embodiments, the selected CD4+ T cell population and the selected CD8+ T cell population may be combined subsequent to the selecting. In some aspects, the selected CD4+ T cell population and the selected CD8+ T cell population may be combined in a bioreactor bag as described herein. In some embodiments, the selected CD4+ T cell population and the selected CD8+ T cell population are separately processed, whereby the selected CD4+ T cell population is enriched in CD4+ T cells and incubated with a stimulatory reagent (e.g., anti-CD3/anti-CD28 magnetic beads), transduced with a viral vector encoding a recombinant protein (e.g., CAR) and cultivated under conditions to expand T cells and the selected CD8+ T cell population is enriched in CD8+ T cell and incubated with a stimulatory reagent (e.g., anti-CD3/anti-CD28 magnetic beads), transduced with a viral vector encoding a recombinant protein (e.g., CAR), such as the same recombinant protein as for engineering of the CD4+ T cells from the same donor, and cultivated under conditions to expand T cells, such as in accord with the provided methods.

In particular embodiments, a biological sample, e.g., a sample of PBMCs or other white blood cells, are subjected to selection of CD4+ T cells, where both the negative and positive fractions are retained. In certain embodiments, CD8+ T cells are selected from the negative fraction. In some embodiments, a biological sample is subjected to selection of CD8+ T cells, where both the negative and positive fractions are retained. In certain embodiments, CD4+ T cells are selected from the negative fraction.

In a particular example, a sample of PBMCs or other white blood cell sample is subjected to selection of CD4+ T cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4+ T helper cells may be sorted into naïve, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, or CD4+ T cells. In some embodiments, central memory CD4+ T cells are CD62L+ and CD45RO+. In some embodiments, effector CD4+ T cells are CD62L- and CD45RO−.

In one example, to enrich for CD4+ T cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, NJ).

In some aspects, the incubated sample or composition of cells to be separated is incubated with a selection reagent containing small, magnetizable or magnetically responsive material, such as magnetically responsive particles or microparticles, such as paramagnetic beads (e.g., such as Dynalbeads or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. Many well-known magnetically responsive materials for use in magnetic separation methods are known, e.g., those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 also may be used.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some aspects, separation is achieved in a procedure in which the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In some embodiments, the affinity-based selection is via magnetic-activated cell sorting (MACS) (Miltenyi Biotech, Auburn, CA). Magnetic Activated Cell Sorting (MACS), e.g., CliniMACS systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the isolation and/or selection results in one or more input compositions of enriched T cells, e.g., CD3+ T cells, CD4+ T cells, and/or CD8+ T cells. In some embodiments, two or more separate input composition are isolated, selected, enriched, or obtained from a single biological sample. In some embodiments, separate input compositions are isolated, selected, enriched, and/or obtained from separate biological samples collected, taken, and/or obtained from the same subject.

In certain embodiments, the one or more input compositions is or includes a composition of enriched T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD3+ T cells. In particular embodiment, the input composition of enriched T cells consists essentially of CD3+ T cells.

In certain embodiments, the one or more input compositions is or includes a composition of enriched CD4+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the input composition of CD4+ T cells includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells. In some embodiments, the composition of enriched T cells consists essentially of CD4+ T cells.

In certain embodiments, the one or more compositions is or includes a composition of CD8+ T cells that is or includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of CD8+ T cells contains less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free of or substantially free of CD4+ T cells. In some embodiments, the composition of enriched T cells consists essentially of CD8+ T cells.

In some embodiments, the one or more input compositions of enriched T cells are frozen, e.g., cryopreserved and/or cryofrozen, after isolation, selection and/or enrichment. In some embodiments, the one or more input compositions of frozen e.g., cryopreserved and/or cryofrozen, prior to any steps of incubating, activating, stimulating, engineering, transducing, transfecting, cultivating, expanding, harvesting, and/or formulating the composition of cells. In particular embodiments, the one or more cryofrozen input compositions are stored, e.g., at or at about −80° C., for between 12 hours and 7 days, between 24 hours and 120 hours, or between 2 days and 5 days. In particular embodiments, the one or more cryofrozen input compositions are stored at or at about −80° C., for an amount of time of less than 10 days, 9 days, 8 days, 7 days, 6 days, or 5 days, 4 days, 3 days, 2 days, or 1 day. In some embodiments, the one or more cryofrozen input compositions are stored at or at about −80° C., for or for about 1 day, 2 days, 3 days, 4 days, 5 days, or 6 days.

B. Activation and Stimulation of Cells

In some embodiments, the provided methods are used in connection with incubating cells under stimulating conditions. In some embodiments, the stimulating conditions include conditions that activate or stimulate, and/or are capable of activating or stimulating a signal in the cell, e.g., a CD4+ T cell or CD8+ T cell, such as a signal generated from a TCR and/or a coreceptor. In some embodiments, the stimulating conditions include one or more steps of culturing, cultivating, incubating, activating, propagating the cells with and/or in the presence of a stimulatory reagent, e.g., a reagent that activates or stimulates, and/or is capable of activating or stimulating a signal in the cell. In some embodiments, the stimulatory reagent stimulates and/or activates a TCR and/or a coreceptor. In particular embodiments, the stimulatory reagent is a reagent described in Section II-B-1.

In certain embodiments, one or more compositions of enriched T cells are incubated under stimulating conditions prior to genetically engineering the cells, e.g., transfecting and/or transducing the cell such as by a technique provided in Section II-C. In particular embodiments, one or more compositions of enriched T cells are incubated under stimulating conditions after the one or more compositions have been isolated, selected, enriched, or obtained from a biological sample. In particular embodiments, the one or more compositions are input compositions. In particular embodiments, the one or more input compositions have been previously cryofrozen and stored, and are thawed prior to the incubation.

In certain embodiments, the one or more compositions of enriched T cells are or include two separate compositions, e.g., separate input compositions, of enriched T cells. In particular embodiments, two separate compositions of enriched T cells, e.g., two separate compositions of enriched T cells selected, isolated, and/or enriched from the same biological sample, are separately incubated under stimulating conditions. In certain embodiments, the two separate compositions include a composition of enriched CD4+ T cells. In particular embodiments, the two separate compositions include a composition of enriched CD8+ T cells. In some embodiments, two separate compositions of enriched CD4+ T cells and enriched CD8+ T cells are separately incubated under stimulating conditions.

In some embodiments, a single composition of enriched T cells is incubated under stimulating conditions. In certain embodiments, the single composition is a composition of enriched CD4+ T cells. In some embodiments, the single composition is a composition of enriched CD4+ and CD8+ T cells that have been combined from separate compositions prior to the incubation.

In some embodiments, the composition of enriched CD4+ T cells that is incubated under stimulating conditions includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the composition of enriched CD4+ T cells that is incubated under stimulating conditions includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells that is incubated under stimulating conditions includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of enriched CD8+ T cells that is incubated under stimulating conditions includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, separate compositions of enriched CD4+ and CD8+ T cells are combined into a single composition and are incubated under stimulating conditions. In certain embodiments, separate stimulated compositions of enriched CD4+ and enriched CD8+ T cells are combined into a single composition after the incubation has been performed and/or completed. In some embodiments, separate stimulated compositions of stimulated CD4+ and stimulated CD8+ T cells are separately processed after the incubation has been performed and/or completed, whereby the stimulated CD4+ T cell population (e.g., incubated with stimulatory an anti-CD3/anti-CD28 magnetic bead stimulatory reagent) is transduced with a viral vector encoding a recombinant protein (e.g., CAR) and cultivated under conditions to expand T cells and the stimulated CD8+ T cell population (e.g., incubated with stimulatory an anti-CD3/anti-CD28 magnetic bead stimulatory reagent) is transduced with a viral vector encoding a recombinant protein (e.g., CAR), such as the same recombinant protein as for engineering of the CD4+ T cells from the same donor, and cultivated under conditions to expand T cells, such as in accord with the provided methods.

In some embodiments, the incubation under stimulating conditions can include culture, cultivation, stimulation, activation, propagation, including by incubation in the presence of stimulating conditions, for example, conditions designed to induce proliferation, expansion, activation, and/or survival of cells in the population, to mimic antigen exposure, and/or to prime the cells for genetic engineering, such as for the introduction of a recombinant antigen receptor. In particular embodiments, the stimulating conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, the stimulation and/or incubation under stimulating conditions is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the cells, e.g., T cells, compositions of cells, and/or cell populations, such as CD4+ and CD8+ T cells or compositions, populations, or subpopulations thereof, are expanded by adding to the culture-initiating composition feeder cells, such as non-dividing peripheral blood mononuclear cells (PBMCs) (e.g., such that the resulting population of cells contains at least about 5, 10, 20, or 40 or more PBMC feeder cells for each T lymphocyte in the initial population to be expanded); and incubating the culture (e.g., for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. In some embodiments, a temperature shift is effected during culture, such as from 37 degrees Celsius to 35 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, populations of CD4+ and CD8+ that are antigen specific can be obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen. Naive T cells may also be used.

In particular embodiments, the stimulating conditions include incubating, culturing, and/or cultivating the cells with a stimulatory reagent. In particular embodiments, the stimulatory reagent is a reagent described in Section II-B-1. In certain embodiments, the stimulatory reagent contains or includes a bead. An exemplary stimulatory reagent is or includes anti-CD3/anti-CD28 magnetic beads. In certain embodiments, the start and/or initiation of the incubation, culturing, and/or cultivating cells under stimulating conditions occurs when the cells come into contact with and/or are incubated with the stimulatory reagent. In particular embodiments, the cells are incubated prior to, during, and/or subsequent to genetically engineering the cells, e.g., introducing a recombinant polynucleotide into the cell such as by transduction or transfection.

In some embodiments, the composition of enriched T cells are incubated at a ratio of stimulatory reagent and/or beads, e.g., anti-CD3/anti-CD28 magnetic beads, to cells at or at about 3:1, 2.5:1, 2:1, 1.5:1, 1.25:1, 1.2:1, 1.1:1, 1:1, 0.9:1, 0.8:1, 0.75:1, 0.67:1, 0.5:1, 0.3:1, or 0.2:1. In particular embodiments, the ratio of stimulatory reagent and/or beads to cells is between 2.5:1 and 0.2:1, between 2:1 and 0.5:1, between 1.5:1 and 0.75:1, between 1.25:1 and 0.8:1, between 1.1:1 and 0.9:1. In particular embodiments, the ratio of stimulatory reagent to cells is about 1:1 or is 1:1.

In particular embodiments, incubating the cells at a ratio of less than 3:1 or less than 3 stimulatory reagents, e.g., anti-CD3/anti-CD28 magnetic beads per cell, such as a ratio of 1:1, reduces the amount of cell death that occurs during the incubation, e.g., such as by activation-induced cell death. In some embodiments, the cells are incubated with the stimulatory reagent, e.g., anti-CD3/anti-CD28 magnetic beads, at a ratio of beads to cells of less than 3 (or 3:1 or less than 3 beads per cell). In particular embodiments, incubating the cells at a ratio of less than 3:1 or less than 3 beads per cell, such as a ratio of 1:1, reduces the amount of cell death that occurs during the incubation, e.g., such as by activation-induced cell death.

In particular embodiments, the composition of enriched T cells is incubated with the stimulatory reagent, e.g., anti-CD3/anti-CD28 magnetic beads, at a ratio of less than 3:1 stimulatory reagents and/or beads per cell, such as a ratio of 1:1, and at least 50%, at least 60%, at least 70%, at least 75%, at least 80%, at least 85%, at least 90%, at least 95%, at least 99%, or at least 99.9% of the T cells survive, e.g., are viable and/or do not undergo necrosis, programed cell death, or apoptosis, during or at least 1 day, 2 days, 3 days, 4 days, 5 days, 6 days, 7 days, or more than 7 days after the incubation is complete. In particular embodiments, the composition of enriched T cells is incubated with the stimulatory reagent at a ratio of less than 3:1 stimulatory reagents and/or beads per cell, e.g., a ratio of 1:1, and less than 50%, less than 40%, less than 30%, less than 25%, less than 20%, less than 15%, less than 10%, less than 5%, less than 1%, less than 0.1% or less than 0.01% of the cells undergo activation induced cell death during the incubation.

In certain embodiments, the composition of enriched T cells is incubated with the stimulatory reagent, e.g., anti-CD3/anti-CD28 magnetic beads, at a ratio of less than 3:1 beads per cell, e.g., a ratio of 1:1, and the cells of the composition have at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-fold, at least 50-fold, or at least 100-fold greater survival as compared to cells undergoing an exemplary and/or alternative process where the composition of enriched T cells in incubated with the stimulatory reagent at a ratio of 3:1 or greater.

In some embodiments, the composition of enriched T cells incubated with the stimulatory reagent comprises from 1.0×10⁵ cells/mL to 1.0×10⁸ cells/mL or from about 1.0×10⁵ cells/mL to about 1.0×10⁸ cells/mL, such as at least or about at least or about 1.0×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL. In some embodiments, the composition of enriched T cells incubated with the stimulatory reagent comprises about 0.5×10⁶ cells/mL, 1×10⁶ cells/mL, 1.5×10⁶ cells/mL, 2×10⁶ cells/mL, 2.5×10⁶ cells/mL, 3×10⁶ cells/mL, 3.5×10⁶ cells/mL, 4×10⁶ cells/mL, 4.5×10⁶ cells/mL, 5×10⁶ cells/mL, 5.5×10⁶ cells/mL, 6×10⁶ cells/mL, 6.5×10⁶ cells/mL, 7×10⁶ cells/mL, 7.5×10⁶ cells/mL, 8×10⁶ cells/mL, 8.5×10⁶ cells/mL, 9×10⁶ cells/mL, 9.5×10⁶ cells/mL, or 10×10⁶ cells/mL, such as about 2.4×10⁶ cells/mL.

In some embodiments, the composition of enriched T cells is incubated with the stimulatory reagent at a temperature from about 25 to about 38° C., such as from about 30 to about 37° C., for example at or about 37° C.±2° C. In some embodiments, the composition of enriched T cells is incubated with the stimulatory reagent at a CO₂ level from about 2.5% to about 7.5%, such as from about 4% to about 6%, for example at or about 5%±0.5%. In some embodiments, the composition of enriched T cells is incubated with the stimulatory reagent at a temperature of or about 37° C. and/or at a CO₂ level of or about 5%.

In particular embodiments, the stimulating conditions include incubating, culturing, and/or cultivating a composition of enriched T cells with and/or in the presence of one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes IL-2. In some embodiments, the stimulating conditions include incubating composition of enriched T cells, such as enriched CD4+ T cells or enriched CD8+ T cells, in the presence of a stimulatory reagent, e.g., anti-CD3/anti-CD28 magnetic beads, as described and in the presence or one or more recombinant cytokines.

In particular embodiments, the composition of enriched CD4+ T cells are incubated with IL-2, e.g., recombinant IL-2. Without wishing to be bound by theory, particular embodiments contemplate that CD4+ T cells that are obtained from some subjects do not produce, or do not sufficiently produce, IL-2 in amounts that allow for growth, division, and expansion throughout the process for generating a composition of output cells, e.g., engineered cells suitable for use in cell therapy. In some embodiments, incubating a composition of enriched CD4+ T cells under stimulating conditions in the presence of recombinant IL-2 increases the probability or likelihood that the CD4+ T cells of the composition will continue to survive, grow, expand, and/or activate during the incubation step and throughout the process. In some embodiments, incubating the composition of enriched CD4+ T cells in the presence of recombinant IL-2 increases the probability and/or likelihood that an output composition of enriched CD4+ T cells, e.g., engineered CD4+ T cells suitable for cell therapy, will be produced from the composition of enriched CD4+ T cells by at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-fold, at least 50-fold, or at least 100-fold as compared to an alternative and/or exemplary method that does not incubate the composition of enriched CD4+ T cells in the presence of recombinant IL-2.

In certain embodiments, the amount or concentration of the one or more cytokines are measured and/or quantified with International Units (IU). International units may be used to quantify vitamins, hormones, cytokines, vaccines, blood products, and similar biologically active substances. In some embodiments, IU are or include units of measure of the potency of biological preparations by comparison to an international reference standard of a specific weight and strength e.g., WHO 1st International Standard for Human IL-2, 86/504. International Units are the only recognized and standardized method to report biological activity units that are published and are derived from an international collaborative research effort. In particular embodiments, the IU for composition, sample, or source of a cytokine may be obtained through product comparison testing with an analogous WHO standard product. For example, in some embodiments, the IU/mg of a composition, sample, or source of human recombinant IL-2, IL-7, or IL-15 is compared to the WHO standard IL-2 product (NIB SC code: 86/500), the WHO standard IL-17 product (NIBSC code: 90/530) and the WHO standard IL-15 product (NIB SC code: 95/554), respectively.

In some embodiments, the biological activity in IU/mg is equivalent to (ED₅₀ in ng/ml)⁻¹×10⁶. In particular embodiments, the ED₅₀ of recombinant human IL-2 or IL-15 is equivalent to the concentration required for the half-maximal stimulation of cell proliferation (XTT cleavage) with CTLL-2 cells. In certain embodiments, the ED₅₀ of recombinant human IL-7 is equivalent to the concentration required for the half-maximal stimulation for proliferation of PHA-activated human peripheral blood lymphocytes. Details relating to assays and calculations of IU for IL-2 are discussed in Wadhwa et al., Journal of Immunological Methods (2013), 379 (1-2): 1-7; and Gearing and Thorpe, Journal of Immunological Methods (1988), 114 (1-2): 3-9; details relating to assays and calculations of IU for IL-15 are discussed in Soman et al. Journal of Immunological Methods (2009) 348 (1-2): 83-94; hereby incorporated by reference in their entirety.

In particular embodiments, a composition of enriched CD8+ T cells is incubated under stimulating conditions in the presence of IL-2 and/or IL-15. In certain embodiments, a composition of enriched CD4+ T cells is incubated under stimulating conditions in the presence of IL-2, IL-7, and/or IL-15. In some embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15. In some aspects, the incubation of the enriched T cell composition also includes the presence of a stimulatory reagent, e.g., anti-CD3/anti-CD28 magnetic beads.

In some embodiments, the cells are incubated with a cytokine, e.g., a recombinant human cytokine, at a concentration of between 1 IU/ml and 1,000 IU/ml, between 10 IU/ml and 50 IU/ml, between 50 IU/ml and 100 IU/ml, between 100 IU/ml and 200 IU/ml, between 100 IU/ml and 500 IU/ml, between 250 IU/ml and 500 IU/ml, or between 500 IU/ml and 1,000 IU/ml.

In some embodiments, a composition of enriched T cells is incubated with IL-2, e.g., human recombinant IL-2, at a concentration between 1 IU/ml and 200 IU/ml, between 10 IU/ml and 200 IU/ml, between 10 IU/ml and 100 IU/ml, between 50 IU/ml and 150 IU/ml, between 80 IU/ml and 120 IU/ml, between 60 IU/ml and 90 IU/ml, or between 70 IU/ml and 90 IU/ml. In particular embodiments, the composition of enriched T cells is incubated with recombinant IL-2 at a concentration at or at about 50 IU/ml, 55 IU/ml, 60 IU/ml, 65 IU/ml, 70 IU/ml, 75 IU/ml, 80 IU/ml, 85 IU/ml, 90 IU/ml, 95 IU/ml, 100 IU/ml, 110 IU/ml, 120 IU/ml, 130 IU/ml, 140 IU/ml, or 150 IU/ml. In some embodiments, the composition of enriched T cells is incubated in the presence of or of about 85 IU/ml recombinant IL-2. In some embodiments, the composition incubated with recombinant IL-2 is enriched for a population of T cells, e.g., CD4+ T cells and/or CD8+ T cells. In some embodiments, the population of T cells is a population of CD4+ T cells. In some embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition. In some embodiments, the composition of enriched T cells is a composition of enriched CD4+ T cells. In particular embodiments, the composition of enriched T cells is enriched for CD4+ T cells, where CD8+ T cells are not enriched for and/or where CD8+ T cells are negatively selected for or depleted from the composition. In some embodiments, an enriched CD4+ T cell composition incubated with recombinant IL-2 may also be incubated with recombinant IL-7 and/or recombinant IL-15, such as in amounts described. In some embodiments, an enriched CD8+ T cell composition incubated with recombinant IL-2 may also be incubated with recombinant IL-15, such as in amounts described.

In some embodiments, a composition of enriched T cells is incubated with recombinant IL-7, e.g., human recombinant IL-7, at a concentration between 100 IU/ml and 2,000 IU/ml, between 500 IU/ml and 1,000 IU/ml, between 100 IU/ml and 500 IU/ml, between 500 IU/ml and 750 IU/ml, between 750 IU/ml and 1,000 RJ/ml, or between 550 IU/ml and 650 IU/ml. In particular embodiments, the composition of enriched T cells is incubated with recombinant IL-7 at a concentration at or at about 50 IU/ml, 100 IU/ml, 150 IU/ml, 200 IU/ml, 250 IU/ml, 300 RJ/ml, 350 IU/ml, 400 IU/ml, 450 IU/ml, 500 IU/ml, 550 IU/ml, 600 IU/ml, 650 IU/ml, 700 IU/ml, 750 IU/ml, 800 IU/ml, 750 IU/ml, 750 IU/ml, 750 IU/ml, or 1,000 IU/ml. In particular embodiments, the composition of enriched T cells is incubated in the presence of or of about 600 IU/ml of recombinant IL-7. In some embodiments, the composition incubated with recombinant IL-7 is enriched for a population of T cells, e.g., CD4+ T cells. In some embodiments, an enriched CD4+ T cell composition incubated with recombinant IL-7 may also be incubated with recombinant IL-2 and/or recombinant IL-15, such as in amounts described. In particular embodiments, the composition of enriched T cells is enriched for CD4+ T cells, where CD8+ T cells are not enriched for and/or where CD8+ T cells are negatively selected for or depleted from the composition. In some embodiments, an enriched CD8+ T cell composition is not incubated with recombinant IL-7.

In some embodiments, a composition of enriched T cells is incubated with recombinant IL-15, e.g., human recombinant IL-15, at a concentration between 0.1 IU/ml and 100 IU/ml, between 1 IU/ml and 100 IU/ml, between 1 IU/ml and 50 IU/ml, between 5 IU/ml and 25 IU/ml, between 25 IU/ml and 50 IU/ml, between 5 IU/ml and 15 IU/ml, or between 10 IU/ml and 100 IU/ml. In particular embodiments, the composition of enriched T cells is incubated with recombinant IL-15 at a concentration at or at about 1 IU/ml, 2 IU/ml, 3 IU/ml, 4 IU/ml, 5 IU/ml, 6 IU/ml, 7 IU/ml, 8 IU/ml, 9 IU/ml, 10 IU/ml, 11 IU/ml, 12 IU/ml, 13 IU/ml, 14 IU/ml, 15 IU/ml, 20 IU/ml, 25 IU/ml, 30 IU/ml, 40 IU/ml, or 50 IU/ml. In some embodiments, the composition of enriched T cells is incubated in or in about 10 IU/ml of recombinant IL-15. In some embodiments, the composition incubated with recombinant IL-15 is enriched for a population of T cells, e.g., CD4+ T cells and/or CD8+ T cells. In some embodiments, the population of T cells is a population of CD4+ T cells. In some embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition. In some embodiments, the composition of enriched T cells is a composition of enriched CD4+ T cells. In particular embodiments, the composition of enriched T cells is enriched for CD4+ T cells, where CD8+ T cells are not enriched for and/or where CD8+ T cells are negatively selected for or depleted from the composition. In some embodiments, an enriched CD4+ T cell composition incubated with recombinant IL-15 may also be incubated with recombinant IL-7 and/or recombinant IL-2, such as in amounts described. In some embodiments, an enriched CD8+ T cell composition incubated with recombinant IL-15 may also be incubated with recombinant IL-2, such as in amounts described.

In particular embodiments, the cells, such as enriched CD4+ T cells and/or enriched CD8+ T cells, are incubated with the stimulatory reagent in the presence of one or more antioxidants. In some embodiments, antioxidants include, but are not limited to, one or more antioxidants comprise a tocopherol, a tocotrienol, alpha-tocopherol, beta-tocopherol, gamma-tocopherol, delta-tocopherol, alpha-tocotrienol, beta-tocotrienol, alpha-tocopherolquinone, Trolox (6-hydroxy-2,5,7,8-tetramethylchroman-2-carboxylic acid), butylated hydroxyanisole (BHA), butylated hydroxytoluene (BHT), a flavonoids, an isoflavone, lycopene, beta-carotene, selenium, ubiquinone, luetin, S-adenosylmethionine, glutathione, taurine, N-acetyl cysteine (NAC), citric acid, L-carnitine, BHT, monothioglycerol, ascorbic acid, propyl gallate, methionine, cysteine, homocysteine, gluthatione, cystamine and cysstathionine, and/or glycine-glycine-histidine. In some aspects, the incubation of the enriched T cell composition, such as enriched CD4+ T cells and/or enriched CD8+ T cells, with an antioxidant also includes the presence of a stimulatory reagent, e.g., anti-CD3/anti-CD28 magnetic beads, and one or more recombinant cytokines, such as described.

In some embodiments, the one or more antioxidants is or includes a sulfur containing oxidant. In certain embodiments, a sulfur containing antioxidant may include thiol-containing antioxidants and/or antioxidants which exhibit one or more sulfur moieties, e.g., within a ring structure. In some embodiments, the sulfur containing antioxidants may include, for example, N-acetylcysteine (NAC) and 2,3-dimercaptopropanol (DMP), L-2-oxo-4-thiazolidinecarboxylate (OTC) and lipoic acid. In particular embodiments, the sulfur containing antioxidant is a glutathione precursor. In some embodiments, the glutathione precursor is a molecule which may be modified in one or more steps within a cell to derived glutathione. In particular embodiments, a glutathione precursor may include, but is not limited to N-acetyl cysteine (NAC), L-2-oxothiazolidine-4-carboxylic acid (Procysteine), lipoic acid, S-allyl cysteine, or methylmethionine sulfonium chloride.

In some embodiments, incubating the cells, such as enriched CD4+ T cells and/or enriched CD8+ T cells, under stimulating conditions includes incubating the cells in the presence of one or more antioxidants. In particular embodiments, the cells are stimulated with the stimulatory reagent in the presence of one or more antioxidants. In some embodiments, the cells are incubated in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1 500 μg/ml and 2 mg/ml, 500 μg/ml and 5 mg/ml, between 1 mg/ml and 10 mg/ml, or between 1 mg/ml and 100 mg/ml of the one or more antioxidants. In some embodiments, the cells are incubated in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of the one or more antioxidant. In some embodiments, the one or more antioxidants is or includes a sulfur containing antioxidant. In particular embodiments, the one or more antioxidants is or includes a glutathione precursor.

In some embodiments, the one or more antioxidants is or includes N-acetyl cysteine (NAC). In some embodiments, incubating the cells, such as enriched CD4+ T cells and/or enriched CD8+ T cells, under stimulating conditions includes incubating the cells in the presence of NAC. In particular embodiments, the cells are stimulated with the stimulatory reagent in the presence of NAC. In some embodiments, the cells are incubated in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1-500 μg/ml and 2 mg/ml, 500 μg/ml and 5 mg/ml, between 1 mg/ml and 10 mg/ml, or between 1 mg/ml and 100 mg/ml of NAC. In some embodiments, the cells are incubated in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of NAC. In some embodiments, the cells are incubated with or with about 0.8 mg/ml.

In particular embodiments, incubating the composition of enriched T cells, such as enriched CD4+ T cells and/or enriched CD8+ T cells, in the presence of one or more antioxidants, e.g., NAC, reduces the activation in the cells as compared to cells that are incubated in alternative and/or exemplary processes without the presence of antioxidants. In certain embodiments, the reduced activation is measured by the expression of one or more activation markers in the cell. In certain embodiments, markers of activation include, but are not limited to, increased intracellular complexity (e.g., as determined by measuring side scatter (SSC)), increased cell size (e.g., as determined by measuring cell diameter and/or forward scatter (FSC)), increased expression of CD27, and/or decreased expression of CD25. In some embodiments, the cells of the composition have negative, reduced, or low expression and/or degree of markers of activation when examined during or after the incubation, engineering, transduction, transfection, expansion, or formulation, or during or after any stage of the process occurring after the incubation. In some embodiments the cells of the composition have negative, reduced, or low expression and/or degree of markers of activation after the process is completed. In particular embodiments, the cells of the output composition have negative, reduced, or low expression and/or degree of markers of activation.

In some embodiments, flow cytometry is used to determine relative size of cells. In particular embodiments, the FSC and SSC parameters are used to analyze cells and distinguish the cells from one another based off of size and internal complexity. In particular embodiments, a particle or bead of a known size can be measured as a standard to determine the actual size of cells. In some embodiments, flow cytometry is used in combination with a stain, e.g., a labeled antibody, to measure or quantify the expression of a surface protein, such as a marker of activation, e.g., CD25 or CD27.

In some embodiments, the composition of enriched T cells, such as enriched CD4+ T cells and/or enriched CD8+ T cells, is incubated in the presence of one or more antioxidants e.g., NAC, and the cell diameter reduced by at least 0.25 μm, 0.5 μm, 0.75 μm, 1.0 μm, 1.5 μm, 2 μm, 2.5 μm, 3 μm, 3.5 μm, 4 μm, 4.5 μm, 5 μm, or more than 5 μm as compared to cells incubated in an alternative and/or exemplary process where the incubation is not performed in the presence of an antioxidant. In particular embodiments, the composition of enriched T cells is incubated in the presence of one or more antioxidants e.g., NAC, and the cell size, as measured by the FSC is reduced by at least 1%, at least 5%, at least 10%, at least 20%, at least 30%, at least 40%, at least 50%, at least 60%, at least 70%, at least 75%, at least 80%, at least 85%, or at least 90% as compared to cells incubated in an alternative and/or exemplary process where the incubation is not performed in the presence of an antioxidant.

In some embodiments, the composition of enriched T cells, such as enriched CD4+ T cells and/or enriched CD8+ T cells, is incubated in the presence of one or more antioxidants e.g., NAC, and the intracellular complexity, as measured by the SSC, is reduced by at least 1%, at least 5%, at least 10%, at least 20%, at least 30%, at least 40%, at least 50%, at least 60%, at least 70%, at least 75%, at least 80%, at least 85%, or at least 90% as compared to cells incubated in an alternative and/or exemplary process where the incubation is not performed in the presence of an antioxidant.

In particular embodiments, the composition of enriched T cells, such as enriched CD4+ T cells and/or enriched CD8+ T cells, is incubated in the presence of one or more antioxidants e.g., NAC, and the expression of CD27, e.g., as measured by the flow cytometry, is reduced by at least 1%, at least 5%, at least 10%, at least 20%, at least 30%, at least 40%, at least 50%, at least 60%, at least 70%, at least 75%, at least 80%, at least 85%, at least 90%, at least 95%, or at least 99% as compared to cells incubated in an alternative and/or exemplary process where the incubation is not performed in the presence of an antioxidant.

In certain embodiments, the composition of enriched T cells, such as enriched CD4+ T cells and/or enriched CD8+ T cells, is incubated in the presence of one or more antioxidants, e.g., NAC, and the expression of CD25, e.g., as measured by the flow cytometry, is increased by at least 1%, at least 5%, at least 10%, at least 20%, at least 30%, at least 40%, at least 50%, at least 60%, at least 70%, at least 75%, at least 80%, at least 85%, at least 90%, at least 100%, at least 150%, at least 1-fold, at least 2-fold, at least 3-fold, at least 4-fold, at least 5-fold, at least 10-fold, at least 25-fold, at least 50-fold, or at least 100-fold as compared to cells incubated in an alternative and/or exemplary process where the incubation is not performed in the presence of an antioxidant.

In particular embodiments, incubating the composition of enriched T cells, such as enriched CD4+ T cells and/or enriched CD8+ T cells, in the presence of one or more antioxidants, e.g., NAC, increases the expansion, e.g., during the incubation or cultivation step or stage as described in Section II-D. In some embodiments, a composition of enriched cells achieves a 2-fold, a 2.5 fold, a 3 fold, a 3.5 fold, a 4 fold, a 4.5 fold a 5 fold, a 6 fold, a 7 fold, an 8 fold, a nine fold, a 10-fold, or greater than a 10 fold expansion within 14 days, 12 days, 10 days, 9 days, 8 days, 7 days, 6 days, 5 days, 4 days, or within 3 days of the start of the cultivation. In some embodiments, the composition of enriched T cells is incubated in the presence of one or more antioxidants and the cells of the compositions undergo at least 10%, at least a 20%, at least a 30%, at least a 40%, at least a 50%, at least a 60%, at least a 70%, at least a 75%, at least an 80%, at least an 85%, at least a 90%, at least a 100%, at least a 150%, at least a 1-fold, at least a 2-fold, at least a 3-fold, at least a 4-fold, at least a 5-fold, at least a 10-fold faster rate of expansion during the cultivation than cultivated cells that were incubated in an alternative and/or exemplary process where the incubation is not performed in the presence of an antioxidant.

In particular embodiments, incubating the composition of enriched cells, such as enriched CD4+ T cells and/or enriched CD8+ T cells, in the presence of one or more antioxidants, e.g., NAC, reduces the amount of cell death, e.g., by apoptosis. In some embodiments, the composition of enriched T cells is incubated in the presence of a one or more antioxidants, e.g., NAC, and at least 50%, at least 60%, at least 70%, at least 75%, at least 80%, at least 85%, at least 90%, at least 95%, at least 99%, or at least 99.9% of the cells survive, e.g., do not undergo apoptosis, during or at least 1 day, 2 days, 3 days, 4 days, 5 days, 6 days, 7 days, or more than 7 days after the incubation is complete. In some embodiments, the composition is incubated in the presence of one or more antioxidants, e.g., NAC, and the cells of the composition have at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-fold, at least 50-fold, or at least 100-fold greater survival as compared to cells undergoing an exemplary and/or alternative process where cells are not incubated in the presence or one or more antioxidants.

In particular embodiments, the composition of enriched T cells, such as enriched CD4+ T cells and/or enriched CD8+ T cells, is incubated in the presence of one or more antioxidants e.g., NAC, and caspase expression, e.g., caspase 3 expression, is reduced by at least 1%, at least 5%, at least 10%, at least 20%, at least 30%, at least 40%, at least 50%, at least 60%, at least 70%, at least 75%, at least 80%, at least 85%, at least 90%, at least 95%, or at least 99% as compared to cells incubated in an alternative and/or exemplary process where the incubation is not performed in the presence of an antioxidant.

In some embodiments, the compositions or cells, such as enriched CD4+ T cells and/or enriched CD8+ T cells, are incubated in the presence of stimulating conditions or a stimulatory agent, such as described. Such conditions include those designed to induce proliferation, expansion, activation, and/or survival of cells in the population, to mimic antigen exposure, and/or to prime the cells for genetic engineering, such as for the introduction of a recombinant antigen receptor. Exemplary stimulatory reagents, such as anti-CD3/anti-CD28 magnetic beads, are described below. The incubation with the stimulatory reagent may also be carried out in the presence of one or more stimulatory cytokine, such as in the presence of one or more of recombinant IL-2, recombinant IL-7 and/or recombinant IL-15 and/or in the presence of at least one antioxidant such as NAC, such as described above. In some embodiments, a composition of enriched CD4+ T cells are incubated under stimulatory conditions with a stimulatory agent, recombinant IL-2, recombinant IL-7, recombinant IL-15 and NAC, such as in amounts as described. In some embodiments, a composition of enriched CD8+ T cells are incubated under stimulatory conditions with a stimulatory agent, recombinant IL-2, recombinant IL-15 and NAC, such as in amounts as described.

In some embodiments, the conditions for stimulation and/or activation can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177 to Riddell et al., Klebanoff et al. (2012) J Immunother. 35(9):651-660, Terakura et al. (2012) Blood. 1:72-82, and/or Wang et al. (2012) J Immunother. 35(9):689-701.

In some embodiments, at least a portion of the incubation in the presence of one or more stimulating conditions or a stimulatory agents is carried out in the internal cavity of a centrifugal chamber, for example, under centrifugal rotation, such as described in International Publication Number WO2016/073602. In some embodiments, at least a portion of the incubation performed in a centrifugal chamber includes mixing with a reagent or reagents to induce stimulation and/or activation. In some embodiments, cells, such as selected cells, are mixed with a stimulating condition or stimulatory agent in the centrifugal chamber. In some aspects of such processes, a volume of cells is mixed with an amount of one or more stimulating conditions or agents that is far less than is normally employed when performing similar stimulations in a cell culture plate or other system.

In some embodiments, the stimulating agent is added to cells in the cavity of the chamber in an amount that is substantially less than (e.g., is no more than 5%, 10%, 20%, 30%, 40%, 50%, 60%, 70% or 80% of the amount) as compared to the amount of the stimulating agent that is typically used or would be necessary to achieve about the same or similar efficiency of selection of the same number of cells or the same volume of cells when selection is performed without mixing in a centrifugal chamber, e.g., in a tube or bag with periodic shaking or rotation. In some embodiments, the incubation is performed with the addition of an incubation buffer to the cells and stimulating agent to achieve a target volume with incubation of the reagent of, for example, about 10 mL to about 200 mL, or about 20 mL to about 125 mL, such as at least or about at least or about 10 mL, 20 mL, 30 mL, 40 mL, 50 mL, 60 mL, 70 mL, 80 mL, 90 mL, 100 mL, 105 mL, 110 mL, 115 mL, 120 mL, 125 mL, 130 mL, 135 mL, 140 mL, 145 mL, 150 mL, 160 mL, 170 mL, 180 mL, 190 mL, or 200 mL. In some embodiments, the incubation buffer and stimulating agent are pre-mixed before addition to the cells. In some embodiments, the incubation buffer and stimulating agent are separately added to the cells. In some embodiments, the stimulating incubation is carried out with periodic gentle mixing condition, which can aid in promoting energetically favored interactions and thereby permit the use of less overall stimulating agent while achieving stimulating and activation of cells.

In some embodiments, the incubation generally is carried out under mixing conditions, such as in the presence of spinning, generally at relatively low force or speed, such as speed lower than that used to pellet the cells, such as from 600 rpm to 1700 rpm or from about 600 rpm to about 1700 rpm (e.g., at or about or at least 600 rpm, 1000 rpm, or 1500 rpm or 1700 rpm), such as at an RCF at the sample or wall of the chamber or other container of from 80 g to 100 g or from about 80 g to about 100 g (e.g., at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, the total duration of the incubation, e.g., with the stimulating agent, is between or between about 1 hour and 96 hours, 1 hour and 72 hours, 1 hour and 48 hours, 4 hours and 36 hours, 8 hours and 30 hours, 18 hours and 30 hours, or 12 hours and 24 hours, such as at least or about at least or about 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

In some embodiments, the cells are cultured, cultivated, and/or incubated under stimulating conditions prior to and/or during a step for introducing a polynucleotide, e.g., a polynucleotide encoding a recombinant receptor, to the cells, e.g., by transduction and/or transfection, such as described by Section II-C. In certain embodiments the cells are cultured, cultivated, and/or incubated under stimulating conditions for an amount of time between 30 minutes and 2 hours, between 1 hour and 8 hours, between 1 hour and 6 hours, between 6 hours and 12 hours, between 12 hours and 18 hours, between 16 hours and 24 hours, between 12 hours and 36 hours, between 24 hours and 48 hours, between 24 hours and 72 hours, between 42 hours and 54 hours, between 60 hours and 120 hours between 96 hours and 120 hours, between 90 hours and between 1 days and 7 days, between 3 days and 8 days, between 1 day and 3 days, between 4 days and 6 days, or between 4 days and 5 days prior to the genetic engineering. In some embodiments, the cells are incubated for or for about 2 days prior to the engineering.

In certain embodiments, the cells are incubated with and/or in the presence of the stimulatory reagent prior to and/or during genetically engineering the cells. In certain embodiments the cells are incubated with and/or in the presence of the stimulatory reagent for an amount of time between 12 hours and 36 hours, between 24 hours and 48 hours, between 24 hours and 72 hours, between 42 hours and 54 hours, between 60 hours and 120 hours between 96 hours and 120 hours, between 90 hours and between 2 days and 7 days, between 3 days and 8 days, between 1 day and 8 days, between 4 days and 6 days, or between 4 days and 5 days. In particular embodiments, the cells are cultured, cultivated, and/or incubated under stimulating conditions prior to and/or during genetically engineering the cells for an amount of time of less than 10 days, 9 days, 8 days, 7 days, 6 days, or 5 days, 4 days, or for an amount of time less than 168 hours, 162 hours, 156 hours, 144 hours, 138 hours, 132 hours, 120 hours, 114 hours, 108 hours, 102 hours, or 96 hours. In particular embodiments, the cells are incubated with and/or in the presence of the stimulatory reagent for or for about 4 days, 5 days, 6 days, or 7 days. In some embodiments, the cells are incubated with and/or in the presence of the stimulatory reagent for or for about 4 days. In particular embodiments, the cells are incubated with and/or in the presence of the stimulatory reagent for or for about 5 days. In certain embodiments, the cells are incubated with and/or in the presence of the stimulatory reagent for less than 7 days.

In some embodiments, incubating the cells under stimulating conditions includes incubating the cells with a stimulatory reagent that is described in Section II-B-1. In some embodiments, the stimulatory reagent contains or includes a bead, such as a paramagnetic bead, and the cells are incubated with the stimulatory reagent at a ratio of less than 3:1 (beads:cells), such as a ratio of 1:1. In particular embodiments, the cells are incubated with the stimulatory reagent in the presence of one or more cytokines and/or one or more antioxidants. In some embodiments, a composition of enriched CD4+ T cells is incubated with the stimulatory reagent at a ratio of 1:1 (beads:cells) in the presence of recombinant IL-2, IL-7, IL-15, and NAC. In certain embodiments, a composition of enriched CD8+ T cells is incubated with the stimulatory reagent at a ratio of 1:1 (beads:cells) in the presence of recombinant IL-2, IL-15, and NAC. In some embodiments, the stimulatory reagent is removed and/or separated from the cells at, within, or within about 6 days, 5 days, or 4 days from the start or initiation of the incubation, e.g., from the time the stimulatory reagent is added to or contacted with the cells.

1. Stimulatory Reagents

In some embodiments, incubating a composition of enriched cells under stimulating conditions is or includes incubating and/or contacting the composition of enriched cells with a stimulatory reagent that is capable of activating and/or expanding T cells. In some embodiments, the stimulatory reagent is capable of stimulating and/or activating one or more signals in the cells. In some embodiments, the one or more signals are mediated by a receptor. In particular embodiments, the one or more signals are or are associated with a change in signal transduction and/or a level or amount of secondary messengers, e.g., cAMP and/or intracellular calcium, a change in the amount, cellular localization, confirmation, phosphorylation, ubiquitination, and/or truncation of one or more cellular proteins, and/or a change in a cellular activity, e.g., transcription, translation, protein degradation, cellular morphology, activation state, and/or cell division. In particular embodiments, the stimulatory reagent activates and/or is capable of activating one or more intracellular signaling domains of one or more components of a TCR complex and/or one or more intracellular signaling domains of one or more costimulatory molecules.

In certain embodiments, the stimulatory reagent contains a particle, e.g., a bead, that is conjugated or linked to one or more agents, e.g., biomolecules, that are capable of activating and/or expanding cells, e.g., T cells. In some embodiments, the one or more agents are bound to a bead. In some embodiments, the bead is biocompatible, i.e., composed of a material that is suitable for biological use. In some embodiments, the beads are non-toxic to cultured cells, e.g., cultured T cells. In some embodiments, the beads may be any particles which are capable of attaching agents in a manner that permits an interaction between the agent and a cell.

In some embodiments, a stimulatory reagent contains one or more agents that are capable of activating and/or expanding cells, e.g., T cells, that are bound to or otherwise attached to a bead, for example to the surface of the bead. In certain embodiments, the bead is a non-cell particle. In particular embodiments, the bead may include a colloidal particle, a microsphere, nanoparticle, a magnetic bead, or the like. In some embodiments the beads are agarose beads. In certain embodiments, the beads are sepharose beads.

In particular embodiments, the stimulatory reagent contains beads that are monodisperse. In certain embodiments, beads that are monodisperse comprise size dispersions having a diameter standard deviation of less than 5% from each other.

In some embodiments, the bead contains one or more agents, such as an agent that is coupled, conjugated, or linked (directly or indirectly) to the surface of the bead. In some embodiments, an agent as contemplated herein can include, but is not limited to, RNA, DNA, proteins (e.g., enzymes), antigens, polyclonal antibodies, monoclonal antibodies, antibody fragments, carbohydrates, lipids lectins, or any other biomolecule with an affinity for a desired target. In some embodiments, the desired target is a T cell receptor and/or a component of a T cell receptor. In certain embodiments, the desired target is CD3. In certain embodiment, the desired target is a T cell costimulatory molecule, e.g., CD28, CD137 (4-1-BB), OX40, or ICOS. The one or more agents may be attached directly or indirectly to the bead by a variety of methods known and available in the art. The attachment may be covalent, noncovalent, electrostatic, or hydrophobic and may be accomplished by a variety of attachment means, including for example, a chemical means, a mechanical means, or an enzymatic means. In some embodiments, a biomolecule (e.g., a biotinylated anti-CD3 antibody) may be attached indirectly to the bead via another biomolecule (e.g., anti-biotin antibody) that is directly attached to the bead.

In some embodiments, the stimulatory reagent contains a bead and one or more agents that directly interact with a macromolecule on the surface of a cell. In certain embodiments, the bead (e.g., a paramagnetic bead) interacts with a cell via one or more agents (e.g., an antibody) specific for one or more macromolecules on the cell (e.g., one or more cell surface proteins). In certain embodiments, the bead (e.g., a paramagnetic bead) is labeled with a first agent described herein, such as a primary antibody (e.g., an anti-biotin antibody) or other biomolecule, and then a second agent, such as a secondary antibody (e.g., a biotinylated anti-CD3 antibody) or other second biomolecule (e.g., streptavidin), is added, whereby the secondary antibody or other second biomolecule specifically binds to such primary antibodies or other biomolecule on the particle.

In some embodiments, the stimulatory reagent contains one or more agents (e.g., antibody) that is attached to a bead (e.g., a paramagnetic bead) and specifically binds to one or more of the following macromolecules on a cell (e.g., a T cell): CD2, CD3, CD4, CD5, CD8, CD25, CD27, CD28, CD29, CD31, CD44, CD45RA, CD45RO, CD54 (ICAM-1), CD127, MHCI, MHCII, CTLA-4, ICOS, PD-1, OX40, CD27L (CD70), 4-1BB (CD137), 4-1BBL, CD30L, LIGHT, IL-2R, IL-12R, IL-1R, IL-15R; IFN-gammaR, TNF-alphaR, IL-4R, IL-10R, CD18/CD11a (LFA-1), CD62L (L-selectin), CD29/CD49d (VLA-4), Notch ligand (e.g., Delta-like 1/4, Jagged 1/2, etc.), CCR1, CCR2, CCR3, CCR4, CCR5, CCR7, and CXCR3 or fragment thereof including the corresponding ligands to these macromolecules or fragments thereof. In some embodiments, an agent (e.g., antibody) attached to the bead specifically binds to one or more of the following macromolecules on a cell (e.g., a T cell): CD28, CD62L, CCR7, CD27, CD127, CD3, CD4, CD8, CD45RA, and/or CD45RO. In some embodiments, one or more of the agents attached to the bead is an antibody. The antibody can include a polyclonal antibody, monoclonal antibody (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)2, and Fv). In some embodiments, the stimulatory reagent is an antibody fragment (including antigen-binding fragment), e.g., a Fab, Fab′-SH, Fv, scFv, or (Fab′)2 fragment. It will be appreciated that constant regions of any isotype can be used for the antibodies contemplated herein, including IgG, IgM, IgA, IgD, and IgE constant regions, and that such constant regions can be obtained from any human or animal species (e.g., murine species).

In some embodiments, the agent is an antibody that binds to and/or recognizes one or more components of a T cell receptor. In particular embodiments, the agent is an anti-CD3 antibody. In certain embodiments, the agent is an antibody that binds to and/or recognizes a co-receptor. In some embodiments, the stimulatory reagent comprises an anti-CD28 antibody. In particular embodiments, the stimulatory agent contains an anti-CD3 antibody and an anti-CD28 antibody. In some embodiments, the antibody is a Fab. In some embodiments, the stimulatory agent contains an anti-CD3 Fab and an anti-CD28 Fab.

In some embodiments, the stimulating agent is an anti-CD3/anti-CD28 streptavidin oligomeric reagent, such as described in PCT publication No. WO/2015/158868 or WO2019/197949.

In some embodiments, the simulating agents are anti-CD3/anti-CD28 beads (e.g., DYNABEADS® M-450 CD3/CD28 T Cell Expander, and/or ExpACT® beads).

In some embodiments, the bead has a diameter of greater than about 0.001 μm, greater than about 0.01 μm, greater than about 0.1 μm, greater than about 1.0 μm, greater than about 10 μm, greater than about 50 μm, greater than about 100 μm or greater than about 1000 μm and no more than about 1500 μm. In some embodiments, the bead has a diameter of about 1.0 μm to about 500 μm, about 1.0 μm to about 150 μm, about 1.0 μm to about 30 μm, about 1.0 μm to about 10 μm, about 1.0 μm to about 5.0 μm, about 2.0 μm to about 5.0 μm, or about 3.0 μm to about 5.0 μm. In some embodiments, the bead has a diameter of about 3 μm to about 5 μm. In some embodiments, the bead has a diameter of at least or at least about or about 0.001 μm, 0.01 μm, 0.1 μm, 0.5 μm, 1.0 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10 μm, 12 μm, 14 μm, 16 μm, 18 μm or 20 μm. In certain embodiments, the bead has a diameter of or about 4.5 μm. In certain embodiments, the bead has a diameter of or about 2.8 μm.

In some embodiments, the beads have a density of greater than 0.001 g/cm³, greater than 0.01 g/cm³, greater than 0.05 g/cm³, greater than 0.1 g/cm³, greater than 0.5 g/cm³, greater than 0.6 g/cm³, greater than 0.7 g/cm³, greater than 0.8 g/cm³, greater than 0.9 g/cm³, greater than 1 g/cm³, greater than 1.1 g/cm³, greater than 1.2 g/cm³, greater than 1.3 g/cm³, greater than 1.4 g/cm³, greater than 1.5 g/cm³, greater than 2 g/cm³, greater than 3 g/cm³, greater than 4 g/cm³, or greater than 5 g/cm³. In some embodiments, the beads have a density of between about 0.001 g/cm³ and about 100 g/cm³, about 0.01 g/cm³ and about 50 g/cm³, about 0.1 g/cm³ and about 10 g/cm³, about 0.1 g/cm³ and about 0.5 g/cm³, about 0.5 g/cm³ and about 1 g/cm³, about 0.5 g/cm³ and about 1.5 g/cm³, about 1 g/cm³ and about 1.5 g/cm³, about 1 g/cm³ and about 2 g/cm³, or about 1 g/cm³ and about 5 g/cm³. In some embodiments, the beads have a density of about 0.5 g/cm³, about 0.5 g/cm³, about 0.6 g/cm³, about 0.7 g/cm³, about 0.8 g/cm³, about 0.9 g/cm³, about 1.0 g/cm³, about 1.1 g/cm³, about 1.2 g/cm³, about 1.3 g/cm³, about 1.4 g/cm³, about 1.5 g/cm³, about 1.6 g/cm³, about 1.7 g/cm³, about 1.8 g/cm³, about 1.9 g/cm³, or about 2.0 g/cm³. In certain embodiments, the beads have a density of about 1.6 g/cm³. In particular embodiments, the beads or particles have a density of about 1.5 g/cm³. In certain embodiments, the particles have a density of about 1.3 g/cm³.

In certain embodiments, a plurality of the beads has a uniform density. In certain embodiments, a uniform density comprises a density standard deviation of less than 10%, less than 5%, or less than 1% of the mean bead density.

In some embodiments, the beads have a surface area of between about 0.001 m² per each gram of particles (m²/g) to about 1,000 m²/g, about 0.010 m²/g to about 100 m²/g, about 0.1 m²/g to about 10 m²/g, about 0.1 m²/g to about 1 m²/g, about 1 m²/g to about 10 m²/g, about 10 m²/g to about 100 m²/g, about 0.5 m²/g to about 20 m²/g, about 0.5 m²/g to about 5 m²/g, or about 1 m²/g to about 4 m²/g. In some embodiments, the particles or beads have a surface area of about 1 m²/g to about 4 m²/g.

In some embodiments, the bead reacts in a magnetic field. In some embodiments, the bead is a magnetic bead. In some embodiments, the magnetic bead is paramagnetic. In particular embodiments, the magnetic bead is superparamagnetic. In certain embodiments, the beads do not display any magnetic properties unless they are exposed to a magnetic field.

In particular embodiments, the bead comprises a magnetic core, a paramagnetic core, or a superparamagnetic core. In some embodiments, the magnetic core contains a metal. In some embodiments, the metal can be, but is not limited to, iron, nickel, copper, cobalt, gadolinium, manganese, tantalum, zinc, zirconium or any combinations thereof. In certain embodiments, the magnetic core comprises metal oxides (e.g., iron oxides), ferrites (e.g., manganese ferrites, cobalt ferrites, nickel ferrites, etc.), hematite and metal alloys (e.g., CoTaZn). In some embodiments, the magnetic core comprises one or more of a ferrite, a metal, a metal alloy, an iron oxide, or chromium dioxide. In some embodiments, the magnetic core comprises elemental iron or a compound thereof. In some embodiments, the magnetic core comprises one or more of magnetite (Fe3O4), maghemite (γFe2O3), or greigite (Fe3S4). In some embodiments, the inner core comprises an iron oxide (e.g., Fe₃O₄).

In certain embodiments, the bead contains a magnetic, paramagnetic, and/or superparamagnetic core that is covered by a surface functionalized coat or coating. In some embodiments, the coat can contain a material that can include, but is not limited to, a polymer, a polysaccharide, a silica, a fatty acid, a protein, a carbon, agarose, sepharose, or a combination thereof. In some embodiments, the polymer can be a polyethylene glycol, poly (lactic-co-glycolic acid), polyglutaraldehyde, polyurethane, polystyrene, or a polyvinyl alcohol. In certain embodiments, the outer coat or coating comprises polystyrene. In particular embodiments, the outer coating is surface functionalized.

In some embodiments, the stimulatory reagent comprises a bead that contains a metal oxide core (e.g., an iron oxide core) and a coat, wherein the metal oxide core comprises at least one polysaccharide (e.g., dextran), and wherein the coat comprises at least one polysaccharide (e.g., amino dextran), at least one polymer (e.g., polyurethane) and silica. In some embodiments the metal oxide core is a colloidal iron oxide core. In certain embodiments, the one or more agents include an antibody or antigen-binding fragment thereof. In particular embodiments, the one or more agents include an anti-CD3 antibody and an anti-CD28 antibody or antigen-binding fragments thereof. In some embodiments, the stimulatory reagent comprises an anti-CD3 antibody, anti-CD28 antibody, and an anti-biotin antibody. In some embodiments, the stimulatory reagent comprises an anti-biotin antibody. In some embodiments, the bead has a diameter of about 3 μm to about 10 In some embodiments, the bead has a diameter of about 3 μm to about 5 μm. In certain embodiments, the bead has a diameter of about 3.5 μm.

In some embodiments, the stimulatory reagent comprises one or more agents that are attached to a bead comprising a metal oxide core (e.g., an iron oxide inner core) and a coat (e.g., a protective coat), wherein the coat comprises polystyrene. In certain embodiments, the beads are monodisperse, paramagnetic (e.g., superparamagnetic) beads comprising a paramagnetic (e.g., superparamagnetic) iron core, e.g., a core comprising magnetite (Fe₃O₄) and/or maghemite (γFe₂O₃) c and a polystyrene coat or coating. In some embodiments, the bead is non-porous. In some embodiments, the beads contain a functionalized surface to which the one or more agents are attached. In certain embodiments, the one or more agents are covalently bound to the beads at the surface. In some embodiments, the one or more agents include an antibody or antigen-binding fragment thereof. In some embodiments, the one or more agents include an anti-CD3 antibody and an anti-CD28 antibody. In some embodiments, the stimulatory reagent is or comprises anti-CD3/anti-CD28 magnetic beads. In some embodiments, the one or more agents include an anti-CD3 antibody and/or an anti-CD28 antibody, and an antibody or antigen fragment thereof capable of binding to a labeled antibody (e.g., biotinylated antibody), such as a labeled anti-CD3 or anti-CD28 antibody. In certain embodiments, the beads have a density of about 1.5 g/cm³ and a surface area of about 1 m²/g to about 4 m²/g. In particular embodiments; the beads are monodisperse superparamagnetic beads that have a diameter of about 4.5 μm and a density of about 1.5 g/cm³. In some embodiments, the beads the beads are monodisperse superparamagnetic beads that have a mean diameter of about 2.8 μm and a density of about 1.3 g/cm³.

In some embodiments, the composition of enriched T cells is incubated with stimulatory reagent a ratio of beads to cells at or at about 3:1, 2.5:1, 2:1, 1.5:1, 1.25:1, 1.2:1, 1.1:1, 1:1, 0.9:1, 0.8:1, 0.75:1, 0.67:1, 0.5:1, 0.3:1, or 0.2:1. In particular embodiments, the ratio of beads to cells is between 2.5:1 and 0.2:1, between 2:1 and 0.5:1, between 1.5:1 and 0.75:1, between 1.25:1 and 0.8:1, between 1.1:1 and 0.9:1. In particular embodiments, the ratio of stimulatory reagent to cells is about 1:1 or is 1:1.

2. Removal of the Stimulatory Reagent from Cells

In certain embodiments, the stimulatory reagent, e.g., anti-CD3/anti-CD28 magnetic beads, is removed and/or separated from the cells. Without wishing to be bound by theory, particular embodiments contemplate that the binding and/or association between a stimulatory reagent and cells may, in some circumstances, be reduced over time during the incubation. In certain embodiments, one or more agents may be added to reduce the binding and/or association between the stimulatory reagent and the cells. In particular embodiments, a change in cell culture conditions, e.g., media temperature of pH, may reduce the binding and/or association between the stimulatory reagent and the cells. Thus, in some embodiments, the stimulatory reagent may be removed from an incubation, cell culture system, and/or a solution separately from the cells, e.g., without removing the cells from the incubation, cell culture system, and/or a solution as well.

Methods for removing stimulatory reagents (e.g., stimulatory reagents that are or contain particles such as bead particles or magnetizable particles) from cells are known. In some embodiments, the use of competing antibodies, such as non-labeled antibodies, can be used, which, for example, bind to a primary antibody of the stimulatory reagent and alter its affinity for its antigen on the cell, thereby permitting for gentle detachment. In some cases, after detachment, the competing antibodies may remain associated with the particle (e.g., bead particle) while the unreacted antibody is or may be washed away and the cell is free of isolating, selecting, enriching and/or activating antibody. Exemplary of such a reagent is DETACaBEAD (Friedl et al. 1995; Entschladen et al. 1997). In some embodiments, particles (e.g., bead particles) can be removed in the presence of a cleavable linker (e.g., DNA linker), whereby the particle-bound antibodies are conjugated to the linker (e.g., CELLection, Dynal). In some cases, the linker region provides a cleavable site to remove the particles (e.g., bead particles) from the cells after isolation, for example, by the addition of DNase or other releasing buffer. In some embodiments, other enzymatic methods can also be employed for release of a particle (e.g., bead particle) from cells. In some embodiments, the particles (e.g., bead particles or magnetizable particles) are biodegradable.

In some embodiments, the stimulatory reagent is magnetic, paramagnetic, and/or superparamagnetic, and/or contains a bead that is magnetic, paramagnetic, and/or superparamagnetic, and the stimulatory reagent may be removed from the cells by exposing the cells to a magnetic field. Examples of suitable equipment containing magnets for generating the magnetic field include DynaMag CTS (Thermo Fisher), Magnetic Separator (Takara) and EasySep Magnet (Stem Cell Technologies).

In particular embodiments, the stimulatory reagent is removed or separated from the cells prior to the completion of the provided methods, e.g., prior to harvesting, collecting, and/or formulating engineered cells produced by the methods provided herein. In some embodiments, the stimulatory reagent is removed and/or separated from the cells prior to engineering, e.g., transducing or transfecting, the cells. In particular embodiments, the stimulatory reagent is removed and/or separated from the cells after the step of engineering the cells. In certain embodiments, the stimulatory reagent is removed prior to the cultivation of the cells, e.g., prior to the cultivation of the engineered, e.g., transfected or transduced, cells under conditions to promote proliferation and/or expansion.

In certain embodiments, the stimulatory reagent is separated and/or removed from the cells after an amount of time. In particular embodiments, the amount of time is an amount of time from the start and/or initiation of the incubation under stimulating conditions. In particular embodiments the start of the incubation is considered at or at about the time the cells are contacted with the stimulatory reagent and/or a media or solution containing the stimulatory reagent. In particular embodiments, the stimulatory reagent is removed or separated from the cells within or within about 10 days, 9 days, 8 days, 7 days, 6 days, 5 days, 4 days, 3 days, or 2 days after the start or initiation of the incubation. In particular embodiments, the stimulatory reagent is removed and/or separated from the cells at or at about 9 days, 8 days, 7 days, 6 days, 5 days, 4 days, 3 days, or 2 days after the start or initiation of the incubation. In certain embodiments, the stimulatory reagent is removed and/or separated from the cells at or at about 168 hours, 162 hours, 156 hours, 144 hours, 138 hours, 132 hours, 120 hours, 114 hours, 108 hours, 102 hours, or 96 hours after the start or initiation of the incubation. In particular embodiments, the stimulatory reagent is removed and/or separated from the cells at or at about 5 days after the start and/or initiation of the incubation. In some embodiments, the stimulatory reagent is removed and/or separated from the cells at or at about 4 days after the start and/or initiation of the incubation.

C. Engineering Cells

In some embodiments, the provided methods involve administering to a subject having a disease or condition cells expressing a recombinant antigen receptor. Various methods for the introduction of genetically engineered components, e.g., recombinant receptors, e.g., CARs or TCRs, are well known and may be used with the provided methods and compositions. Exemplary methods include those for transfer of nucleic acids encoding the receptors, including via viral, e.g., retroviral or lentiviral, transduction, transposons, and electroporation.

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

In some embodiments, the methods provided herein are used in association with engineering one or more compositions of enriched T cells. In certain embodiments, the engineering is or includes the introduction of a polynucleotide, e.g., a recombinant polynucleotide encoding a recombinant protein. In particular embodiments, the recombinant proteins are recombinant receptors, such as any described in Section II. Introduction of the nucleic acid molecules encoding the recombinant protein, such as recombinant receptor, in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g., retroviral or lentiviral, transduction, transposons, and electroporation. In some embodiments, the engineering produces one or more engineered compositions of enriched T cells.

In certain embodiments, one or more compositions of enriched T cells are engineered, e.g., transduced or transfected, prior to cultivating the cells, e.g., under conditions that promote proliferation and/or expansion, such as by a method provided in Section II-D. In particular embodiments, one or more compositions of enriched T cells are engineered after the one or more compositions have been stimulated, activated, and/or incubated under stimulating conditions, such as described in methods provided in Section II-B. In particular embodiments, the one or more compositions are stimulated compositions. In particular embodiments, the one or more stimulated compositions have been previously cryofrozen and stored, and are thawed prior to engineering.

In certain embodiments, the one or more compositions of stimulated T cells are or include two separate stimulated compositions of enriched T cells. In particular embodiments, two separate compositions of enriched T cells, e.g., two separate compositions of enriched T cells that have been selected, isolated, and/or enriched from the same biological sample, are separately engineered. In certain embodiments, the two separate compositions include a composition of enriched CD4+ T cells. In particular embodiments, the two separate compositions include a composition of enriched CD8+ T cells. In some embodiments, two separate compositions of enriched CD4+ T cells and enriched CD8+ T cells, such as following incubation under stimulating conditions as described above, are genetically engineered separately. In some embodiments, a single composition of enriched T cells is genetically engineered. In certain embodiments, the single composition is a composition of enriched CD4+T cells. In some embodiments, the single composition is a composition of enriched CD4+ and CD8+ T cells that have been combined from separate compositions prior to the engineering.

In some embodiments, the composition of enriched CD4+ T cells, such as stimulated CD4+ T cells, that is engineered, e.g., transduced or transfected, includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the composition of enriched CD4+ T cells, such as stimulated CD4+ T cells, that is engineered includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells, such as stimulated CD8+ T cells, that is engineered, e.g., transduced or transfected, includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of enriched CD8+ T cells that, such as stimulated CD8+ T cells, that is engineered includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, separate compositions of enriched CD4+ and CD8+ T cells are combined into a single composition and are genetically engineered, e.g., transduced or transfected. In certain embodiments, separate engineered compositions of enriched CD4+ and enriched CD8+ T cells are combined into a single composition after the genetic engineering has been performed and/or completed. In particular embodiments, separate compositions of enriched CD4+ and CD8+ T cells, such as separate compositions of stimulated CD4+ and CD8+ T cells are separately engineered and are separately processed for cultivation and/or expansion of T cells after the genetic engineering and been performed and/or completed.

In some embodiments, the introduction of a polynucleotide, e.g., a recombinant polynucleotide encoding a recombinant protein, is carried out by contacting enriched CD4+ or CD8+ T cells, such as stimulated CD4+ or CD8+ T cells, with a viral particles containing the polynucleotide. In some embodiments, contacting can be effected with centrifugation, such as spinoculation (e.g., centrifugal inoculation). In some embodiments, the composition containing cells, viral particles and reagent can be rotated, generally at relatively low force or speed, such as speed lower than that used to pellet the cells, such as from 600 rpm to 1700 rpm or from about 600 rpm to about 1700 rpm (e.g., at or about or at least 600 rpm, 1000 rpm, or 1500 rpm or 1700 rpm). In some embodiments, the rotation is carried at a force, e.g., a relative centrifugal force, of from 100 g to 3200 g or from about 100 g to about 3200 g (e.g., at or about or at least at or about 100 g, 200 g, 300 g, 400 g, 500 g, 1000 g, 1500 g, 2000 g, 2500 g, 3000 g or 3200 g), such as at or about 693 g, as measured for example at an internal or external wall of the chamber or cavity. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured). In some embodiments, at least a portion of the contacting, incubating, and/or engineering of the cells, e.g., cells from an stimulated composition of enriched CD4+ T cell or enriched CD8+ T cells, with the virus is performed with a rotation of between about 100 g and 3200 g, 1000 g and 2000 g, 1000 g and 3200 g, 500 g and 1000 g, 400 g and 1200 g, 600 g and 800 g, 600 and 700 g, or 500 g and 700 g. In some embodiments, the rotation is between 600 g and 700 g, e.g., at or about 693 g.

In certain embodiments, at least a portion of the engineering, transduction, and/or transfection is performed with rotation, e.g., spinoculation and/or centrifugation. In some embodiments, the rotation is performed for, for about, or for at least or about 5 minutes, 10 minutes, 15 minutes, 30 minutes, 60 minutes, 90 minutes, 1 hour, 2 hours, 3 hours, 4 hours, 6 hours, 8 hours, 12 hours, 24 hours, 48 hours, 72 hours, 2 days, 3 days, 4 days, 5 days, 6 days, or for at least 7 days. In some embodiments, the rotation is performed for or for about 60 minutes. In certain embodiments, the rotation is performed for about 30 minutes. In some embodiments, the rotation performed for about 30 minutes at between 600 g and 700 g, e.g., at or about 693 g.

In certain embodiments, the number of viable cells to be engineered, transduced, and/or transfected ranges from about 5×10⁶ cells to about 100×10⁷ cells, such as from about 10×10⁶ cells to about 100×10⁶ cells, from about 100×10⁶ cells to about 200×10⁶ cells, from about 200×10⁶ cells to about 300×10⁶ cells, from about 300×10⁶ cells to about 400×10⁶ cells, from about 400×10⁶ cells to about 500×10⁶ cells, or from about 500×10⁶ cells to about 100×10⁷ cells. In particular examples, the number of viable cells to be engineered, transduced, and/or transfected is about or less than about 300×10⁶ cells.

In certain embodiments, at least a portion of the engineering, transduction, and/or transfection is conducted at a volume (e.g., the spinoculation volume) from about 5 mL to about 100 mL, such as from about 10 mL to about 50 mL, from about 15 mL to about 45 mL, from about 20 mL to about 40 mL, from about 25 mL to about 35 mL, or at or at about 30 mL. In certain embodiments, the cell pellet volume after spinoculation ranges from about 1 mL to about 25 mL, such as from about 5 mL to about 20 mL, from about 5 mL to about 15 mL, from about 5 mL to about 10 mL, or at or at about 10 mL.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications. In certain embodiments, the gene transfer is accomplished by first incubating the cells under stimulating conditions, such as by any of the methods described in Section I-B.

In some embodiments, methods for genetic engineering are carried out by contacting one or more cells of a composition with a nucleic acid molecule encoding the recombinant protein, e.g., recombinant receptor. In some embodiments, the contacting can be effected with centrifugation, such as spinoculation (e.g., centrifugal inoculation). Such methods include any of those as described in International Publication Number WO2016/073602. Exemplary centrifugal chambers include those produced and sold by Biosafe SA, including those for use with the Sepax® and Sepax® 2 system, including an A-200/F and A-200 centrifugal chambers and various kits for use with such systems. Exemplary chambers, systems, and processing instrumentation and cabinets are described, for example, in U.S. Pat. Nos. 6,123,655, 6,733,433 and Published U.S. patent Application, Publication No.: US 2008/0171951, and published international patent application, publication no. WO 00/38762, the contents of each of which are incorporated herein by reference in their entirety. Exemplary kits for use with such systems include, but are not limited to, single-use kits sold by BioSafe SA under product names CS-430.1, CS-490.1, CS-600.1 or CS-900.2.

In some embodiments, the system is included with and/or placed into association with other instrumentation, including instrumentation to operate, automate, control and/or monitor aspects of the transduction step and one or more various other processing steps performed in the system, e.g., one or more processing steps that can be carried out with or in connection with the centrifugal chamber system as described herein or in International Publication Number WO2016/073602. This instrumentation in some embodiments is contained within a cabinet. In some embodiments, the instrumentation includes a cabinet, which includes a housing containing control circuitry, a centrifuge, a cover, motors, pumps, sensors, displays, and a user interface. An exemplary device is described in U.S. Pat. Nos. 6,123,655, 6,733,433 and US 2008/0171951.

In some embodiments, the system comprises a series of containers, e.g., bags, tubing, stopcocks, clamps, connectors, and a centrifuge chamber. In some embodiments, the containers, such as bags, include one or more containers, such as bags, containing the cells to be transduced and the viral vector particles, in the same container or separate containers, such as the same bag or separate bags. In some embodiments, the system further includes one or more containers, such as bags, containing medium, such as diluent and/or wash solution, which is pulled into the chamber and/or other components to dilute, resuspend, and/or wash components and/or compositions during the methods. The containers can be connected at one or more positions in the system, such as at a position corresponding to an input line, diluent line, wash line, waste line and/or output line.

In some embodiments, the chamber is associated with a centrifuge, which is capable of effecting rotation of the chamber, such as around its axis of rotation. Rotation may occur before, during, and/or after the incubation in connection with transduction of the cells and/or in one or more of the other processing steps. Thus, in some embodiments, one or more of the various processing steps is carried out under rotation, e.g., at a particular force. The chamber is typically capable of vertical or generally vertical rotation, such that the chamber sits vertically during centrifugation and the side wall and axis are vertical or generally vertical, with the end wall(s) horizontal or generally horizontal.

In some embodiments, the composition containing cells and composition containing viral vector particles, and optionally air, can be combined or mixed prior to providing the compositions to the cavity. In some embodiments, the composition containing cells and composition containing viral vector particles, and optionally air, are provided separately and combined and mixed in the cavity. In some embodiments, a composition containing cells, a composition containing viral vector particles, and optionally air, can be provided to the internal cavity in any order. In any of such some embodiments, a composition containing cells and viral vector particles is the input composition once combined or mixed together, whether such is combined or mixed inside or outside the centrifugal chamber and/or whether cells and viral vector particles are provided to the centrifugal chamber together or separately, such as simultaneously or sequentially.

In some embodiments, intake of a volume of gas, such as air, occurs prior to the incubating the cells and viral vector particles, such as rotation, in the transduction method. In some embodiments, intake of the volume of gas, such as air, occurs during the incubation of the cells and viral vector particles, such as rotation, in the transduction method.

In some embodiments, the liquid volume of the cells or viral vector particles that make up the transduction composition, and optionally the volume of air, can be a predetermined volume. The volume can be a volume that is programmed into and/or controlled by circuitry associated with the system.

In some embodiments, intake of the transduction composition, and optionally gas, such as air, is controlled manually, semi-automatically and/or automatically until a desired or predetermined volume has been taken into the internal cavity of the chamber. In some embodiments, a sensor associated with the system can detect liquid and/or gas flowing to and from the centrifuge chamber, such as via its color, flow rate and/or density, and can communicate with associated circuitry to stop or continue the intake as necessary until intake of such desired or predetermined volume has been achieved. In some aspects, a sensor that is programmed or able only to detect liquid in the system, but not gas (e.g., air), can be made able to permit passage of gas, such as air, into the system without stopping intake. In some such embodiments, a non-clear piece of tubing can be placed in the line near the sensor while intake of gas, such as air, is desired. In some embodiments, intake of gas, such as air, can be controlled manually.

In aspects of the provided methods, the internal cavity of the centrifuge chamber is subjected to high speed rotation. In some embodiments, rotation is effected prior to, simultaneously, subsequently or intermittently with intake of the liquid input composition, and optionally air. In some embodiments, rotation is effected subsequent to intake of the liquid input composition, and optionally air. In some embodiments, rotation is by centrifugation of the centrifugal chamber at a relative centrifugal force at the inner surface of side wall of the internal cavity and/or at a surface layer of the cells of at or about or at least at or about 800 g, 1000 g, 1100 g, 1500, 1600 g, 1800 g, 2000 g, 2200 g, 2500 g, 3000 g, 3500 g or 4000 g. In some embodiments, rotation is by centrifugation at a force that is greater than or about 1100 g, such as by greater than or about 1200 g, greater than or about 1400 g, greater than or about 1600 g, greater than or about 1800 g, greater than or about 2000 g, greater than or about 2400 g, greater than or about 2800 g, greater than or about 3000 g or greater than or about 3200 g. In some embodiments, rotation is by centrifugation at a force that is or is about 1600 g.

In some embodiments, the method of transduction includes rotation or centrifugation of the transduction composition, and optionally air, in the centrifugal chamber for greater than or about 5 minutes, such as greater than or about 10 minutes, greater than or about 15 minutes, greater than or about 20 minutes, greater than or about 30 minutes, greater than or about 45 minutes, greater than or about 60 minutes, greater than or about 90 minutes or greater than or about 120 minutes. In some embodiments, the transduction composition, and optionally air, is rotated or centrifuged in the centrifugal chamber for greater than 5 minutes, but for no more than 60 minutes, no more than 45 minutes, no more than 30 minutes or no more than 15 minutes. In particular embodiments, the transduction includes rotation or centrifugation for or for about 60 minutes.

In some embodiments, the method of transduction includes rotation or centrifugation of the transduction composition, and optionally air, in the centrifugal chamber for between or between about 10 minutes and 60 minutes, 15 minutes and 60 minutes, 15 minutes and 45 minutes, 30 minutes and 60 minutes or 45 minutes and 60 minutes, each inclusive, and at a force at the internal surface of the side wall of the internal cavity and/or at a surface layer of the cells of at least or greater than or about 1000 g, 1100 g, 1200 g, 1400 g, 1500 g, 1600 g, 1800 g, 2000 g, 2200 g, 2400 g, 2800 g, 3200 g or 3600 g. In particular embodiments, the method of transduction includes rotation or centrifugation of the transduction composition, e.g., the cells and the viral vector particles, at or at about 1600 g for or for about 60 minutes.

In some embodiments, the gas, such as air, in the cavity of the chamber is expelled from the chamber. In some embodiments, the gas, such as air, is expelled to a container that is operably linked as part of the closed system with the centrifugal chamber. In some embodiments, the container is a free or empty container. In some embodiments, the air, such as gas, in the cavity of the chamber is expelled through a filter that is operably connected to the internal cavity of the chamber via a sterile tubing line. In some embodiments, the air is expelled using manual, semi-automatic or automatic processes. In some embodiments, air is expelled from the chamber prior to, simultaneously, intermittently or subsequently with expressing the output composition containing incubated cells and viral vector particles, such as cells in which transduction has been initiated or cells have been transduced with a viral vector, from the cavity of the chamber.

In some embodiments, the transduction and/or other incubation is performed as or as part of a continuous or semi-continuous process. In some embodiments, a continuous process involves the continuous intake of the cells and viral vector particles, e.g., the transduction composition (either as a single pre-existing composition or by continuously pulling into the same vessel, e.g., cavity, and thereby mixing, its parts), and/or the continuous expression or expulsion of liquid, and optionally expelling of gas (e.g., air), from the vessel, during at least a portion of the incubation, e.g., while centrifuging. In some embodiments, the continuous intake and continuous expression are carried out at least in part simultaneously. In some embodiments, the continuous intake occurs during part of the incubation, e.g., during part of the centrifugation, and the continuous expression occurs during a separate part of the incubation. The two may alternate. Thus, the continuous intake and expression, while carrying out the incubation, can allow for a greater overall volume of sample to be processed, e.g., transduced.

In some embodiments, the incubation is part of a continuous process, the method including, during at least a portion of the incubation, effecting continuous intake of said transduction composition into the cavity during rotation of the chamber and during a portion of the incubation, effecting continuous expression of liquid and, optionally expelling of gas (e.g., air), from the cavity through the at least one opening during rotation of the chamber.

In some embodiments, the semi-continuous incubation is carried out by alternating between effecting intake of the composition into the cavity, incubation, expression of liquid from the cavity and, optionally expelling of gas (e.g., air) from the cavity, such as to an output container, and then intake of a subsequent (e.g., second, third, etc.) composition containing more cells and other reagents for processing, e.g., viral vector particles, and repeating the process. For example, in some embodiments, the incubation is part of a semi-continuous process, the method including, prior to the incubation, effecting intake of the transduction composition into the cavity through said at least one opening, and subsequent to the incubation, effecting expression of fluid from the cavity; effecting intake of another transduction composition comprising cells and the viral vector particles into said internal cavity; and incubating the another transduction composition in said internal cavity under conditions whereby said cells in said another transduction composition are transduced with said vector. The process may be continued in an iterative fashion for a number of additional rounds. In this respect, the semi-continuous or continuous methods may permit production of even greater volume and/or number of cells.

In some embodiments, a portion of the transduction incubation is performed in the centrifugal chamber, which is performed under conditions that include rotation or centrifugation.

In some embodiments, the method includes an incubation in which a further portion of the incubation of the cells and viral vector particles is carried out without rotation or centrifugation, which generally is carried out subsequent to the at least portion of the incubation that includes rotation or centrifugation of the chamber. In certain embodiments, the incubation of the cells and viral vector particles is carried out without rotation or centrifugation for at least 1 hour, 6 hours, 12 hours, 24 hours, 32 hours, 48 hours, 60 hours, 72 hours, 90 hours, 96 hours, 3 days, 4 days, 5 days, or greater than 5 days. In certain embodiments, the incubation is carried out for or for about 72 hours.

In some such embodiments, the further incubation is effected under conditions to result in integration of the viral vector into a host genome of one or more of the cells. It is within the level of a skilled artisan to assess or determine if the incubation has resulted in integration of viral vector particles into a host genome, and hence to empirically determine the conditions for a further incubation. In some embodiments, integration of a viral vector into a host genome can be assessed by measuring the level of expression of a recombinant protein, such as a heterologous protein, encoded by a nucleic acid contained in the genome of the viral vector particle following incubation. A number of well-known methods for assessing expression level of recombinant molecules may be used, such as detection by affinity-based methods, e.g., immunoaffinity-based methods, e.g., in the context of cell surface proteins, such as by flow cytometry. In some examples, the expression is measured by detection of a transduction marker and/or reporter construct. In some embodiments, nucleic acid encoding a truncated surface protein is included within the vector and used as a marker of expression and/or enhancement thereof.

In some embodiments, the composition containing cells, the vector, e.g., viral particles, and reagent can be rotated, generally at relatively low force or speed, such as speed lower than that used to pellet the cells, such as from 600 rpm to 1700 rpm or from about 600 rpm to about 1700 rpm (e.g., at or about or at least 600 rpm, 1000 rpm, or 1500 rpm or 1700 rpm). In some embodiments, the rotation is carried at a force, e.g., a relative centrifugal force, of from 100 g to 3200 g or from about 100 g to about 3200 g (e.g., at or about or at least at or about 100 g, 200 g, 300 g, 400 g, 500 g, 1000 g, 1500 g, 2000 g, 2500 g, 3000 g or 3200 g), as measured for example at an internal or external wall of the chamber or cavity. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured).

In some embodiments, during at least a part of the genetic engineering, e.g., transduction, and/or subsequent to the genetic engineering the cells are transferred to the bioreactor bag assembly for culture of the genetically engineered cells, such as for cultivation or expansion of the cells, as described above.

In certain embodiments, a composition of enriched T cells in engineered, e.g., transduced or transfected, in the presence of a transduction adjuvant. In some embodiments, a composition of enriched T cells is engineered in the presence of one or more polycations. In some embodiments, a composition of enriched T cells is transduced, e.g., incubated with a viral vector particle, in the presence of one or more transduction adjuvants. In particular embodiments, a composition of enriched T cells is transfected, e.g., incubated with a non-viral vector, in the presence of one or more transduction adjuvants. In certain embodiments, the presence of one or more transduction adjuvants increases the efficiency of gene delivery, such as by increasing the amount, portion, and/or percentage of cells of the composition that are engineered (e.g., transduced or transfected). In certain embodiments, the presence of one or more transduction adjuvants increases the efficiency of transfection. In certain embodiments, the presence of one or more transduction adjuvants increases the efficiency of transduction. In particular embodiments, at least 25%, at least 30%, at least 40%, at least 50%, at least 60%, at least 70% at least 75%, at least 80%, at least 85%, at least 90%, at least 95%, or at least 99% of the cells that are engineered in the presence of a polycation contain or express the recombinant polynucleotide. In some embodiments, at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-fold, at least 50-fold, or at least 100-fold more cells of a composition are engineered to contain or express the recombinant transduction adjuvants in the presence of a polycation as compared to an alternative and/or exemplary method of engineering cells without the presence of a transduction adjuvant.

In some embodiments, the composition of enriched cells are engineered in the presence of less than 100 μg/ml, less than 90 μg/ml, less than 80 μg/ml, less than 75 μg/ml, less than 70 μg/ml, less than 60 μg/ml, less than 50 μg/ml, less than 40 μg/ml, less than 30 μg/ml, less than 25 μg/ml, less than 20 μg/ml, or less than μg/ml, less than 10 μg/ml of a transduction adjuvant. In certain embodiments, transduction adjuvants suitable for use with the provided methods include, but are not limited to polycations, fibronectin or fibronectin-derived fragments or variants, RetroNectin, and combinations thereof.

In some embodiments, the cells are engineered in the presence of a cytokine, e.g., a recombinant human cytokine, at a concentration of between 1 IU/ml and 1,000 IU/ml, between 10 IU/ml and 50 IU/ml, between 50 IU/ml and 100 IU/ml, between 100 IU/ml and 200 IU/ml, between 100 IU/ml and 500 IU/ml, between 250 IU/ml and 500 IU/ml, or between 500 IU/ml and 1,000 IU/ml.

In some embodiments, a composition of enriched T cells is engineered in the presence of IL-2, e.g., human recombinant IL-2, at a concentration between 1 IU/ml and 200 IU/ml, between 10 IU/ml and 100 IU/ml, between 50 IU/ml and 150 IU/ml, between 80 IU/ml and 120 IU/ml, between 60 IU/ml and 90 IU/ml, or between 70 IU/ml and 90 IU/ml. In particular embodiments, the composition of enriched T cells is engineered in the presence of recombinant IL-2 at a concentration at or at about 50 IU/ml, 55 IU/ml, 60 IU/ml, 65 IU/ml, 70 IU/ml, 75 IU/ml, 80 IU/ml, 85 IU/ml, 90 IU/ml, 95 IU/ml, 100 RJ/ml, 110 IU/ml, 120 RJ/ml, 130 IU/ml, 140 IU/ml, or 150 IU/ml. In some embodiments, the composition of enriched T cells is engineered in the presence of or of about 85 IU/ml. In some embodiments, the population of T cells is a population of CD4+ T cells. In particular embodiments, the composition of enriched T cells is enriched for CD4+ T cells, where CD8+ T cells are not enriched for and/or where CD8+ T cells are negatively selected for or depleted from the composition. In particular embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition.

In some embodiments, a composition of enriched T cells is engineered in the presence of recombinant IL-7, e.g., human recombinant IL-7, at a concentration between 100 IU/ml and 2,000 IU/ml, between 500 IU/ml and 1,000 IU/ml, between 100 IU/ml and 500 IU/ml, between 500 IU/ml and 750 IU/ml, between 750 RJ/ml and 1,000 IU/ml, or between 550 IU/ml and 650 IU/ml. In particular embodiments, the composition of enriched T cells is engineered in the presence of IL-7 at a concentration at or at about 50 IU/ml, 100 IU/ml, 150 IU/ml, 200 IU/ml, 250 IU/ml, 300 IU/ml, 350 IU/ml, 400 IU/ml, 450 IU/ml, 500 IU/ml, 550 IU/ml, 600 IU/ml, 650 IU/ml, 700 IU/ml, 750 IU/ml, 800 IU/ml, 750 IU/ml, 750 IU/ml, 750 IU/ml, or 1,000 IU/ml. In particular embodiments, the composition of enriched T cells is engineered in the presence of or of about 600 IU/ml of IL-7. In some embodiments, the composition engineered in the presence of recombinant IL-7 is enriched for a population of T cells, e.g., CD4+ T cells. In particular embodiments, the composition of enriched T cells is enriched for CD4+ T cells, where CD8+ T cells are not enriched for and/or where CD8+ T cells are negatively selected for or depleted from the composition.

In some embodiments, a composition of enriched T cells is engineered in the presence of recombinant IL-15, e.g., human recombinant IL-15, at a concentration between 0.1 IU/ml and 100 IU/ml, between 1 IU/ml and 50 IU/ml, between 5 IU/ml and 25 IU/ml, between 25 IU/ml and 50 IU/ml, between 5 IU/ml and 15 IU/ml, or between 10 IU/ml and 100 IU/ml. In particular embodiments, the composition of enriched T cells is engineered in the presence of IL-15 at a concentration at or at about 1 IU/ml, 2 IU/ml, 3 IU/ml, 4 IU/ml, 5 IU/ml, 6 IU/ml, 7 IU/ml, 8 IU/ml, 9 IU/ml, 10 IU/ml, 11 IU/ml, 12 IU/ml, 13 IU/ml, 14 IU/ml, 15 IU/ml, 20 IU/ml, 25 IU/ml, 30 IU/ml, 40 IU/ml, or 50 IU/ml. In some embodiments, the composition of enriched T cells is engineered in or in about 10 IU/ml of IL-15. In some embodiments, the composition of enriched T cells is incubated in or in about 10 IU/ml of recombinant IL-15. In some embodiments, the composition engineered in the presence of recombinant IL-15 is enriched for a population of T cells, e.g., CD4+ T cells and/or CD8+ T cells. In some embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition. In some embodiments, the composition of enriched T cells is a composition of enriched CD4+ T cells. In particular embodiments, the composition of enriched T cells is enriched for CD4+ T cells, where CD8+ T cells are not enriched for and/or where CD8+ T cells are negatively selected for or depleted from the composition.

In particular embodiments, a composition of enriched CD8+ T cells is engineered in the presence of IL-2 and/or IL-15. In certain embodiments, a composition of enriched CD4+ T cells is engineered in the presence of IL-2, IL-7, and/or IL-15. In some embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15.

In particular embodiments, the cells are engineered in the presence of one or more antioxidants. In some embodiments, antioxidants include, but are not limited to, one or more antioxidants comprise a tocopherol, a tocotrienol, alpha-tocopherol, beta-tocopherol, gamma-tocopherol, delta-tocopherol, alpha-tocotrienol, beta-tocotrienol, alpha-tocopherolquinone, Trolox (6-hydroxy-2,5,7,8-tetramethylchroman-2-carboxylic acid), butylated hydroxyanisole (BHA), butylated hydroxytoluene (BHT), a flavonoids, an isoflavone, lycopene, beta-carotene, selenium, ubiquinone, luetin, S-adenosylmethionine, glutathione, taurine, N-acetyl cysteine (NAC), citric acid, L-carnitine, BHT, monothioglycerol, ascorbic acid, propyl gallate, methionine, cysteine, homocysteine, gluthatione, cystamine and cystathionine, and/or glycine-glycine-histidine.

In some embodiments, the one or more antioxidants is or includes a sulfur containing oxidant. In certain embodiments, a sulfur containing antioxidant may include thiol-containing antioxidants and/or antioxidants which exhibit one or more sulfur moieties, e.g., within a ring structure. In some embodiments, the sulfur containing antioxidants may include, for example, N-acetylcysteine (NAC) and 2,3-dimercaptopropanol (DMP), L-2-oxo-4-thiazolidinecarboxylate (OTC) and lipoic acid. In particular embodiments, the sulfur containing antioxidant is a glutathione precursor. In some embodiments, the glutathione precursor is a molecule which may be modified in one or more steps within a cell to derived glutathione. In particular embodiments, a glutathione precursor may include, but is not limited to N-acetyl cysteine (NAC), L-2-oxothiazolidine-4-carboxylic acid (Procysteine), lipoic acid, S-allyl cysteine, or methylmethionine sulfonium chloride.

In some embodiments, the cells are engineered in the presence of one or more antioxidants. In some embodiments, the cells are engineered in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 500 μg/ml and 2 mg/ml, 500 μg/ml and 5 mg/ml, between 1 mg/ml and 10 mg/ml, or between 1 mg/ml and 100 mg/ml of the one or more antioxidants. In some embodiments, the cells are engineered in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of the one or more antioxidant. In some embodiments, the one or more antioxidants is or includes a sulfur containing antioxidant. In particular embodiments, the one or more antioxidants is or includes a glutathione precursor.

In some embodiments, the cells are engineered in the presence of NAC. In some embodiments, the cells are engineered in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1,500 μg/ml and 2 mg/ml, 500 μg/ml and 5 mg/ml, between 1 mg/ml and 10 mg/ml, or between 1 mg/ml and 100 mg/ml of NAC. In some embodiments, the cells are engineered in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of NAC. In some embodiments, the cells are engineered with or with about 0.8 mg/ml.

In some embodiments, a composition of enriched T cells, such as stimulated T cells, e.g., stimulated CD4+ T cells or stimulated CD8+ T cells, is engineered in the presence of one or more polycations. In some embodiments, a composition of enriched T cells, such as stimulated T cells, e.g., stimulated CD4+ T cells or stimulated CD8+ T cells, is transduced, e.g., incubated with a viral vector particle, in the presence of one or more polycations. In particular embodiments, a composition of enriched T cells, such as stimulated T cells, e.g., stimulated CD4+ T cells or stimulated CD8+ T cells, is transfected, e.g., incubated with a non-viral vector, in the presence of one or more polycations. In certain embodiments, the presence of one or more polycations increases the efficiency of gene delivery, such as by increasing the amount, portion, and/or percentage of cells of the composition that are engineered (e.g., transduced or transfected). In certain embodiments, the presence of one or more polycations increases the efficiency of transfection. In certain embodiments, the presence of one or more polycations increases the efficiency of transduction. In particular embodiments, at least 25%, at least 30%, at least 40%, at least 50%, at least 60%, at least 70% at least 75%, at least 80%, at least 85%, at least 90%, at least 95%, or at least 99% of the cells that are engineered in the presence of a polycation contain or express the recombinant polynucleotide. In some embodiments, at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-fold, at least 50-fold, or at least 100-fold more cells of a composition are engineered to contain or express the recombinant polynucleotide in the presence of a polycation as compared to an alternative and/or exemplary method of engineering cells without the presence of a polycation.

In certain embodiments, the composition of enriched cells, e.g., the composition of enriched CD4+ T cells or enriched CD8+ T cells, such as stimulated T cells thereof, is engineered in the presence of a low concentration or amount of a polycation, e.g., relative to an exemplary and/or alternative method of engineering cells in the presence of a polycation. In certain embodiments, the composition of enriched cells, such as stimulated T cells, e.g., stimulated CD4+ T cells or stimulated CD8+ T cells, is engineered in the presence of less than 90%, less than 80%, less than 75%, less than 70%, less than 60%, less than 50%, less than 40%, less than 30%, less than 25%, less than 20%, less than 10%, less than 5%, less than 1%, less than 0.1%, of less than 0.01% of the amount and/or concentration of the polycation of an exemplary and/or alternative process for engineering cells. In some embodiments, the composition of enriched cells, such as stimulated T cells, e.g., stimulated CD4+ T cells or stimulated CD8+ T cells, are engineered in the presence of less than 100 μg/ml, less than 90 μg/ml, less than 80 μg/ml, less than 75 μg/ml, less than 70 μg/ml, less than 60 μg/ml, less than 50 μg/ml, less than 40 μg/ml, less than 30 μg/ml, less than 25 μg/ml, less than 20 μg/ml, or less than μg/ml, less than 10 μg/ml of the polycation. In particular embodiments, the composition of enriched cells, such as stimulated T cells, e.g., stimulated CD4+ T cells or stimulated CD8+ T cells, is engineered in the presence of or of about 1 μg/ml, 5 μg/ml, 10 μg/ml, 15 μg/ml, 20 μg/ml, 25 μg/ml, 30 μg/ml, 35 μg/ml, 40 μg/ml, 45 μg/ml, or 50 μg/ml, of the polycation.

In particular embodiments, engineering the composition of enriched cells, such as stimulated T cells, e.g., stimulated CD4+ T cells or stimulated CD8+ T cells, in the presence of a polycation reduces the amount of cell death, e.g., by necrosis, programed cell death, or apoptosis. In some embodiments, the composition of enriched T cells, such as stimulated T cells, e.g., stimulated CD4+ T cells or stimulated CD8+ T cells, is engineered in the presence of a low amount of a polycation, e.g., less than 100 μg/ml, 50 μg/ml, or 10 μg/ml, and at least 50%, at least 60%, at least 70%, at least 75%, at least 80%, at least 85%, at least 90%, at least 95%, at least 99%, or at least 99.9% of the cells survive, e.g., do not undergo necrosis, programed cell death, or apoptosis, during or at least 1 day, 2 days, 3 days, 4 days, 5 days, 6 days, 7 days, or more than 7 days after the engineering step is complete. In some embodiments, the composition is engineered in the presence of a low concentration or amount of polycation as compared to the alternative and/or exemplary method of engineering cells in the presence of higher amount or concentration of polycation, e.g., more than 50 μg/ml, 100 μg/ml, 500 μg/ml, or 1,000 μg/ml, and the cells of the composition have at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-fold, at least 50-fold, or at least 100-fold greater survival as compared to cells undergoing the exemplary and/or alternative process.

In some embodiments, the polycation is positively-charged. In certain embodiments, the polycation reduces repulsion forces between cells and vectors, e.g., viral or non-viral vectors, and mediates contact and/or binding of the vector to the cell surface. In some embodiments, the polycation is polybrene, DEAE-dextran, protamine sulfate, poly-L-lysine, or cationic liposomes.

In particular embodiments, the polycation is protamine sulfate. In some embodiments, the composition of enriched T cells, such as stimulated T cells, e.g., stimulated CD4+ T cells or stimulated CD8+ T cells, are engineered in the presence of less than or about 500 μg/ml, less than or about 400 μg/ml, less than or about 300 μg/ml, less than or about 200 μg/ml, less than or about 150 μg/ml, less than or about 100 μg/ml, less than or about 90 μg/ml, less than or about 80 μg/ml, less than or about 75 μg/ml, less than or about 70 μg/ml, less than or about 60 μg/ml, less than or about 50 μg/ml, less than or about 40 μg/ml, less than or about 30 μg/ml, less than or about 25 μg/ml, less than or about 20 μg/ml, or less than or about 15 μg/ml, or less than or about 10 μg/ml of protamine sulfate. In particular embodiments, the composition of enriched cells, such as stimulated T cells, e.g., stimulated CD4+ T cells or stimulated CD8+ T cells, is engineered in the presence of or of about 1 μg/ml, 5 μg/ml, 10 μg/ml, 15 μg/ml, 20 μg/ml, 25 μg/ml, 30 μg/ml, 35 μg/ml, 40 μg/ml, 45 μg/ml, 50 μg/ml, 55 μg/ml, 60 μg/ml, 75 μg/ml, 80 μg/ml, 85 μg/ml, 90 μg/ml, 95 μg/ml, 100 μg/ml, 105 μg/ml, 110 μg/ml, 115 μg/ml, 120 μg/ml, 125 μg/ml, 130 μg/ml, 135 μg/ml, 140 μg/ml, 145 μg/ml, or 150 μg/ml of protamine sulfate.

In some embodiments, the engineered composition of enriched CD4+ T cells, such as stimulated T cells, e.g., stimulated CD4+ T cells, includes at least 40, at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the composition of enriched CD4+ T cells, such as stimulated T cells, e.g., stimulated CD4+ T cells, that is engineered includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells, such as stimulated T cells, e.g., stimulated CD8+ T cells, that is engineered includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of enriched CD8+ T cells, such as stimulated T cells, e.g., stimulated CD8+ T cells, that is engineered includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, engineering the cells includes a culturing, contacting, or incubation with the vector, e.g., the viral vector of the non-viral vector. In certain embodiments, the engineering includes culturing, contacting, and/or incubating the cells with the vector is performed for, for about, or for at least 4 hours, 6 hours, 8 hours, 12 hours, 16 hours, 18 hours, 24 hours, 30 hours, 36 hours, 40 hours, 48 hours, 54 hours, 60 hours, 72 hours, 84 hours, 1 day, 2 days, 3 days, 4 days, 5 days, 6 days, or 7 days, or more than 7 days. In particular embodiments, the engineering includes culturing, contacting, and/or incubating the cells with the vector for or for about 24 hours, 36 hours, 48 hours, 60 hours, 72 hours, or 84 hours, or for or for about 2 days, 3 days, 4 days, or 5 days. In some embodiments, the engineering step is performed for or for about 24 hours, 36 hours, 48 hours, 60 hours, 72 hours, or 84 hours. In certain embodiments, the engineering is performed for about 60 hours or about 84 hours, for or for about 72 hours, or for or for about 2 days.

In some embodiments, the engineering is performed at a temperature from about 25 to about 38° C., such as from about 30 to about 37° C., from about 36 to about 38° C., or at or about 37° C.±2° C. In some embodiments, the composition of enriched T cells is engineered at a CO₂ level from about 2.5% to about 7.5%, such as from about 4% to about 6%, for example at or about 5%±0.5%. In some embodiments, the composition of enriched T cells is engineered at a temperature of or about 37° C. and/or at a CO₂ level of or about 5%.

In some embodiments, the cells, e.g., the CD4+ and/or the CD8+ T cells, are cultivated, after one or more steps are performed for genetic engineering, e.g., transducing or transfection the cells to contain a polynucleotide encoding a recombinant receptor. In some embodiments, the cultivation may include culture, incubation, stimulation, activation, expansion, and/or propagation. In some such embodiments, the further cultivation is effected under conditions to result in integration of the viral vector into a host genome of one or more of the cells. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

In some embodiments, the further incubation is carried out at temperatures greater than room temperature, such as greater than or greater than about 25° C., such as generally greater than or greater than about 32° C., 35° C. or 37° C. In some embodiments, the further incubation is effected at a temperature of at or about 37° C.±2° C., such as at a temperature of at or about 37° C.

In some embodiments, the further incubation is performed under conditions for stimulation and/or activation of cells, which conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent (e.g., stimulatory and/or accessory agents), e.g., ligand, which is capable of activating an intracellular signaling domain of a TCR complex. In some aspects, the agent turns on or initiates TCR/CD3 intracellular signaling cascade in a T cell, such as agents suitable to deliver a primary signal, e.g., to initiate activation of an ITAM-induced signal, such as those specific for a TCR component, and/or an agent that promotes a costimulatory signal, such as one specific for a T cell costimulatory receptor, e.g., anti-CD3, anti-CD28, or anti-41-BB, for example, optionally bound to solid support such as a bead, and/or one or more cytokines. Among the stimulating agents are anti-CD3/anti-CD28 beads (e.g., DYNABEADS® M-450 CD3/CD28 T Cell Expander, and/or ExpACT® beads). Optionally, the expansion method may further comprise the step of adding anti-CD3 and/or anti-CD28 antibody to the culture medium. In some embodiments, the stimulating agents include IL-2 and/or IL-15, for example, an IL-2 concentration of at least about 10 units/mL.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/ml). In some embodiments, the stimulating agents include IL-2 and/or IL-15, for example, an IL-2 concentration of at least about 10 units/mL, at least about 50 units/mL, at least about 100 units/mL or at least about 200 units/mL.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the further incubation is carried out in the same container or apparatus in which the contacting occurred. In some embodiments, the further incubation is carried out without rotation or centrifugation, which generally is carried out subsequent to the at least portion of the incubation done under rotation, e.g., in connection with centrifugation or spinoculation. In some embodiments, the further incubation is carried out outside of a stationary phase, such as outside of a chromatography matrix, for example, in solution.

In some embodiments, the further incubation is carried out in a different container or apparatus from that in which the contacting occurred, such as by transfer, e.g., automatic transfer, of the cell composition into a different container or apparatus subsequent to contacting with the viral particles and reagent.

In some embodiments, the further culturing or incubation, e.g., to facilitate ex vivo expansion, is carried out of for greater than or greater than about 24 hours, 2 days, 3 days, 4 days, 5 days, 6 days, 7 days, 8 days, 9 days, 10 days, 11 days, 12 days, 13 days or 14 days. In some embodiments, the further culturing or incubation is carried out for no more than 6 days, no more than 5 days, no more than 4 days, no more than 3 days, no more than 2 days or no more than 24 hours.

In some embodiments, the total duration of the incubation, e.g., with the stimulating agent, is between or between about 1 hour and 96 hours, 1 hour and 72 hours, 1 hour and 48 hours, 4 hours and 36 hours, 8 hours and 30 hours or 12 hours and 24 hours, such as at least or about at least or about 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

In some embodiments, the methods provided herein do not include further culturing or incubation, e.g., do not include ex vivo expansion step, or include a substantially shorter ex vivo expansion step.

In some embodiments, the stimulatory reagent is removed and/or separated from the cells prior to the engineering. In particular embodiments, the stimulatory reagent is removed and/or separated from the cells after the engineering. In certain embodiments, the stimulatory agent is removed and/or separated from the cells subsequent to the engineering and prior to cultivating the engineered cells, e.g., under conditions that promote proliferation and/or expansion. In certain embodiments, the stimulatory reagent is a stimulatory reagent that is described in Section I-B-1. In particular embodiments, the stimulatory reagent is removed and/or separated from the cells as described in Section I-B-2.

1. Vectors and Methods

In some embodiments, the cells, e.g., T cells, are genetically engineered to express a recombinant receptor. In some embodiments, the engineering is carried out by introducing one or more polynucleotide(s) that encode the recombinant receptor or portions or components thereof. Also provided are polynucleotides encoding a recombinant receptor, and vectors or constructs containing such nucleic acids and/or polynucleotides.

In particular embodiments, the vector is a viral vector a non-viral vector. In some cases, the vector is a viral vector, such as a retroviral vector, e.g., a lentiviral vector or a gammaretroviral vector.

In some embodiments, the polynucleotide encoding the recombinant receptor contains at least one promoter that is operatively linked to control expression of the recombinant receptor. In some examples, the polynucleotide contains two, three, or more promoters operatively linked to control expression of the recombinant receptor. In some embodiments, polynucleotide can contain regulatory sequences, such as transcription and translation initiation and termination codons, which are specific to the type of host (e.g., bacterium, fungus, plant, or animal) into which the polynucleotide is to be introduced, as appropriate and taking into consideration whether the polynucleotide is DNA- or RNA-based. In some embodiments, the polynucleotide can contain regulatory/control elements, such as a promoter, an enhancer, an intron, a polyadenylation signal, a Kozak consensus sequence, internal ribosome entry sites (IRES), a 2A sequence, and splice acceptor or donor. In some embodiments, the polynucleotide can contain a nonnative promoter operably linked to the nucleotide sequence encoding the recombinant receptor and/or one or more additional polypeptide(s). In some embodiments, the promoter is selected from among an RNA pol I, pol II or pol III promoter. In some embodiments, the promoter is recognized by RNA polymerase II (e.g., a CMV, SV40 early region or adenovirus major late promoter). In another embodiment, the promoter is recognized by RNA polymerase III (e.g., a U6 or H1 promoter). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1a promoter (EF1α), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (see Challita et al. (1995) J. Virol. 69(2):748-755). In some embodiments, the promoter is a tissue-specific promoter. In another embodiment, the promoter is a viral promoter. In another embodiment, the promoter is a non-viral promoter. In some embodiments, exemplary promoters can include, but are not limited to, human elongation factor 1 alpha (EF1α) promoter or a modified form thereof or the MND promoter.

In another embodiment, the promoter is a regulated promoter (e.g., inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof. In some embodiments, the polynucleotide does not include a regulatory element, e.g., promoter.

In some cases, the nucleic acid sequence encoding the recombinant receptor, e.g., chimeric antigen receptor (CAR) contains a signal sequence that encodes a signal peptide. Non-limiting exemplary examples of signal peptides include, for example, the GMCSFR alpha chain signal peptide set forth in SEQ ID NO: 10 and encoded by the nucleotide sequence set forth in SEQ ID NO: 9, the CD8 alpha signal peptide set forth in SEQ ID NO: 11, or the CD33 signal peptide set forth in SEQ ID NO: 12.

In some embodiments, the polynucleotide contains a nucleic acid sequence encoding one or more additional polypeptides, e.g., one or more marker(s) and/or one or more effector molecules. In some embodiments, the one or more marker(s) includes a transduction marker, a surrogate marker and/or a resistance marker or selection marker. Among additional nucleic acid sequences introduced, e.g., encoding for one or more additional polypeptide(s), include nucleic acid sequences that can improve the efficacy of therapy, such as by promoting viability and/or function of transferred cells; nucleic acid sequences to provide a genetic marker for selection and/or evaluation of the cells, such as to assess in vivo survival or localization; nucleic acid sequences to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also WO 1992008796 and WO 1994028143 describing the use of bifunctional selectable fusion genes derived from fusing a dominant positive selectable marker with a negative selectable marker, and U.S. Pat. No. 6,040,177.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g., CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence. Extrinsic marker genes may in some cases be utilized in connection with engineered cell to permit detection or selection of cells and, in some cases, also to promote cell elimination and/or cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO: 2 or 3) or a prostate-specific membrane antigen (PSMA) or modified form thereof, such as a truncated PSMA (tPSMA). In some aspects, tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g., surrogate marker, includes all or part (e.g., truncated form) of CD34, a NGFR, a CD19 or a truncated CD19, e.g., a truncated non-human CD19. An exemplary polypeptide for a truncated EGFR (e.g., tEGFR) comprises the sequence of amino acids set forth in SEQ ID NO: 2 or 3 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 2 or 3.

In some embodiments, the marker is or comprises a detectable protein, such as a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, codon-optimized, stabilized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof. In some aspects, expression of the enzyme can be detected by addition of a substrate that can be detected upon the expression and functional activity of the enzyme.

In some embodiments, the marker is a resistance maker or selection marker. In some embodiments, the resistance maker or selection marker is or comprises a polypeptide that confers resistance to exogenous agents or drugs. In some embodiments, the resistance marker or selection marker is an antibiotic resistance gene. In some embodiments, the resistance marker or selection marker is an antibiotic resistance gene confers antibiotic resistance to a mammalian cell. In some embodiments, the resistance marker or selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

Any of the recombinant receptors and/or the additional polypeptide(s) described herein can be encoded by one or more polynucleotides containing one or more nucleic acid sequences encoding recombinant receptors, in any combinations, orientation or arrangements. For example, one, two, three or more polynucleotides can encode one, two, three or more different polypeptides, e.g., recombinant receptors or portions or components thereof, and/or one or more additional polypeptide(s), e.g., a marker and/or an effector molecule. In some embodiments, one polynucleotide contains a nucleic acid sequence encoding a recombinant receptor, e.g., CAR, or portion or components thereof, and a nucleic acid sequence encoding one or more additional polypeptide(s). In some embodiments, one vector or construct contains a nucleic acid sequence encoding a recombinant receptor, e.g., CAR, or portion or components thereof, and a separate vector or construct contains a nucleic acid sequence encoding one or more additional polypeptide(s). In some embodiments, the nucleic acid sequence encoding the recombinant receptor and the nucleic acid sequence encoding the one or more additional polypeptide(s) are operably linked to two different promoters. In some embodiments, the nucleic acid encoding the recombinant receptor is present upstream of the nucleic acid encoding the one or more additional polypeptide(s). In some embodiments, the nucleic acid encoding the recombinant receptor is present downstream of the nucleic acid encoding one or more additional polypeptide(s).

In certain cases, one polynucleotide contains nucleic acid sequences encode two or more different polypeptide chains, e.g., a recombinant receptor and one or more additional polypeptide(s), e.g., a marker and/or an effector molecule. In some embodiments, the nucleic acid sequences encoding two or more different polypeptide chains, e.g., a recombinant receptor and one or more additional polypeptide(s), are present in two separate polynucleotides. For example, two separate polynucleotides are provided, and each can be individually transferred or introduced into the cell for expression in the cell. In some embodiments, the nucleic acid sequences encoding the marker and the nucleic acid sequences encoding the recombinant receptor are present or inserted at different locations within the genome of the cell. In some embodiments, the nucleic acid sequences encoding the marker and the nucleic acid sequences encoding the recombinant receptor are operably linked to two different promoters.

In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, the nucleic acid sequences encoding the recombinant receptor and the nucleic acid sequences encoding the one or more additional polypeptide(s) are operably linked to the same promoter and are optionally separated by an internal ribosome entry site (IRES), or a nucleic acid encoding a self-cleaving peptide or a peptide that causes ribosome skipping, such as a 2A element. For example, an exemplary marker, and optionally a ribosome skipping sequence sequence, can be any as disclosed in PCT Pub. No. WO2014031687.

In some embodiments, transcription units can be engineered as a bicistronic unit containing an IRES, which allows coexpression of gene products (e.g., encoding the recombinant receptor and the additional polypeptide) by a message from a single promoter. Alternatively, in some cases, a single promoter may direct expression of an RNA that contains, in a single open reading frame (ORF), two or three genes (e.g., encoding the marker and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as a T2A, can cause the ribosome to skip (ribosome skipping) synthesis of a peptide bond at the C-terminus of a 2A element, leading to separation between the end of the 2A sequence and the next peptide downstream (see, e.g., de Felipe, Genetic Vaccines and Ther. 2:13 (2004) and de Felipe et al. Traffic 5:616-626 (2004)). Various 2A elements are known. Examples of 2A sequences that can be used in the methods and system disclosed herein, without limitation, 2A sequences from the foot-and-mouth disease virus (F2A, e.g., SEQ ID NO: 8), equine rhinitis A virus (E2A, e.g., SEQ ID NO: 7), Thosea asigna virus (T2A, e.g., SEQ ID NO: 1 or 4), and porcine teschovirus-1 (P2A, e.g., SEQ ID NO: 5 or 6) as described in U.S. Patent Pub. No. 20070116690.

In some embodiments, the polynucleotide encoding the recombinant receptor and/or additional polypeptide is contained in a vector or can be cloned into one or more vector(s). In some embodiments, the one or more vector(s) can be used to transform or transfect a host cell, e.g., a cell for engineering. Exemplary vectors include vectors designed for introduction, propagation and expansion or for expression or both, such as plasmids and viral vectors. In some aspects, the vector is an expression vector, e.g., a recombinant expression vector. In some embodiments, the recombinant expression vectors can be prepared using standard recombinant DNA techniques.

In some embodiments, the vector can be a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λ610, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech).

In some embodiments, the polynucleotide encoding the recombinant receptor and/or one or more additional polypeptide(s), is introduced into a composition containing cultured cells, such as by retroviral transduction, transfection, or transformation.

In some embodiments, the vector is a viral vector, such as a retroviral vector. In some embodiments, the polynucleotide encoding the recombinant receptor and/or additional polypeptide(s) are introduced into the cell via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy 18:1748-1757; and Hackett et al. (2010) Molecular Therapy 18:674-683).

In some embodiments, the vectors include viral vectors, e.g., retroviral or lentiviral, non-viral vectors or transposons, e.g., Sleeping Beauty transposon system, vectors derived from simian virus 40 (SV40), adenoviruses, adeno-associated virus (AAV), lentiviral vectors or retroviral vectors, such as gamma-retroviral vectors, retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV) or adeno-associated virus (AAV).

In some embodiments, one or more polynucleotide(s) are introduced into a T cell using electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material, e.g., polynucleotides and/or vectors, into immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987) and other approaches described in, e.g., International Pat. App. Pub. No. WO 2014055668, and U.S. Pat. No. 7,446,190.

In some embodiments, the one or more polynucleotide(s) or vector(s) encoding a recombinant receptor and/or additional polypeptide(s) may be introduced into cells, e.g., T cells, either during or after expansion. This introduction of the polynucleotide(s) or vector(s) can be carried out with any suitable retroviral vector, for example. Resulting genetically engineered cells can then be liberated from the initial stimulus (e.g., anti-CD3/anti-CD28 stimulus) and subsequently be stimulated in the presence of a second type of stimulus (e.g., via a de novo introduced recombinant receptor). This second type of stimulus may include an antigenic stimulus in form of a peptide/MHC molecule, the cognate (cross-linking) ligand of the genetically introduced receptor (e.g., natural antigen and/or ligand of a CAR) or any ligand (such as an antibody) that directly binds within the framework of the new receptor (e.g., by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

a. Viral Vector Particles

In some embodiments, one or more polynucleotide(s) are introduced into cells using recombinant infectious virus particles, such as, e.g., vectors derived from simian virus 40 (SV40), adenoviruses, adeno-associated virus (AAV). In some embodiments, one or more polynucleotide(s) are introduced into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557.

In some embodiments, the vector is a retroviral vector. In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, the viral vector particles contain a genome derived from a retroviral genome based vector, such as derived from a lentiviral genome based vector. In some aspects of the provided viral vectors, the heterologous nucleic acid encoding a recombinant receptor, such as an antigen receptor, such as a CAR, is contained and/or located between the 5′ LTR and 3′ LTR sequences of the vector genome.

In some embodiments, the viral vector genome is a lentivirus genome, such as an HIV-1 genome or an SIV genome. For example, lentiviral vectors have been generated by multiply attenuating virulence genes, for example, the genes env, vif, vpu and nef can be deleted, making the vector safer for therapeutic purposes. Lentiviral vectors are known. See Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

Non-limiting examples of lentiviral vectors include those derived from a lentivirus, such as Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (E1AV). For example, lentiviral vectors have been generated by multiply attenuating the HIV virulence genes, for example, the genes env, vif, vpr, vpu and nef are deleted, making the vector safer for therapeutic purposes. Lentiviral vectors are known in the art, see Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

In some embodiments, the viral genome vector can contain sequences of the 5′ and 3′ LTRs of a retrovirus, such as a lentivirus. In some aspects, the viral genome construct may contain sequences from the 5′ and 3′ LTRs of a lentivirus, and in particular can contain the R and U5 sequences from the 5′ LTR of a lentivirus and an inactivated or self-inactivating 3′ LTR from a lentivirus. The LTR sequences can be LTR sequences from any lentivirus from any species. For example, they may be LTR sequences from HIV, SIV, FIV or BIV. Typically, the LTR sequences are HIV LTR sequences.

In some embodiments, the nucleic acid of a viral vector, such as an HIV viral vector, lacks additional transcriptional units. The vector genome can contain an inactivated or self-inactivating 3′ LTR (Zufferey et al. J Virol 72: 9873, 1998; Miyoshi et al., J Virol 72:8150, 1998). For example, deletion in the U3 region of the 3′ LTR of the nucleic acid used to produce the viral vector RNA can be used to generate self-inactivating (SIN) vectors. This deletion can then be transferred to the 5′ LTR of the proviral DNA during reverse transcription. A self-inactivating vector generally has a deletion of the enhancer and promoter sequences from the 3′ long terminal repeat (LTR), which is copied over into the 5′ LTR during vector integration. In some embodiments enough sequence can be eliminated, including the removal of a TATA box, to abolish the transcriptional activity of the LTR. This can prevent production of full-length vector RNA in transduced cells. In some aspects, the U3 element of the 3′ LTR contains a deletion of its enhancer sequence, the TATA box, Sp1, and NF-kappa B sites. As a result of the self-inactivating 3′ LTR, the provirus that is generated following entry and reverse transcription contains an inactivated 5′ LTR. This can improve safety by reducing the risk of mobilization of the vector genome and the influence of the LTR on nearby cellular promoters. The self-inactivating 3′ LTR can be constructed by any method known in the art. In some embodiments, this does not affect vector titers or the in vitro or in vivo properties of the vector.

Optionally, the U3 sequence from the lentiviral 5′ LTR can be replaced with a promoter sequence in the viral construct, such as a heterologous promoter sequence. This can increase the titer of virus recovered from the packaging cell line. An enhancer sequence can also be included. Any enhancer/promoter combination that increases expression of the viral RNA genome in the packaging cell line may be used. In one example, the CMV enhancer/promoter sequence is used (U.S. Pat. Nos. 5,385,839 and 5,168,062).

In certain embodiments, the risk of insertional mutagenesis can be minimized by constructing the retroviral vector genome, such as lentiviral vector genome, to be integration defective. A variety of approaches can be pursued to produce a non-integrating vector genome. In some embodiments, a mutation(s) can be engineered into the integrase enzyme component of the pol gene, such that it encodes a protein with an inactive integrase. In some embodiments, the vector genome itself can be modified to prevent integration by, for example, mutating or deleting one or both attachment sites, or making the 3′ LTR-proximal polypurine tract (PPT) non-functional through deletion or modification. In some embodiments, non-genetic approaches are available; these include pharmacological agents that inhibit one or more functions of integrase. The approaches are not mutually exclusive; that is, more than one of them can be used at a time. For example, both the integrase and attachment sites can be non-functional, or the integrase and PPT site can be non-functional, or the attachment sites and PPT site can be non-functional, or all of them can be non-functional. Such methods and viral vector genomes are known and available (see Philpott and Thrasher, Human Gene Therapy 18:483, 2007; Engelman et al. J Virol 69:2729, 1995; Brown et al J Virol 73:9011 (1999); WO 2009/076524; McWilliams et al., J Virol 77:11150, 2003; Powell and Levin J Virol 70:5288, 1996).

In some embodiments, the vector contains sequences for propagation in a host cell, such as a prokaryotic host cell. In some embodiments, the nucleic acid of the viral vector contains one or more origins of replication for propagation in a prokaryotic cell, such as a bacterial cell. In some embodiments, vectors that include a prokaryotic origin of replication also may contain a gene whose expression confers a detectable or selectable marker such as drug resistance.

The viral vector genome is typically constructed in a plasmid form that can be transfected into a packaging or producer cell line. Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g., vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences, e.g., recombinant nucleic acids, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, i.e. a nucleic acid encoding an antigen receptor, such as a CAR; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

In some embodiments, the retroviral vector particle, such as lentiviral vector particle, is pseudotyped to increase the transduction efficiency of host cells. For example, a retroviral vector particle, such as a lentiviral vector particle, in some embodiments is pseudotyped with a VSV-G glycoprotein, which provides a broad cell host range extending the cell types that can be transduced. In some embodiments, a packaging cell line is transfected with a plasmid or polynucleotide encoding a non-native envelope glycoprotein, such as to include xenotropic, polytropic or amphotropic envelopes, such as Sindbis virus envelope, GALV or VSV-G.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10) and Cf2Th (ATCC CRL 1430) cells.

In some embodiments, the packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line produces viral vector particles that contain the viral vector genome. Methods for transfection or infection are well known. Non-limiting examples include calcium phosphate, DEAE-dextran and lipofection methods, electroporation and microinjection.

When a recombinant plasmid and the retroviral LTR and packaging sequences are introduced into a special cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a packaging cell line, such as an exemplary HEK 293T cell line, by introduction of plasmids to allow generation of lentiviral particles. In some embodiments, a packaging cell is transfected and/or contains a polynucleotide encoding gag and pol, and a polynucleotide encoding a recombinant receptor, such as an antigen receptor, for example, a CAR. In some embodiments, the packaging cell line is optionally and/or additionally transfected with and/or contains a polynucleotide encoding a rev protein. In some embodiments, the packaging cell line is optionally and/or additionally transfected with and/or contains a polynucleotide encoding a non-native envelope glycoprotein, such as VSV-G. In some such embodiments, approximately two days after transfection of cells, e.g., HEK 293T cells, the cell supernatant contains recombinant lentiviral vectors, which can be recovered and titered.

Recovered and/or produced retroviral vector particles can be used to transduce target cells using the methods as described. Once in the target cells, the viral RNA is reverse-transcribed, imported into the nucleus and stably integrated into the host genome. One or two days after the integration of the viral RNA, the expression of the recombinant protein, e.g., antigen receptor, such as CAR, can be detected.

In some embodiments, the provided methods involve methods of transducing cells by contacting, e.g., incubating, a cell composition comprising a plurality of cells with a viral particle. In some embodiments, the cells to be transfected or transduced are or comprise primary cells obtained from a subject, such as cells enriched and/or selected from a subject.

In some embodiments, the concentration of cells to be transduced of the composition is from 1.0×10⁵ cells/mL to 1.0×10⁸ cells/mL or from about 1.0×10⁵ cells/mL to about 1.0×10⁸ cells/mL, such as at least or about at least or about 1.0×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL.

In some embodiments, the viral particles are provided at a certain ratio of copies of the viral vector particles or infectious units (IU) thereof, per total number of cells to be transduced (IU/cell). For example, in some embodiments, the viral particles are present during the contacting at or about or at least at or about 0.5, 1, 2, 3, 4, 5, 10, 15, 20, 30, 40, 50, or 60 IU of the viral vector particles per one of the cells.

In some embodiments, the titer of viral vector particles is between or between about 1×10⁶ IU/mL and 1×10⁸ IU/mL, such as between or between about 5×10⁶ IU/mL and 5×10⁷ IU/mL, such as at least 6×10⁶ IU/mL, 7×10⁶ IU/mL, 8×10⁶ IU/mL, 9×10⁶ IU/mL, 1×10⁷ IU/mL, 2×10⁷ IU/mL, 3×10⁷ IU/mL, 4×10⁷ IU/mL, or 5×10⁷ IU/mL.

In some embodiments, transduction can be achieved at a multiplicity of infection (MOI) of less than 100, such as generally less than 60, 50, 40, 30, 20, 10, 5 or less.

In some embodiments, the method involves contacting or incubating, the cells with the viral particles. In some embodiments, the contacting is for 30 minutes to 72 hours, such as 30 minute to 48 hours, 30 minutes to 24 hours or 1 hour to 24 hours, such as at least or about at least or about 30 minutes, 1 hour, 2 hours, 6 hours, 12 hours, 24 hours, 36 hours or more.

In some embodiments, contacting is performed in solution. In some embodiments, the cells and viral particles are contacted in a volume of from 0.5 mL to 500 mL or from about 0.5 mL to about 500 mL, such as from or from about 0.5 mL to 200 mL, 0.5 mL to 100 mL, 0.5 mL to 50 mL, 0.5 mL to 10 mL, 0.5 mL to 5 mL, 5 mL to 500 mL, 5 mL to 200 mL, 5 mL to 100 mL, 5 mL to 50 mL, 5 mL to 10 mL, 10 mL to 500 mL, 10 mL to 200 mL, 10 mL to 100 mL, 10 mL to 50 mL, 50 mL to 500 mL, 50 mL to 200 mL, 50 mL to 100 mL, 100 mL to 500 mL, 100 mL to 200 mL or 200 mL to 500 mL.

In certain embodiments, the input cells are treated, incubated, or contacted with particles that comprise binding molecules that bind to or recognize the recombinant receptor that is encoded by the viral DNA.

In some embodiments, the incubation of the cells with the viral vector particles results in or produces an output composition comprising cells transduced with the viral vector particles.

b. Non-Viral Vectors

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, recombinant nucleic acids are transferred into T cells via transposons. Transposons (transposable elements), are mobile segments of DNA that can move from one locus to another within genomes. These elements move via a conservative, “cut-and-paste” mechanism: the transposase catalyzes the excision of the transposon from its original location and promotes its reintegration elsewhere in the genome. Transposase-deficient elements can be mobilized if the transposase is provided by another transposase gene. Thus, transposons can be utilized to incorporate a foreign DNA into a host genome without the use of a viral transduction system. Examples of transposons suitable for use with mammalian cells, e.g., human primary leukocytes, include but are not limited to Sleeping Beauty and PiggyBacs.

Transposon-based transfection is a two-component system consisting of a transposase and a transposon. In some embodiments, the system comprises a transposon is engineered to comprise a foreign DNA (also referred herein as cargo DNA), e.g., a gene encoding a recombinant receptor, that is flanked by inverted repeat/direct repeat (IR/DR) sequences that are recognized by an accompanying transposase. In some embodiments, a non-viral plasmid encodes a transposase under the control of a promoter. Transfection of the plasmid into a host cell results in a transitory expression of the transposase, thus for an initial period following transfection, the transposase is expressed at sufficiently levels to integrate the transposon into the genomic DNA. In some embodiments, the transposase itself is not integrated into the genomic DNA, and therefor expression of the transposase decreases over time. In some embodiments, the transposase expression is expressed by the host cell at levels sufficient to integrate a corresponding transposon for less than about 4 hours, less than about 8 hours, less than about 12 hours, less than about 24 hours, less than about 2 days, less than about 3 days, less than about 4 days, less than about 5 days, less than about 6 days, less than about 7 days, less than about 2 weeks, less than about 3 weeks, less than about 4 weeks, less than about weeks, or less than about 8 weeks. In some embodiments, the cargo DNA that is introduced into the host's genome is not subsequently removed from the host's genome, at least because the host dose not express an endogenous transposase capable of excising the cargo DNA.

Sleeping Beauty (SB) is a synthetic member of the Tc/1-mariner superfamily of transposons, reconstructed from dormant elements harbored in the salmonid fish genome. SB transposon-based transfection is a two-component system consisting of a transposase and a transposon containing inverted repeat/direct repeat (IR/DR) sequences that result in precise integration into a TA dinucleotide. The transposon is designed with an expression cassette of interest flanked by IR/DRs. The SB transposase binds specific binding sites that are located on the IR of the Sleeping beauty transposon. The SB transposase mediates integration of the transposon, a mobile element encoding a cargo sequence flanked on both sides by inverted terminal repeats that harbor binding sites for the catalytic enzyme (SB). Stable expression results when SB inserts gene sequences into vertebrate chromosomes at a TA target dinucleotide through a cut-and-paste mechanism. This system has been used to engineer a variety of vertebrate cell types, including primary human peripheral blood leukocytes. In some embodiments, the cells are contacted, incubated, and/or treated with an SB transposon comprising a cargo gene, e.g., a gene encoding a recombinant receptor or a CAR, flanked by SB IR sequences. In particular embodiments, the cells to be transfected are contacted, incubated, and/or treated with a plasmid comprising an SB transposon comprising a cargo gene, e.g., a gene encoding a CAR, flanked by SB IR sequences. In certain embodiments, the plasmid further comprises a gene encoding an SB transposase that is not flanked by SB IR sequences.

PiggyBac (PB) is another transposon system that can be used to integrate cargo DNA into a host's, e.g., a human's, genomic DNA. The PB transposase recognizes PB transposon-specific inverted terminal repeat sequences (ITRs) located on both ends of the transposon and efficiently moves the contents from the original sites and efficiently integrates them into TTAA chromosomal sites. The PB transposon system enables genes of interest between the two ITRs in the PB vector to be mobilized into target genomes. The PB system has been used to engineer a variety of vertebrate cell types, including primary human cells. In some embodiments, the cells to be transfected are contacted, incubated, and/or treated with a PB transposon comprising a cargo gene, e.g., a gene encoding a CAR, flanked by PB IR sequences. In particular embodiments, the cells to be transfected are contacted, incubated, and/or treated with a plasmid comprising a PB transposon comprising a cargo gene, e.g., a gene encoding a CAR, flanked by PB IR sequences. In certain embodiments, the plasmid further comprises a gene encoding an SB transposase that is not flanked by PB IR sequences.

In some embodiments, the various elements of the transposon/transposase the employed in the subject methods, e.g., SB or PB vector(s), may be produced by standard methods of restriction enzyme cleavage, ligation and molecular cloning. One protocol for constructing the subject vectors includes the following steps. First, purified nucleic acid fragments containing desired component nucleotide sequences as well as extraneous sequences are cleaved with restriction endonucleases from initial sources, e.g., a vector comprising the transposase gene. Fragments containing the desired nucleotide sequences are then separated from unwanted fragments of different size using conventional separation methods, e.g., by agarose gel electrophoresis. The desired fragments are excised from the gel and ligated together in the appropriate configuration so that a circular nucleic acid or plasmid containing the desired sequences, e.g., sequences corresponding to the various elements of the subject vectors, as described above is produced. Where desired, the circular molecules so constructed are then amplified in a prokaryotic host, e.g., E. coli. The procedures of cleavage, plasmid construction, cell transformation and plasmid production involved in these steps are well known to one skilled in the art and the enzymes required for restriction and ligation are available commercially. (See, for example, R. Wu, Ed., Methods in Enzymology, Vol. 68, Academic Press, N.Y. (1979); T. Maniatis, E. F. Fritsch and J. Sambrook, Molecular Cloning: A Laboratory Manual, Cold Spring Harbor Laboratory Press, Cold Spring Harbor, N.Y. (1982); Catalog 1982-83, New England Biolabs, Inc.; Catalog 1982-83, Bethesda Research Laboratories, Inc. An example of how to construct the vectors employed in the subject methods is provided in the Experimental section, infra. The preparation of a representative Sleeping Beauty transposon system is also disclosed in WO 98/40510 and WO 99/25817).

In some embodiments, transduction with transposons is performed with a plasmid that comprises a transposase gene and a plasmid that comprises a transposon that contains a cargo DNA sequence that is flanked by inverted repeat/direct repeat (IR/DR) sequences that are recognized by the transposase. In certain embodiments, the cargo DNA sequence encodes a heterologous protein, e.g., a recombinant T cell receptor or a CAR. In some embodiments, the plasmid comprises transposase and the transposon. In some embodiments, the transposase is under control of a ubiquitous promoter, or any promoter suitable to drive expression of the transposase in the target cell. Ubiquitous promoters include, but are not limited to, EF1a, CMB, SV40, PGK1, Ubc, human β-actin, CAG, TRE, UAS, Ac5, CaMKIIa, and U6. In some embodiments, the cargo DNA comprises a selection cassette allowing for the selection of cells with stable integration of the cargo DNA into the genomic DNA. Suitable selection cassettes include, but are not limited to, selection cassettes encoding a kanamycin resistance gene, spectinomycin resistance gene, streptomycin resistance gene, ampicillin resistance gene, carbenicillin resistance gene, hygromycin resistance gene, bleomycin resistance gene, erythromycin resistance gene, and polymyxin B resistance gene.

In some embodiments, the components for transduction with a transposon, e.g., plasmids comprising an SB transposase and SB transposon, are introduced into the target cell. Any convenient protocol may be employed, where the protocol may provide for in vitro or in vivo introduction of the system components into the target cell, depending on the location of the target cell. For example, where the target cell is an isolated cell, the system may be introduced directly into the cell under cell culture conditions permissive of viability of the target cell, e.g., by using standard transformation techniques. Such techniques include, but are not necessarily limited to: viral infection, transformation, conjugation, protoplast fusion, electroporation, particle gun technology, calcium phosphate precipitation, direct microinjection, viral vector delivery, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

In some embodiments, the SB transposon and the SB transposase source are introduced into a target cell of a multicellular organism, e.g., a mammal or a human, under conditions sufficient for excision of the inverted repeat flanked nucleic acid from the vector carrying the transposon and subsequent integration of the excised nucleic acid into the genome of the target cell. Some embodiments further comprise a step of ensuring that the requisite transposase activity is present in the target cell along with the introduced transposon. Depending on the structure of the transposon vector itself, i.e. whether or not the vector includes a region encoding a product having transposase activity, the method may further include introducing a second vector into the target cell which encodes the requisite transposase activity.

In some embodiments, the amount of vector nucleic acid comprising the transposon and the amount of vector nucleic acid encoding the transposase that is introduced into the cell is sufficient to provide for the desired excision and insertion of the transposon nucleic acid into the target cell genome. As such, the amount of vector nucleic acid introduced should provide for a sufficient amount of transposase activity and a sufficient copy number of the nucleic acid that is desired to be inserted into the target cell. The amount of vector nucleic acid that is introduced into the target cell varies depending on the efficiency of the particular introduction protocol that is employed, e.g., the particular ex vivo administration protocol that is employed.

Once the vector DNA has entered the target cell in combination with the requisite transposase, the nucleic acid region of the vector that is flanked by inverted repeats, i.e. the vector nucleic acid positioned between the Sleeping Beauty transposase recognized inverted repeats, is excised from the vector via the provided transposase and inserted into the genome of the targeted cell. As such, introduction of the vector DNA into the target cell is followed by subsequent transposase mediated excision and insertion of the exogenous nucleic acid carried by the vector into the genome of the targeted cell. In particular embodiments, the vector is integrated into the genomes of at least 1%, at least 2%, at least 3%, at least 4%, at least 5%, at least 6% at least 7% at least 8%, at least 9%, at least 10%, at least 15%, or at least 20% of the cells that are transfected with the SB transposon and/or SB transposase. In some embodiments, integration of the nucleic acid into the target cell genome is stable, i.e., the vector nucleic acid remains present in the target cell genome for more than a transient period of time and is passed on a part of the chromosomal genetic material to the progeny of the target cell.

In certain embodiments, the transposons are used to integrate nucleic acids, i.e. polynucleotides, of various sizes into the target cell genome. In some embodiments, the size of DNA that is inserted into a target cell genome using the subject methods ranges from about 0.1 kb to 200 kb, from about 0.5 kb to 100 kb, from about 1.0 kb to about 8.0 kb, from about 1.0 to about 200 kb, from about 1.0 to about 10 kb, from about 10 kb to about 50 kb, from about 50 kb to about 100 kb, or from about 100 kb to about 200 kb. In some embodiments, the size of DNA that is inserted into a target cell genome using the subject methods ranges from about from about 1.0 kb to about 8.0 kb. In some embodiments, the size of DNA that is inserted into a target cell genome using the subject methods ranges from about 1.0 to about 200 kb. In particular embodiments, the size of DNA that is inserted into a target cell genome using the subject methods ranges from about 1.0 kb to about 8.0 kb.

D. Cultivation and/or Expansion of Cells

In some embodiments, the provided methods include one or more steps for cultivating cells, e.g., cultivating cells under conditions that promote proliferation and/or expansion. In some embodiments, cells are cultivated under conditions that promote proliferation and/or expansion subsequent to a step of genetically engineering, e.g., introducing a recombinant polypeptide to the cells by transduction or transfection. In particular embodiments, the cells are cultivated after the cells have been incubated under stimulating conditions and transduced or transfected with a recombinant polynucleotide, e.g., a polynucleotide encoding a recombinant receptor. In some embodiments, the cultivation produces one or more cultivated compositions of enriched T cells.

In certain embodiments, one or more compositions of enriched T cells, including stimulated and transduced T cells, such as separate compositions of such CD4+ and CD8+ T cells, are cultivated, e.g., under conditions that promote proliferation and/or expansion, prior to formulating the cells. In some aspects, the methods of cultivation, such as for promoting proliferation and/or expansion include methods provided herein, such as in Section I-F. In particular embodiments, one or more compositions of enriched T cells are cultivated after the one or more compositions have been engineered, e.g., transduced or transfected. In particular embodiments, the one or more compositions are engineered compositions. In particular embodiments, the one or more engineered compositions have been previously cryofrozen and stored, and are thawed prior to cultivating.

In certain embodiments, the one or more compositions of engineered T cells are or include two separate compositions of enriched T cells. In particular embodiments, two separate compositions of enriched T cells, e.g., two separate compositions of enriched T cells selected, isolated, and/or enriched from the same biological sample, that are introduced with a recombinant receptor (e.g., CAR), are separately cultivated under conditions that promote proliferation and/or expansion of the cells. In some embodiments, the conditions are stimulating conditions. In certain embodiments, the two separate compositions include a composition of enriched CD4+ T cells, such as engineered CD4+ T cells that were introduced with the nucleic acid encoding the recombinant receptor and/or that express the recombinant receptor. In particular embodiments, the two separate compositions include a composition of enriched CD8+ T cells, such as engineered CD8+ T cells that were introduced with the nucleic acid encoding the recombinant receptor and/or that express the recombinant receptor. In some embodiments, two separate compositions of enriched CD4+ T cells and enriched CD8+ T cells, such as engineered CD4+ T cells and engineered CD8+ T cells, are separately cultivated, e.g., under conditions that promote proliferation and/or expansion. In some embodiments, a single composition of enriched T cells is cultivated. In certain embodiments, the single composition is a composition of enriched CD4+ T cells. In some embodiments, the single composition is a composition of enriched CD4+ and CD8+ T cells that have been combined from separate compositions prior to the cultivation.

In some embodiments, the composition of enriched CD4+ T cells, such as engineered CD4+ T cells, that is cultivated, e.g., under conditions that promote proliferation and/or expansion, includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In some embodiments, the composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD4+ T cells that express the recombinant receptor and/or have been transduced or transfected with the recombinant polynucleotide encoding the recombinant receptor. In certain embodiments, the composition of enriched CD4+ T cells that is cultivated includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells, such as engineered CD8+t cells, that is cultivated, e.g., under conditions that promote proliferation and/or expansion, includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In particular embodiments, the composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD8+ T cells that express the recombinant receptor and/or have been transduced or transfected with the recombinant polynucleotide encoding the recombinant receptor. In certain embodiments, the composition of enriched CD8+ T cells that is incubated under stimulating conditions includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, separate compositions of enriched CD4+ and CD8+ T cells, such as separate compositions of engineered CD4+ and engineered CD8+ T cells, are combined into a single composition and are cultivated, e.g., under conditions that promote proliferation and/or expansion. In certain embodiments, separate cultivated compositions of enriched CD4+ and enriched CD8+ T cells are combined into a single composition after the cultivation has been performed and/or completed. In particular embodiments, separate compositions of enriched CD4+ and CD8+ T cells, such as separate compositions of engineered CD4+ and engineered CD8+ T cells, are separately cultivated, e.g., under conditions that promote proliferation and/or expansion.

In some embodiments, the cells, e.g., the engineered cells are cultivated in a volume of media that is, is about, or is at least 100 mL, 200 mL, 300 mL, 400 mL, 500 mL, 600 mL, 700 mL, 800 mL, 900 mL, 1,000 mL, 1,200 mL, 1,400 mL, 1,600 mL, 1,800 mL, 2,000 mL, 2,200 mL, or 2,400 mL. In some embodiments, the cells are cultivated at an initial volume that is later adjusted to a different volume. In particular embodiments, the volume is later adjusted during the cultivation. In particular embodiments, the volume is increased from the initial volume during the cultivation. In certain embodiments, the volume is increased when the cells achieve a density during the cultivation. In certain embodiment, the initial volume is or is about 500 mL.

In particular embodiments, the volume is increased from the initial volume when the cells achieve a density or concentration during the cultivation. In particular embodiments, the volume is increased when the cells achieve a density and/or concentration of, of about, or of at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In some embodiments, the volume is increased from the initial volume when the cells achieve a density and/or concentration of, of at least, or of about 0.6×10⁶ cells/ml. In some embodiments, the density and/or concentration is of viable cells in the culture. In particular embodiments, the volume is increased when the cells achieve a density and/or concentration of, of about, or of at least 0.1×10⁶ viable cells/ml, 0.2×10⁶ viable cells/ml, 0.4×10⁶ viable cells/ml, 0.6×10⁶ viable cells/ml, 0.8×10⁶ viable cells/ml, 1×10⁶ viable cells/ml, 1.2×10⁶ viable cells/ml, 1.4×10⁶ viable cells/ml, 1.6×10⁶ viable cells/ml, 1.8×10⁶ viable cells/ml, 2.0×10⁶ viable cells/ml, 2.5×10⁶ viable cells/ml, 3.0×10⁶ viable cells/ml, 3.5×10⁶ viable cells/ml, 4.0×10⁶ viable cells/ml, 4.5×10⁶ viable cells/ml, 5.0×10⁶ viable cells/ml, 6×10⁶ viable cells/ml, 8×10⁶ viable cells/ml, or 10×10⁶ viable cells/ml. In some embodiments, the volume is increased from the initial volume when the viable cells achieve a density and/or concentration of, of at least, or of about 0.6×10⁶ viable cells/ml. In some embodiments, density and/or concentration of the cells or viable cells can be determined or monitored during the cultivation, such as by using methods as described, including optical methods, including digital holography microscopy (DHM) or differential digital holography microscopy (DDHM).

In some embodiments, the cells achieve a density and/or concentration, and the volume is increased by, by about, or by at least 100 mL, 200 mL, 300 mL, 400 mL, 500 mL, 600 mL, 700 mL, 800 mL, 900 mL, 1,000 mL, 1,200 mL, 1,400 mL, 1,600 mL, 1,800 mL, 2,000 mL, 2,200 mL or 2,400 mL. In some embodiments, the volume is increased by 500 mL. In particular embodiments, the volume is increased to a volume of, of about, or of at least 500 mL, 600 mL, 700 mL, 800 mL, 900 mL, 1,000 mL, 1,200 mL, 1,400 mL, 1,600 mL, 1,800 mL, 2,000 mL, 2,200 mL or 2,400 mL. In certain embodiments, the volume is increased to a volume of 1,000 mL. In certain embodiments, the volume is increase at a rate of, of at least, or of about 5 mL, 10 mL, 20 mL, 25 mL, 30 mL, 40 mL, 50 mL, 60 mL, 70 mL, 75 mL, 80 mL, 90 mL, or 100 mL, every 1, 2, 3, 4, 5, 6, 7, 8, 9, or 10 minutes. In certain embodiments, the rate is or is about 50 mL every 8 minutes.

In some embodiments, a composition of enriched T cells, such as engineered T cells, is cultivated under conditions that promote proliferation and/or expansion. In some embodiments, such conditions may be designed to induce proliferation, expansion, activation, and/or survival of cells in the population. In particular embodiments, the stimulating conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to promote growth, division, and/or expansion of the cells.

In some embodiments, the cultivation is performed under conditions that generally include a temperature suitable for the growth of primary immune cells, such as human T lymphocytes, for example, at least about 25 degrees Celsius, generally at least about 30 degrees, and generally at or about 37 degrees Celsius. In some embodiments, the composition of enriched T cells is incubated at a temperature of 25 to 38° C., such as 30 to 37° C., for example at or about 37° C.±2° C. In some embodiments, the incubation is carried out for a time period until the culture, e.g., cultivation or expansion, results in a desired or threshold density, concentration, number or dose of cells. In some embodiments, the incubation is carried out for a time period until the culture, e.g., cultivation or expansion, results in a desired or threshold density, concentration, number or dose of viable cells. In some embodiments, the incubation is greater than or greater than about or is for about or 24 hours, 48 hours, 72 hours, 96 hours, 5 days, 6 days, 7 days, 8 days, 9 days or more. In some embodiments, density, concentration and/or number or dose of the cells can be determined or monitored during the cultivation, such as by using methods as described, including optical methods, including digital holography microscopy (DHM) or differential digital holography microscopy (DDHM).

In some embodiments, the stimulatory reagent is removed and/or separated from the cells prior to the cultivation. In certain embodiments, the stimulatory agent is removed and/or separated from the cells subsequent to the engineering and prior to cultivating the engineered cells, e.g., under conditions that promote proliferation and/or expansion. In some embodiments, the stimulatory reagent is a stimulatory reagent that is described herein, e.g., in Section I-B-1. In particular embodiments, the stimulatory reagent is removed and/or separated from the cells as described herein, e.g., in Section I-B-2.

In particular embodiments, a composition of enriched T cells, such as engineered T cells, for example separate compositions of engineered CD4+ T cells and engineered CD8+ T cells, is cultivated in the presence of one or more cytokines. In certain embodiments, the one or more cytokines are recombinant cytokines. In particular embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes recombinant IL-2.

In particular embodiments, the composition of enriched CD4+ T cells, such as engineered CD4+ T cells, is cultivated with recombinant IL-2. In some embodiments, cultivating a composition of enriched CD4+ T cells, such as engineered CD4+ T cells, in the presence of recombinant IL-2 increases the probability or likelihood that the CD4+ T cells of the composition will continue to survive, grow, expand, and/or activate during the cultivation step and throughout the process. In some embodiments, cultivating the composition of enriched CD4+ T cells, such as engineered CD4+ T cells, in the presence of recombinant IL-2 increases the probability and/or likelihood that an output composition of enriched CD4+ T cells, e.g., engineered CD4+ T cells suitable for cell therapy, will be produced from the composition of enriched CD4+ T cells by at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 30%, at least 40%, at least 50%, at least 60%, at least 70%, at least 80%, at least 90%, at least 95%, at least 100%, or at least 200% CD4+ as compared to an alternative and/or exemplary method that does not cultivate the composition of enriched CD4+ T cells in the presence of recombinant IL-2.

In some embodiments, the cells, such as separate compositions of engineered CD4+ T cells and engineered CD8+ T cells, are cultivated with a cytokine, e.g., a recombinant human cytokine, at a concentration of between 1 IU/ml and 2,000 IU/ml, between 10 IU/ml and 100 IU/ml, between 50 IU/ml and 500 IU/ml, between 100 IU/ml and 200 IU/ml, between 500 IU/ml and 1400 IU/ml, between 250 IU/ml and 500 IU/ml, or between 500 IU/ml and 2,500 IU/ml.

In some embodiments, a composition of enriched of T cells, such as separate compositions of engineered CD4+ T cells and CD8+ T cells, is cultivated with recombinant IL-2, e.g., human recombinant IL-2, at a concentration between 2 IU/ml and 500 IU/ml, between 10 IU/ml and 250 IU/ml, between 100 IU/ml and 500 IU/ml, or between 100 IU/ml and 400 IU/ml. In particular embodiments, the composition of enriched T cells is cultivated with IL-2 at a concentration at or at about 50 IU/ml, 75 IU/ml, 100 IU/ml, 125 IU/ml, 150 IU/ml, 175 IU/ml, 200 IU/ml, 225 RJ/ml, 250 IU/ml, 300 RJ/ml, or 400 IU/ml. In some embodiments, the composition of enriched T cells is cultivated with recombinant IL-2 at a concentration of 200 IU/ml. In some embodiments, the composition of enriched T cells is a composition of enriched CD4+ T cells, such as a composition of engineered CD4+ T cells. In particular embodiments, the composition of enriched T cells is a composition of enriched CD8+ T cells, such as a composition of engineered CD8+ T cells.

In some embodiments, a composition of enriched T cells, such as separate compositions of engineered CD4+ T cells and CD8+ T cells, is cultivated with IL-7, e.g., human recombinant IL-7, at a concentration between 10 IU/ml and 5,000 IU/ml, between 500 IU/ml and 2,000 IU/ml, between 600 IU/ml and 1,500 IU/ml, between 500 IU/ml and 2,500 IU/ml, between 750 IU/ml and 1,500 IU/ml, or between 1,000 IU/ml and 2,000 IU/ml. In particular embodiments, the composition of enriched T cells is cultivated with IL-7 at a concentration at or at about 100 IU/ml, 200 IU/ml, 300 IU/ml, 400 IU/ml, 500 IU/ml, 600 IU/ml, 700 IU/ml, 800 IU/ml, 900 IU/ml, 1,000 IU/ml, 1,200 IU/ml, 1,400 IU/ml, or 1,600 IU/ml. In some embodiments, the cells are cultivated in the presence of recombinant IL-7 at a concertation of or of about 1,200 IU/ml. In some embodiments, the composition of enriched T cells is a composition of enriched CD4+ T cells, such as engineered CD4+ T cells.

In some embodiments, a composition of enriched T cells, such as separate compositions of engineered CD4+ T cells and CD8+ T cells, is cultivated with IL-15, e.g., human recombinant IL-15, at a concentration between 0.1 IU/ml and 200 IU/ml, between 1 IU/ml and 50 IU/ml, between 5 IU/ml and 25 IU/ml, between 25 IU/ml and 50 IU/ml, between 5 IU/ml and 15 IU/ml, or between 10 IU/ml and 100 IU/ml. In particular embodiments, the composition of enriched T cells is cultivated with IL-15 at a concentration at or at about 1 IU/ml, 2 IU/ml, 3 IU/ml, 4 IU/ml, 5 IU/ml, 6 IU/ml, 7 IU/ml, 8 IU/ml, 9 IU/ml, 10 IU/ml, 11 IU/ml, 12 IU/ml, 13 IU/ml, 14 IU/ml, 15 IU/ml, 20 IU/ml, 25 IU/ml, 30 IU/ml, 40 IU/ml, 50 IU/ml, 100 IU/ml, or 200 IU/ml. In particular embodiments, a composition of enriched T cells is cultivated with recombinant IL-15 at a concentration of 20 IU/ml. In some embodiments, the composition of enriched T cells is a composition of enriched CD4+ T cells, such as engineered CD4+ T cells. In particular embodiments, the composition of enriched T cells is a composition of enriched CD8+ T cells, such as engineered CD8+ T cells.

In particular embodiments, a composition of enriched CD8+ T cells, such as engineered CD8+ T cells, is cultivated in the presence of IL-2 and/or IL-15, such as in amounts as described. In certain embodiments, a composition of enriched CD4+ T cells, such as engineered CD4+ T cells, is cultivated in the presence of IL-2, IL-7, and/or IL-15, such as in amounts as described. In some embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15.

In particular embodiments, the cultivation is performed in a closed system. In certain embodiments, the cultivation is performed in a closed system under sterile conditions. In particular embodiments, the cultivation is performed in the same closed system as one or more steps of the provided systems. In some embodiments the composition of enriched T cells is removed from a closed system and placed in and/or connected to a bioreactor for the cultivation. Examples of suitable bioreactors for the cultivation include, but are not limited to, GE Xuri W25, GE Xuri W5, Sartorius BioSTAT RM 20|50, Finesse SmartRocker Bioreactor Systems, and Pall XRS Bioreactor Systems. In some embodiments, the bioreactor is used to perfuse and/or mix the cells during at least a portion of the cultivation step.

In some embodiments, cells cultivated while enclosed, connected, and/or under control of a bioreactor undergo expansion during the cultivation more rapidly than cells that are cultivated without a bioreactor, e.g., cells that are cultivated under static conditions such as without mixing, rocking, motion, and/or perfusion. In some embodiments, cells cultivated while enclosed, connected, and/or under control of a bioreactor reach or achieve a threshold expansion, cell count, and/or density within 14 days, 10 days, 9 days, 8 days, 7 days, 6 days, 5 days, 4 days, 3 days, 2 days, 60 hours, 48 hours, 36 hours, 24 hours, or 12 hours. In some embodiments, cells cultivated while enclosed, connected, and/or under control of a bioreactor reach or achieve a threshold expansion, cell count, and/or density at least 50%, at least 60%, at least 70%, at least 80%, at least 90%, at least 95%, at least 100%, at least 150%, at least 1-fold, at least 2-fold, at least 3-fold, at least 4-fold, at least 5-fold than cells cultivated in an exemplary and/or alternative process where cells are not cultivated while enclosed, connected, and/or under control of a bioreactor.

In some embodiments, the mixing is or includes rocking and/or motioning. In some cases, the bioreactor can be subject to motioning or rocking, which, in some aspects, can increase oxygen transfer. Motioning the bioreactor may include, but is not limited to rotating along a horizontal axis, rotating along a vertical axis, a rocking motion along a tilted or inclined horizontal axis of the bioreactor or any combination thereof. In some embodiments, at least a portion of the incubation is carried out with rocking. The rocking speed and rocking angle may be adjusted to achieve a desired agitation. In some embodiments the rock angle is 20°, 19°, 18°, 17°, 16°, 15°, 14°, 13°, 12°, 11°, 10°, 9°, 8°, 7°, 6°, 5°, 4°, 30°, 2° or 1°.

In certain embodiments, the rock angle is between 6-16°. In other embodiments, the rock angle is between 7-16°. In other embodiments, the rock angle is between 8-12°. In some embodiments, the rock rate is 1, 2, 3, 4, 5, 6, 7, 8, 9, 10, 11, 1 12, 13, 14 15, 16, 17, 18, 19, 20, 21, 22, 23, 24, 25, 26, 27, 28, 29, 30, 31, 32, 33, 34, 35, 36, 37, 38, 39, 40 rpm. In some embodiments, the rock rate is between 4 and 12 rpm, such as between 4 and 6 rpm, inclusive.

In some embodiments, the bioreactor maintains the temperature at or near 37° C. and CO₂ levels at or near 5% with a steady air flow at, at about, or at least 0.01 L/min, 0.05 L/min, 0.1 L/min, 0.2 L/min, 0.3 L/min, 0.4 L/min, 0.5 L/min, 1.0 L/min, 1.5 L/min, or 2.0 L/min or greater than 2.0 L/min. In certain embodiments, at least a portion of the cultivation is performed with perfusion, such as with a rate of 290 ml/day, 580 ml/day, and/or 1160 ml/day, e.g., depending on the timing in relation to the start of the cultivation and/or density of the cultivated cells. In some embodiments, at least a portion of the cell culture expansion is performed with a rocking motion, such as at an angle of between 5° and 10°, such as 6°, at a constant rocking speed, such as a speed of between 5 and 15 RPM, such as 6 RPM or 10 RPM.

In some embodiments, the at least a portion of the cultivation step is performed under constant perfusion, e.g., a perfusion at a slow steady rate. In some embodiments, the perfusion is or include an outflow of liquid e.g., used media, and an inflow of fresh media. In certain embodiments, the perfusion replaces used media with fresh media. In some embodiments, at least a portion of the cultivation is performed under perfusion at a steady rate of or of about or of at least 100 ml/day, 200 ml/day, 250 ml/day, 275 ml/day, 290 ml/day, 300 ml/day, 350 ml/day, 400 ml/day, 450 ml/day, 500 ml/day, 550 ml/day, 575 ml/day, 580 ml/day, 600 ml/day, 650 ml/day, 700 ml/day, 750 ml/day, 800 ml/day, 850 ml/day, 900 ml/day, 950 ml/day, 1000 ml/day, 1100 ml/day, 1160 ml/day, 1200 ml/day, 1400 ml/day, 1600 ml/day, 1800 ml/day, 2000 ml/day, 2200 ml/day, or 2400 ml/day.

In particular embodiments, cultivation is started under conditions with no perfusion, and perfusion started after a set and/or predetermined amount of time, such as or as about or at least 12 hours, 24 hours, 36 hours, 48 hours, 60 hours, 72 hours, or more than 72 hours after the start or initiation of the cultivation. In particular embodiments, perfusion is started when the density or concentration of the cells reaches a set or predetermined density or concentration. In some embodiments, the perfusion is started when the cultivated cells reach a density or concentration of, of about, or at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In particular embodiments, perfusion is started when the density or concentration of viable cells reaches a set or predetermined density or concentration. In some embodiments, the perfusion is started when the cultivated viable cells reach a density or concentration of, of about, or at least 0.1×10⁶ viable cells/ml, 0.2×10⁶ viable cells/ml, 0.4×10⁶ viable cells/ml, 0.6×10⁶ viable cells/ml, 0.8×10⁶ viable cells/ml, 1×10⁶ viable cells/ml, 1.2×10⁶ viable cells/ml, 1.4×10⁶ viable cells/ml, 1.6×10⁶ viable cells/ml, 1.8×10⁶ viable cells/ml, 2.0×10⁶ viable cells/ml, 2.5×10⁶ viable cells/ml, 3.0×10⁶ viable cells/ml, 3.5×10⁶ viable cells/ml, 4.0×10⁶ viable cells/ml, 4.5×10⁶ viable cells/ml, 5.0×10⁶ viable cells/ml, 6×10⁶ viable cells/ml, 8×10⁶ viable cells/ml, or 10×10⁶ viable cells/ml.

In particular embodiments, the perfusion is performed at different speeds during the cultivation. For example, in some embodiments, the rate of the perfusion depends on the density and/or concentration of the cultivated cells. In certain embodiments, the rate of perfusion is increased when the cells reach a set or predetermined density or concentration. The perfusion rate may change, e.g., change from one steady perfusion rate to an increased steady perfusion rate, once, twice, three times, four times, five times, more than five times, more than ten times, more than 15 times, more than 20 times, more than 25 times, more than 50 times, or more than 100 times during the cultivation. In some embodiments, the steady perfusion rate increases when the cells reach a set or predetermined cell density or concentration of, of about, or at least 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In some embodiments, the steady perfusion rate increases when the cells reach a set or predetermined viable cell density or concentration of, of about, or at least 0.6×10⁶ viable cells/ml, 0.8×10⁶ viable cells/ml, 1×10⁶ viable cells/ml, 1.2×10⁶ viable cells/ml, 1.4×10⁶ viable cells/ml, 1.6×10⁶ viable cells/ml, 1.8×10⁶ viable cells/ml, 2.0×10⁶ viable cells/ml, 2.5×10⁶ viable cells/ml, 3.0×10⁶ viable cells/ml, 3.5×10⁶ viable cells/ml, 4.0×10⁶ viable cells/ml, 4.5×10⁶ viable cells/ml, 5.0×10⁶ viable cells/ml, 6×10⁶ viable cells/ml, 8×10⁶ viable cells/ml, or 10×10⁶ viable cells/ml. In some embodiments, density and/or concentration of the cells or of the viable cells during the cultivation, such as under perfusion, can be determined or monitored, such as by using methods as described, including optical methods, including digital holography microscopy (DHM) or differential digital holography microscopy (DDHM).

In some embodiments, cultivation is started under conditions with no perfusion, and, perfusion is started when the density or concentration of the cells reaches a set or predetermined density or concentration. In some embodiments, the perfusion is started at a rate of, of about, or of at least 100 ml/day, 200 ml/day, 250 ml/day, 275 ml/day, 290 ml/day, 300 ml/day, 350 ml/day, 400 ml/day, 450 ml/day, 500 ml/day, 550 ml/day, 575 ml/day, 580 ml/day, 600 ml/day, 650 ml/day, 700 ml/day, 750 ml/day, 800 ml/day, 850 ml/day, 900 ml/day, 950 ml/day, 1000 ml/day, 1100 ml/day, 1160 ml/day, 1200 ml/day, 1400 ml/day, 1600 ml/day, 1800 ml/day, 2000 ml/day, 2200 ml/day, or 2400 ml/day when the density or concentration of the cells reaches a set or predetermined density or concentration. In some embodiments, the perfusion is started when the cultivated cells or cultivated viable cells reach a density or concentration of, of about, or at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml.

In certain embodiments, at least part of the cultivation is performed with perfusion at a certain rate, and the perfusion rate is increased to, to about, or to at least 100 ml/day, 200 ml/day, 250 ml/day, 275 ml/day, 290 ml/day, 300 ml/day, 350 ml/day, 400 ml/day, 450 ml/day, 500 ml/day, 550 ml/day, 575 ml/day, 580 ml/day, 600 ml/day, 650 ml/day, 700 ml/day, 750 ml/day, 800 ml/day, 850 ml/day, 900 ml/day, 950 ml/day, 1000 ml/day, 1100 ml/day, 1160 ml/day, 1200 ml/day, 1400 ml/day, 1600 ml/day, 1800 ml/day, 2000 ml/day, 2200 ml/day, or 2400 ml/day when the density or concentration of the cells reaches a set or predetermined density or concentration. In some embodiments, the perfusion is started when the cultivated cells or cultivated viable cells reach a density or concentration of, of about, or at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In some embodiments, the perfusion is performed when the cells are cultivated in a volume of, of about, or at least 300 mL, 400 mL, 500 mL, 600 mL, 700 mL, 800 mL, 900 mL, or 1000 mL. In some embodiments, the volume is 1000 mL.

In certain embodiments, cultivation is started under conditions with either no perfusion or perfusion at a certain rate, and the perfusion rate is increased to, to about, or to at 290 ml/day when the density or concentration of the cells reaches a concentration of, of about, or of at least 0.61×10⁶ cells/ml. In certain embodiments, the cells are perfused at a rate of, of about, or at least 290 ml/day when the density or concentration of the cells reaches a concentration of, of about, or of at least 0.61×10⁶ cells/ml when the cells are cultivated at a volume of, of about, or at least 1000 mL. In some embodiments, the perfusion rate is increased to, to about, or to at 580 ml/day when the density or concentration of the cells reaches a concentration of, of about, or of at least 0.81×10⁶ cells/ml. In certain embodiments, the perfusion rate is increased to, to about, or to at 1160 ml/day when the density or concentration of the cells reaches a concentration of, of about, or of at least 1.01×10⁶ cells/ml. In some embodiments, the perfusion rate is increased to, to about, or to at 1160 ml/day when the density or concentration of the cells reaches a concentration of, of about, or of at least 1.2×10⁶ cells/ml.

In aspects of the provided embodiments, the rate of perfusion, including the timing of when it is started or increased as described herein and above, is determined from assessing density and/or concentration of the cells or assessing the density and/or concentration of viable cells during the cultivation. In some embodiments, density and/or concentration of the cells can be determined using methods as described, including optical methods, including digital holography microscopy (DHM) or differential digital holography microscopy (DDHM).

In some embodiments, a composition of enriched cells, such as engineered T cells, e.g., engineered CD4+ T cells or engineered CD8+ T cells, is cultivated in the presence of a surfactant. In particular embodiments, cultivating the cells of the composition reduces the amount of shear stress that may occur during the cultivation, e.g., due to mixing, rocking, motion, and/or perfusion. In particular embodiments, the composition of enriched T cells, such as engineered T cells, e.g., engineered CD4+ T cells or engineered CD8+ T cells, is cultivated with the surfactant and at least 50%, at least 60%, at least 70%, at least 75%, at least 80%, at least 85%, at least 90%, at least 95%, at least 99%, or at least 99.9% of the T cells survive, e.g., are viable and/or do not undergo necrosis, programed cell death, or apoptosis, during or at least 1 day, 2 days, 3 days, 4 days, 5 days, 6 days, 7 days, or more than 7 days after the cultivation is complete. In particular embodiments, the composition of enriched T cells, such as engineered T cells, e.g., engineered CD4+ T cells or engineered CD8+ T cells, is cultivated in the presence of a surfactant and less than 50%, less than 40%, less than 30%, less than 25%, less than 20%, less than 15%, less than 10%, less than 5%, less than 1%, less than 0.1% or less than 0.01% of the cells undergo cell death, e.g., programmed cell death, apoptosis, and/or necrosis, such as due to shearing or shearing-induced stress.

In particular embodiments, a composition of enriched T cells, such as engineered T cells, e.g., engineered CD4+ T cells or engineered CD8+ T cells, is cultivated in the presence of between 0.1 μl/ml and 10.0 between 0.2 μl/ml and 2.5 between 0.5 μl/ml and 5 μl/ml, between 1 μl/ml and 3 μl/ml, or between 2 μl/ml and 4 μl/ml of the surfactant. In some embodiments, the composition of enriched T cells, such as engineered T cells, e.g., engineered CD4+ T cells or engineered CD8+ T cells, is cultivated in the presence of, of about, or at least 0.1 μl/ml, 0.2 μl/ml, 0.4 μl/ml, 0.6 μl/ml, 0.8 μl/ml, 1 μl/ml, 1.5 μl/ml, 2 μl/ml, 2.5 μl/ml, 5 μl/ml, 10 μl/ml, 25 μl/ml, or 50 μl/ml of the surfactant. In certain embodiments, the composition of enriched T cells is cultivated in the presence of or of about 2 μl/ml of the surfactant.

In some embodiments, a surfactant is or includes an agent that reduces the surface tension of liquids and/or solids. For example, a surfactant includes a fatty alcohol (e.g., steryl alcohol), a polyoxyethylene glycol octylphenol ether (e.g., Triton X-100), or a polyoxyethylene glycol sorbitan alkyl ester (e.g., polysorbate 20, 40, 60). In certain embodiments the surfactant is selected from the group consisting of Polysorbate 80 (PS80), polysorbate 20 (PS20), poloxamer 188 (P188). In an exemplary embodiment, the concentration of the surfactant in chemically defined feed media is about 0.0025% to about 0.25% (v/v) of PS80; about 0.0025% to about 0.25% (v/v) of PS20; or about 0.1% to about 5.0% (w/v) of P188.

In some embodiments, the surfactant is or includes an anionic surfactant, a cationic surfactant, a zwitterionic surfactant, or a nonionic surfactant added thereto. Suitable anionic surfactants include but are not limited to alkyl sulfonates, alkyl phosphates, alkyl phosphonates, potassium laurate, triethanolamine stearate, sodium lauryl sulfate, sodium dodecylsulfate, alkyl polyoxyethylene sulfates, sodium alginate, dioctyl sodium sulfosuccinate, phosphatidyl glycerol, phosphatidyl inosine, phosphatidylinositol, diphosphatidylglycerol, phosphatidylserine, phosphatidic acid and their salts, sodium carboxymethylcellulose, cholic acid and other bile acids (e.g., cholic acid, deoxycholic acid, glycocholic acid, taurocholic acid, glycodeoxycholic acid) and salts thereof (e.g., sodium deoxycholate).

In some embodiments, suitable nonionic surfactants include: glyceryl esters, polyoxyethylene fatty alcohol ethers, polyoxyethylene sorbitan fatty acid esters (polysorbates), polyoxyethylene fatty acid esters, sorbitan esters, glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), poloxamines, methylcellulose, hydroxymethyl cellulose, hydroxypropyl cellulose, hydroxypropylmethyl cellulose, noncrystalline cellulose, polysaccharides including starch and starch derivatives such as hydroxyethylstarch (HES), polyvinyl alcohol, and polyvinylpyrrolidone. In certain embodiments, the nonionic surfactant is a polyoxyethylene and polyoxypropylene copolymer and preferably a block copolymer of propylene glycol and ethylene glycol. Such polymers are sold under the tradename POLOXAMER, also sometimes referred to as PLURONIC® F68 or Kolliphor® P188. Among polyoxyethylene fatty acid esters is included those having short alkyl chains. One example of such a surfactant is SOLUTOL® HS 15, polyethylene-660-hydroxystearate.

In some embodiments, suitable cationic surfactants may include, but are not limited to, natural phospholipids, synthetic phospholipids, quaternary ammonium compounds, benzalkonium chloride, cetyltrimethyl ammonium bromide, chitosans, lauryl dimethyl benzyl ammonium chloride, acyl carnitine hydrochlorides, dimethyl dioctadecyl ammonium bromide (DDAB), dioleyoltrimethyl ammonium propane (DOTAP), dimyristoyl trimethyl ammonium propane (DMTAP), dimethyl amino ethane carbamoyl cholesterol (DC-Chol), 1,2-diacylglycero-3-(O-alkyl) phosphocholine, O-alkylphosphatidylcholine, alkyl pyridinium halides, or long-chain alkyl amines such as, for example, n-octylamine and oleylamine.

Zwitterionic surfactants are electrically neutral but possess local positive and negative charges within the same molecule. Suitable zwitterionic surfactants include but are not limited to zwitterionic phospholipids. Suitable phospholipids include phosphatidylcholine, phosphatidylethanolamine, diacyl-glycero-phosphoethanolamine (such as dimyristoyl-glycero-phosphoethanolamine (DMPE), dipalmitoyl-glycero-phosphoethanolamine (DPPE), distearoyl-glycero-phosphoethanolamine (DSPE), and dioleolyl-glycero-phosphoethanolamine (DOPE)). Mixtures of phospholipids that include anionic and zwitterionic phospholipids may be employed in this invention. Such mixtures include but are not limited to lysophospholipids, egg or soybean phospholipid or any combination thereof. The phospholipid, whether anionic, zwitterionic or a mixture of phospholipids, may be salted or desalted, hydrogenated or partially hydrogenated or natural semi-synthetic or synthetic.

In certain embodiments, the surfactant is poloxamer, e.g., poloxamer 188. In some embodiments, a composition of enriched T cells is cultivated in the presence of between 0.1 μl/ml and 10.0 μl/ml, between 0.2 μl/ml and 2.5 μl/ml, between 0.5 μl/ml and 5 μl/ml, between 1 μl/ml and 3 μl/ml, or between 2 μl/ml and 4 μl/ml of poloxamer. In some embodiments, the composition of enriched T cells is cultivated in the presence of, of about, or at least 0.1 μl/ml, 0.2 μl/ml, 0.4 μl/ml, 0.6 μl/ml, 0.8 μl/ml, 1 μl/ml, 1.5 μl/ml, 2 μl/ml, 2.5 μl/ml, 5 μl/ml, 10 μl/ml, 25 μl/ml, or 50 μl/ml of the surfactant. In certain embodiments, the composition of enriched T cells is cultivated in the presence of or of about 2 μl/ml of poloxamer.

In particular embodiments, the cultivation ends, such as by harvesting cells, when cells achieve a threshold amount, concentration, and/or expansion. In particular embodiments, the cultivation ends when the cell achieve or achieve about or at least a 1.5-fold expansion, a 2-fold expansion, a 2.5-fold expansion, a 3-fold expansion, a 3.5-fold expansion, a 4-fold expansion, a 4.5-fold expansion, a 5-fold expansion, a 6-fold expansion, a 7-fold expansion, a 8-fold expansion, a 9-fold expansion, a 10-fold expansion, or greater than a 10-fold expansion, e.g., with respect and/or in relation to the amount of density of the cells at the start or initiation of the cultivation. In some embodiments, the threshold expansion is a 4-fold expansion, e.g., with respect and/or in relation to the amount of density of the cells at the start or initiation of the cultivation.

In some embodiments, the cultivation ends, such as by harvesting cells, when the cells achieve a threshold total amount of cells, e.g., threshold cell count. In some embodiments, the cultivation ends when the cells achieve a threshold total nucleated cell (TNC) count. In some embodiments, the cultivation ends when the cells achieve a threshold viable amount of cells, e.g., threshold viable cell count. In some embodiments, the threshold cell count is or is about or is at least of 50×10⁶ cells, 100×10⁶ cells, 200×10⁶ cells, 300×10⁶ cells, 400×10⁶ cells, 600×10⁶ cells, 800×10⁶ cells, 1000×10⁶ cells, 1200×10⁶ cells, 1400×10⁶ cells, 1600×10⁶ cells, 1800×10⁶ cells, 2000×10⁶ cells, 2500×10⁶ cells, 3000×10⁶ cells, 4000×10⁶ cells, 5000×10⁶ cells, 10,000×10⁶ cells, 12,000×10⁶ cells, 15,000×10⁶ cells or 20,000×10⁶ cells, or any of the foregoing threshold of viable cells. In particular embodiments, the cultivation ends when the cells achieve a threshold cell count. In some embodiments, the cultivation ends at, at about, or within 6 hours, 12 hours, 24 hours, 36 hours, 1 day, 2 days, 3 days, 4 days, 5 days, 6 days, or 7 or more days, after the threshold cell count is achieved. In particular embodiments, the cultivation is ended at or about 1 day after the threshold cell count is achieved. In certain embodiments, the threshold density is, is about, or is at least 0.1×10⁶ cells/ml, 0.5×10⁶ cells/ml, 1×10⁶ cells/ml, 1.2×10⁶ cells/ml, 1.5×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml, or any of the foregoing threshold of viable cells. In particular embodiments, the cultivation ends when the cells achieve a threshold density. In some embodiments, the cultivation ends at, at about, or within 6 hours, 12 hours, 24 hours, 36 hours, 1 day, 2 days, 3 days, 4 days, 5 days, 6 days, or 7 or more days, after the threshold density is achieved. In particular embodiments, the cultivation is ended at or about 1 day after the threshold density is achieved.

In some embodiments, the cultivation step is performed for the amount of time required for the cells to achieve a threshold amount, density, and/or expansion. In some embodiments, the cultivation is performed for or for about, or for less than, 6 hours, 12 hours, 18 hours, 24 hours, 36 hours, 48 hours, 60 hours, 72 hours, 2 days, 3 days 4 days, 5 days, 6 days, 7 days, 7 days, 8 days, 9 days, 10 days, 1 week, 2 weeks, 3 weeks, or 4 weeks. In particular embodiments, the mean amount of time required for the cells of a plurality of separate compositions of enriched T cells that were isolated, enriched, and/or selected from different biological samples to achieve the threshold density is, is about, or is less than 6 hours, 12 hours, 18 hours, 24 hours, 36 hours, 48 hours, 60 hours, 72 hours, 2 days, 3 days 4 days, 5 days, 6 days, 7 days, 7 days, 8 days, 9 days, 10 days, 1 week, 2 weeks, 3 weeks, or 4 weeks. In certain embodiments, the mean amount of time required for the cells of a plurality of separate compositions of enriched T cells that were isolated, enriched, and/or selected from different biological samples to achieve the threshold density is, is about, or is less than 6 hours, 12 hours, 18 hours, 24 hours, 36 hours, 48 hours, 60 hours, 72 hours, 2 days, 3 days 4 days, 5 days, 6 days, 7 days, 7 days, 8 days, 9 days, 10 days, 1 week, 2 weeks, 3 weeks, or 4 weeks.

In certain embodiments, the cultivation step is performed for a minimum of 4 days, 5 days, 6 days, 7 days, 7 days, 8 days, 9 days, or 10 days, and/or until 12 hours, 24 hours, 36 hours, 1 day, 2 days, or 3 days after the cells active a threshold cell count (or number) or threshold viable cell count (or number) of or of about 1000×10⁶ cells, 1200×10⁶ cells, 1400×10⁶ cells, 1600×10⁶ cells, 1800×10⁶ cells, 2000×10⁶ cells, 2500×10⁶ cells, 3000×10⁶ cells, 4000×10⁶ cells, or 5000×10⁶ cells. In some embodiments, the cultivation step is performed until 1 day after the cells achieve a threshold cell count of or of about 1200×10⁶ cells and are cultured for a minimum of 10 days, and/or until 1 day after the cells achieve a threshold cell count of or of about 5000×10⁶ cells. In some embodiments, the cultivation step is performed until 1 day after the cells achieve a threshold cell count of or of about 1200×10⁶ cells and are cultured for a minimum of 9 days, and/or until 1 day after the cells achieve a threshold cell count of or of about 5000×10⁶ cells. In some embodiments, the cultivation step is performed until 1 day after the cells achieve a threshold cell count of or of about 1000×10⁶ cells and are cultured for a minimum of 8 days, and/or until 1 day after the cells achieve a threshold cell count of or of about 4000×10⁶ cells. In certain embodiments, the cultivation is an expansion step and is performed for a minimum of 4 days, 5 days, 6 days, 7 days, 7 days, 8 days, 9 days, or 10 days, and/or until 12 hours, 24 hours, 36 hours, 1 day, 2 days, or 3 days after the cells active a threshold cell count (or number) or threshold viable cell count (or number) of or of about 1000×10⁶ cells, 1200×10⁶ cells, 1400×10⁶ cells, 1600×10⁶ cells, 1800×10⁶ cells, 2000×10⁶ cells, 2500×10⁶ cells, 3000×10⁶ cells, 4000×10⁶ cells, or 5000×10⁶ cells. In some embodiments, the expansion step is performed until 1 day after the cells achieve a threshold cell count of or of about 1200×10⁶ cells and are expanded for a minimum of 10 days, and/or until 1 day after the cells achieve a threshold cell count of or of about 5000×10⁶ cells. In some embodiments, the expansion step is performed until 1 day after the cells achieve a threshold cell count of or of about 1200×10⁶ cells and are expanded for a minimum of 9 days, and/or until 1 day after the cells achieve a threshold cell count of or of about 5000×10⁶ cells. In some embodiments, the expansion step is performed until 1 day after the cells achieve a threshold cell count of or of about 1000×10⁶ cells and are expanded for a minimum of 8 days, and/or until 1 day after the cells achieve a threshold cell count of or of about 4000×10⁶ cells. In some embodiments, the expansion step is performed until 1 day after the cells achieve a threshold cell count of or of about 1400×10⁶ cells and are expanded for a minimum of 5 days, and/or until 1 day after the cells achieve a threshold cell count of or of about 4000×10⁶ cells.

In some embodiments, the cultivation is performed for at least a minimum amount of time. In some embodiments, the cultivation is performed for at least 14 days, at least 12 days, at least 10 days, at least 7 days, at least 6 days, at least 5 days, at least 4 days, at least 3 days, at least 2 days, at least 36 hours, at least 24 hours, at least 12 hours, or at least 6 hours, even if the threshold is achieved prior to the minimum amount of time. In some embodiments, increasing the minimum amount of time that the cultivation is performed, may, in some cases, reduce the activation and/or reduce the level or one or more activation markers, in the cultivated cells, formulated cells, and/or cells of the output composition. In some embodiments, the minimum cultivation time counts from a determined point an exemplary process (e.g., a selection step; a thaw step; and/or an activation step) to the day the cells are harvested.

In aspects of the provided embodiments, the density and/or concentration of the cells or of the viable cells during the cultivation is monitored or carried out during the cultivation, such as until a threshold amount, density, and/or expansion is achieved as described. In some embodiments such methods include those as described, including optical methods, including digital holography microscopy (DHM) or differential digital holography microscopy (DDHM).

In certain embodiments, the cultivated cells are output cells. In some embodiments, a composition of enriched T cells, such as engineered T cells, that has been cultivated is an output composition of enriched T cells. In particular embodiments, CD4+ T cells and/or CD8+ T cells that have been cultivated are output CD4+ and/or CD8+ T cells. In particular embodiments, a composition of enriched CD4+ T cells, such as engineered CD4+ T cells, that has been cultivated is an output composition of enriched CD4+ T cells. In some embodiments, a composition of enriched CD8+ T cells, such as engineered CD8+ T cells, that has been cultivated is an output composition of enriched CD8+ T cells.

In some embodiments, the cells are cultivated under conditions that promote proliferation and/or expansion in presence of one or more cytokines. In particular embodiments, at least a portion of the cultivation is performed with constant mixing and/or perfusion, such as mixing or perfusion controlled by a bioreactor. In some embodiments, the cells are cultivated in the presence or one or more cytokines and with a surfactant, e.g., poloxamer, such as poloxamer 188, to reduce shearing and/or shear stress from constant mixing and/or perfusion. In some embodiments, a composition of enriched CD4+ T cells, such as engineered CD4+ T cells, is cultivated in the presence of recombinant IL-2, IL-7, IL-15, and poloxamer, wherein at least a portion of the cultivating is performed with constant mixing and/or perfusion. In certain embodiments, a composition of enriched CD8+ T cells, such as engineered CD8+ T cells, is cultivated in the presence of recombinant IL-2, IL-15, and poloxamer, wherein at least a portion of the cultivating is performed with constant mixing and/or perfusion. In some embodiments, the cultivation is performed until the cells reach as threshold expansion of at least 4-fold e.g., as compared to the start of the cultivation.

1. Monitoring Cells During Cultivation

In some embodiments, the cells are monitored during the cultivation step. Monitoring may be performed, for example, to ascertain (e.g., measure, quantify) cell morphology, cell viability, cell death, and/or cell concentration (e.g., viable cell concentration). In some embodiments, the monitoring is performed manually, such as by a human operator. In some embodiments, the monitoring is performed by an automated system. The automated system may require minimal or no manual input to monitor the cultivated cells. In some embodiments, the monitoring is performed both manually and by an automated system.

In certain embodiments, the cells are monitored by an automated system requiring no manual input. In some embodiments, the automated system is compatible with a bioreactor, for example a bioreactor as described herein, such that cells undergoing cultivation can be removed from the bioreactor, monitored, and subsequently returned to the bioreactor. In some embodiments, the monitoring and cultivation occur in a closed loop configuration. In some aspects, in a closed loop configuration, the automated system and bioreactor remain sterile. In embodiments, the automated system is sterile. In some embodiments, the automated system is an in-line system.

In some embodiments, the automated system includes the use of optical techniques (e.g., microscopy) for detecting cell morphology, cell viability, cell death, and/or cell concentration (e.g., viable cell concentration). Any optical technique suitable for determining, for example, cell features, viability, and concentration are contemplated herein. Non-limiting examples of useful optical techniques include bright field microscopy, fluorescence microscopy, differential interference contrast (DIC) microscopy, phase contrast microscopy, digital holography microscopy (DHM), differential digital holography microscopy (DDHM), or a combination thereof. Differential digital holography microscopy, DDHM, and differential DHM may be used herein interchangeably. In certain embodiments, the automated system includes a differential digital holography microscope. In certain embodiments, the automated system includes a differential digital holography microscope including illumination means (e.g., laser, led). Descriptions of DDHM methodology and use may be found, for example, in U.S. Pat. No. 7,362,449; EP 1,631,788; U.S. Pat. Nos. 9,904,248; and 9,684,281, which are incorporated herein by reference in their entirety.

DDHM permits label-free, non-destructive imaging of cells, resulting in high-contrast holographic images. The images may undergo object segmentation and further analysis to obtain a plurality of morphological features that quantitatively describe the imaged objects (e.g., cultivated cells, cellular debris). As such, various features (e.g., cell morphology, cell viability, cell concentration) may be directly assessed or calculated from DDHM using, for example, the steps of image acquisition, image processing, image segmentation, and feature extraction. In some embodiments, the automated system includes a digital recording device to record holographic images. In some embodiments, the automated system includes a computer including algorithms for analyzing holographic images. In some embodiments, the automated system includes a monitor and/or computer for displaying the results of the holographic image analysis. In some embodiments, the analysis is automated (i.e., capable of being performed in the absence of user input). An example of a suitable automated system for monitoring cells during the cultivating step includes, but is not limited to, Ovizio iLine F (Ovizio Imaging Systems NV/SA, Brussels, Belgium).

In certain embodiments, the monitoring is performed continuously during the cultivation step. In some embodiments, the monitoring is performed in real-time during the cultivation step. In some embodiments, the monitoring is performed at discrete time points during the cultivation step. In some embodiments, the monitoring is performed at least every 15 minutes for the duration of the cultivating step. In some embodiments, the monitoring is performed at least every 30 minutes for the duration of the cultivating step. In some embodiments, the monitoring is performed at least every 45 minutes for the duration of the cultivating step. In some embodiments, the monitoring is performed at least every hour for the duration of the cultivating step. In some embodiments, the monitoring is performed at least every 2 hours for the duration of the cultivating step. In some embodiments, the monitoring is performed at least every 4 hours for the duration of the cultivating step. In some embodiments, the monitoring is performed at least every 6 hours for the duration of the cultivating step. In some embodiments, the monitoring is performed at least every 8 hours for the duration of the cultivating step. In some embodiments, the monitoring is performed at least every 10 hours for the duration of the cultivating step. In some embodiments, the monitoring is performed at least every 12 hours for the duration of the cultivating step. In some embodiments, the monitoring is performed at least every 14 hours for the duration of the cultivating step. In some embodiments, the monitoring is performed at least every 16 hours for the duration of the cultivating step. In some embodiments, the monitoring is performed at least every 18 hours for the duration of the cultivating step. In some embodiments, the monitoring is performed at least every 20 hours for the duration of the cultivating step. In some embodiments, the monitoring is performed at least every 22 hours for the duration of the cultivating step. In some embodiments, the monitoring is performed at least once a day for the duration of the cultivating step. In some embodiments, the monitoring is performed at least once every second day for the duration of the cultivating step. In some embodiments, the monitoring is performed at least once every third day for the duration of the cultivating step. In some embodiments, the monitoring is performed at least once every fourth day for the duration of the cultivating step. In some embodiments, the monitoring is performed at least once every fifth day for the duration of the cultivating step. In some embodiments, the monitoring is performed at least once every sixth day for the duration of the cultivating step. In some embodiments, the monitoring is performed at least once every seventh day for the duration of the cultivating step. In some embodiments, the monitoring is performed at least once every eighth day for the duration of the cultivating step. In some embodiments, the monitoring is performed at least once every ninth day for the duration of the cultivating step. In some embodiments, the monitoring is performed at least once every tenth day for the duration of the cultivating step. In some embodiments, the monitoring is performed at least once during the cultivating step.

In some embodiments, features of the cells that can be determined by the monitoring, including using optical techniques such as DHM or DDHM, include cell viability, cell concentration, cell number and/or cell density. In some embodiments, cell viability is characterized or determined. In some embodiments, cell concentration, density and/or number is characterized or determined. In some embodiments, viable cell concentration, viable cell number and/or viable cell density is characterized or determined. In some embodiments, the cultivated cells are monitored by the automated system until a threshold of expansion is reached, such as described above. In some embodiments, once a threshold of expansion is reached, the cultivated cells are harvested, such as by automatic or manual methods, for example, by a human operator. The threshold of expansion may depend on the total concentration, density and/or number of cultured cells determined by the automated system. Alternatively, the threshold of expansion may depend on the viable cell concentration, density and/or number.

In some embodiments, the harvested cells are formulated as described, such as in the presence of a pharmaceutically acceptable carrier. In some embodiments, the harvested cells are formulated in the presence of a cryoprotectant. In some embodiments, the potency of the harvested cells of the therapeutic composition are assessed for potency according to the methods provided in Section I above. In some embodiments, the potency of the harvested cells of the therapeutic composition are assessed for potency prior to cryopreservation. In some embodiments, the potency of the harvested cells of the therapeutic composition are assessed for potency after cryopreservation.

E. Formulating the Cells and Therapeutic Compositions of Recombinant Receptor Engineered Cells

Also provided are compositions containing the therapeutic cell compositions for which potency is assessed according to the methods provided above (Section I), including pharmaceutical compositions and formulations thereof. In some embodiments, the provided methods for manufacturing, generating or producing a cell therapy and/or engineered cells may include formulation of genetically engineered cells resulting from the provided processing steps to produce a therapeutic cell composition containing cells expressing a recombinant receptor. In some embodiments, the provided methods associated with formulation of cells include processing transduced cells, such as cells transduced and/or expanded using the processing steps described above, in a closed system. In some embodiments, the dose of cells comprising cells engineered with a recombinant antigen receptor, e.g., CAR or TCR, is provided as a composition or formulation, such as a pharmaceutical composition or formulation. Such compositions can be used in accord with the provided methods, such as in the prevention or treatment of diseases, conditions, and disorders, or in detection, diagnostic, and prognostic methods.

In some cases, the cells are processed in one or more steps (e.g., carried out in the centrifugal chamber and/or closed system) for manufacturing, generating or producing a cell therapy and/or engineered cells may include formulation of cells, such as formulation of genetically engineered cells resulting from the provided transduction processing steps prior to or after the culturing, e.g., cultivation and expansion, and/or one or more other processing steps as described. In some cases, the cells can be formulated in an amount for dosage administration, such as for a single unit dosage administration or multiple dosage administration. In some embodiments, the potency of the cells of the therapeutic composition, determined according to the methods provided in Section I above, is used to determine a unit dose and/or dosage administration. In some embodiments, the potency of the cells of the therapeutic composition is assessed according to the methods provided in Section I for purposes of determining a unit dose and/or dosage administration. In some embodiments, the provided methods associated with formulation of cells include processing transduced cells, such as cells transduced and/or expanded using the processing steps described above, in a closed system.

In certain embodiments, one or more compositions of enriched T cells, such as engineered and cultivated T cells, e.g., output T cells, therapeutic cell composition, are formulated. In particular embodiments, one or more compositions of enriched T cells, such as engineered and cultivated T cells, e.g., output T cells, therapeutic cell composition, are formulated after the one or more compositions have been engineered and/or cultivated. In particular embodiments, the one or more compositions are input compositions. In some embodiments, the one or more input compositions have been previously cryofrozen and stored, and are thawed prior to the incubation.

In certain embodiments, the one or more therapeutic compositions of enriched T cells, such as engineered and cultivated T cells, e.g., output T cells, are or include two separate compositions, e.g., separate engineered and/or cultivated compositions, of enriched T cells. In particular embodiments, two separate therapeutic compositions of enriched T cells, e.g., two separate compositions of enriched CD4+ T cells and CD8+ T cells selected, isolated, and/or enriched from the same biological sample, separately engineered and separately cultivated, are separately formulated. In certain embodiments, the two separate therapeutic cell compositions include a composition of enriched CD4+ T cells, such as a composition of engineered and/or cultivated CD4+ T cells. In particular embodiments, the two separate therapeutic cell compositions include a composition of enriched CD8+ T cells, such as a composition of engineered and/or cultivated CD8+ T cells. In some embodiments, two separate therapeutic compositions of enriched CD4+ T cells and enriched CD8+ T cells, such as separate compositions of engineered and cultivated CD4+ T cells and engineered and cultivated CD8+ T cells, are separately formulated. In some embodiments, a single therapeutic composition of enriched T cells is formulated. In certain embodiments, the single therapeutic composition is a composition of enriched CD4+ T cells, such as a composition of engineered and/or cultivated CD4+ T cells. In some embodiments, the single therapeutic composition is a composition of enriched CD4+ and CD8+ T cells that have been combined from separate compositions prior to the formulation.

In some embodiments, separate therapeutic compositions of enriched CD4+ and CD8+ T cells, such as separate compositions of engineered and cultivated CD4+ and CD8+ T cells, are combined into a single therapeutic composition and are formulated. In certain embodiments, separate formulated therapeutic compositions of enriched CD4+ and enriched CD8+ T cells are combined into a single therapeutic composition after the formulation has been performed and/or completed. In particular embodiments, separate therapeutic compositions of enriched CD4+ and CD8+ T cells, such as separate compositions of engineered and cultivated CD4+ and CD8+ T cells, are separately formulated as separate compositions.

In some embodiments, the therapeutic composition of enriched CD4+ T cells, such as an engineered and cultivated CD4+ T cells, e.g., output CD4+ T cells, that is formulated, includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In some embodiments, the composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD4+ T cells that express a recombinant receptor and/or have been transduced or transfected with the recombinant polynucleotide. In certain embodiments, the therapeutic composition of enriched CD4+ T cells, such as an engineered and cultivated CD4+ T cells, e.g., output CD4+ T cells, that is formulated includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the therapeutic composition of enriched CD8+ T cells, such as an engineered and cultivated CD8+ T cells, e.g., output CD8+ T cells, that is formulated, includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the therapeutic composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD8+ T cells that express the recombinant receptor and/or have been transduced or transfected with the recombinant polynucleotide. In certain embodiments, the therapeutic composition of enriched CD8+ T cells, such as an engineered and cultivated CD8+ T cells, e.g., output CD8+ T cells, that is incubated under stimulating conditions includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In certain embodiments, the formulated cells are output cells. In some embodiments, a formulated therapeutic composition of enriched T cells, such as a formulated composition of engineered and cultivated T cells, is an output composition of enriched T cells. In particular embodiments, the formulated CD4+ T cells and/or formulated CD8+ T cells are the output CD4+ and/or CD8+ T cells. In particular embodiments, a formulated composition of enriched CD4+ T cells is an output composition of enriched CD4+ T cells. In some embodiments, a formulated composition of enriched CD8+ T cells is an output composition of enriched CD8+ T cells.

In some embodiments, cells can be formulated into a container, such as a bag or vial. In some embodiments, the cells are formulated between 0 days and 10 days, between 0 and 5 days, between 2 days and 7 days, between 0.5 days and 4 days, or between 1 day and 3 days after the cells after the threshold cell count, density, and/or expansion has been achieved during the cultivation. In certain embodiments, the cells are formulated at or at or about or within 12 hours, 18 hours, 24 hours, 1 day, 2 days, or 3 days after the threshold cell count, density, and/or expansion has been achieved during the cultivation. In some embodiments, the cells are formulated within or within about 1 day after the threshold cell count, density, and/or expansion has been achieved during the cultivation.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the transduced and/or expanded cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains the cells in amounts effective to treat or prevent the disease or condition, such as a therapeutically effective or prophylactically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The pharmaceutical composition in some embodiments contains cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells may be administered using standard administration techniques, formulations, and/or devices. Provided are formulations and devices, such as syringes and vials, for storage and administration of the compositions. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes

In some embodiments, the formulation buffer contains a cryopreservative. In some embodiments, the cell are formulated with a cryopreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the transduced and/or expanded cells to replace the cells in a cryopreservative solution. In some embodiments, the cells are frozen, e.g., cryofrozen or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryofrozen or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

In particular embodiments, the therapeutic composition of enriched T cells, e.g., T cells that have been stimulated, engineered, and/or cultivated, are formulated, cryofrozen, and then stored for an amount of time. In certain embodiments, the formulated, cryofrozen cells are stored, typically in multiple vials or containers, until the cells are released for infusion. In some embodiments, a vial or container of a particular therapeutic composition may be used to carry out the provided potency assay prior to infusion of the therapeutic cell composition. In particular embodiments, the formulated cryofrozen cells are stored for between 1 day and 6 months, between 1 month and 3 months, between 1 day and 14 days, between 1 day and 7 days, between 3 days and 6 days, between 6 months and 12 months, or longer than 12 months. In some embodiments, the cells are cryofrozen and stored for, for about, or for less than 1 day, 2 days, 3 days, 4 days, 5 days, 6 days, or 7 days. In certain embodiments, the cells are thawed and administered to a subject after the storage. In certain embodiments, the cells are stored for or for about 5 days.

In some embodiments, the cells are formulated in a pharmaceutically acceptable buffer, optionally including a cryoprotectant, in a volume that is from 10 mL to 1000 mL or from about 10 mL to about 1000 mL, such as at least or about at least or about 50 mL, 100 mL, 200 mL, 300 mL, 400 mL, 500 mL, 600 mL, 700 mL, 800 mL, 900 mL or 1000 mL. In some embodiments, the therapeutic cell composition is stored in a container, such as one or more vials or bags. In some embodimetns, the vials or bags generally contain the cells to be administered, e.g., one or more unit doses thereof. The unit dose may be an amount or number of the cells to be administered to the subject or twice the number (or more) of the cells to be administered. It may be the lowest dose or lowest possible dose of the cells that would be administered to the subject.

In some embodiments, each of the containers, e.g., bags of vials individually comprises a unit dose of the cells. Thus in some embodiments, each of the containers comprises the same or approximately or substantially the same number of cells. In some embodiments, each unit dose contains at least or about at least 1×10⁶, 2×10⁶, 5×10⁶, 1×10₇, 5×10⁷, or 1×10⁸ engineered cells, total cells, T cells, or PBMCs. In some embodiments, the volume of the formulated cell composition in each container, e.g., bag or vial, is 10 mL to 100 mL, such as at least or about at least or about 20 mL, 30 mL, 40 mL, 50 mL, 60 mL, 70 mL, 80 mL, 90 mL or 100 mL. In some embodiments, the cells in the container, e.g., bag or vials, can be cryopreserved. In some embodiments, the container, e.g., vials, can be stored in liquid nitrogen until further use.

In some embodiments, the potency, such as relative potency, of cells of a composition containing recombinant receptor-expressing cells (e.g., CAR-expressing cells), such as produced by a method including on or more of the described steps, is determined or measured using the potency assay described herein. In some embodiments, cells of the composition containing recombinant receptor-expressing cells (e.g., CAR-expressing cells), such as produced by a method including on or more of the described steps and/or for which potency has been determined may be administered to a subject for treating a disease or condition.

### III. RECOMBINANT RECEPTORS

In some embodiments, the provided methods for determining potency, such as relative potency, of a therapeutic cell composition, are performed or carried out on cells from a composition that contain or express, or are engineered to contain or express, a recombinant recombinant receptor, e.g., a chimeric antigen receptor (CAR), or a T cell receptor (TCR). In certain embodiments, the methods provided herein produce and/or a capable of producing cells, or populations or compositions containing and/or enriched for cells, that are engineered to express or contain a recombinant protein, and for which potency of such produced engineered cells can be determined or measured. In various embodiments, the provided methods are carried out on cells compositions, such as immune cells, for example T cells, that are engineered, transformed, transduced, or transfected cells, to express one or more recombinant receptor(s).

In some embodiments, the provided methods are for assessing potency of engineered cells, such as immune cells, such as T cells, that express one or more recombinant receptor(s). Among the receptors are antigen receptors and receptors containing one or more component thereof. The recombinant receptors may include chimeric receptors, such as those containing ligand-binding domains or binding fragments thereof and intracellular signaling domains or regions, functional non-TCR antigen receptors, chimeric antigen receptors (CARs), T cell receptors (TCRs), such as recombinant or transgenic TCRs, chimeric autoantibody receptor (CAAR) and components of any of the foregoing. The recombinant receptor, such as a CAR, generally includes the extracellular antigen (or ligand) binding domain linked to one or more intracellular signaling components, in some aspects via linkers and/or transmembrane domain(s). In some embodiments, the engineered cells express two or more receptors that contain different components, domains or regions. In some aspects, two or more receptors allows spatial or temporal regulation or control of specificity, activity, antigen (or ligand) binding, function and/or expression of the recombinant receptors.

A. Chimeric Antigen Receptors (CARs)

In some embodiments of the provided methods, chimeric receptors, such as a chimeric antigen receptors, contain one or more domains that combine a ligand-binding domain (e.g., antibody or antibody fragment) that provides specificity for a desired target (e.g., antigen (e.g., tumor antigen)) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, WO2015/168613, WO2016/030414, U.S. patent application publication numbers US2002131960, US2013287748, US20130149337, US20190389925, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, 8,479,118, 10,266,580 and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

The chimeric receptors, such as CARs, generally include an extracellular target binding domain (e.g., an antigen binding domain), such as, e.g., a portion of an antibody molecule, generally a variable heavy (VH) chain region and/or variable light (VL) chain region of the antibody, e.g., an scFv antibody fragment.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the antigen is or includes avβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen that comprises or is associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30. In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

In some embodiments, the antibody is an antigen-binding fragment, such as a scFv, that includes one or more linkers joining two antibody domains or regions, such as a heavy chain variable (VH) region and a light chain variable (VL) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline. In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 22) or GGGS (3GS; SEQ ID NO: 23), such as between 2, 3, 4, and 5 repeats of such a sequence. Exemplary linkers include those having or consisting of a sequence set forth in SEQ ID NO: 24 (GGGGSGGGGSGGGGS), SEQ ID NO: 25 (GSTSGSGKPGSGEGSTKG) or SEQ ID NO: 26 (SRGGGGSGGGGSGGGGSLEMA).

Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30.

In some embodiments, the antigen or antigen binding domain is CD19. In some embodiments, the scFv contains a VH and a VL derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, heavy chain variable (VH) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 1 lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the antibodies included in the provided CARs are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region; and multispecific antibodies formed from antibody fragments. In some embodiments, the antigen-binding domain in the provided CARs is or comprises an antibody fragment comprising a variable heavy chain (V_{H}) and a variable light chain (V_{L}) region. In particular embodiments, the antibodies are single-chain antibody fragments comprising a heavy chain variable (V_{H}) region and/or a light chain variable (V_{L}) region, such as scFvs.

In some embodiments, the scFv is derived from FMC63. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the FMC63 antibody comprises CDRH1 and H2 set forth in SEQ ID NOS: 27 and 28, respectively, and CDRH3 set forth in SEQ ID NO: 29 or 30 and CDRL1 set forth in SEQ ID NO: 55 and CDR L2 set forth in SEQ ID NO: 32 or 33 and CDR L3 set forth in SEQ ID NO: 34 or 35. In some embodiments, the FMC63 antibody comprises the heavy chain variable region (V_{H}) comprising the amino acid sequence of SEQ ID NO: 36 and the light chain variable region (V_{L}) comprising the amino acid sequence of SEQ ID NO: 37.

In some embodiments, the scFv comprises a variable light chain containing the CDRL1 sequence of SEQ ID NO: 31, a CDRL2 sequence of SEQ ID NO: 32, and a CDRL3 sequence of SEQ ID NO: 34 and/or a variable heavy chain containing a CDRH1 sequence of SEQ ID NO: 27, a CDRH2 sequence of SEQ ID NO: 28, and a CDRH3 sequence of SEQ ID NO: 29. In some embodiments, the scFv comprises a variable heavy chain region set forth in SEQ ID NO: 36 and a variable light chain region set forth in SEQ ID NO: 37. In some embodiments, the variable heavy and variable light chains are connected by a linker. In some embodiments, the linker is set forth in SEQ ID NO: 25. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO: 38 or a sequence that exhibits at least 85%, 86%, 87%, 88%, 89%, 90%, 91%, 92%, 93%, 94%, 95%, 96%, 97%, 98%, or 99% sequence identity to SEQ ID NO: 38. In some embodiments, the scFv comprises the sequence of amino acids set forth in SEQ ID NO: 39 or a sequence that exhibits at least 85%, 86%, 87%, 88%, 89%, 90%, 91%, 92%, 93%, 94%, 95%, 96%, 97%, 98%, or 99% sequence identity to SEQ ID NO: 39.

In some embodiments the scFv is derived from SJ25C1. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises CDRH1, H2 and H3 set forth in SEQ ID NOS: 51-53, respectively, and CDRL1, L2 and L3 sequences set forth in SEQ ID NOS: 48-50, respectively. In some embodiments, the SJ25C1 antibody comprises the heavy chain variable region (V_{H}) comprising the amino acid sequence of SEQ ID NO: 46 and the light chain variable region (V_{L}) comprising the amino acid sequence of SEQ ID NO: 47.

In some embodiments, the scFv comprises a variable light chain containing the CDRL1 sequence of SEQ ID NO: 48, a CDRL2 sequence of SEQ ID NO: 49, and a CDRL3 sequence of SEQ ID NO: 50 and/or a variable heavy chain containing a CDRH1 sequence of SEQ ID NO: 51, a CDRH2 sequence of SEQ ID NO: 52, and a CDRH3 sequence of SEQ ID NO: 53. In some embodiments, the scFv comprises a variable heavy chain region set forth in SEQ ID NO: 46 and a variable light chain region set forth in SEQ ID NO: 47. In some embodiments, the variable heavy and variable light chain are connected by a linker. In some embodiments, the linker is set forth in SEQ ID NO: 24. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO: 54 or a sequence that exhibits at least 85%, 86%, 87%, 88%, 89%, 90%, 91%, 92%, 93%, 94%, 95%, 96%, 97%, 98%, or 99% sequence identity to SEQ ID NO: 54.

In some embodiments, the antibody or an antigen-binding fragment (e.g., scFv or V_{H} domain) specifically recognizes an antigen, such as BCMA. In some embodiments, the antibody or antigen-binding fragment is derived from, or is a variant of, antibodies or antigen-binding fragment that specifically binds to BCMA.

In some embodiments, the CAR is an anti-BCMA CAR that is specific for BCMA, e.g., human BCMA. Chimeric antigen receptors containing anti-BCMA antibodies, including mouse anti-human BCMA antibodies and human anti-human antibodies, and cells expressing such chimeric receptors have been previously described. See Carpenter et al., Clin Cancer Res., 2013, 19(8):2048-2060, WO 2016/090320, WO2016090327, WO2010104949A2 and WO2017173256. In some embodiments, the antigen or antigen binding domain is BCMA. In some embodiments, the scFv contains a VH and a VL derived from an antibody or an antibody fragment specific to BCMA. In some embodiments, the antibody or antibody fragment that binds BCMA is or contains a VH and a VL from an antibody or antibody fragment set forth in International Patent Applications, Publication Number WO 2016/090327 and WO 2016/090320.

In some embodiments, the anti-BCMA CAR contains an antigen-binding domain, such as an scFv, containing a variable heavy (V_{H}) and/or a variable light (V_{L}) region derived from an antibody described in WO 2016/090320 or WO2016090327. In some embodiments, the antigen-binding domain, such as an scFv, contains a V_{H} set forth in SEQ ID NO: 55 and a V_{L} set forth in SEQ ID NO: 56. In some embodiments, the antigen-binding domain, such as an scFv, contains a V_{H} set forth in SEQ ID NO: 57 and a V_{L} set forth in SEQ ID NO: 58. In some embodiments, the antigen-binding domain, such as an scFv, contains a V_{H} set forth in SEQ ID NO: 59 and a V_{L} set forth in SEQ ID NO: 60. In some embodiments, the antigen-binding domain, such as an scFv, contains a V_{H} set forth in SEQ ID NO: 61 and a V_{L} set forth in SEQ ID NO: 62. In some embodiment the antigen-binding domain, such as an scFv, contains a V_{H} set forth in SEQ ID NO: 63 and a V_{L} set forth in SEQ ID NO: 64. In some embodiments, the antigen-binding domain, such as an scFv, contains a V_{H} set forth in SEQ ID NO: 65 and a V_{L} set forth in SEQ ID NO: 66. In some embodiments, the antigen-binding domain, such as an scFv, contains a V_{H} set forth in SEQ ID NO: 67 and a V_{L} set forth in SEQ ID NO: 68. In some embodiments, the V_{H} or V_{L} has a sequence of amino acids that exhibits at least 85%, 86%, 87%, 88%, 89%, 90%, 91%, 92%, 93%, 94%, 95%, 96%, 97%, 98%, 99% or more sequence identity to any of the foregoing V_{H} or V_{L} sequences, and retains binding to BCMA. In some embodiments, the V_{H} region is amino-terminal to the V_{L} region. In some embodiments, the V_{H} region is carboxy-terminal to the V_{L} region.

In some embodiments, the antigen or antigen binding domain is GPRC5D. In some embodiments, the scFv contains a V_{H} and a V_{L} derived from an antibody or an antibody fragment specific to GPRC5D. In some embodiments, the antibody or antibody fragment that binds GPRC5D is or contains a VH and a VL from an antibody or antibody fragment set forth in International Patent Applications, Publication Number WO 2016/090329 and WO 2016/090312.

In some aspects, the CAR contains a ligand- (e.g., antigen-) binding domain that binds or recognizes, e.g., specifically binds, a universal tag or a universal epitope. In some aspects, the binding domain can bind a molecule, a tag, a polypeptide and/or an epitope that can be linked to a different binding molecule (e.g., antibody or antigen-binding fragment) that recognizes an antigen associated with a disease or disorder. Exemplary tag or epitope includes a dye (e.g., fluorescein isothiocyanate) or a biotin. In some aspects, a binding molecule (e.g., antibody or antigen-binding fragment) linked to a tag, that recognizes the antigen associated with a disease or disorder, e.g., tumor antigen, with an engineered cell expressing a CAR specific for the tag, to effect cytotoxicity or other effector function of the engineered cell. In some aspects, the specificity of the CAR to the antigen associated with a disease or disorder is provided by the tagged binding molecule (e.g., antibody), and different tagged binding molecule can be used to target different antigens. Exemplary CARs specific for a universal tag or a universal epitope include those described, e.g., in U.S. Pat. No. 9,233,125, WO 2016/030414, Urbanska et al., (2012) Cancer Res 72: 1844-1852, and Tamada et al., (2012). Clin Cancer Res 18:6436-6445.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens. In some embodiments, the CAR contains a TCR-like antibody, such as an antibody or an antigen-binding fragment (e.g., scFv) that specifically recognizes an intracellular antigen, such as a tumor-associated antigen, presented on the cell surface as a major histocompatibility complex (MHC)-peptide complex. In some embodiments, an antibody or antigen-binding portion thereof that recognizes an MHC-peptide complex can be expressed on cells as part of a recombinant receptor, such as an antigen receptor. Among the antigen receptors are functional non-T cell receptor (TCR) antigen receptors, such as chimeric antigen receptors (CARs). In some embodiments, a CAR containing an antibody or antigen-binding fragment that exhibits TCR-like specificity directed against peptide-MHC complexes also may be referred to as a TCR-like CAR. In some embodiments, the CAR is a TCR-like CAR and the antigen is a processed peptide antigen, such as a peptide antigen of an intracellular protein, which, like a TCR, is recognized on the cell surface in the context of an MHC molecule. In some embodiments, the extracellular antigen-binding domain specific for an MHC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

Reference to “Major histocompatibility complex” (MHC) refers to a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e., MHC-peptide complex, for presentation of an antigen in a conformation recognizable by an antigen receptor on T cells, such as a TCRs or TCR-like antibody. Generally, MHC class I molecules are heterodimers having a membrane spanning a chain, in some cases with three a domains, and a non-covalently associated β2 microglobulin. Generally, MHC class II molecules are composed of two transmembrane glycoproteins, α and β, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8⁺ T cells, but in some cases CD4⁺ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, a TCR-like antibody or antigen-binding portion, are known or can be produced by known methods (see e.g., US Published Application Nos. US 2002/0150914; US 2003/0223994; US 2004/0191260; US 2006/0034850; US 2007/00992530; US20090226474; US20090304679; and International App. Pub. No. WO 03/068201).

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen or other antigen as described below. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See e.g., US Pat. App. Pub. No. US20020150914, US20140294841; and Cohen C J. et al. (2003) J Mol. Recogn. 16:324-332.

In some embodiments, the antigen is CD20. In some embodiments, the scFv contains a VH and a VL derived from an antibody or an antibody fragment specific to CD20. In some embodiments, the antibody or antibody fragment that binds CD20 is an antibody that is or is derived from Rituximab, such as is Rituximab scFv.

In some embodiments, the antigen is CD22. In some embodiments, the scFv contains a VH and a VL derived from an antibody or an antibody fragment specific to CD22. In some embodiments, the antibody or antibody fragment that binds CD22 is an antibody that is or is derived from m971, such as is m971 scFv.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv.

In some embodiments, the antibody portion of the recombinant receptor, e.g., CAR, further includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers include, but are not limited to, those described in Hudecek et al. (2013) Clin. Cancer Res., 19:3153, international patent application publication number WO2014031687, U.S. Pat. No. 8,822,647 or published app. No. US2014/0271635.

In some embodiments, the constant region or portion is of a human IgG, such as IgG4 or IgG1. In some embodiments, the spacer has the sequence ESKYGPPCPPCP (set forth in SEQ ID NO: 69), and is encoded by the sequence set forth in SEQ ID NO: 70. In some embodiments, the spacer has the sequence set forth in SEQ ID NO: 71. In some embodiments, the spacer has the sequence set forth in SEQ ID NO: 72. In some embodiments, the constant region or portion is of IgD. In some embodiments, the spacer is a portion of an immunoglobulin constant region that is or comprises the hinge sequence. In some embodiments, the spacer has the sequence set forth in SEQ ID NO: 73. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 69, 70, 71, 72, or 73. In some embodiments, the spacer has the sequence set forth in SEQ ID NOS: 74-82. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 74-82.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g., extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g., scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28.

In some embodiments, the extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD3 transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR (e.g., iCAR) and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAGS, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

An exemplary polypeptide for a truncated EGFR (e.g., tEGFR) comprises the sequence of amino acids set forth in SEQ ID NO: 2 or 3 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 2 or 3. An exemplary T2A linker sequence comprises the sequence of amino acids set forth in SEQ ID NO: 1 or 4 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 1 or 4.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

For example, in some embodiments, the CAR contains an antibody, e.g., an antibody fragment, such as an scFv, specific to an antigen including any as described, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, such as an scFv, specific to an antigen including any as described, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g., an IgG4 hinge, such as a hinge-only spacer.

In some embodiments, the transmembrane domain of the recombinant receptor, e.g., the CAR, is or includes a transmembrane domain of human CD28 (e.g., Accession No. P01747.1) or variant thereof, such as a transmembrane domain that comprises the sequence of amino acids set forth in SEQ ID NO: 83 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 83; in some embodiments, the transmembrane-domain containing portion of the recombinant receptor comprises the sequence of amino acids set forth in SEQ ID NO: 84 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the intracellular signaling component(s) of the recombinant receptor, e.g., the CAR, contains an intracellular costimulatory signaling domain of human CD28 or a functional variant or portion thereof, such as a domain with an LL to GG substitution at positions 186-187 of a native CD28 protein. For example, the intracellular signaling domain can comprise the sequence of amino acids set forth in SEQ ID NO: 85 or 86 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 85 or 86. In some embodiments, the intracellular domain comprises an intracellular costimulatory signaling domain of 4-1BB (e.g., (Accession No. Q07011.1) or functional variant or portion thereof, such as the sequence of amino acids set forth in SEQ ID NO: 87 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 87.

In some embodiments, the intracellular signaling domain of the recombinant receptor, e.g., the CAR, comprises a human CD3 zeta stimulatory signaling domain or functional variant thereof, such as an 112 AA cytoplasmic domain of isoform 3 of human CD3ζ (Accession No.: P20963.2) or a CD3 zeta signaling domain as described in U.S. Pat. No. 7,446,190 or U.S. Pat. No. 8,911,993. For example, in some embodiments, the intracellular signaling domain comprises the sequence of amino acids as set forth in SEQ ID NO: 88, 89 or 90 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 88, 89 or 90.

In some aspects, the spacer contains only a hinge region of an IgG, such as only a hinge of IgG4 or IgG1, such as the hinge only spacer set forth in SEQ ID NO: 69. In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains, such as set forth in SEQ ID NO: 72. In some embodiments, the spacer is an Ig hinge, e.g., an IgG4 hinge, linked to a CH3 domain only, such as set forth in SEQ ID NO: 71. In some embodiments, the spacer is or comprises a glycine-serine rich sequence or other flexible linker such as known flexible linkers.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in 2 or 3) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered to express the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g., surrogate marker, includes all or part (e.g., truncated form) of CD34, a NGFR, a CD19 or a truncated CD19, e.g., a truncated non-human CD19, or epidermal growth factor receptor (e.g., tEGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a resistance marker or selection marker. In some embodiments, the resistance marker or selection marker is or comprises a polypeptide that confers resistance to exogenous agents or drugs. In some embodiments, the resistance marker or selection marker is an antibiotic resistance gene. In some embodiments, the resistance marker or selection marker is an antibiotic resistance gene confers antibiotic resistance to a mammalian cell. In some embodiments, the resistance marker or selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No. WO2014031687.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 1 or 4, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 1 or 4.

In some embodiments, T cells expressing an antigen receptor (e.g., CAR) can also be generated to express a truncated EGFR (tEGFR) as a non-immunogenic selection epitope (e.g., by introduction of a construct encoding the CAR and tEGFR separated by a T2A ribosome switch to express two proteins from the same construct), which then can be used as a marker to detect such cells (see e.g., U.S. Pat. No. 8,802,374). In some embodiments, the sequence encodes an tEGFR sequence set forth in SEQ ID NO: 2 or 3, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 2 or 3. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and nucleic acids disclosed herein, without limitation, 2A sequences from the foot-and-mouth disease virus (F2A, e.g., SEQ ID NO: 8), equine rhinitis A virus (E2A, e.g., SEQ ID NO: 7), Thosea asigna virus (T2A, e.g., SEQ ID NO: 1 or 4), and porcine teschovirus-1 (P2A, e.g., SEQ ID NO: 5 or 6) as described in U.S. Patent Publication No. 20070116690.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

B. Chimeric Auto-Antibody Receptor (CAAR)

In some embodiments, the recombinant receptor is a chimeric autoantibody receptor (CAAR). In some embodiments, the CAAR binds, e.g., specifically binds, or recognizes, an autoantibody. In some embodiments, a cell expressing the CAAR, such as a T cell engineered to express a CAAR, can be used to bind to and kill autoantibody-expressing cells, but not normal antibody expressing cells. In some embodiments, CAAR-expressing cells can be used to treat an autoimmune disease associated with expression of self-antigens, such as autoimmune diseases. In some embodiments, CAAR-expressing cells can target B cells that ultimately produce the autoantibodies and display the autoantibodies on their cell surfaces, mark these B cells as disease-specific targets for therapeutic intervention. In some embodiments, CAAR-expressing cells can be used to efficiently targeting and killing the pathogenic B cells in autoimmune diseases by targeting the disease-causing B cells using an antigen-specific chimeric autoantibody receptor. In some embodiments, the recombinant receptor is a CAAR, such as any described in U.S. Patent Application Pub. No. US 2017/0051035.

In some embodiments, the CAAR comprises an autoantibody binding domain, a transmembrane domain, and one or more intracellular signaling region or domain (also interchangeably called a cytoplasmic signaling domain or region). In some embodiments, the intracellular signaling region comprises an intracellular signaling domain. In some embodiments, the intracellular signaling domain is or comprises a primary signaling domain, a signaling domain that is capable of stimulating and/or inducing a primary activation signal in a T cell, a signaling domain of a T cell receptor (TCR) component (e.g., an intracellular signaling domain or region of a CD3-zeta (CD3ζ) chain or a functional variant or signaling portion thereof), and/or a signaling domain comprising an immunoreceptor tyrosine-based activation motif (ITAM).

In some embodiments, the autoantibody binding domain comprises an autoantigen or a fragment thereof. The choice of autoantigen can depend upon the type of autoantibody being targeted. For example, the autoantigen may be chosen because it recognizes an autoantibody on a target cell, such as a B cell, associated with a particular disease state, e.g., an autoimmune disease, such as an autoantibody-mediated autoimmune disease. In some embodiments, the autoimmune disease includes pemphigus vulgaris (PV). Exemplary autoantigens include desmoglein 1 (Dsg1) and Dsg3.

C. T Cell Receptors (TCRs)

In some embodiments, engineered cells, such as T cells, are provided that express a T cell receptor (TCR) or antigen-binding portion thereof that recognizes an peptide epitope or T cell epitope of a target polypeptide, such as an antigen of a tumor, viral or autoimmune protein.

In some embodiments, a “T cell receptor” or “TCR” is a molecule that contains a variable α and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or Cβ, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g., CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g., cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ T cells. In some embodiments, the TCRs can be amplified from a T cell source of a normal of healthy subject, i.e. normal TCR libraries. In some embodiments, the TCRs can be amplified from a T cell source of a diseased subject, i.e., diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g., present on the antigen-specific T cells, may be selected, such as by binding activity, e.g., particular affinity or avidity for the antigen.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007).

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known, See e.g., Soo Hoo, W. F. et al. PNAS (USA) 89, 4759 (1992); Wülfing, C. and Plückthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see e.g., International published PCT No. WO 03/020763). In some embodiments, a scTCR is a non-disulfide linked truncated TCR in which heterologous leucine zippers fused to the C-termini thereof facilitate chain association (see e.g., International published PCT No. WO99/60120). In some embodiments, a scTCR contain a TCRα variable domain covalently linked to a TCRβ variable domain via a peptide linker (see e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence α chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from 10 to 45 amino acids or from about 10 to about 45 amino acids, such as 10 to 30 amino acids or 26 to 41 amino acids residues, for example 29, 30, 31 or 32 amino acids. In some embodiments, the linker has the formula -PGGG-(SGGGG)5-P- wherein P is proline, G is glycine and S is serine (SEQ ID NO: 91). In some embodiments, the linker has the sequence GSADDAKKDAAKKDGKS (SEQ ID NO: 92).

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10-5 and 10-12 M and all individual values and ranges therein. In some embodiments, the target antigen is an MHC-peptide complex or ligand.

In some embodiments, nucleic acid or nucleic acids encoding a TCR, such as α and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λ610, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g., lentiviral, vector.

### IV. Methods of Administration

Also provided are methods of using and uses of the compositions, such as in the treatment of diseases, conditions, and disorders, for example cancers.

Provided are methods of administering the therapeutic cell compositions for which potency has been assessed according to the methods provide herein (e.g., Section I), and uses of such cells, populations, and compositions to treat or prevent diseases, conditions, and disorders, including cancers. Also provided are methods of using and uses of the therapeutic cell compositions for which potency has been assessed according to the methods provide herein (e.g., Section I), and uses of such therapeutic cell compositions to treat or prevent diseases, conditions, and disorders, including cancers. In particular embodiments, the cells, populations and compositions are those as produced and engineered in accord with any of the provided methods. In some embodiments, the cells, populations, and compositions are administered to a subject or patient having the particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in a cancer expressing an antigen recognized by an engineered T cell.

Such methods and uses include therapeutic methods and uses, for example, involving administration of cells and compositions prepared by the provided methods, such as engineered compositions and end-of-production compositions following incubation and/or other processing steps, to a subject having a disease, condition or disorder, such as a cancer, to effect treatment of the disease or disorder. In some embodiments, the potency of the composition is determined according to the methods provided herein. Uses include uses of the compositions in such methods and treatments, and uses of such compositions in the preparation of a medicament in order to carry out such therapeutic methods. In some embodiments, the methods and uses thereby treat the disease or condition or disorder, such as a tumor or cancer, in the subject.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

As used herein, a “subject” is a mammal, such as a human or other animal, and typically is human. In some embodiments, the subject, e.g., patient, to whom the cells, cell populations, or compositions are administered is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts. In some embodiments, the determined potency of the therapeutic cell composition is used to determine an effect amount, e.g., therapeutically effective amount.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In some embodiments, the determined potency of the therapeutic cell composition is used to determine a prophylactically effective amount.

The disease or condition that is treated can be any in which expression of an antigen is associated with and/or involved in the etiology of a disease condition or disorder, e.g., causes, exacerbates or otherwise is involved in such disease, condition, or disorder. Exemplary diseases and conditions can include diseases or conditions associated with malignancy or transformation of cells (e.g., cancer), autoimmune or inflammatory disease, or an infectious disease, e.g., caused by a bacterial, viral or other pathogen. Exemplary antigens, which include antigens associated with various diseases and conditions that can be treated, are described above. In particular embodiments, the chimeric antigen receptor or transgenic TCR specifically binds to an antigen associated with the disease or condition.

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition, ameliorate one or more symptom of the disease or condition.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject. The cells can be administered by any suitable means. Dosing and administration may depend in part on whether the administration is brief or chronic. Various dosing schedules include but are not limited to single or multiple administrations over various time-points, bolus administration, and pulse infusion.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Again, dosages may vary depending on attributes particular to the disease or disorder and/or patient and/or other treatments. In some embodiments, dosage may depend on the potency of the therapeutic cell composition. In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents include a cytokine, such as IL-2, for example, to enhance persistence. In some embodiments, the methods comprise administration of a chemotherapeutic agent.

Following administration of the cells, the biological activity of the engineered cell populations (e.g., therapeutic cell compositions) in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3: 1 1 1 (1995), and U.S. Pat. No. 5,087,616. In some embodiments, confirmation of increased therapeutic or prophylactic efficacy is determined using the methods of assessing potency described herein (e.g., Section I).

### IV. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compounds, including cells. It may be a solution, a suspension, liquid, powder, a paste, aqueous, non-aqueous or any combination thereof.

As used herein, a “subject” is a mammal, such as a human or other animal, and typically is human.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

### V. EXEMPLARY EMBODIMENTS

Among the provided embodiments are:

1. A method of determining potency of a therapeutic cell composition, the method comprising:
performing a plurality of incubations, each of said plurality of incubations comprising culturing cells of a therapeutic cell composition, said therapeutic composition comprising cells engineered to express a recombinant receptor, with a recombinant receptor stimulating agent, wherein:
binding of the recombinant receptor stimulating agent to the recombinant receptor stimulates a recombinant receptor-dependent activity in the cell; and
each of the plurality of incubations comprises a different titrated ratio of the cells of the therapeutic cell composition to the recombinant receptor stimulating agent;
measuring the recombinant receptor-dependent activity from each of the plurality of incubations;
determining, based on the recombinant receptor-dependent activity measured from each of the plurality of incubations, the titrated ratio that results in a half-maximal recombinant receptor-dependent activity.

2. The method of embodiment 1, further comprising determining a relative potency of the therapeutic cell composition by comparing the titrated ratio resulting in the half-maximal recombinant receptor-dependent activity of the therapeutic cell composition to a titrated ratio resulting in a half-maximal recombinant receptor-dependent activity of a reference standard.

3. A method of determining potency of a therapeutic cell composition, the method comprising:
performing a plurality of incubations, each of said plurality of incubations comprising culturing cells of a therapeutic cell composition, said therapeutic composition comprising cells engineered to express a recombinant receptor, with a recombinant receptor stimulating agent, wherein:
   binding of the recombinant receptor stimulating agent to the recombinant receptor stimulates a recombinant receptor-dependent activity in the cell; and
   each of the plurality of incubations comprises a different titrated ratio of the cells of the therapeutic cell composition to the recombinant receptor stimulating agent;
measuring the recombinant receptor-dependent activity from each of the plurality of incubations; and
determining a relative potency of the therapeutic cell composition by comparing a half-maximal recombinant receptor-dependent activity of the therapeutic cell composition to a half-maximal recombinant receptor-dependent activity of a reference standard.

4. The method of any of embodiments 1-3, wherein each of the plurality of incubations comprises culturing a constant number of cells of the therapeutic composition with differing amounts of the recombinant receptor stimulating agent to generate a plurality of different titrated ratios.

5. The method of any of embodiments 1-3, wherein each of the plurality of incubations comprises culturing a constant amount of binding molecule with differing numbers of cells of the therapeutic composition to generate a plurality of different titrated ratios.

6. The method of any of embodiments 1-5, wherein the plurality of incubations are performed for two or more, optionally 3, 4, 5, 6, 7, 8, 9, 10, or more, therapeutic cell compositions.

7. The method of embodiment 6, wherein the two or more therapeutic cell compositions each comprise the same recombinant receptor.

8. The method of embodiment 6, wherein the two or more therapeutic cell compositions each comprise different recombinant receptors.

9. The method of embodiment 6, wherein at least one of the two or more therapeutic cell compositions comprises a different recombinant receptor than the other therapeutic compositions.

10. The method of any of embodiments 6-9, wherein the two or more therapeutic cell compositions are each manufactured using the same manufacturing process.

11. The method of any of embodiments 6-9, wherein the two or more therapeutic cell compositions are each manufactured using a different manufacturing process.

12. The method of any of embodiments 6-9, wherein at least one of the two or more therapeutic cell compositions is manufactured using a different manufacturing process than those used to manufacture the other therapeutic cell compositions.

13. The method of any of embodiments 6-12, wherein the two or more therapeutic cell compositions are produced from cells from a single subject.

14. The method of any of embodiments 6-12, wherein the two or more therapeutic cell compositions are produced from cells from different subjects.

15. The method of embodiment 13 or embodiment 14, wherein the subject is a healthy subject or a subject having a disease or condition.

16. The method of any of embodiments 1-15, wherein the plurality of incubations is at least three incubations.

17. The methods of any of embodiments 1-16, wherein the plurality of incubations is at least five incubations.

18. The methods of any of embodiments 1-17, wherein the plurality of incubations is at least seven incubations.

19. The methods of any of embodiments 1-18, wherein the plurality of incubations is at least ten incubations.

20. The method of any of embodiments 1-19, wherein the recombinant receptor-dependent activity comprises one or more of a cytokine expression, cytolytic activity, receptor upregulation, receptor downregulation, proliferation, gene upregulation, gene down regulation, or cell health.

21. The method of any of embodiments 1-20, wherein the recombinant receptor-dependent activity comprises or is a cytokine expression or production.

22. The method of any of embodiments 1-21, wherein the recombinant receptor-dependent activity comprises or is a cytokine expression or production, wherein the cytokine is TNF-alpha, IFNgamma (IFNg), or IL-2.

23. The method of any of embodiments 1-22, wherein the recombinant receptor-dependent activity comprises or is a cytolytic activity.

24. The method of any of embodiments 1-23, wherein the recombinant receptor-dependent activity comprises or is a receptor upregulation.

25. The method of any of embodiments 1-24, wherein the recombinant receptor-dependent activity comprises or is a receptor downregulation.

26. The method of any of embodiments 1-25, wherein the recombinant receptor-dependent activity comprises or is a proliferation.

27. The method of any of embodiments 1-26, wherein the recombinant receptor-dependent activity comprises or is a gene upregulation.

28. The method of any of embodiments 1-27, wherein the recombinant receptor-dependent activity comprises or is a gene downregulation.

29. The method of any of embodiments 1-28, wherein the recombinant receptor-dependent activity comprises or is a cell health.

30. The method of any of embodiments 1-29, wherein the recombinant receptor-dependent activity comprises or is a cell health, wherein the cell health comprises one or more of cell death, cell diameter, viable cell concentration, and cell count.

31. The method of any of embodiments 1-30, wherein the recombinant receptor-dependent activity measured at each of the plurality of incubations is normalized to a maximum receptor-dependent activity measured for the therapeutic cell composition.

32. The method of any of embodiments 1-31, wherein the reference standard is a therapeutic cell composition comprising a validated titrated ratio resulting in a half-maximal recombinant receptor-dependent activity, a commercially available therapeutic cell composition, a therapeutic cell composition manufactured using a manufacturing process that is identical to a manufacturing process used to manufacture the therapeutic cell composition, a therapeutic cell composition manufactured using a manufacturing process that is different from a manufacturing process used to manufacture the therapeutic cell composition, a therapeutic cell composition comprising an identical recombinant receptor as the therapeutic cell composition, a therapeutic cell composition comprising a different recombinant receptor as the therapeutic cell composition, a therapeutic cell composition manufactured from the same subject, or a therapeutic cell composition manufactured from a different subject.

33. The method of any of embodiments 6-32, wherein the reference standard is one of the two or more therapeutic compositions.

34. The method of any of embodiments 1-33, wherein the recombinant receptor stimulating agent comprises a target antigen or an extracellular domain binding portion thereof, optionally a recombinant antigen, of the recombinant receptor.

35. The method of embodiment 34, wherein the recombinant receptor stimulating agent comprises an extracellular domain binding portion of the antigen and the extracellular domain binding portion comprises an epitope recognized by the recombinant receptor.

36. The method of any of embodiments 1-33, wherein the recombinant receptor stimulating agent is an anti-idiotypic antibody specific to an extracellular antigen binding domain of the recombinant receptor.

37. The method of any of embodiments 1-36, wherein the recombinant receptor stimulating agent is immobilized or attached to a solid support.

38. The method of embodiment 37, wherein the solid support is a surface of the vessel, optionally a well of microwell plate, in which the plurality of incubations are performed.

39. The method of embodiment 37, wherein the solid support is a bead.

40. The method of any of embodiments 1-33, wherein the recombinant receptor stimulating agent is an antigen-expressing cell, optionally wherein the cell is a clone, from a cell line, or a primary cell taken from a subject.

41. The method of embodiment 40, wherein the antigen-expressing cell is a cell line.

42. The method of embodiment 41, wherein the cell line is a tumor cell line.

43. The method of embodiment 40, wherein the antigen-expressing cell is a cell that has been introduced, optionally by transduction, to express the antigen of the recombinant receptor.

44. The method of any of embodiments 1-43, wherein the titrated ratio achieves a linear dose-response range of the recombinant receptor-dependent activity of the reference standard.

45. The method of embodiment 44, wherein the titrated ratio comprises a lower asymptote (minimal) recombinant receptor-dependent activity and an upper asymptote (maximal) recombinant receptor-dependent activity of the reference standard.

46. The method of any of embodiments 1-35, wherein the therapeutic cell composition comprises a single cell subtype enriched or purified from a biological sample or a population of mixed cell subtypes, optionally obtained by mixing cell subtypes enriched or purified from a biological sample.

47. The method of embodiment 46, wherein the biological sample comprises a whole blood sample, a buffy coat sample, a peripheral blood mononuclear cell (PBMC) sample, an unfractionated cell sample, a lymphocyte sample, a white blood cell sample, an apheresis product, or a leukapheresis product.

48. The method of any of embodiments 1-47, wherein the therapeutic cell composition comprises primary cells.

49. The method of any of embodiments 1-38, wherein the therapeutic cell composition comprises autologous cells from a subject to be treated.

50. The method of any of embodiments 1-49, wherein the therapeutic cell composition comprises allogeneic cells.

51. The method of any of embodiments 1-50, wherein the therapeutic cell composition comprises CD3+, CD4+, and/or CD8+ T cells.

52. The method any of embodiments 1-51, wherein the therapeutic cell composition comprises or is CD4+ T cells.

53. The method any of embodiments 1-52, wherein the therapeutic cell composition comprises or is CD8+ T cells.

54. The method of any of embodiments 1-53, wherein the recombinant receptor is a chimeric antigen receptor (CAR).

55. The method of any of embodiments 1-54, wherein the plurality of incubations are performed in a flask, a tube, or a multi-well plate.

56. The method of any of embodiments 1-55, wherein the plurality of incubations are each performed individually in a well of a multi-well plate.

57. The method of embodiment 55 or embodiment 56, wherein the multi-well plate is a 96-well plate, 48-well plate, 12-well plate or 6-well plate.

58. The method of any of embodiments 1 and 4-57, further comprising determining, based on the titrated ratio that results in a half-maximal recombinant receptor-dependent activity a dose of cells of the therapeutic composition for administering to a subject in need thereof.

59. The method of any of embodiments 2-57, further comprising determining, based on the relative potency, a dose of cells of the therapeutic composition for administering to a subject in need thereof.

60. The method of embodiment 58 or embodiment 59, wherein the subject has a disease or condition.

61. The method of embodiment 60, wherein the disease or condition is cancer.

62. The method of any of embodiments 2-61, further comprising determining, based on the relative potency, a manufacturing process that produces an optimal therapeutic cell composition potency, wherein the optimal therapeutic cell composition potency correlates with complete and/or durable response and/or reduced toxicity.

63. The method of any of embodiments 2-62, further comprising determining, based on the relative potency, a manufacturing process that produces a therapeutic cell composition with reduced or low variance in potency, wherein the reduced or low variance is determined compared to the variance in a different manufacturing process.

### VI. EXAMPLES

The following examples are included for illustrative purposes only and are not intended to limit the scope of the invention.

### Example 1: Assay for Assessing Cell Therapy Product Sensitivity to Antigen Stimulation

An assay was designed to measure the sensitivity of a therapeutic cell composition containing cells expressing a recombinant receptor (e.g., chimeric antigen receptor) to antigen stimulation.

Primary CD4+ and CD8+ T cells from three patients or healthy human donors were selected from isolated PBMCs from donor leukapheresis samples. The CD4+ and CD8+ T cells were stimulated in the presence of anti-CD3 and anti-CD28 antibodies or binding fragments in serum-free media in the presence of recombinant IL-2, IL-7, and IL-15. The stimulation was carried out by incubation for between 18 to 30 hours. The cells were transduced with a lentiviral vector encoding a chimeric antigen receptor (CAR) directed against a specific antigen (e.g., CD19 or BCMA) The CAR contained an scFv antigen-binding domain specific for an antigen (e.g., CD19 or BCMA), an immunoglobulin spacer, a transmembrane domain (e.g., transmembrane domain from human CD28), and an intracellular signaling domain containing a human CD3-zeta intracellular signaling domain and a costimulatory signaling domain (e.g., a human 4-1BB intracellular signaling domain). The transduced cells were then cultivated for expansion in the presence of the recombinant cytokines. Although this example is exemplified with the generated CAR-expressing T cells, the assay can be used to assess the antigen-specific sensitivity of any antigen-directed recombinant receptor.

To measure the sensitivity of the exemplary therapeutic cell compositions to antigen-specific stimulation, a fixed concentration of the therapeutic cell composition was incubated with a titrated amount of antigen-expressing target cells. Approximately 50,000 CAR+ T cells of the therapeutic cell composition, which serve as the effector cells in this assay, were added to each well of a multi-well plate. Antigen-expressing target cells were added at titrated amounts from a 12:1 to 0.012:1 ratio of antigen-expressing target cells to effector cells (T:E ratio). The cells were co-cultured for between 2 and 48 hours and then antigen-specific response was assessed by monitoring functional activity of the T cells. In this exemplary assay, supernatant was collected to assess antigen-specific cytokine production after 16 hours.

FIG. 1A shows exemplary secreted cytokine IFNγ concentration at each T:E ratio by donor for three exemplary generated cell products. FIG. 1B shows cytokine secretion (y-axis) normalized to the maximum cytokine concentration observed at the upper asymptote (Vmax).

The T:E ratio that yielded 50% cytokine secretion (e.g., 50% Effective Stimulation or ES50) was determined for each donor, and a Relative Potency calculated relative to Donor 1 (reference standard). For comparison, the three different cell composition products were assessed using a traditional assay format in which cytokine secretion at maximum antigen-stimulation of the drug product was measured, and Relative Potency also was calculated relative to Donor 1. The results are shown in Table E1. The results described in Table E1 demonstrate a different relationship between the amount of cytokine secreted by the cells of the therapeutic cell composition at maximum antigen-specific stimulation and their sensitivity to antigen-specific stimulation as determined by the T:E ratio at 50% Effective Stimulation (ES50). These results indicate that traditional assays may result in saturating level of antigen-stimulation that may not provide an accurate measure of the antigen-sensitivity to antigen stimulation, whereas the provided assay can more reliably measure the sensitivity of antigen-directed therapeutic cell compositions to stimulation by antigen.

TABLE E1
Relative Potency determined by assay.

| | | | | Titrated Assay Format | |
|---|---|---|---|---|---|
| | | Traditional Assay Format | | T:E Ratio | |
| | | | Relative | at 50% | |
| | | Cytokine | Potency by | Effective | Relative |
| | Sample | Secretion | Cytokine | Stimulation | Potency |
| Sample | Type | (pg/mL) | Secretion | (ES50) | by ES50 |
| Donor 1 | Reference | 2,583,535 | N.A. | 0.57 | N.A. |
| | Standard | | | | |
| Donor 2 | Test | 3,781,911 | 146% | 0.15 | 26% |
| | Sample | | | | |
| Donor 3 | Test | 2,758,740 | 107% | 0.26 | 46% |
| | Sample | | | | |

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | ANNOTATION |
|---|---|---|
| 1 | LEGGGEGRGSLLTCGDVEENPGPR | T2A |
| 2 | MLLLVTSLLLCELPHPAFLLIPRKVCNGIGIGEFKDSLSINATNI | tEGFR |
| | KHFKNCTSISGDLHILPVAFRGDSFTHTPPLDPQELDILKTVKEI | |
| | TGFLLIQAWPENRTDLHAFENLEIIRGRTKQHGQFSLAVVSLNIT | |
| | SLGLRSLKEISDGDVIISGNKNLCYANTINWKKLFGTSGQKTKII | |
| | SNRGENSCKATGQVCHALCSPEGCWGPEPRDCVSCRNVSRGRECV | |
| | DKCNLLEGEPREFVENSECIQCHPECLPQAMNITCTGRGPDNCIQ | |
| | CAHYIDGPHCVKTCPAGVMGENNTLVWKYADAGHVCHLCHPNCTY | |
| | GCTGPGLEGCPTNGPKIPSIATGMVGALLLLLVVALGIGLFM | |
| 3 | RKVCNGIGIGEFKDSLSINATNIKHFKNCTSISGDLHILPVAFRG | tEGFR |
| | DSFTHTPPLDPQELDILKTVKEITGFLLIQAWPENRTDLHAFENL | |
| | EIIRGRTKQHGQFSLAVVSLNITSLGLRSLKEISDGDVIISGNKN | |
| | LCYANTINWKKLFGTSGQKTKIISNRGENSCKATGQVCHALCSPE | |
| | GCWGPEPRDCVSCRNVSRGRECVDKCNLLEGEPREFVENSECIQC | |
| | HPECLPQAMNITCTGRGPDNCIQCAHYIDGPHCVKTCPAGVMGEN | |
| | NTLVWKYADAGHVCHLCHPNCTYGCTGPGLEGCPTNGPKIPSIAT | |
| | GMVGALLLLLVVALGIGLFM | |
| 4 | EGRGSLLTCGDVEENPGP | T2A |
| 5 | GSGATNFSLLKQAGDVEENPGP | P2A |
| 6 | ATNFSLLKQAGDVEENPGP | P2A |
| 7 | QCTNYALLKLAGDVESNPGP | E2A |
| 8 | VKQTLNFDLLKLAGDVESNPGP | F2A |
| 9 | atgcttctcctggtgacaagccttctgctctgtgagttaccacac | GMCSFR alpha |
| | ccagcattcctcctgatccca | chain signal |
| | | sequence |
| 10 | MLLLVTSLLLCELPHPAFLLIP | GMCSFR alpha |
| | | chain signal |
| | | sequence |
| 11 | MALPVTALLLPLALLLHA | CD8 alpha |
| | | signal peptide |
| 12 | MPLLLLLPLLWAGALA | CD33 signal |
| | | peptide |
| 13 | MLQMAGQCSQNEYFDSLLHACIPCQLRCSSNTPPLTCQRYCNASV | Extracellular |
| | TNSVKGTNA | domain of human |
| | | BCMA (GenBank |
| | | No. |
| | | NP_001183.2) |
| 14 | DSSKWVFEHPETLYAWEGACVWIPCTYRALDGDLESFILFHNPEY | Human CD22 |
| | NKNTSKFDGTRLYESTKDGKVPSEQKRVQFLGDKNKNCTLSIHPV | extracellular |
| | HLNDSGQLGLRMESKTEKWMERIHLNVSERPFPPHIQLPPEIQES | domain |
| | QEVTLTCLLNFSCYGYPIQLQWLLEGVPMRQAAVTSTSLTIKSVF | |
| | TRSELKFSPQWSHHGKIVTCQLQDADGKFLSNDTVQLNVKHTPKL | |
| | EIKVTPSDAIVREGDSVTMTCEVSSSNPEYTTVSWLKDGTSLKKQ | |
| | NTFTLNLREVTKDQSGKYCCQVSNDVGPGRSEEVFLQVQYAPEPS | |
| | TVQILHSPAVEGSQVEFLCMSLANPLPTNYTWYHNGKEMQGRTEE | |
| | KVHIPKILPWHAGTYSCVAENILGTGQRGPGAELDVQYPPKKVTT | |
| | VIQNPMPIREGDTVTLSCNYNSSNPSVTRYEWKPHGAWEEPSLGV | |
| | LKIQNVGWDNTTIACAACNSWCSWASPVALNVQYAPRDVRVRKIK | |
| | PLSEIHSGNSVSLQCDFSSSHPKEVQFFWEKNGRLLGKESQLNFD | |
| | SISPEDAGSYSCWVNNSIGQTASKAWTLEVLYAPRRLRVSMSPGD | |
| | QVMEGKSATLTCESDANPPVSHYTWFDWNNQSLPYHSQKLRLEPV | |
| | KVQHSGAYWCQGTNSVGKGRSPLSTLTVYYSPETIGRR | |
| 15 | MPPPRLLFFLLFLTPMEVRPEEPLVVKVEEGDNAVLQCLKGTSDG | CD19 |
| | PTQQLTWSRESPLKPFLKLSLGLPGLGIHMRPLAIWLFIFNVSQQ | Accession No. |
| | MGGFYLCQPGPPSEKAWQPGWTVNVEGSGELFRWNVSDLGGLGCG | P15391 |
| | LKNRSSEGPSSPSGKLMSPKLYVWAKDRPEIWEGEPPCLPPRDSL | Homo Sapiens |
| | NQSLSQDLTMAPGSTLWLSCGVPPDSVSRGPLSWTHVHPKGPKSL | |
| | LSLELKDDRPARDMWVMETGLLLPRATAQDAGKYYCHRGNLTMSF | |
| | HLEITARPVLWHWLLRTGGWKVSAVTLAYLIFCLCSLVGILHLQR | |
| | ALVLRRKRKRMTDPTRRFFKVTPPPGSGPQNQYGNVLSLPTPTSG | |
| | LGRAQRWAAGLGGTAPSYGNPSSDVQADGALGSRSPPGVGPEEEE | |
| | GEGYEEPDSEEDSEFYENDSNLGQDQLSQDGSGYENPEDEPLGPE | |
| | DEDSFSNAESYENEDEELTQPVARTMDFLSPHGSAWDPSREATSL | |
| | GSQSYEDMRGILYAAPQLRSIRGQPGPNHEEDADSYENMDNPDGP | |
| | DPAWGGGGRMGTWSTR | |
| 16 | EPKSCDKTHTCPPCPAPELLGGPSVFLFPPKPKDTLMISRTPEVT | Human IgG1 Fc |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSREEMTKNQVSLTCLVKGFYPSDIAVEWESNGQPENNYKTTP | |
| | PVLDSDGSFFLYSKLTVDKSRWQQGNVFSCSVMHEALHNHYTQKS | |
| | LSLSPGK | |
| 17 | PKSSDKTHTCPPCPAPEAEGAPSVFLFPPKPKDTLMISRTPEVTC | Modified Human |
| | VVVDVSHEDPEVKFNWYVDGVEVHNAKTKPREEQYNSTYRVVSVL | IgG1 Fc |
| | TVLHQDWLNGKEYKCKVSNKALPSSIEKTISKAKGQPREPQVYTL | |
| | PPSRDELTKNQVSLTCLVKGFYPSDIAVEWESNGQPENNYKTTPP | |
| | VLDSDGSFFLYSKLTVDKSRWQQGNVFSCSVMHEALHNHYTQKSL | |
| | SLSPGK | |
| 18 | MLQMAGQCSQNEYFDSLLHACIPCQLRCSSNTPPLTCQRYCNASV | BCMA-Fc fusion |
| | TNSVKGTNAGGGGSPKSSDKTHTCPPCPAPEAEGAPSVFLFPPKP | polypeptide |
| | KDTLMISRTPEVTCVVVDVSHEDPEVKFNWYVDGVEVHNAKTKPR | |
| | EEQYNSTYRVVSVLTVLHQDWLNGKEYKCKVSNKALPSSIEKTIS | |
| | KAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSV | |
| | MHEALHNHYTQKSLSLSPGK | |
| 19 | QETELSVSAELVPTSSWNISSELNKDSYLTLDEPMNNITTSLGQT | Human ROR1 |
| | AELHCKVSGNPPPTIRWFKNDAPVVQEPRRLSFRSTIYGSRLRIR | fragment |
| | NLDTTDTGYFQCVATNGKEVVSSTGVLFVKFGPPPTASPGYSDEY | |
| | EEDGFCQPYRGIACARFIGNRTVYMESLHMQGEIENQITAAFTMI | |
| | GTSSHLSDKCSQFAIPSLCHYAFPYCDETSSVPKPRDLCRDECEI | |
| | LENVLCQTEYIFARSNPMILMRLKLPNCEDLPQPESPEAANCIRI | |
| | GIPMADPINKNHKCYNSTGVDYRGTVSVTKSGRQCQPWNSQYPHT | |
| | HTFTALRFPELNGGHSYCRNPGNQKEAPWCFTLDENFKSDLCDIP | |
| | ACDSKDSKEKNKMEILY | |
| 20 | QETELSVSAELVPTSSWNISSELNKDSYLTLDEPMNNITTSLGQT | ROR1-Fc fusion |
| | AELHCKVSGNPPPTIRWFKNDAPVVQEPRRLSFRSTIYGSRLRIR | polypeptide |
| | NLDTTDTGYFQCVATNGKEVVSSTGVLFVKFGPPPTASPGYSDEY | |
| | EEDGFCQPYRGIACARFIGNRTVYMESLHMQGEIENQITAAFTMI | |
| | GTSSHLSDKCSQFAIPSLCHYAFPYCDETSSVPKPRDLCRDECEI | |
| | LENVLCQTEYIFARSNPMILMRLKLPNCEDLPQPESPEAANCIRI | |
| | GIPMADPINKNHKCYNSTGVDYRGTVSVTKSGRQCQPWNSQYPHT | |
| | HTFTALRFPELNGGHSYCRNPGNQKEAPWCFTLDENFKSDLCDIP | |
| | ACDSKDSKEKNKMEILYGGGGSPKSSDKTHTCPPCPAPEAEGAPS | |
| | VFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALP | |
| | SSIEKTISKAKGQPREPQVYTLPPSRDELTKNQVSLTCLVKGFYP | |
| | SDIAVEWESNGQPENNYKTTPPVLDSDGSFFLYSKLTVDKSRWQQ | |
| | GNVFSCSVMHEALHNHYTQKSLSLSPGK | |
| 21 | DSSKWVFEHPETLYAWEGACVWIPCTYRALDGDLESFILFHNPEY | CD22-Fc fusion |
| | NKNTSKFDGTRLYESTKDGKVPSEQKRVQFLGDKNKNCTLSIHPV | polypeptide |
| | HLNDSGQLGLRMESKTEKWMERIHLNVSERPFPPHIQLPPEIQES | |
| | QEVTLTCLLNFSCYGYPIQLQWLLEGVPMRQAAVTSTSLTIKSVF | |
| | TRSELKFSPQWSHHGKIVTCQLQDADGKFLSNDTVQLNVKHTPKL | |
| | EIKVTPSDAIVREGDSVTMTCEVSSSNPEYTTVSWLKDGTSLKKQ | |
| | NTFTLNLREVTKDQSGKYCCQVSNDVGPGRSEEVFLQVQYAPEPS | |
| | TVQILHSPAVEGSQVEFLCMSLANPLPTNYTWYHNGKEMQGRTEE | |
| | KVHIPKILPWHAGTYSCVAENILGTGQRGPGAELDVQYPPKKVTT | |
| | VIQNPMPIREGDTVTLSCNYNSSNPSVTRYEWKPHGAWEEPSLGV | |
| | LKIQNVGWDNTTIACAACNSWCSWASPVALNVQYAPRDVRVRKIK | |
| | PLSEIHSGNSVSLQCDFSSSHPKEVQFFWEKNGRLLGKESQLNFD | |
| | SISPEDAGSYSCWVNNSIGQTASKAWTLEVLYAPRRLRVSMSPGD | |
| | QVMEGKSATLTCESDANPPVSHYTWFDWNNQSLPYHSQKLRLEPV | |
| | KVQHSGAYWCQGTNSVGKGRSPLSTLTVYYSPETIGRRGGGGSPK | |
| | SSDKTHTCPPCPAPEAEGAPSVFLFPPKPKDTLMISRTPEVTCVV | |
| | VDVSHEDPEVKFNWYVDGVEVHNAKTKPREEQYNSTYRVVSVLTV | |
| | LHQDWLNGKEYKCKVSNKALPSSIEKTISKAKGQPREPQVYTLPP | |
| | SRDELTKNQVSLTCLVKGFYPSDIAVEWESNGQPENNYKTTPPVL | |
| | DSDGSFFLYSKLTVDKSRWQQGNVFSCSVMHEALHNHYTQKSLSL | |
| | SPGK | |
| 22 | GGGGS | Linker |
| 23 | GGGS | Linker |
| 24 | GGGGSGGGGSGGGGS | Linker |
| 25 | GSTSGSGKPGSGEGSTKG | Linker |
| 26 | SRGGGGSGGGGSGGGGSLEMA | Linker |
| 27 | DYGVS | CDR H1 |
| 28 | VIWGSETTYYNSALKS | CDR H2 |
| 29 | YAMDYWG | CDR H3 |
| 30 | HYYYGGSYAMDY | CDR H3 |
| 31 | RASQDISKYLN | CDR L1 |
| 32 | SRLHSGV | CDR L2 |
| 33 | HTSRLHS | CDR L2 |
| 34 | GNTLPYTFG | CDR L3 |
| 35 | QQGNTLPYT | CDR L3 |
| 36 | EVKLQESGPGLVAPSQSLSVTCTVSGVSLPDYGVSWIRQPP | VH |
| | RKGLEWLGVIWGSETTYYNSALKSRLTIIKDNSKSQVFLKM | |
| | NSLQTDDTAIYYCAKHYYYGGSYAMDYWGQGTSVTVSS | |
| 37 | DIQMTQTTSSLSASLGDRVTISCRASQDISKYLNWYQQKPD | VL |
| | GTVKLLIYHTSRLHSGVPSRFSGSGSGTDYSLTISNLEQED | |
| | IATYFCQQGNTLPYTFGGGTKLEIT | |
| 38 | gacatccagatgacccagaccacctccagcctgagcgccagcctg | Sequence |
| | ggcgaccgggtgacc | encoding scFv |
| | atcagctgccgggccagccaggacatcagcaagtacctgaactgg | |
| | tatcagcagaagccc | |
| | gacggcaccgtcaagctgctgatctaccacaccagccggctgcac | |
| | agcggcgtgcccagc | |
| | cggtttagcggcagcggctccggcaccgactacagcctgaccatc | |
| | tccaacctggaacagg | |
| | aagatatcgccacctacttttgccagcagggcaacacactgccct | |
| | acacctttggcggcgga | |
| | acaaagctggaaatcaccggcagcacctccggcagcggcaagcct | |
| | ggcagcggcgagg | |
| | gcagcaccaagggcgaggtgaagctgcaggaaagcggccctggcc | |
| | tggtggcccccagc | |
| | cagagcctgagcgtgacctgcaccgtgagcggcgtgagcctgccc | |
| | gactacggcgtgagc | |
| | tggatccggcagccccccaggaagggcctggaatggctgggcgtg | |
| | atctggggcagcga | |
| | gaccacctactacaacagcgccctgaagagccggctgaccatcat | |
| | caaggacaacagcaa | |
| | gagccaggtgttcctgaagatgaacagcctgcagaccgacgacac | |
| | cgccatctactactgc | |
| | gccaagcactactactacggcggcagctacgccatggactactgg | |
| | ggccagggcaccagc | |
| | gtgaccgtgagcagc | |
| 39 | DIQMTQTTSSLSASLGDRVTISCRASQDISKYLNWYQQKPD | scFv |
| | GTVKLLIYHTSRLHSGVPSRFSGSGSGTDYSLTISNLEQED | |
| | IATYFCQQGNTLPYTFGGGTKLEITGSTSGSGKPGSGEGST | |
| | KGEVKLQESGPGLVAPSQSLSVTCTVSGVSLPDYGVSWIRQ | |
| | PPRKGLEWLGVIWGSETTYYNSALKSRLTIIKDNSKSQVFL | |
| | KMNSLQTDDTAIYYCAKHYYYGGSYAMDYWGQGTSVTVSS | |
| 40 | SYWMN | CDR H1 |
| 41 | QIYPGDGDTNYNGKFKG | CDR H2 |
| 42 | KTISSVVDFYFDY | CDR H3 |
| 43 | QQYNRYPYT | CDR L1 |
| 44 | SYWMN | CDR L2 |
| 45 | QIYPGDGDTNYNGKFKG | CDR L3 |
| 46 | EVKLQQSGAELVRPGSSVKISCKASGYAFSSYWMNWVKQR | VH |
| | PGQGLEWIGQIYPGDGDTNYNGKFKGQATLTADKSSSTAY | |
| | MQLSGLTSEDSAVYFCARKTISSVVDFYFDYWGQGTTVTV | |
| | SS | |
| 47 | DIELTQSPKFMSTSVGDRVSVTCKASQNVGTNVAWYQQKP | VL |
| | GQSPKPLIYSATYRNSGVPDRFTGSGSGTDFTLTITNVQS | |
| | KDLADYFCQQYNRYPYTSGGGTKLEIKR | |
| 48 | KASQNVGTNVA | CDR L1 |
| 49 | SATYRNS | CDR L2 |
| 50 | QQYNRYPYT | CDR L3 |
| 51 | SYWMN | CDR H1 |
| 52 | QIYPGDGDTNYNGKFKG | CDR H2 |
| 53 | KTISSVVDFYFDY | CDR H3 |
| 54 | EVKLQQSGAELVRPGSSVKISCKASGYAFSSYWMNWVKQ | scFv |
| | RPGQGLEWIGQIYPGDGDTNYNGKFKGQATLTADKSSSTA | |
| | YMQLSGLTSEDSAVYFCARKTISSVVDFYFDYWGQGTTVT | |
| | VSSGGGGSGGGGSGGGGSDIELTQSPKFMSTSVGDRVSVT | |
| | CKASQNVGTNVAWYQQKPGQSPKPLIYSATYRNSGVPDR | |
| | FTGSGSGTDFTLTITNVQSKDLADYFCQQYNRYPYTSGGG | |
| | TKLEIKR | |
| 55 | QIQLVQSGPELKKPGETVKISCKASGYTFTDYSINWVKRAP | VH |
| | GKGLKWMGWINTETREPAYAYDERGRFAFSLETSASTAY | |
| | LQINNLKYEDTATYFCALDYSYAMDYWGQGTSVTVSS | |
| 56 | DIVLTQSPPSLAMSLGKRATISCRASESVTILGSHLIHWYQQ | VL |
| | KPGQPPTLLIQLASNVQTGVPARFSGSGSRTDFTLTIDPVEE | |
| | DDVAVYYCLQSRTIPRTFGGGTKLEIK | |
| 57 | QIQLVQSGPDLKKPGETVKLSCKASGYTFTNFGMNWVKQ | VH |
| | APGKGFKWMAWINTYTGESYFADDFKGRFAFSVETSATT | |
| | AYLQINNLKTEDTATYFCARGEIYYGYDGGFAYWGQGTL | |
| | VTVSA | |
| 58 | DVVMTQSHRFMSTSVGDRVSITCRASQDVNTAVSWYQQK | VL |
| | PGQSPKLLIFSASYRYTGVPDRFTGSGSGADFTLTISSVQAE | |
| | DLAVYYCQQHYSTPWTFGGGTKLDIK | |
| 59 | EVQLVQSGAEVKKPGESLKISCKGSGYSFTSYWIGWVRQM | VH |
| | PGKGLEWMGIIYPGDSDTRYSPSFQGHVTISADKSISTAYL | |
| | QWSSLKASDTAMYYCARYSGSFDNWGQGTLVTVSS | |
| 60 | SYELTQPPSASGTPGQRVTMSCSGTSSNIGSHSVNWYQQLP | VL |
| | GTAPKLLIYTNNQRPSGVPDRFSGSKSGTSASLAISGLQSED | |
| | EADYYCAAWDGSLNGLVFGGGTKLTVLG | |
| 61 | EVQLVQSGAEMKKPGASLKLSCKASGYTFIDYYVYWMRQ | VH |
| | APGQGLESMGWINPNSGGTNYAQKFQGRVTMTRDTSISTA | |
| | YMELSRLRSDDTAMYYCARSQRDGYMDYWGQGTLVTVS | |
| | S | |
| 62 | QSALTQPASVSASPGQSIAISCTGTSSDVGWYQQHPGKAPK | VL |
| | LMIYEDSKRPSGVSNRFSGSKSGNTASLTISGLQAEDEADY | |
| | YCSSNTRSSTLVFGGGTKLTVLG | |
| 63 | EVQLVQSGAEVKKPGSSVKVSCKASGGTFSSYAISWVRQA | VH |
| | PGQGLEWMGRIIPILGIANYAQKFQGRVTMTEDTSTDTAY | |
| | MELSSLRSEDTAVYYCARSGYSKSIVSYMDYWGQGTLVT | |
| | VSS | |
| 64 | LPVLTQPPSTSGTPGQRVTVSCSGSSSNIGSNVVFWYQQLP | VL |
| | GTAPKLVIYRNNQRPSGVPDRFSVSKSGTSASLAISGLRSE | |
| | DEADYYCAAWDDSLSGYVFGTGTKVTVLG | |
| 65 | QVQLVQSGAEVKKPGSSVKVSCKASGGTFSSYAISWVRQA | VH |
| | PGQGLEWMGRIIPILGTANYAQKFQGRVTITADESTSTAYM | |
| | ELSSLRSEDTAVYYCARSGYGSYRWEDSWGQGTLVTVSS | |
| 66 | QAVLTQPPSASGTPGQRVTISCSGSSSNIGSNYVFWYQQLP | VL |
| | GTAPKLLIYSNNQRPSGVPDRFSGSKSGTSASLAISGLRSED | |
| | EADYYCAAWDDSLSASYVFGTGTKVTVLG | |
| 67 | QVQLVQSGAEVKKPGASVKVSCKASGYTFTDYYMHWVR | VH |
| | QAPGQRLEWMGWINPNSGGTNYAQKFQDRITVTRDTSSN | |
| | TGYMELTRLRSDDTAVYYCARSPYSGVLDKWGQGTLVTV | |
| | SS | |
| 68 | QSVLTQPPSVSGAPGQRVTISCTGSSSNIGAGFDVHWYQQL | VL |
| | PGTAPKLLIYGNSNRPSGVPDRFSGSKSGTSASLAITGLQAE | |
| | DEADYYCQSYDSSLSGYVFGTGTKVTVLG | |
| 69 | ESKYGPPCPPCP | spacer |
| | | (IgG4hinge) |
| | | (aa) |
| 70 | GAATCTAAGTACGGACCGCCCTGCCCCCCTTGCCCT | spacer |
| | | (IgG4hinge) |
| | | (nt) |
| 71 | ESKYGPPCPPCPGQPREPQVYTLPPSQEEMTKNQVSLTCLV | Hinge-CH3 |
| | KGFYPSDIAVEWESNGQPENNYKTTPPVLDSDGSFFLYSRL | spacer |
| | TVDKSRWQEGNVFSCSVMHEALHNHYTQKSLSLSLGK | Homo sapiens |
| 72 | ESKYGPPCPPCPAPEFLGGPSVFLFPPKPKDTLMISRTPEVT | Hinge-CH2-CH3 |
| | CVVVDVSQEDPEVQFNWYVDGVEVHNAKTKPREEQFNST | spacer |
| | YRVVSVLTVLHQDWLNGKEYKCKVSNKGLPSSIEKTISKA | Homo sapiens |
| | KGQPREPQVYTLPPSQEEMTKNQVSLTCLVKGFYPSDIAV | |
| | EWESNGQPENNYKTTPPVLDSDGSFFLYSRLTVDKSRWQE | |
| | GNVFSCSVMHEALHNHYTQKSLSLSLGK | |
| 73 | RWPESPKAQASSVPTAQPQAEGSLAKATTAPATTRNTGRG | IgD-hinge-Fc |
| | GEEKKKEKEKEEQEERETKTPECPSHTQPLGVYLLTPAVQ | Homo sapiens |
| | DLWLRDKATFTCFVVGSDLKDAHLTWEVAGKVPTGGVEE | |
| | GLLERHSNGSQSQHSRLTLPRSLWNAGTSVTCTLNHPSLPP | |
| | QRLMALREPAAQAPVKLSLNLLASSDPPEAASWLLCEVSG | |
| 74 | Glu Val Val Val Lys Tyr Gly Pro Pro Cys Pro | Exemplary IgG |
| | Pro Cys Pro | Hinge |
| 75 | X1PPX2P | X1 is glycine, |
| | | cysteine or |
| | | arginine |
| | | X2 is cysteine |
| | | or threonine |
| | | Exemplary IgG |
| | | Hinge |
| 76 | Glu Pro Lys Ser Cys Asp Lys Thr His Thr Cys | Exemplary IgG |
| | Pro Pro Cys Pro | Hinge |
| 77 | Glu Arg Lys Cys Cys Val Glu Cys Pro Pro Cys | Exemplary IgG |
| | Pro | Hinge |
| 78 | ELKTPLGDTHTCPRCPEPKSCDTPPPCPRCPEPKSCDTPPPC | Exemplary IgG |
| | PRCPEPKSCDTPPPCPRCP | Hinge |
| 79 | Glu Ser Lys Tyr Gly Pro Pro Cys Pro Ser Cys | Exemplary IgG |
| | Pro | Hinge |
| 80 | Glu Ser Lys Tyr Gly Pro Pro Cys Pro Pro Cys | Exemplary IgG |
| | Pro | Hinge |
| 81 | Tyr Gly Pro Pro Cys Pro Pro Cys Pro | Exemplary IgG |
| | | Hinge |
| 82 | Lys Tyr Gly Pro Pro Cys Pro Pro Cys Pro | Exemplary IgG |
| | | Hinge |
| 83 | FWVLVVVGGVLACYSLLVTVAFIIFWV | CD28 (amino |
| | | acids 153-179 |
| | | of |
| | | Accession No. |
| | | P10747) |
| | | Homo sapiens |
| 84 | IEVMYPPPYLDNEKSNGTIIHVKGKHLCPSPLFPGPSKPFW | CD28 (amino |
| | VLVVVGGVLACYSLLVTVAFIIFWV | acids 114-179 |
| | | of |
| | | Accession No. |
| | | P10747) |
| | | Homo sapiens |
| 85 | RSKRSRLLHSDYMNMTPRRPGPTRKHYQPYAPPRDFAAY | CD28 (amino |
| | RS | acids 180-220 |
| | | of |
| | | P10747) |
| | | Homo sapiens |
| 86 | RSKRSRGGHSDYMNMTPRRPGPTRKHYQPYAPPRDFAAY | CD28 (LL to GG) |
| | RS | Homo sapiens |
| 87 | KRGRKKLLYIFKQPFMRPVQTTQEEDGCSCRFPEEEEGGCEL | 4-1BB (amino |
| | | acids 214-255 |
| | | of |
| | | Q07011.1) |
| | | Homo sapiens |
| 88 | RVKFSRSADAPAYQQGQNQLYNELNLGRREEYDVLDKRR | CD3 zeta |
| | GRDPEMGGKPRRKNPQEGLYNELQKDKMAEAYSEIGMK | Homo sapiens |
| | GERRRGKGHDGLYQGLSTATKDTYDALHMQALPPR | |
| 89 | RVKFSRSAEPPAYQQGQNQLYNELNLGRREEYDVLDKRR | CD3 zeta |
| | GRDPEMGGKPRRKNPQEGLYNELQKDKMAEAYSEIGMK | Homo sapiens |
| | GERRRGKGHDGLYQGLSTATKDTYDALHMQALPPR | |
| 90 | RVKFSRSADAPAYKQGQNQLYNELNLGRREEYDVLDKRR | CD3 zeta |
| | GRDPEMGGKPRRKNPQEGLYNELQKDKMAEAYSEIGMK | Homo sapiens |
| | GERRRGKGHDGLYQGLSTATKDTYDALHMQALPPR | |
| 91 | -PGGG-(SGGGG)5-P- | Linker; P is |
| | | proline, G is |
| | | glycine and S |
| | | is serine |
| 92 | GSADDAKKDAAKKDGKS | Linker |

1. A method of determining potency of a therapeutic cell composition, the method comprising:
performing a plurality of incubations, each of said plurality of incubations comprising culturing cells of a therapeutic cell composition comprising cells engineered to express a recombinant receptor with a recombinant receptor stimulating agent, wherein:
binding of the recombinant receptor stimulating agent to the recombinant receptor stimulates a recombinant receptor-dependent activity in the cell; and
each of the plurality of incubations comprises a different titrated ratio of the cells of the therapeutic cell composition to the recombinant receptor stimulating agent;
measuring the recombinant receptor-dependent activity from each of the plurality of incubations; and
determining, based on the recombinant receptor-dependent activity measured from each of the plurality of incubations, the titrated ratio that results in a half-maximal recombinant receptor-dependent activity.

2. The method of claim 1, further comprising determining a relative potency of the therapeutic cell composition by comparing the titrated ratio resulting in the half-maximal recombinant receptor-dependent activity of the therapeutic cell composition to a titrated ratio resulting in a half-maximal recombinant receptor-dependent activity of a reference standard.

3. A method of determining potency of a therapeutic cell composition, the method comprising:
performing a plurality of incubations, each of said plurality of incubations comprising culturing cells of a therapeutic cell composition comprising cells engineered to express a recombinant receptor with a recombinant receptor stimulating agent, wherein:
binding of the recombinant receptor stimulating agent to the recombinant receptor stimulates a recombinant receptor-dependent activity in the cell; and
each of the plurality of incubations comprises a different titrated ratio of the cells of the therapeutic cell composition to the recombinant receptor stimulating agent;
measuring the recombinant receptor-dependent activity from each of the plurality of incubations; and
determining a relative potency of the therapeutic cell composition by comparing a half-maximal recombinant receptor-dependent activity of the therapeutic cell composition to a half-maximal recombinant receptor-dependent activity of a reference standard.

4. The method of any of claims 1-3, wherein each of the plurality of incubations comprises culturing a constant number of cells of the therapeutic composition with differing amounts of the recombinant receptor stimulating agent to generate a plurality of different titrated ratios.

5. The method of any of claims 1-3, wherein each of the plurality of incubations comprises culturing a constant amount of recombinant receptor stimulating agent with differing numbers of cells of the therapeutic composition to generate a plurality of different titrated ratios.

6. The method of any of claims 1-5, wherein the plurality of incubations are performed for two or more, optionally 3, 4, 5, 6, 7, 8, 9, 10, or more, therapeutic cell compositions.

7. The method of claim 6, wherein the two or more therapeutic cell compositions each comprise the same recombinant receptor.

8. The method of claim 6, wherein the two or more therapeutic cell compositions each comprise different recombinant receptors.

9. The method of claim 6, wherein at least one of the two or more therapeutic cell compositions comprises a different recombinant receptor than the other therapeutic compositions.

10. The method of any of claims 6-9, wherein the two or more therapeutic cell compositions are each manufactured using the same manufacturing process.

11. The method of any of claims 6-9, wherein the two or more therapeutic cell compositions are each manufactured using a different manufacturing process.

12. The method of any of claims 6-9, wherein at least one of the two or more therapeutic cell compositions is manufactured using a different manufacturing process than those used to manufacture the other therapeutic cell compositions.

13. The method of any of claims 6-12, wherein the two or more therapeutic cell compositions are produced from cells from a single subject.

14. The method of any of claims 6-12, wherein the two or more therapeutic cell compositions are produced from cells from different subjects.

15. The method of claim 13, wherein the subject is a healthy subject or a subject having a disease or condition.

16. The method of claim 14, wherein each of the different subjects have the same disease or condition.

17. The method of claim 14, wherein each of the different subjects are to be treated with the same therapeutic cell composition for treating a disease or condition in the subject.

18. The method of any of claims 1-17, wherein the plurality of incubations is at least three incubations.

19. The methods of any of claims 1-18, wherein the plurality of incubations is at least five incubations.

20. The methods of any of claims 1-19, wherein the plurality of incubations is at least seven incubations.

21. The methods of any of claims 1-20, wherein the plurality of incubations is at least ten incubations.

22. The method of any of claims 1-21, wherein the recombinant receptor-dependent activity comprises one or more of a cytokine expression, cytolytic activity, receptor upregulation, receptor downregulation, proliferation, gene upregulation, gene down regulation, or cell health.

23. The method of any of claims 1-22, wherein the recombinant receptor-dependent activity comprises or is a cytokine expression or production.

24. The method of any of claims 1-23, wherein the recombinant receptor-dependent activity comprises or is a cytokine expression or production, wherein the cytokine is TNF-alpha, IFNgamma (IFNg), or IL-2.

25. The method of any of claims 1-24, wherein the recombinant receptor-dependent activity comprises or is a cytolytic activity.

26. The method of any of claims 1-25, wherein the recombinant receptor-dependent activity comprises or is a receptor upregulation.

27. The method of any of claims 1-26, wherein the recombinant receptor-dependent activity comprises or is a receptor downregulation.

28. The method of any of claims 1-27, wherein the recombinant receptor-dependent activity comprises or is a proliferation.

29. The method of any of claims 1-28, wherein the recombinant receptor-dependent activity comprises or is a gene upregulation.

30. The method of any of claims 1-29, wherein the recombinant receptor-dependent activity comprises or is a gene downregulation.

31. The method of any of claims 1-30, wherein the recombinant receptor-dependent activity comprises or is a cell health.

32. The method of any of claims 1-31, wherein the recombinant receptor-dependent activity comprises or is a cell health, wherein the cell health comprises one or more of cell death, cell diameter, viable cell concentration, and cell count.

33. The method of any of claims 1-32, wherein the recombinant receptor-dependent activity measured at each of the plurality of incubations is normalized to a maximum receptor-dependent activity measured for the therapeutic cell composition.

34. The method of any of claims 1-33, wherein the reference standard is a therapeutic cell composition comprising a validated titrated ratio resulting in a half-maximal recombinant receptor-dependent activity, a commercially available therapeutic cell composition, a therapeutic cell composition manufactured using a manufacturing process that is identical to a manufacturing process used to manufacture the therapeutic cell composition, a therapeutic cell composition manufactured using a manufacturing process that is different from a manufacturing process used to manufacture the therapeutic cell composition, a therapeutic cell composition comprising an identical recombinant receptor as the therapeutic cell composition, a therapeutic cell composition comprising a different recombinant receptor as the therapeutic cell composition, a therapeutic cell composition manufactured from the same subject, or a therapeutic cell composition manufactured from a different subject.

35. The method of any of claims 6-34, wherein the reference standard is one of the two or more therapeutic compositions.

36. The method of any of claims 1-35, wherein the recombinant receptor stimulating agent comprises a target antigen or an extracellular domain binding portion thereof, optionally a recombinant antigen, of the recombinant receptor.

37. The method of claim 36, wherein the recombinant receptor stimulating agent comprises an extracellular domain binding portion of the antigen and the extracellular domain binding portion comprises an epitope recognized by the recombinant receptor.

38. The method of any of claims 1-35, wherein the recombinant receptor stimulating agent is an antibody specific to an extracellular binding domain of the recombinant receptor.

39. The method of any of claims 1-35 and 38, wherein the the recombinant receptor stimulating agent is an anti-idiotypic antibody specific to an extracellular antigen binding domain of the recombinant receptor.

40. The method of any of claims 1-39, wherein the recombinant receptor stimulating agent is immobilized or attached to a solid support.

41. The method of claim 40, wherein the solid support is a surface of the vessel, optionally a well of microwell plate, in which the plurality of incubations are performed.

42. The method of claim 40, wherein the solid support is a bead.

43. The method of any of claims 1-35, wherein the recombinant receptor stimulating agent is an antigen-expressing cell, optionally wherein the cell is a clone, from a cell line, or a primary cell taken from a subject.

44. The method of claim 43, wherein the antigen-expressing cell is a cell line.

45. The method of claim 44, wherein the cell line is a tumor cell line.

46. The method of claim 43, wherein the antigen-expressing cell is a cell that has been introduced, optionally by transduction, to express the antigen of the recombinant receptor.

47. The method of any of claims 1-46, wherein the titrated ratio achieves a linear dose-response range of the recombinant receptor-dependent activity of the reference standard.

48. The method of claim 47, wherein the titrated ratio comprises a lower asymptote (minimal) recombinant receptor-dependent activity and an upper asymptote (maximal) recombinant receptor-dependent activity of the reference standard.

49. The method of any of claims 1-37, wherein the therapeutic cell composition comprises a single cell subtype enriched or purified from a biological sample or a population of mixed cell subtypes, optionally obtained by mixing cell subtypes enriched or purified from a biological sample.

50. The method of claim 49, wherein the biological sample comprises a whole blood sample, a buffy coat sample, a peripheral blood mononuclear cell (PBMC) sample, an unfractionated cell sample, a lymphocyte sample, a white blood cell sample, an apheresis product, or a leukapheresis product.

51. The method of any of claims 1-50, wherein the therapeutic cell composition comprises primary cells.

52. The method of any of claims 1-51, wherein the therapeutic cell composition comprises autologous cells from a subject to be treated.

53. The method of any of claims 1-52, wherein the therapeutic cell composition comprises allogeneic cells.

54. The method of any of claims 1-53, wherein the therapeutic cell composition comprises CD3+, CD4+, and/or CD8+ T cells.

55. The method any of claims 1-54, wherein the therapeutic cell composition comprises CD4+ T cells and CD8+ T cells.

56. The method of any of claims 1-55, wherein the recombinant receptor is a chimeric antigen receptor (CAR).

57. The method of any of claims 1-56, wherein the plurality of incubations are performed in a flask, a tube, or a multi-well plate.

58. The method of any of claims 1-57, wherein the each of the plurality of incubations are performed individually in a well of a multi-well plate.

59. The method of claim 57 or claim 58, wherein the multi-well plate is a 96-well plate, 48-well plate, 12-well plate or 6-well plate.

60. The method of any of claims 1 and 4-59, further comprising determining, based on the titrated ratio that results in a half-maximal recombinant receptor-dependent activity, a dose of cells of the therapeutic composition for administering to a subject in need thereof.

61. The method of any of claims 2-59, further comprising determining, based on the relative potency, a dose of cells of the therapeutic composition for administering to a subject in need thereof.

62. The method of claim 60 or claim 61, wherein the subject has a disease or condition.

63. The method of any of claims 15-62, wherein the disease or condition is cancer.

64. The method of any of claims 2-63, further comprising determining, based on the relative potency, a manufacturing process that produces an optimal therapeutic cell composition potency, wherein the optimal therapeutic cell composition potency correlates with complete and/or durable response and/or reduced toxicity.

65. The method of any of claims 2-64, further comprising determining, based on the relative potency, a manufacturing process that produces a therapeutic cell composition with reduced or low variance in potency, wherein the reduced or low variance is determined compared to the variance in a different manufacturing process.


SEQUENCE LISTING (92 sequences)

SEQ ID NO: 1 - protein, 24 aa, Artificial Sequence
  T2A
LEGGGEGRGSLLTCGDVEENPGPR

SEQ ID NO: 2 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 3 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 4 - protein, 18 aa, Artificial Sequence
  T2A
EGRGSLLTCGDVEENPGP

SEQ ID NO: 5 - protein, 22 aa, Artificial Sequence
  P2A
GSGATNFSLLKQAGDVEENPGP

SEQ ID NO: 6 - protein, 19 aa, Artificial Sequence
  P2A
ATNFSLLKQAGDVEENPGP

SEQ ID NO: 7 - protein, 20 aa, Artificial Sequence
  E2A
QCTNYALLKLAGDVESNPGP

SEQ ID NO: 8 - protein, 22 aa, Artificial Sequence
  F2A
VKQTLNFDLLKLAGDVESNPGP

SEQ ID NO: 9 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 10 - protein, 22 aa, Artificial Sequence
  GMCSFR alpha chain signal sequence
MLLLVTSLLLCELPHPAFLLIP

SEQ ID NO: 11 - protein, 18 aa, Artificial Sequence
  CD8 alpha signal peptide
MALPVTALLLPLALLLHA

SEQ ID NO: 12 - protein, 16 aa, Artificial Sequence
  CD33 signal peptide
MPLLLLLPLLWAGALA

SEQ ID NO: 13 - protein, 54 aa, Artificial Sequence
  Extracellular domain of human BCMA (GenBank No. NP_001183.2)
MLQMAGQCSQNEYFDSLLHACIPCQLRCSSNTPPLTCQRYCNASVTNSVKGTNA

SEQ ID NO: 14 - protein, 668 aa, Artificial Sequence
  Human CD22 extracellular domain
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 15 - protein, 556 aa, Homo Sapiens [sequence omitted]

SEQ ID NO: 16 - protein, 232 aa, Artificial Sequence
  Human IgG1 Fc
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 231 aa, Artificial Sequence
  Modified Human IgG1 Fc
PKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 290 aa, Artificial Sequence
  BCMA-Fc fusion polypeptide
MLQMAGQCSQNEYFDSLLHACIPCQLRCSSNTPPLTCQRYCNASVTNSVKGTNAGGGGSPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 377 aa, Artificial Sequence
  Human ROR1 fragment
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILY

SEQ ID NO: 20 - protein, 613 aa, Artificial Sequence
  ROR1-Fc fusion polypeptide
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYGGGGSPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 904 aa, Artificial Sequence
  CD22-Fc fusion polypeptide
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRGGGGSPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 5 aa, Artificial Sequence
  Linker
GGGGS

SEQ ID NO: 23 - protein, 4 aa, Artificial Sequence
  Linker
GGGS

SEQ ID NO: 24 - protein, 15 aa, Artificial Sequence
  Linker
GGGGSGGGGSGGGGS

SEQ ID NO: 25 - protein, 18 aa, Artificial Sequence
  Linker
GSTSGSGKPGSGEGSTKG

SEQ ID NO: 26 - protein, 21 aa, Artificial Sequence
  Linker
SRGGGGSGGGGSGGGGSLEMA

SEQ ID NO: 27 - protein, 5 aa, Artificial Sequence
  CDR H1
DYGVS

SEQ ID NO: 28 - protein, 16 aa, Artificial Sequence
  CDR H2
VIWGSETTYYNSALKS

SEQ ID NO: 29 - protein, 7 aa, Artificial Sequence
  CDR H3
YAMDYWG

SEQ ID NO: 30 - protein, 12 aa, Artificial Sequence
  CDR H3
HYYYGGSYAMDY

SEQ ID NO: 31 - protein, 11 aa, Artificial Sequence
  CDR L1
RASQDISKYLN

SEQ ID NO: 32 - protein, 7 aa, Artificial Sequence
  CDR L2
SRLHSGV

SEQ ID NO: 33 - protein, 7 aa, Artificial Sequence
  CDR L2
HTSRLHS

SEQ ID NO: 34 - protein, 9 aa, Artificial Sequence
  CDR L3
GNTLPYTFG

SEQ ID NO: 35 - protein, 9 aa, Artificial Sequence
  CDR L3
QQGNTLPYT

SEQ ID NO: 36 - protein, 120 aa, Artificial Sequence
  VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 37 - protein, 107 aa, Artificial Sequence
  VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 38 - DNA, 735 bp, Artificial Sequence
  Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 39 - protein, 245 aa, Artificial Sequence
  scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 40 - protein, 5 aa, Artificial Sequence
  CDR H1
SYWMN

SEQ ID NO: 41 - protein, 17 aa, Artificial Sequence
  CDR H2
QIYPGDGDTNYNGKFKG

SEQ ID NO: 42 - protein, 13 aa, Artificial Sequence
  CDR H3
KTISSVVDFYFDY

SEQ ID NO: 43 - protein, 9 aa, Artificial Sequence
  CDR L1
QQYNRYPYT

SEQ ID NO: 44 - protein, 5 aa, Artificial Sequence
  CDR L2
SYWMN

SEQ ID NO: 45 - protein, 17 aa, Artificial Sequence
  CDR L3
QIYPGDGDTNYNGKFKG

SEQ ID NO: 46 - protein, 122 aa, Artificial Sequence
  VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 47 - protein, 108 aa, Artificial Sequence
  VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 48 - protein, 11 aa, Artificial Sequence
  CDR L1
KASQNVGTNVA

SEQ ID NO: 49 - protein, 7 aa, Artificial Sequence
  CDR L2
SATYRNS

SEQ ID NO: 50 - protein, 9 aa, Artificial Sequence
  CDR L3
QQYNRYPYT

SEQ ID NO: 51 - protein, 5 aa, Artificial Sequence
  CDR H1
SYWMN

SEQ ID NO: 52 - protein, 17 aa, Artificial Sequence
  CDR H2
QIYPGDGDTNYNGKFKG

SEQ ID NO: 53 - protein, 13 aa, Artificial Sequence
  CDR H3
KTISSVVDFYFDY

SEQ ID NO: 54 - protein, 245 aa, Artificial Sequence
  scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 55 - protein, 117 aa, Artificial Sequence
  VH
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 56 - protein, 111 aa, Artificial Sequence
  VL
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 57 - protein, 122 aa, Artificial Sequence
  VH
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 58 - protein, 107 aa, Artificial Sequence
  VL
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 59 - protein, 116 aa, Artificial Sequence
  VH
EVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSS

SEQ ID NO: 60 - protein, 111 aa, Artificial Sequence
  VL
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLG

SEQ ID NO: 61 - protein, 118 aa, Artificial Sequence
  VH
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 62 - protein, 105 aa, Artificial Sequence
  VL
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLG

SEQ ID NO: 63 - protein, 122 aa, Artificial Sequence
  VH
EVQLVQSGAEVKKPGSSVKVSCKASGGTFSSYAISWVRQAPGQGLEWMGRIIPILGIANYAQKFQGRVTMTEDTSTDTAYMELSSLRSEDTAVYYCARSGYSKSIVSYMDYWGQGTLVTVSS

SEQ ID NO: 64 - protein, 111 aa, Artificial Sequence
  VL
LPVLTQPPSTSGTPGQRVTVSCSGSSSNIGSNVVFWYQQLPGTAPKLVIYRNNQRPSGVPDRFSVSKSGTSASLAISGLRSEDEADYYCAAWDDSLSGYVFGTGTKVTVLG

SEQ ID NO: 65 - protein, 120 aa, Artificial Sequence
  VH
QVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSS

SEQ ID NO: 66 - protein, 112 aa, Artificial Sequence
  VL
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLG

SEQ ID NO: 67 - protein, 118 aa, Artificial Sequence
  VH
QVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSS

SEQ ID NO: 68 - protein, 112 aa, Artificial Sequence
  VL
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLG

SEQ ID NO: 69 - protein, 12 aa, Artificial Sequence
  spacer (IgG4hinge) (aa)
ESKYGPPCPPCP

SEQ ID NO: 70 - DNA, 36 bp, Artificial Sequence
  spacer (IgG4hinge) (nt)
gaatctaagtacggaccgccctgccccccttgccct

SEQ ID NO: 71 - protein, 119 aa, Homo Sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 72 - protein, 229 aa, Homo Sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 73 - protein, 200 aa, Homo Sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSG

SEQ ID NO: 74 - protein, 14 aa, Artificial Sequence
  Exemplary IgG Hinge
EVVVKYGPPCPPCP

SEQ ID NO: 75 - protein, 5 aa, Artificial Sequence
  Exemplary IgG Hinge
  VARIANT 1: Xaa is glycine, cysteine or arginine
  VARIANT 4: Xaa is cysteine or threonine
XPPXP

SEQ ID NO: 76 - protein, 15 aa, Artificial Sequence
  Exemplary IgG Hinge
EPKSCDKTHTCPPCP

SEQ ID NO: 77 - protein, 12 aa, Artificial Sequence
  Exemplary IgG Hinge
ERKCCVECPPCP

SEQ ID NO: 78 - protein, 61 aa, Artificial Sequence
  Exemplary IgG Hinge
ELKTPLGDTHTCPRCPEPKSCDTPPPCPRCPEPKSCDTPPPCPRCPEPKSCDTPPPCPRCP

SEQ ID NO: 79 - protein, 12 aa, Artificial Sequence
  Exemplary IgG Hinge
ESKYGPPCPSCP

SEQ ID NO: 80 - protein, 12 aa, Artificial Sequence
  Exemplary IgG Hinge
ESKYGPPCPPCP

SEQ ID NO: 81 - protein, 9 aa, Artificial Sequence
  Exemplary IgG Hinge
YGPPCPPCP

SEQ ID NO: 82 - protein, 10 aa, Artificial Sequence
  Exemplary IgG Hinge
KYGPPCPPCP

SEQ ID NO: 83 - protein, 27 aa, Homo Sapiens
  CD28 (amino acids 153-179 of Accession No. P10747)
FWVLVVVGGVLACYSLLVTVAFIIFWV

SEQ ID NO: 84 - protein, 66 aa, Homo Sapiens
  CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 85 - protein, 41 aa, Homo Sapiens
  CD28 (amino acids 180-220 of P10747)
RSKRSRLLHSDYMNMTPRRPGPTRKHYQPYAPPRDFAAYRS

SEQ ID NO: 86 - protein, 41 aa, Homo Sapiens
  CD28 (LL to GG)
RSKRSRGGHSDYMNMTPRRPGPTRKHYQPYAPPRDFAAYRS

SEQ ID NO: 87 - protein, 42 aa, Homo Sapiens
  4-1BB (amino acids 214-255 of Q07011.1)
KRGRKKLLYIFKQPFMRPVQTTQEEDGCSCRFPEEEEGGCEL

SEQ ID NO: 88 - protein, 112 aa, Homo Sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 112 aa, Homo Sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 112 aa, Homo Sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 30 aa, Artificial Sequence
  Linker; P is proline, G is glycine, and S is serine
PGGGSGGGGSGGGGSGGGGSGGGGSGGGGP

SEQ ID NO: 92 - protein, 17 aa, Artificial Sequence
  Linker
GSADDAKKDAAKKDGKS